(19) United States
(12) Patent Application Publication
(10) Pub. No.: US 2023/0257779 A1
(43) Pub. Date: Aug. 17, 2023

(54) GENE EDITING TO IMPROVE JOINT FUNCTION

(71) Applicant: Orthobio Therapeutics, Inc., Edwards, CO (US)

(72) Inventors: Peter J. Millett, Edwards, CO (US); Iain Alasdair Russell, Cambridge (GB); Matthew J. Allen, Cambs (GB); George Gentsch, Cambridge (GB)

(73) Assignee: Orthobio Therapeutics, Inc., Edwards, CO (US)

(21) Appl. No.: 18/005,544

(22) PCT Filed: Jul. 16, 2021
(86) PCT No.: PCT/US2021/042100

§ 371 Date: Jan. 13, 2023

Related U.S. Application Data
(60) Provisional application No. 63/052,881, filed on Jul. 16, 2020.
(60) Provisional application No. 63/055,808, filed on Jul. 23, 2020.

(51) Int. Cl. C12N 15/90; C12N 15/86; C12N 15/11; C12N 9/22

(52) U.S. Cl. CPC C12N 15/907; C12N 15/86; C12N 15/11; C12N 9/22; C12N 2750/14143; C12N 2750/14145; C12N 2750/14171; C12N 2310/20; C12N 2800/80

(57) ABSTRACT

The present disclosure provides compositions and methods for treating joint disorders that are characterized by an inflammatory component. In some aspects, the compositions and methods are to prevent the progression of osteoarthritis and other arthritides and to treat osteoarthritis and other arthritides in a mammalian joint.

### CROSS REFERENCE TO RELATED APPLICATIONS

This application claims priority to U.S. Provisional Patent Application No. 63/052,881, filed Jul. 16, 2020, and U.S. Provisional Patent Application No. 63/055,808, filed Jul. 23, 2020, the contents of which are hereby incorporated by reference, in their entireties, for all purposes.

### FIELD

Compositions and Methods for treating synovial joint dysfunction are described herein. In addition, methods for gene-editing synovial cells and/or synoviocytes, chondrocytes, synovial macrophages, and synovial fibroblasts, and uses of gene-edited synovial cells and/or synoviocytes, chondrocytes, synovial macrophages, and synovial fibroblasts, in the treatment of diseases such as osteoarthritis are disclosed herein.

### BACKGROUND

Treatment of osteoarthritis, degenerative joint disease, and other joint dysfunction is complex and there are few long term options for either symptomatic relief or restoring joint function. Osteoarthritis (OA) is the leading cause of disability due to pain. Neogi, Osteoarthritis Cartilage 2013; 21:1145-53. All mammal species are affected: working animals, domestic pets, and their owners all suffer OA-related discomfort, pain, and disability, depending on the degree of disease progression.

OA is a complex disease characterized by a progressive course of disability. Systemic inflammation is associated with OA and with OA disease progression. Inflammation is driven by increased levels of pro-inflammatory cytokines. New methods and compositions to treat this disease are acutely needed. Disclosed herein are compositions and methods useful for treating OA as well as other inflammatory joint disorders.

### BRIEF SUMMARY

The present disclosure provides compositions and methods for treating joint disorders that are characterized by an inflammatory component. In some aspects, the compositions and methods are to prevent the progression of osteoarthritis and other arthritides and to treat osteoarthritis and other arthritides in a mammalian joint. According to exemplary embodiments, at least a portion of the joint synovial cells and/or synoviocytes, chondrocytes, synovial macrophages, or synovial fibroblasts are gene-edited to reduce the expression of inflammatory cytokines. In some aspects, at least a portion of the joint synovial cells and/or synoviocytes, chondrocytes, synovial macrophages, or synovial fibroblasts, are gene-edited to reduce the expression of IL-1α, IL-1β, or both IL-1α, IL-1β.

In some embodiments, the gene-editing causes expression of one or more cytokine and/or growth factor genes to be silenced or reduced in at least a portion of the cells comprising a mammalian joint. In some aspects, the cells are synovial cells. In some aspects, the cells are synovial fibroblasts. In some aspects, the cells are synoviocytes. In some aspects, the cells are chondrocytes. In some aspects, the cells are synovial macrophages.

In some embodiments, the one or more cytokine and/or growth factor genes is/are selected from the group comprising IL-1α, and IL-1β.

In some embodiments, the gene-editing comprises the use of a programmable nuclease that mediates the generation of a double-strand or single-strand break at said one or more cytokine and/or growth factor genes.

In some embodiments, the gene-editing comprises one or more methods selected from a CRISPR method, a TALE method, a zinc finger method, and a combination thereof.

In some embodiments, the gene-editing comprises a CRISPR method.

In some embodiments, the CRISPR method is a CRISPR-Cas9 method.

In some embodiments, the gene-editing comprises a TALE method.

In some embodiments, the gene-editing comprises a zinc finger method.

In some embodiments, the gene-editing causes expression of one or more cytokine and/or growth factor genes to be silenced or reduced in at least a portion of the cells comprising the joint. In some embodiments, the portion of cells edited are synoviocytes. In an aspect, the portion of cells edited are synovial fibroblasts. In some embodiments, the portion of cells edited are synoviocytes. In some embodiments, the portion of cells edited are chondrocytes. In some embodiments, the portion of cells edited are synovial macrophages.

In some embodiments, an adeno-associated virus (AAV) delivery system is used to deliver the gene-editing system. In some embodiments, the AAV delivery system is injected into a joint.

Some aspects of the present disclosure provide a pharmaceutical composition for the treatment or prevention of a joint disease or condition comprising a gene-editing system and a pharmaceutically acceptable carrier. In an aspect, the gene-editing system comprises one or more nucleic acids targeting one or more genetic locus selected from the group consisting of IL-1α, IL-1β, TNF-α, IL-6, IL-8, and IL-18.

An embodiment provides a method of treating canine lameness, the method comprising administering a gene-editing composition, wherein the composition causes expression of IL-1α and IL-1β to be silenced or reduced in a portion of a lame joint's synoviocytes, chondrocytes, synovial macrophages, or synovial fibroblasts.

In some embodiments, the above method further comprises one or more features recited in any of the methods and compositions described herein.

### DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1A illustrates an agarose gel electrophoresis analysis of 100 ng mouse DNA (gBlocks, Integrated DNA Technologies) designed against the Mus musculus Il1a and Il1b genes, cleaved by 0.5 μg SpyCas9 (TrueCut™ Cas9 protein v2, ThermoFisher Scientific) and 200 ng Phosphorothioate-modified single guide (sg)RNAs targeted against the Il1a gene (#43-46) and Il1b gene (#47-50) in vitro.

FIG. 1B illustrates an agarose gel electrophoresis analysis of 100 ng mouse DNA (gBlocks, Integrated DNA Technologies) designed against the Mus musculus Il1a and Il1b genes, cleaved by 0.5 μg SauCas9 (GeneSnipper™ Cas9, BioVision) and 200 ng Phosphorothioate-modified guide sgRNAs against the Il1a (#51-53) and IL1b (#54-56) genes.

FIGS. 2A, 2B, 2C, and 2D collectively illustrate graphs displaying editing efficiencies of SpyCas9 and SauCas9 used with a range of guide RNA's in J774.2 (“J”) and NIH3T3 (“N”) cells; FIG. 2A: in vivo cleavage of Il1a, edited with 4×sgRNAs (Spy Cas9) in two separate pools (Pool 1 and 2), across two cell lines, NIH 3T3 (“N”), and J774.2 (“J”); FIG. 2B: in vivo cleavage of Il1b, edited with 4×sgRNAs (Spy Cas9) in two separate pools (Pool 1 and 2), across two cell lines, NIH 3T3 (“N”), and J774.2 (“J”); FIG. 2C: in vivo cleavage of Il1a, edited with 3×sgRNAs (Sau Cas9) in two separate pools (Pool 1 and 2), across two cell lines, NIH 3T3 (“N”), and J774.2 (“J”); FIG. 2D: in vivo cleavage of Il1b, edited with 3×sgRNAs (saCas9) in two separate pools (Pool 1 and 2), across two cell lines, NIH 3T3 (“N”), and J774.2 (“J”); editing efficiencies determined using deconvolution of Sanger sequencing traces (ICE tool, Synthego) of each pool.

FIG. 3 illustrates GFP expression measured using the IVIS system. Flux values were based on a region of interest centred on the animal's injected knee joint. Data are presented as mean (SD) for four specimens per group.

FIG. 4 illustrates the design of a study as described in Example 5 of the present disclosure.

FIG. 5 illustrates the in-life outcome measurements obtained in a study as described in Example 5 of the present disclosure.

FIG. 6 illustrates the change in body weight of mice treated with an intra-articular (IA) injection of PBS, AAV-6 with a scambled vector, AAV-6 with CRISPR-Cas guides 1 and 2, AAV-5 with a scambled vector, or AAV-5 with CRISPR-Cas guides 1 and 2 in a study as described in Example 5 of the present disclosure.

FIGS. 7A and 7B collectively illustrate (A) change in knee caliper measurements from baseline of mouse joints over time, and (B) mean difference in ankle caliper measurements with AUC in mice treated with an intra-articular (IA) injection of PBS, AAV-6 with a scambled vector, AAV-6 with CRISPR-Cas guides 1 and 2, AAV-5 with a scambled vector, or AAV-5 with CRISPR-Cas guides 1 and 2 in a study as described in Example 5 of the present disclosure.

FIGS. 8A and 8B collectively illustrate (A) change in von Frey measurements, and (B) mean absolute threshold in von Frey measurements obtained from mice treated with an intra-articular (IA) injection of PBS, AAV-6 with a scambled vector, AAV-6 with CRISPR-Cas guides 1 and 2, AAV-5 with a scambled vector, or AAV-5 with CRISPR-Cas guides 1 and 2 in a study as described in Example 5 of the present disclosure.

FIG. 9 illustrate results of a qPCR assay for IL-1β expression in synovial fluid obtained from mice treated with an intra-articular (IA) injection of PBS, AAV-6 with a scambled vector, AAV-6 with CRISPR-Cas guides 1 and 2, AAV-5 with a scambled vector, or AAV-5 with CRISPR-Cas guides 1 and 2 in a study as described in Example 5 of the present disclosure.

FIGS. 10A, 10B, 10C, and 10D collectively illustrate immunohistochemistry for murine IL-1β in synovial tissue of MSU injected animals (A, B) pre-treated with PBS, and (C, D) treated with CRISPR. FIGS. 10B and 10D show isotype controls for each of FIGS. 10A and 10C, respectively.

FIGS. 11A, 11B, and 11C collectively illustrate an alignment between the mouse, human, equine, feline, and canine IL-1 alpha genes.

FIGS. 12A, 12B, 12C, and 12D collectively illustrate an alignment between the mouse, human, equine, feline, and canine IL-1 beta genes.

FIGS. 13A, 13B, 13C, and 13D collectively illustrate example CRISPR/Cas9 crRNA sequences designed for editing the human IL-1 alpha gene.

FIGS. 14A, 14B, 14C, 14D, and 14E collectively illustrate example CRISPR/Cas9 crRNA sequences designed for editing the human IL-1 beta gene.

FIGS. 15A, 15B, and 15C collectively illustrate example CRISPR/Cas9 crRNA sequences designed for editing the canine IL-1 alpha gene.

FIGS. 16A and 16B collectively illustrate example CRISPR/Cas9 crRNA sequences designed for editing the canine IL-1 beta gene.

FIGS. 17A, 17B, 17C, and 17D collectively illustrate the results of cell-based and in-silico gene editing analysis of crRNA sequences targeting the human IL-1 alpha gene (FIG. 7A), human IL-1 beta gene (FIG. 7B), canine IL-1 alpha gene (FIG. 7C), and canine IL-1 beta gene (FIG. 7D), as described in Example 8. ° CRISPR cut position within the translation frame of amino acids (AA). * Optimized score from Doench, Fusi et al. (2016). This score is optimized for 20 bp guides with NGG. Score spans from 0 to 100. Higher is better. ** Specificity score from Hsu et al. (2013). Score spans from 0 to 100. Higher is better. *** This score is based on experiments in U205. A high precision score (>0.4) implies that DNA repair outcomes are uniform and enriched for just a handful of unique genotypes. **** This score is based on experiments in U205. A high (>80%) frameshift frequency will tend to knock a protein-coding gene out of frame. The typical genomic frameshift frequency is above 66% because 1-bp insertions and 1-2 bp deletions are particularly common repair outcomes. \hat{ } Combined score=(Off-target score+Precision score*100+Frameshift)/3. † Pipe symbol ‘|’ indicates CRISPR cut site. Curly braces ‘{ }’ indicate insertion. Hyphen ‘-’ indicates deletion. $ Potential off-target sites. Scoring according to Hsu et al. (2013). The on-target site has a score of 100.

FIGS. 18A, 18B, 18C, and 18D collectively illustrate canine IL-1 alpha (FIGS. 18A and 18B) and canine IL-1 beta (FIGS. 18C and 18D) release from non-edited (control) and double IL-1α/IL-1β KO (edited) canine chondrocytes 6 hours (FIGS. 18A and 18C) and 24 hours (FIGS. 18B and 18D) after exposure to PBS or LPS, as described in Example 9.

FIGS. 19A, 19B, 19C, and 19D collectively illustrate human IL-1 alpha (FIGS. 19A and 19B) and canine IL-1 beta (FIGS. 19C and 19D) release from non-edited (control) and double IL-1α/IL-1β KO (edited) canine chondrocytes 6 hours (FIGS. 19A and 19C) and 24 hours (FIGS. 19B and 19D) after exposure to PBS or LPS, as described in Example 9.

While the above-identified drawing sets forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

As described herein, embodiments of the present disclosure provide compositions and methods for improving joint function and treating joint disease. In particular embodiments, compositions and methods are provided to gene-edit synovial fibroblasts, synoviocytes, chondrocytes, or synovial macrophages to reduce expression of inflammatory cytokines, for example, IL-1α, IL-1β, TNF-α, IL-6, IL-8, IL-18, one or more matrix metalloproteinases (MMPs), or one or more component of the NLRP3 inflammasome. Embodiments are used for treating osteoarthritis and other inflammatory joint diseases. Embodiments are further useful for treating canine lameness due to osteoarthritis. Embodiments are further useful for treating equine lameness due to joint disease. Embodiments are also useful for treating post-traumatic arthritis, gout, pseudogout, and other inflammation-mediated or immune-mediated joint diseases.

### Definitions

Unless defined otherwise, all technical and scientific terms used herein have the same meaning as is commonly understood by one of skill in the art to which this disclosure belongs. All patents and publications referred to herein are incorporated by reference in their entireties.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “IL-1” (also referred to herein as “IL1”) refers to the pro-inflammatory cytokine known as interleukin-1, and includes all forms of IL-1, including IL1-α and IL-1β, human and mammalian forms, conservative amino acid substitutions, glycoforms, biosimilars, and variants thereof. IL-1α and IL-1β bind to the same receptor molecule, which is called type I IL-1 receptor (IL-1RI). There is a third ligand of this receptor: Interleukin 1 receptor antagonist (IL-1Ra), which does not activate downstream signaling; therefore, acting as an inhibitor of IL-1α and IL-1β signaling by competing with them for binding sites of the receptor. See, e.g., Dinarello, Blood 117: 3720-32 (2011) and Weber et al., Science Signaling 3(105): cm1, doi:10.1126/scisignal.3105cm1. IL-1 is described, e.g., in Dinarello, Cytokine Growth Factor Rev. 8:253-65 (1997), the disclosures of which are incorporated by reference herein. For example, the term IL-1 encompasses human, recombinant forms of IL-1.

TABLE 1
Amino acid sequences of interleukins.

| Identifier | Sequence (One-Letter Amino Acid Symbols) |
|---|---|
| SEQ ID NO: 1 | 10 20 30 40 50 |
| recombinant | MAKVPDMFED LKNCYSENEE DSSSIDHLSL NQKSFYHVSY GPLHEGCMDQ |
| human IL-1alpha | 60 70 80 90 100 |
| (rhIL-1α) | SVSLSISETS KTSKLTFKES MVVVATNGKV LKKRRLSLSQ SITDDDLEAI |
| | 110 120 130 140 150 |
| | ANDSEEEIIK PRSAPFSFLS NVKYNFMRII KYEFILNDAL NQSIIRANDQ |
| | 160 170 180 190 200 |
| | YLTAAALHNL DEAVKFDMGA YKSSKDDAKI TVILRISKTQ LYVTAQDEDQ |
| | 210 220 230 240 250 |
| | PVLLKEMPEI PKTITGSETN LLFFWETHGT KNYFTSVAHP NLFIATKQDY |
| | 260 270 |
| | WVCLAGGPPS ITDFQILENQ A |
| SEQ ID NO: 2 | 10 20 30 40 50 |
| recombinant | MAEVPELASE MMAYYSGNED DLFFEADGPK QMKCSFQDLD LCPLDGGIQL |
| human IL-1beta | 60 70 80 90 100 |
| (rhIL-1ß) | RISDHHYSKG FRQAASVVVA MDKLRKMLVP CPQTFQENDL STFFPFIFEE |
| | 110 120 130 140 150 |
| | EPIFFDTWDN EAYVHDAPVR SLNCTLRDSQ QKSLVMSGPY ELKALHLQGQ |
| | 160 170 180 190 200 |
| | DMEQQVVFSM SFVQGEESND KIPVALGLKE KNLYLSCVLK DDKPTLQLES |
| | 210 220 230 240 250 |
| | VDPKNYPKKK MEKRFVFNKI EINNKLEFES AQFPNWYIST SQAENMPVFL |
| | 260 |
| | GGTKGGQDIT DFTMQFVSS |
| SEQ ID NO: 3 | 10 20 30 40 50 |
| recombinant | MAKVPDLFED LKNCYSENED YSSAIDHLSL NQKSFYDASY GSLHETCTDQ |
| mouse IL-1alpha | 60 70 80 90 100 |
| (rmIL-1α) | FVSLRTSETS KMSNFTFKES RVTVSATSSN GKILKKRRLS FSETFTEDDL |
| | 110 120 130 140 150 |
| | QSITHDLEET IQPRSAPYTY QSDLRYKLMK LVRQKFVMND SLNQTIYQDV |
| | 160 170 180 190 200 |
| | DKHYLSTTWL NDLQQEVKFD MYAYSSGGDD SKYPVTLKIS DSQLFVSAQG |
| | 210 220 230 240 250 |
| | EDQPVLLKEL PETPKLITGS ETDLIFFWKS INSKNYFTSA AYPELFIATK |
| | 260 270 |
| | EQSRVHLARG LPSMTDFQIS |
| SEQ ID NO: 4 | 10 20 30 40 50 |
| recombinant | MATVPELNCE MPPFDSDEND LFFEVDGPQK MKGCFQTFDL GCPDESIQLQ |
| mouse IL-1beta | 60 70 80 90 100 |
| (rmIL-1ß) | ISQQHINKSF RQAVSLIVAV EKLWQLPVSF PWTFQDEDMS TFFSFIFEEE |
| | 110 120 130 140 150 |
| | PILCDSWDDD DNLLVCDVPI RQLHYRLRDE QQKSLVLSDP YELKALHLNG |
| | 160 170 180 190 200 |
| | QNINQQVIFS MSFVQGEPSN DKIPVALGLK GKNLYLSCVM KDGTPTLQLE |
| | 210 220 230 240 250 |
| | SVDPKQYPKK KMEKRFVFNK IEVKSKVEFE SAEFPNWYIS TSQAEHKPVF |
| | 260 |
| | LGNNSGQDII DFTMESVSS |
| SEQ ID NO: 5 | 10 20 30 40 50 |
| recombinant | MEICRGLRSH LITLLLFLFH SETICRPSGR KSSKMQAFRI WDVNQKTFYL |
| human IL-1 | 60 70 80 90 100 |
| receptor | RNNQLVAGYL QGPNVNLEEK IDVVPIEPHA LFLGIHGGKM CLSCVKSGDE |
| antagonist | 110 120 130 140 150 |
| (rhIL-1Ra) | TRLQLEAVNI TDLSENRKQD KRFAFIRSDS GPTTSFESAA CPGWFLCTAM |
| | 160 170 |
| | EADQPVSLTN MPDEGVMVTK FYFQEDE |
| SEQ ID NO: 6 | 10 20 30 40 50 |
| recombinant | MEICWGPYSH LISLLLILLF HSEAACRPSG KRPCKMQAFR IWDTNQKTFY |
| mouse IL-1 | 60 70 80 90 100 |
| receptor | LRNNQLIAGY LQGPNIKLEE KIDMVPIDLH SVELGIHGGK LCLSCAKSGD |
| antagonist | 110 120 130 140 150 |
| (rmIL-1Ra) | DIKLQLEEVN ITDLSKNKEE DKRFTFIRSE KGPTTSFESA ACPGWFLCTT |
| | 160 170 |
| | LEADRPVSLT NTPEEPLIVT KFYFQEDQ |

The term “NLRP3 inflammasome” refers to the multiprotein complex responsible for the activation of some inflammatory responses. The NMRP3 inflammasome promotes the production of functional pro-inflammatory cytokines, for example, IL-1β and IL-18. Core components of the NLRP3 inflammasome are NLRP3, ASC (apoptosis-associated speck-like protein containing a CARD), and caspase-1, as described by Lee et al., Lipids Health Dis. 16:271 (2017) and Groslambert and Py, J. Inflamm. Res. 11:359-374 (2018).

The terms “matrix metalloproteinase” and “MMP” are defined to be any one of the members of the matrix metalloproteinase family of zinc-endopeptidaes, for example, as characterized by Fanjul-Femandez et al., Biochem. Biophys. Acta 1803:3-19 (2010). In the art, family members are frequently referred to as archetypical MMPs, gelatinases, matrilysins, and/or furin-activatable MMPs. As used herein, the “matrix metalloproteinase” and “MMP” encompass the entire MMP family, including, but not limited to MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-25, MMP-26, MMP-27 and MMP-28.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present disclosure, for example, at least one anti-inflammatory compound in combination with a viral vector functionally engineered to deliver a gene-editing nucleic acid as described herein) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “effective amount” or “therapeutically effective amount” refers to that amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compositions chosen, the dosing regimen to be followed, whether the composition is administered in combination with other compositions or compounds, timing of administration, the tissue to which it is administered, and the physical delivery system in which the composition is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The term “homologous” in terms of a nucleotide sequence includes a nucleotide (nucleic acid) sequence that is either identical or substantially similar to a known reference sequence. In one embodiment, the term “homologous nucleotide sequence” is used to characterize a sequence having nucleic acid sequence that is at least 70%, at least 75%, at least 80%, at least 85%, at least 90%, at least 95%, at least 96%, at least 97%, at least 98%, at least 99%, or 100% identical to a known reference sequence.

“Heterologous” means derived from a genotypically distinct entity from the rest of the entity to which it is being compared to. For example, a polynucleotide introduced by genetic engineering techniques into a plasmid or vector derived from a different species is a heterologous polynucleotide. A promoter removed from its native coding sequence and operatively linked to a coding sequence it is not naturally found linked to a heterologous promoter. Although the term “heterologous” is not always used herein in reference to polynucleotides, reference to a polynucleotide even in the absence of the modifier “heterologous” is intended to include heterologous polynucleotides in spite of the omission.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site.

Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. ClustalW and ClustalX may be used to produce alignments, Larkin et al., Bioinformatics 23:2947-2948 (2007); Goujon et al., Nucleic Acids Research, 38 Suppl:W 695-9 (2010); and, McWilliam et al., Nucleic Acids Research 41 (Web Server issue):W 597-600 (2013). One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

“Joint disease” is defined as measurable abnormalities in the cells or tissues of the joint that could lead to illness, for example, metabolic and molecular derangements triggering anatomical and/or physiological changes in the joint. Including, but not limited to, radiographic detection of joint space narrowing, subchondral sclerosis, subchondral cysts, and osteophyte formation.

“Joint illness” is defined in human subjects as symptoms that drive the subject to seek medical intervention, for example, subject reported pain, stiffness, swelling, or immobility. For non-human mammals, “joint illness” is defined, for example, as lameness, observable changes in gait, weight bearing, allodynia, or exploratory behavior.

As used herein, a sgRNA (single guide RNA) is a RNA, preferably a synthetic RNA, composed of a targeting sequence and scaffold. It is used to guide Cas9 to a specific genomic locus in genome engineering experiments. The sgRNA can be administered or formulated, e.g., as a synthetic RNA, or as a nucleic acid comprising a sequence encoding the gRNA, which is then expressed in the target cells. As would be evident to one of ordinary skill in the art, various tools may be used to design and/or optimize the sequence of a sgRNA, for example to increase the specificity and/or precision of genomic editing. In general, candidate sgRNAs may be designed by identifying a sequence within the target region that has a high predicted on-target efficiency and low off-target efficiency based on any of the available web-based tools. Candidate sgRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop. See, e.g., Safari, et al. Current Pharma. Biotechol. (2017) 18(13), which is incorporated by reference herein in its entirety for all purposes. Such tools are also described, for example, in PCT Publication No. WO2014093701A1 and Liu, et al., “Computational approached for effective CRISPR guide RNA design and evaluation”, Comput Struct Biotechnol J., 2020; 18: 35-44, each of which is incorporated by reference herein in its entirety for all purposes.

As used herein, “Cas9” refers to CRISPR Associated Protein; the Cas9 nuclease is the active enzyme for the Type II CRISPR system. “nCas9” refers to a Cas9 that has one of the two nuclease domains inactivated, i.e., either the RuvC or HNH domain. nCas9 is capable of cleaving only one strand of target DNA (a “nickase”). The term “Cas9” refers to an RNA-guided double-stranded DNA-binding nuclease protein or nickase protein, or a variant thereof. Herein, “Cas9” refers to both naturally-occurring and recombinant Cas9s. Wild-type Cas9 nuclease has two functional domains, e.g., RuvC and HNH, that cut different DNA strands. Cas9 enzymes described herein can comprise a HNH or HNH-like nuclease domain and/or a RuvC or RuvC-like nuclease domain. Cas9 can induce double-strand breaks in genomic DNA (target locus) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for Cas9 to bind target DNA, and immediately follows the target sequence. The Cas9 can be administered or formulated, e.g., as a protein (e.g., a recombinant protein), or as a nucleic acid comprising a sequence encoding the Cas9 protein, which is then expressed in the target cells. Naturally occurring Cas9 molecules recognize specific PAM sequences (e.g., the PAM recognition sequences for S. pyogenes, S. thermophilus, S. mutans, S. aureus and N. meningitidis). In an embodiment, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

An “expression cassette” is a nucleic acid construct, generated recombinantly or synthetically, with a series of specified nucleic acid elements that permit transcription of a particular polynucleotide sequence in a host cell. An expression cassette or vector may be part of a plasmid, viral genome, or nucleic acid fragment. Typically, an expression cassette or vector includes a polynucleotide to be transcribed, operably linked to a promoter.

The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

The term “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

An “AAV vector” as used herein refers to an AAV vector nucleic acid sequence encoding for various nucleic acid sequences, including in some embodiments a variant or chimeric capsid polypeptide (i.e., the AAV vector comprises a nucleic acid sequence encoding for a variant or chimeric capsid polypeptide). AAV vectors can also comprise a heterologous nucleic acid sequence not of AAV origin as part of the nucleic acid insert. This heterologous nucleic acid sequence typically comprises a sequence of interest for the genetic transformation of a cell. In general, the heterologous nucleic acid sequence is flanked by at least one, and generally by two AAV inverted terminal repeat sequences (ITRs). In certain embodiments, a Cas sequence, a guide RNA sequence, and any other genetic element (e.g., a promoter sequence, a PAM sequence, and the like) may be on the same AAV vector or on two or more different AAV vectors when administered to a subject. In certain embodiments, a Cas sequence, a guide RNA sequence, and any other genetic element (e.g., a promoter sequence, a PAM sequence, and the like) may be on two or more different AAV vectors when administered to a subject, and the AAV may be the same serotype, or the AAV may be two or more different serotypes (e.g., AAV5 and AAV6).

An “AAV virion” or “AAV virus” or “AAV viral particle” or “AAV vector particle” refers to a viral particle composed of at least one AAV capsid polypeptide and an encapsidated polynucleotide AAV transfer vector. If the particle comprises a heterologous nucleic acid (i.e. a polynucleotide other than a wild-type AAV genome, such as a transgene to be delivered to a cell), it can be referred to as an “AAV vector particle” or simply an “AAV vector”. Thus, production of AAV virion or AAV particle necessarily includes production of AAV vector as such a vector is contained within an AAV virion or AAV particle.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of” and “consisting of.”

A subject treated by any of the methods or compositions described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., without limitation, between 2 and 20 years old, between 20 and 40 years old, or between 40 and 90 years old). The subject can be a human or non-human subject. A particular class of subjects that can benefit from the compositions and methods of the present disclosure include subjects over the age of 40, 50, or 60 years. Another class of subjects that can benefit from the compositions and methods of the present disclosure are subjects that have arthritis (e.g., osteoarthritis).

Any of the compositions disclosed herein can be administered to a non-human subject, such as a laboratory or farm animal. Non-limiting examples of a non-human subject include laboratory or research animals, pets, wild or domestic animals, farm animals, etc., e.g., a dog, a goat, a guinea pig, a hamster, a mouse, a pig, a non-human primate (e.g., a gorilla, an ape, an orangutan, a lemur, a baboon, etc.), a rat, a sheep, a horse, a cow, or the like.

The present disclosure provides compositions useful for treating joint disorders with an inflammatory component. In some aspects, the compositions are useful to prevent the progression of osteoarthritis and to treat osteoarthritis in a mammalian joint.

In some aspects, the pharmaceutical composition comprises a gene-editing system, wherein the gene-editing system causes expression the at least one genetic locus related to joint function to be silenced or reduced in at least a portion of the cells comprising the joint.

In an aspect, the pharmaceutical composition comprises a gene-editing system, wherein the gene-editing system targets one or more of IL-1α, and IL-1β. In some aspects, the pharmaceutical composition comprises a gene-editing system, wherein the gene-editing system targets one or more of TNF-α, IL-6, IL-8, IL-18, a matrix metalloproteinase (MMP), or components of the NLRP3 inflammasome.

In some aspects, the pharmaceutical composition comprises a gene-editing system, wherein the gene-editing comprises the use of a programmable nuclease that mediates the generation of a double-strand or single-strand break at the at least one locus related to joint function. In some embodiments, the gene-editing system reduces the gene expression of the targeted locus or targeted loci. In some embodiments, the at least one locus related to joint tissue is silenced or reduced in at least a portion of the cells comprising the joint.

In some aspects, the cells comprising the joint are synoviocytes. In some aspects, the cells are synovial macrophages. In some aspects, the cells are synovial fibroblasts. In some aspects at least a portion of the synoviocytes are edited. In some aspects, the cells comprising the joint are chondrocytes.

In an aspect, the pharmaceutical composition targets the one or more cytokine and/or growth factor genes is/are selected from the group comprising IL-1α, IL-1β, TNF-α, IL-6, IL-8, IL-18, a matrix metalloproteinase (MMP), or a component of the NLRP3 inflammasome. In some embodiments, the component of the NLRP3 inflammasome comprises NLRP3, ASC (apoptosis-associated speck-like protein containing a CARD), caspase-1, and combinations thereof.

Pharmaceutical compositions are also provided, wherein the gene-editing causes expression of one or more cytokine and/or growth factor genes to be enhanced in at least a portion of the cells comprising the joint, the cytokine and/or growth factor gene(s) being selected from the group comprising IL-1Ra, TIMP-1, TIMP-2, TIMP-3, TIMP-4, and combinations thereof.

In some embodiments, the pharmaceutical composition provides for gene-editing, wherein the gene-editing comprises the use of a programmable nuclease that mediates the generation of a double-strand or single-strand break at said one or more cytokine and/or growth factor genes. In some embodiments, the gene-editing comprises one or more methods selected from a CRISPR method, a TALE method, a zinc finger method, and a combination thereof.

In an aspect, the gene-editing comprises a CRISPR method. In yet other aspects, the CRISPR method is a CRISPR-Cas9 method. In some aspects, the Cas9 is mutated to enhance function.

### Animal Models of Osteoarthritis

Several animal models for osteoarthritis are known to the art. Exemplary nonlimiting animal models are summarized; however, it is understood that various models may be used. Many different species of animals are used to mimic OA, for example, studies have been conducted on mice, rats, rabbits, guinea pigs, dogs, pigs, horses, and even other animals. See, e.g., Kuyinu et al., J Orthop Surg Res. 11:19 (2016) (hereinafter “Kuyinu, 2016”).

It is understood that the various methods for inducing OA may be used in any mammal. In the mouse, spontaneous, chemically induced, surgically induced, and non-invasive induction are commonly used. E.g., Kuyinu, 2016; Bapat et al., Clin Transl Med. 7:36 (2018) (hereinafter “Bapat, 2018”); and Poulet, Curr Rheumatol Rep 18:40 (2016). In the horse, osteochondral fragment-exercise model, chemical induction, traumatic induction, and induction through overuse are commonly used. In sheep, surgical induction is most common; in the guinea pig, surgical induction, chemical induction, and spontaneous (Durkin Hartley) methods are frequently used. E.g. Bapat, 2018.

The destabilized medial meniscus (DMM) is frequently used in mice to model posttraumatic osteoarthritis, e.g. Culley et al., Methods Mol Biol.1226:143-73 (2015). The DMM model mimics clinical meniscal injury, a known predisposing factor for the development of human OA, and permits the study of structural and biological changes over the course of the disease. Mice are an attractive model organism, because mouse strains with defined genetic backgrounds may be used. Additionally, knock-out or other genetically manipulated mouse strains may be used to evaluate the importance of various molecular pathways in the response to various OA treatment modalities and regimens. For example, STR/ort mice have features that make the strain particularly susceptible to developing OA, including, increased levels of the inflammatory cytokine IL1β, Bapat et al., Clin Transl Med. 7:36 (2018). These mice commonly develop OA in knee, ankle, elbow, and temporo-mandibular joints, Jaeger et al., Osteoarthritis Cartilage 16:607-614 (2008). Other useful mutant strains of mice are known to the skilled artisan, for example, Col9a1(−/−) mice, Allen et al., Arthritis Rheum, 60:2684-2693 (2009).

Another commonly used surgical model for OA is anterior cruciate ligament transection (ACLT) model. Little and Hunter, Nat Rev Rheumatol., 9(8):485-497 (2013). The subject's ACL is surgically transected causing joint destabilization. The anterior drawer test with the joint flexed is used to confirm that transection of the ligament has occurred. In some cases, other ligaments such as the posterior cruciate ligament, medial collateral ligament, lateral collateral ligament, and/or either meniscus may be transected. As with the DMM model, a variety of mouse strains may be used to investigate various molecular pathways.

Depending on the technical objective, animals of varying size may be selected for use. Rodents are useful because of the short time needed for skeletal maturity and consequently shorter time to develop OA following surgical or other technique to induce OA. Larger animals are particularly useful to evaluate therapeutic interventions. The anatomy in larger animals is very similar to humans; for example, in dogs the cartilage thickness is less than about half the thickness of humans; this striking similarity is exemplary of why such cartilage degeneration and osteochondral defects studies are much more useful in large animal models. E.g. McCoy, Vet. Pathol., 52:803-18 (2015); and, Pelletier et al., Therapy, 7:621-34(2010).

### Gene-Editing Processes

### Overview: Compositions to Gene-Edit Synovial Cells

Embodiments of the present disclosure are directed to methods for gene-editing synovial cells (synoviocytes), the methods comprising one or more steps of gene-editing at least a portion of the synoviocytes in a joint to treat osteoarthritis or other joint disorder. As used herein, “gene-editing,” “gene editing,” and “genome editing” refer to a type of genetic modification in which DNA is permanently modified in the genome of a cell, e.g., DNA is inserted, deleted, modified or replaced within the cell's genome. In some embodiments, gene-editing causes the expression of a DNA sequence to be silenced (sometimes referred to as a gene knockout) or inhibited/reduced (sometimes referred to as a gene knockdown). In other embodiments, gene-editing causes the expression of a DNA sequence to be enhanced (e.g., by causing over-expression). In accordance with embodiments of the present disclosure, gene-editing technology is used to reduce the expression or silence pro-inflammatory genes and/or to enhance the expression of regenerative genes.

### Interleukins

According to additional embodiments, gene-editing methods of the present disclosure may be used to increase the expression of certain interleukins, such as one or more of IL-1α, IL-1β, IL-4, IL-6, IL-8, IL-9, IL-10, IL-13, IL-18, and TNF-α. Certain interleukins have been demonstrated to augment inflammatory responses in joint tissue and are linked to disease progression.

### Expression Constructs

Expression constructs encoding one or both of guide RNAs and/or Cas9 editing enzymes can be administered in any effective carrier, e.g., any formulation or composition capable of effectively delivering the component gene to cells in vivo. Approaches include, for example, electroporation and/or insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells. In some instances, the transferred nucleic acids are stably integrated into the chromosomal DNA of the host. In other instances, particularly for adeno-associated virus vectors, stable integration into the host DNA may be a rare event, resulting into episomal expression of the transgene and transient expression of the transgene.

The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see, e.g., Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573, each of which is incorporated by reference herein in its entirety for all purposes).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors may be derived from any strain of adenovirus (e.g., Ad2, Ad3, Ad5, or Ad7 etc.), including Adenovirus serotypes from other species (e.g., mouse, dog, human, etc.) that are known to those skilled in the art. The virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Helper-dependent (HDAd) vectors can also be produced with all adenoviral sequences deleted except the origin of DNA replication at each end of the viral DNA along with packaging signal at 5-prime end of the genome downstream of the left packaging signal. HDAd vectors are constructed and propagated in the presence of a replication-competent helper adenovirus that provides the required early and late proteins necessary for replication.

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

In some embodiments, nucleic acids encoding a CRISPR IL-1α or IL-1β gene editing complex (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In clinical settings, the gene delivery systems for the nucleic acids encoding a CRISPR IL-1α or IL-1β gene editing complex can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the nucleic acids encoding a CRISPR IL-1α or IL-1β gene editing complex is more limited, with introduction into the subject being quite localized. For example, the nucleic acids encoding a CRISPR IL-1α or IL-1β gene editing complex can be introduced by intra-articular injection into a joint exhibiting joint disease (e.g., osteoarthritis). In some embodiments, the nucleic acids encoding a CRISPR IL-1α or IL-1β gene editing complex are administered during or after surgery; in some embodiments, a controlled-release hydrogel comprising the nucleic acids encoding a CRISPR IL-1α or IL-1β gene editing complex is administered at the conclusion of surgery before closure to prevent reduce or eliminate osteoarthritis by providing a steady dose of the nucleic acids encoding a CRISPR IL-1α or IL-1β gene editing complex over time.

A pharmaceutical preparation of the nucleic acids encoding a CRISPR IL-1α or IL-1β gene editing complex can consist essentially of the gene delivery system (e.g., viral vector(s)) in an acceptable diluent, or can comprise a slow release matrix in which the gene delivery vehicle is embedded. Alternatively, where the complete gene delivery system can be produced intact from recombinant cells, e.g., adeno-associated viral vectors, the pharmaceutical preparation can comprise one or more cells, which produce the gene delivery system.

Preferably, the CRISPR IL-1α or IL-1β editing complex is specific, i.e., induces genomic alterations preferentially at the target site (IL-1α or IL-1β), and does not induce alterations at other sites, or only rarely induces alterations at other sites. In certain embodiments, the CRISPR IL-1α or IL-1β editing complex has an editing efficiency of at least 50%, at least 60%, at least 70%, at least 80%, at least 90%, at least 95%, or at least 99%.

The sgRNAs for use in the CRISPR/Cas system for HR typically include a guide sequence (e.g., crRNA) that is complementary to a target nucleic acid sequence (target gene locus) and a scaffold sequence (e.g., tracrRNA) that interacts with a Cas nuclease (e.g., Cas9 polypeptide) or a variant or fragment thereof. A single guide RNA (sgRNA) can include a crRNA and a tracrRNA.

Exemplary target sequences for inducing genomic alterations in the IL-1α or IL-1β gene by the CRISPR-Cas editing complex are provided in Tables 2 and 12. Exemplary guide RNAs for use with the compositions, methods, and systems of the present disclosure are provided in Tables 3 and 13.

TABLE 12
Exemplary Target IL-1α and IL-1ß Gene Sequences

| Ident- | | | Ex- | Target | |
|---|---|---|---|---|---|
| ifier | Genome | Gene | on | Sequence 5′-3′ | PAM |
| SEQ ID | Homo | IL-1α | 4 | GCCATAGCTTACATGATAGA | AGG |
| NO: 37 | sapiens | | | | |
| SEQ ID | Homo | IL-1α | 4 | TCCTTCTATCATGTAAGCTA | TGG |
| NO: 38 | sapiens | | | | |
| SEQ ID | Homo | IL-1α | 4 | CCATGCAGCCTTCATGGAGT | GGG |
| NO: 39 | sapiens | | | | |
| SEQ ID | Homo | IL-1α | 4 | TCCATGCAGCCTTCATGGAG | TGG |
| NO: 40 | sapiens | | | | |
| SEQ ID | Homo | IL-1α | 4 | AGCTATGGCCCACTCCATGA | AGG |
| NO: 41 | sapiens | | | | |
| SEQ ID | Homo | IL-1α | 4 | ATTGATCCATGCAGCCTTCA | TGG |
| NO: 42 | sapiens | | | | |
| SEQ ID | Homo | IL-1α | 4 | CCCACTCCATGAAGGCTGCA | TGG |
| NO: 43 | sapiens | | | | |
| SEQ ID | Homo | IL-1α | 4 | GCTCTCCTTGAAGGTAAGCT | TGG |
| NO: 44 | sapiens | | | | |
| SEQ ID | Homo | IL-1α | 4 | TACCACCATGCTCTCCTTGA | AGG |
| NO: 45 | sapiens | | | | |
| SEQ ID | Homo | IL-1α | 4 | GCTTACCTTCAAGGAGAGCA | TGG |
| NO: 46 | sapiens | | | | |
| SEQ ID | Homo | IL-1α | 4 | TACCTTCAAGGAGAGCATGG | TGG |
| NO: 47 | sapiens | | | | |
| SEQ ID | Homo | IL-1α | 4 | ATGGTGGTAGTAGCAACCAA | CGG |
| NO: 48 | sapiens | | | | |
| SEQ ID | Homo | IL-1α | 4 | TGGTGGTAGTAGCAACCAAC | GGG |
| NO: 49 | sapiens | | | | |
| SEQ ID | Homo | IL-1α | 4 | CTTCTTCAGAACCTTCCCGT | TGG |
| NO: 50 | sapiens | | | | |
| SEQ ID | Homo | IL-1α | 4 | GGTAGTAGCAACCAACGGGA | AGG |
| NO: 51 | sapiens | | | | |
| SEQ ID | Homo | IL-1α | 4 | GGAAGGTTCTGAAGAAGAGA | CGG |
| NO: 52 | sapiens | | | | |
| SEQ ID | Homo | IL-1α | 4 | CTCCAGGTCATCATCAGTGA | TGG |
| NO: 53 | sapiens | | | | |
| SEQ ID | Homo | IL-1α | 4 | CATCACTGATGATGACCTGG | AGG |
| NO: 54 | sapiens | | | | |
| SEQ ID | Homo | IL-1α | 4 | AGTCATTGGCGATGGCCTCC | AGG |
| NO: 55 | sapiens | | | | |
| SEQ ID | Homo | IL-1α | 4 | TTCCTCTGAGTCATTGGCGA | TGG |
| NO: 56 | sapiens | | | | |
| SEQ ID | Homo | IL-1ß | 4 | TCCCATGTGTCGAAGAAGAT | AGG |
| NO: 57 | sapiens | | | | |
| SEQ ID | Homo | IL-1ß | 4 | AACCTATCTTCTTCGACACA | TGG |
| NO: 58 | sapiens | | | | |
| SEQ ID | Homo | IL-1ß | 4 | ACCTATCTTCTTCGACACAT | GGG |
| NO: 59 | sapiens | | | | |
| SEQ ID | Homo | IL-1ß | 4 | CTTCGACACATGGGATAACG | AGG |
| NO: 60 | sapiens | | | | |
| SEQ ID | Homo | IL-1ß | 4 | GTGCAGTTCAGTGATCGTAC | AGG |
| NO: 61 | sapiens | | | | |
| SEQ ID | Homo | IL-1ß | 4 | GATCACTGAACTGCACGCTC | CGG |
| NO: 62 | sapiens | | | | |
| SEQ ID | Homo | IL-1ß | 4 | ATCACTGAACTGCACGCTCC | GGG |
| NO: 63 | sapiens | | | | |
| SEQ ID | Homo | IL-1ß | 4 | CAAAAAAGCTTGGTGATGTC | TGG |
| NO: 64 | sapiens | | | | |
| SEQ ID | Homo | IL-1ß | 4 | CCATATCCTGTCCCTGGAGG | TGG |
| NO: 65 | sapiens | | | | |
| SEQ ID | Homo | IL-1ß | 4 | CTGAAAGCTCTCCACCTCCA | GGG |
| NO: 66 | sapiens | | | | |
| SEQ ID | Homo | IL-1ß | 4 | GCTCCATATCCTGTCCCTGG | AGG |
| NO: 67 | sapiens | | | | |
| SEQ ID | Homo | IL-1ß | 4 | AGCTCTCCACCTCCAGGGAC | AGG |
| NO: 68 | sapiens | | | | |
| SEQ ID | Homo | IL-1ß | 4 | GTTGCTCCATATCCTGTCCC | TGG |
| NO: 69 | sapiens | | | | |
| SEQ ID | Homo | IL-1ß | 4 | GGACAGGATATGGAGCAACA | AGG |
| NO: 70 | sapiens | | | | |
| SEQ ID | Canis | IL-1α | 3 | GTCACAGCTCATATCATAGA | AGG |
| NO: 71 | familiaris | | | | |
| SEQ ID | Canis | IL-1α | 3 | ACATGCAGTCCTCATGAAGT | GGG |
| NO: 72 | familiaris | | | | |
| SEQ ID | Canis | IL-1α | 3 | GACATGCAGTCCTCATGAAG | TGG |
| NO: 73 | familiaris | | | | |
| SEQ ID | Canis | IL-1α | 3 | GAGCTGTGACCCACTTCATG | AGG |
| NO: 74 | familiaris | | | | |
| SEQ ID | Canis | IL-1α | 3 | GGATGTCTTTGAGATTTCAG | AGG |
| NO: 75 | familiaris | | | | |
| SEQ ID | Canis | IL-1α | 3 | ATTTTCCTTGAAGGTAAGCT | GGG |
| NO: 76 | familiaris | | | | |
| SEQ ID | Canis | IL-1α | 3 | GACATCCCAGCTTACCTTCA | AGG |
| NO: 77 | familiaris | | | | |
| SEQ ID | Canis | IL-1α | 3 | CTTCAAGGAAAATGTGGTAG | TGG |
| NO: 78 | familiaris | | | | |
| SEQ ID | Canis | IL-1α | 3 | GTGGTAGTGGTGGCAGCCAA | TGG |
| NO: 79 | familiaris | | | | |
| SEQ ID | Canis | IL-1α | 3 | TGGTAGTGGTGGCAGCCAAT | GGG |
| NO: 80 | familiaris | | | | |
| SEQ ID | Canis | IL-1α | 3 | CTTCTTTAGAATCTTCCCAT | TGG |
| NO: 81 | familiaris | | | | |
| SEQ ID | Canis | IL-1α | 3 | GGAAGATTCTAAAGAAGAGA | CGG |
| NO: 82 | familiaris | | | | |
| SEQ ID | Canis | IL-1α | 3 | AATGTCTTCCAGGTCATCAT | CGG |
| NO: 83 | familiaris | | | | |
| SEQ ID | Canis | IL-1α | 3 | ATTCATCACCGATGATGACC | TGG |
| NO: 84 | familiaris | | | | |
| SEQ ID | Canis | IL-1α | 3 | ATTGCCAATGACACAGAAGA | AGG |
| NO: 85 | familiaris | | | | |
| SEQ ID | Canis | IL-1ß | 4 | CCTCATCTACCAGAGAACTG | TGG |
| NO: 86 | familiaris | | | | |
| SEQ ID | Canis | IL-1ß | 4 | CCACAGTTCTCTGGTAGATG | AGG |
| NO: 87 | familiaris | | | | |
| SEQ ID | Canis | IL-1ß | 4 | CACAGTTCTCTGGTAGATGA | GGG |
| NO: 88 | familiaris | | | | |
| SEQ ID | Canis | IL-1ß | 4 | GCTGGTGGGAGACTTGCAAC | TGG |
| NO: 89 | familiaris | | | | |
| SEQ ID | Canis | IL-1ß | 4 | ACTCTTGTTACAGAGCTGGT | GGG |
| NO: 90 | familiaris | | | | |
| SEQ ID | Canis | IL-1ß | 4 | GACTCTTGTTACAGAGCTGG | TGG |
| NO: 91 | familiaris | | | | |
| SEQ ID | Canis | IL-1ß | 4 | TCAGACTCTTGTTACAGAGC | TGG |
| NO: 92 | familiaris | | | | |
| SEQ ID | Canis | IL-1ß | 4 | AGCTCTGTAACAAGAGTCTG | AGG |
| NO: 93 | familiaris | | | | |
| SEQ ID | Canis | IL-1ß | 4 | CGTGTCAGTCATTGTAGCTT | TGG |
| NO: 94 | familiaris | | | | |
| SEQ ID | Canis | IL-1ß | 4 | TCCTGGAGGACCTGTGGGCA | GGG |
| NO: 95 | familiaris | | | | |
| SEQ ID | Canis | IL-1ß | 4 | GCTGAAGAAGCCCTGCCCAC | AGG |
| NO: 96 | familiaris | | | | |
| SEQ ID | Canis | IL-1ß | 4 | CATCCTCCTGGAGGACCTGT | GGG |
| NO: 97 | familiaris | | | | |
| SEQ ID | Canis | IL-1ß | 4 | TCATCCTCCTGGAGGACCTG | TGG |
| NO: 98 | familiaris | | | | |
| SEQ ID | Canis | IL-1ß | 4 | GCCCTGCCCACAGGTCCTCC | AGG |
| NO: 99 | familiaris | | | | |
| SEQ ID | Canis | IL-1ß | 4 | CTGCCCACAGGTCCTCCAGG | AGG |
| NO: 100 | familiaris | | | | |
| SEQ ID | Canis | IL-1ß | 4 | TCTTCAGGTCATCCTCCTGG | AGG |
| NO: 101 | familiaris | | | | |
| SEQ ID | Canis | IL-1ß | 4 | TGCTCTTCAGGTCATCCTCC | TGG |
| NO: 102 | familiaris | | | | |
| SEQ ID | Canis | IL-1ß | 4 | TGTAGCAAAAGATGCTCTTC | AGG |
| NO: 103 | familiaris | | | | |
| SEQ ID | Canis | IL-1ß | 4 | TTTTGCTACATCTTTGAAGA | AGG |
| NO: 104 | familiaris | | | | |
| SEQ ID | Equus | IL-1α | 4 | GTCATAGCTTGCATCATAGA | AGG |
| NO: 105 | caballus | | | | |
| SEQ ID | Equus | IL-1α | 4 | CCATGCAGTCCTCAGGAAGT | GGG |
| NO: 106 | caballus | | | | |
| SEQ ID | Equus | IL-1α | 4 | TCCATGCAGTCCTCAGGAAG | TGG |
| NO: 107 | caballus | | | | |
| SEQ ID | Equus | IL-1α | 4 | AAGCTATGACCCACTTCCTG | AGG |
| NO: 108 | caballus | | | | |
| SEQ ID | Equus | IL-1α | 4 | AATGTATCCATGCAGTCCTC | AGG |
| NO: 109 | caballus | | | | |
| SEQ ID | Equus | IL-1α | 4 | CCCACTTCCTGAGGACTGCA | TGG |
| NO: 110 | caballus | | | | |
| SEQ ID | Equus | IL-1α | 4 | GGATGTCTTAGAGGTTTCAG | AGG |
| NO: 111 | caballus | | | | |
| SEQ ID | Equus | IL-1α | 4 | GTTCAGCTTGGATGTCTTAG | AGG |
| NO: 112 | caballus | | | | |
| SEQ ID | Equus | IL-1α | 4 | GCTCTCCTTGAAGTTCAGCT | TGG |
| NO: 113 | caballus | | | | |
| SEQ ID | Equus | IL-1α | 4 | GACATCCAAGCTGAACTTCA | AGG |
| NO: 114 | caballus | | | | |
| SEQ ID | Equus | IL-1α | 4 | GCTGAACTTCAAGGAGAGCG | TGG |
| NO: 115 | caballus | | | | |
| SEQ ID | Equus | IL-1α | 4 | CTTCAAGGAGAGCGTGGTGC | TGG |
| NO: 116 | caballus | | | | |
| SEQ ID | Equus | IL-1α | 4 | CAAGGAGAGCGTGGTGCTGG | TGG |
| NO: 117 | caballus | | | | |
| SEQ ID | Equus | IL-1α | 4 | GTGGTGCTGGTGGCAGCCAA | CGG |
| NO: 118 | caballus | | | | |
| SEQ ID | Equus | IL-1α | 4 | TGGTGCTGGTGGCAGCCAAC | GGG |
| NO: 119 | caballus | | | | |
| SEQ ID | Equus | IL-1α | 4 | CTTCTTCAGAGTCTTCCCGT | TGG |
| NO: 120 | caballus | | | | |
| SEQ ID | Equus | IL-1α | 4 | GGAAGACTCTGAAGAAGAGA | CGG |
| NO: 121 | caballus | | | | |
| SEQ ID | Equus | IL-1α | 4 | AATGGCTTCCAGGTCATCAT | TGG |
| NO: 122 | caballus | | | | |
| SEQ ID | Equus | IL-1α | 4 | GTTCATCACCAATGATGACC | TGG |
| NO: 123 | caballus | | | | |
| SEQ ID | Equus | IL-1α | 4 | TTCTTCTGGATCATTGGCAA | TGG |
| NO: 124 | caballus | | | | |
| SEQ ID | Equus | IL-1ß | 4 | GGTGGTGGGAGATTTGCAAC | TGG |
| NO: 125 | caballus | | | | |
| SEQ ID | Equus | IL-1ß | 4 | AGTCTTGTTGTAGAGGTGGT | GGG |
| NO: 126 | caballus | | | | |
| SEQ ID | Equus | IL-1ß | 4 | AAGTCTTGTTGTAGAGGTGG | TGG |
| NO: 127 | caballus | | | | |
| SEQ ID | Equus | IL-1ß | 4 | TGAAAGTCTTGTTGTAGAGG | TGG |
| NO: 128 | caballus | | | | |
| SEQ ID | Equus | IL-1ß | 4 | GTTTGAAAGTCTTGTTGTAG | AGG |
| NO: 129 | caballus | | | | |
| SEQ ID | Equus | IL-1ß | 4 | ACATGCCATGTCAATCATTG | TGG |
| NO: 130 | caballus | | | | |
| SEQ ID | Equus | IL-1ß | 4 | CATGTCAATCATTGTGGCTG | TGG |
| NO: 131 | caballus | | | | |
| SEQ ID | Mus | IL-1α | 4 | GCCATAGCTTGCATCATAGA | AGG |
| NO: 132 | musculus | | | | |
| SEQ ID | Mus | IL-1α | 4 | TCCTTCTATGATGCAAGCTA | TGG |
| NO: 133 | musculus | | | | |
| SEQ ID | Mus | IL-1α | 4 | GGACATCTTTGACGTTTCAG | AGG |
| NO: 134 | musculus | | | | |
| SEQ ID | Mus | IL-1α | 4 | GATGTCCAACTTCACCTTCA | AGG |
| NO: 135 | musculus | | | | |
| SEQ ID | Mus | IL-1α | 4 | TGTCACCCGGCTCTCCTTGA | AGG |
| NO: 136 | musculus | | | | |
| SEQ ID | Mus | IL-1α | 4 | CTTCACCTTCAAGGAGAGCC | GGG |
| NO: 137 | musculus | | | | |
| SEQ ID | Mus | IL-1α | 4 | ACGTTGCTGATACTGTCACC | CGG |
| NO: 138 | musculus | | | | |
| SEQ ID | Mus | IL-1α | 4 | GTATCAGCAACGTCAAGCAA | CGG |
| NO: 139 | musculus | | | | |
| SEQ ID | Mus | IL-1α | 4 | TATCAGCAACGTCAAGCAAC | GGG |
| NO: 140 | musculus | | | | |
| SEQ ID | Mus | IL-1α | 4 | GGAAGATTCTGAAGAAGAGA | CGG |
| NO: 141 | musculus | | | | |
| SEQ ID | Mus | IL-1α | 4 | CTGCAGGTCATCTTCAGTGA | AGG |
| NO: 142 | musculus | | | | |
| SEQ ID | Mus | IL-1α | 4 | ACCTTCCAGATCATGGGTTA | TGG |
| NO: 143 | musculus | | | | |
| SEQ ID | Mus | IL-1α | 4 | CTCCTTACCTTCCAGATCAT | GGG |
| NO: 144 | musculus | | | | |
| SEQ ID | Mus | IL-1α | 4 | TCCATAACCCATGATCTGGA | AGG |
| NO: 145 | musculus | | | | |
| SEQ ID | Mus | IL-1α | 4 | AACCCATGATCTGGAAGGTA | AGG |
| NO: 146 | musculus | | | | |
| SEQ ID | Mus | IL-1ß | 4 | GACAGCCCAGGTCAAAGGTT | TGG |
| NO: 147 | musculus | | | | |
| SEQ ID | Mus | IL-1ß | 4 | ATCAGGACAGCCCAGGTCAA | AGG |
| NO: 148 | musculus | | | | |
| SEQ ID | Mus | IL-1ß | 4 | TGCTTCCAAACCTTTGACCT | GGG |
| NO: 149 | musculus | | | | |
| SEQ ID | Mus | IL-1ß | 4 | TGCTCTCATCAGGACAGCCC | AGG |
| NO: 150 | musculus | | | | |
| SEQ ID | Mus | IL-1ß | 4 | TGAAGCTGGATGCTCTCATC | AGG |
| NO: 151 | musculus | | | | |
| SEQ ID | Mus | IL-1ß | 4 | GCTGCTGCGAGATTTGAAGC | TGG |
| NO: 152 | musculus | | | | |
| SEQ ID | Mus | IL-1ß | 4 | CATCAACAAGAGCTTCAGGC | AGG |
| NO: 153 | musculus | | | | |
| SEQ ID | Mus | IL-1ß | 4 | GCAGGCAGTATCACTCATTG | TGG |
| NO: 154 | musculus | | | | |
| SEQ ID | Mus | IL-1ß | 4 | AGTATCACTCATTGTGGCTG | TGG |
| NO: 155 | musculus | | | | |
| SEQ ID | Mus | IL-1ß | 4 | TTGTGGCTGTGGAGAAGCTG | TGG |
| NO: 156 | musculus | | | | |
| SEQ ID | Mus | IL-1ß | 4 | AAGGTCCACGGGAAAGACAC | AGG |
| NO: 157 | musculus | | | | |
| SEQ ID | Mus | IL-1ß | 4 | AGCTACCTGTGTCTTTCCCG | TGG |
| NO: 158 | musculus | | | | |
| SEQ ID | Mus | IL-1ß | 4 | CCTCATCCTGGAAGGTCCAC | GGG |
| NO: 159 | musculus | | | | |
| SEQ ID | Mus | IL-1ß | 4 | TCCTCATCCTGGAAGGTCCA | CGG |
| NO: 160 | musculus | | | | |
| SEQ ID | Mus | IL-1ß | 4 | GCTCATGTCCTCATCCTGGA | AGG |
| NO: 161 | musculus | | | | |
| SEQ ID | Mus | IL-1ß | 4 | CCCGTGGACCTTCCAGGATG | AGG |
| NO: 162 | musculus | | | | |
| SEQ ID | Mus | IL-1ß | 4 | AGGTGCTCATGTCCTCATCC | TGG |
| NO: 163 | musculus | | | | |
| SEQ ID | Mus | IL-1ß | 4 | TTCAAAGATGAAGGAAAAGA | AGG |
| NO: 164 | musculus | | | | |
| SEQ ID | Mus | IL-1ß | 4 | AGTACCTTCTTCAAAGATGA | AGG |
| NO: 165 | musculus | | | | |
| SEQ ID | Mus | IL-1ß | 4 | TTTTCCTTCATCTTTGAAGA | AGG |
| NO: 167 | musculus | | | | |

TABLE 13
Exemplary CRISPR Guide RNAs

| Ident- | | | Ex- | Target | |
|---|---|---|---|---|---|
| ifier | Genome | Gene | on | Sequence 5′-3′ | PAM |
| SEQ ID | Homo | IL-1α | 4 | GCCAUAGCUUACAUGAUAGA | AGG |
| NO: 168 | sapiens | | | | |
| SEQ ID | Homo | IL-1α | 4 | UCCUUCUAUCAUGUAAGCUA | UGG |
| NO: 169 | sapiens | | | | |
| SEQ ID | Homo | IL-1α | 4 | CCAUGCAGCCUUCAUGGAGU | GGG |
| NO: 170 | sapiens | | | | |
| SEQ ID | Homo | IL-1α | 4 | UCCAUGCAGCCUUCAUGGAG | UGG |
| NO: 171 | sapiens | | | | |
| SEQ ID | Homo | IL-1α | 4 | AGCUAUGGCCCACUCCAUGA | AGG |
| NO: 172 | sapiens | | | | |
| SEQ ID | Homo | IL-1α | 4 | AUUGAUCCAUGCAGCCUUCA | UGG |
| NO: 173 | sapiens | | | | |
| SEQ ID | Homo | IL-1α | 4 | CCCACUCCAUGAAGGCUGCA | UGG |
| NO: 174 | sapiens | | | | |
| SEQ ID | Homo | IL-1α | 4 | GCUCUCCUUGAAGGUAAGCU | UGG |
| NO: 175 | sapiens | | | | |
| SEQ ID | Homo | IL-1α | 4 | UACCACCAUGCUCUCCUUGA | AGG |
| NO: 176 | sapiens | | | | |
| SEQ ID | Homo | IL-1α | 4 | GCUUACCUUCAAGGAGAGCA | UGG |
| NO: 177 | sapiens | | | | |
| SEQ ID | Homo | IL-1α | 4 | UACCUUCAAGGAGAGCAUGG | UGG |
| NO: 178 | sapiens | | | | |
| SEQ ID | Homo | IL-1α | 4 | AUGGUGGUAGUAGCAACCAA | CGG |
| NO: 179 | sapiens | | | | |
| SEQ ID | Homo | IL-1α | 4 | UGGUGGUAGUAGCAACCAAC | GGG |
| NO: 180 | sapiens | | | | |
| SEQ ID | Homo | IL-1α | 4 | CUUCUUCAGAACCUUCCCGU | UGG |
| NO: 181 | sapiens | | | | |
| SEQ ID | Homo | IL-1α | 4 | GGUAGUAGCAACCAACGGGA | AGG |
| NO: 182 | sapiens | | | | |
| SEQ ID | Homo | IL-1α | 4 | GGAAGGUUCUGAAGAAGAGA | CGG |
| NO: 183 | sapiens | | | | |
| SEQ ID | Homo | IL-1α | 4 | CUCCAGGUCAUCAUCAGUGA | UGG |
| NO: 184 | sapiens | | | | |
| SEQ ID | Homo | IL-1α | 4 | CAUCACUGAUGAUGACCUGG | AGG |
| NO: 185 | sapiens | | | | |
| SEQ ID | Homo | IL-1α | 4 | AGUCAUUGGCGAUGGCCUCC | AGG |
| NO: 186 | sapiens | | | | |
| SEQ ID | Homo | IL-1α | 4 | UUCCUCUGAGUCAUUGGCGA | UGG |
| NO: 187 | sapiens | | | | |
| SEQ ID | Homo | IL-1ß | 4 | UCCCAUGUGUCGAAGAAGAU | AGG |
| NO: 188 | sapiens | | | | |
| SEQ ID | Homo | IL-1ß | 4 | AACCUAUCUUCUUCGACACA | UGG |
| NO: 189 | sapiens | | | | |
| SEQ ID | Homo | IL-1ß | 4 | ACCUAUCUUCUUCGACACAU | GGG |
| NO: 190 | sapiens | | | | |
| SEQ ID | Homo | IL-1ß | 4 | CUUCGACACAUGGGAUAACG | AGG |
| NO: 191 | sapiens | | | | |
| SEQ ID | Homo | IL-1ß | 4 | GUGCAGUUCAGUGAUCGUAC | AGG |
| NO: 192 | sapiens | | | | |
| SEQ ID | Homo | IL-1ß | 4 | GAUCACUGAACUGCACGCUC | CGG |
| NO: 193 | sapiens | | | | |
| SEQ ID | Homo | IL-1ß | 4 | AUCACUGAACUGCACGCUCC | GGG |
| NO: 194 | sapiens | | | | |
| SEQ ID | Homo | IL-1ß | 4 | CAAAAAAGCUUGGUGAUGUC | UGG |
| NO: 195 | sapiens | | | | |
| SEQ ID | Homo | IL-1ß | 4 | CCAUAUCCUGUCCCUGGAGG | UGG |
| NO: 196 | sapiens | | | | |
| SEQ ID | Homo | IL-1ß | 4 | CUGAAAGCUCUCCACCUCCA | GGG |
| NO: 197 | sapiens | | | | |
| SEQ ID | Homo | IL-1ß | 4 | GCUCCAUAUCCUGUCCCUGG | AGG |
| NO: 198 | sapiens | | | | |
| SEQ ID | Homo | IL-1ß | 4 | AGCUCUCCACCUCCAGGGAC | AGG |
| NO: 199 | sapiens | | | | |
| SEQ ID | Homo | IL-1ß | 4 | GUUGCUCCAUAUCCUGUCCC | UGG |
| NO: 200 | sapiens | | | | |
| SEQ ID | Homo | IL-1ß | 4 | GGACAGGAUAUGGAGCAACA | AGG |
| NO: 201 | sapiens | | | | |
| SEQ ID | Canis | IL-1α | 3 | GUCACAGCUCAUAUCAUAGA | AGG |
| NO: 202 | familiaris | | | | |
| SEQ ID | Canis | IL-1α | 3 | ACAUGCAGUCCUCAUGAAGU | GGG |
| NO: 203 | familiaris | | | | |
| SEQ ID | Canis | IL-1α | 3 | GACAUGCAGUCCUCAUGAAG | UGG |
| NO: 204 | familiaris | | | | |
| SEQ ID | Canis | IL-1α | 3 | GAGCUGUGACCCACUUCAUG | AGG |
| NO: 205 | familiaris | | | | |
| SEQ ID | Canis | IL-1α | 3 | GGAUGUCUUUGAGAUUUCAG | AGG |
| NO: 206 | familiaris | | | | |
| SEQ ID | Canis | IL-1α | 3 | AUUUUCCUUGAAGGUAAGCU | GGG |
| NO: 207 | familiaris | | | | |
| SEQ ID | Canis | IL-1α | 3 | GACAUCCCAGCUUACCUUCA | AGG |
| NO: 208 | familiaris | | | | |
| SEQ ID | Canis | IL-1α | 3 | CUUCAAGGAAAAUGUGGUAG | UGG |
| NO: 209 | familiaris | | | | |
| SEQ ID | Canis | IL-1α | 3 | GUGGUAGUGGUGGCAGCCAA | UGG |
| NO: 210 | familiaris | | | | |
| SEQ ID | Canis | IL-1α | 3 | UGGUAGUGGUGGCAGCCAAU | GGG |
| NO: 211 | familiaris | | | | |
| SEQ ID | Canis | IL-1α | 3 | CUUCUUUAGAAUCUUCCCAU | UGG |
| NO: 212 | familiaris | | | | |
| SEQ ID | Canis | IL-1α | 3 | GGAAGAUUCUAAAGAAGAGA | CGG |
| NO: 213 | familiaris | | | | |
| SEQ ID | Canis | IL-1α | 3 | AAUGUCUUCCAGGUCAUCAU | CGG |
| NO: 214 | familiaris | | | | |
| SEQ ID | Canis | IL-1α | 3 | AUUCAUCACCGAUGAUGACC | UGG |
| NO: 215 | familiaris | | | | |
| SEQ ID | Canis | IL-1α | 3 | AUUGCCAAUGACACAGAAGA | AGG |
| NO: 216 | familiaris | | | | |
| SEQ ID | Canis | IL-1ß | 4 | CCUCAUCUACCAGAGAACUG | UGG |
| NO: 217 | familiaris | | | | |
| SEQ ID | Canis | IL-1ß | 4 | CCACAGUUCUCUGGUAGAUG | AGG |
| NO: 218 | familiaris | | | | |
| SEQ ID | Canis | IL-1ß | 4 | CACAGUUCUCUGGUAGAUGA | GGG |
| NO: 219 | familiaris | | | | |
| SEQ ID | Canis | IL-1ß | 4 | GCUGGUGGGAGACUUGCAAC | UGG |
| NO: 220 | familiaris | | | | |
| SEQ ID | Canis | IL-1ß | 4 | ACUCUUGUUACAGAGCUGGU | GGG |
| NO: 221 | familiaris | | | | |
| SEQ ID | Canis | IL-1ß | 4 | GACUCUUGUUACAGAGCUGG | UGG |
| NO: 222 | familiaris | | | | |
| SEQ ID | Canis | IL-1ß | 4 | UCAGACUCUUGUUACAGAGC | UGG |
| NO: 223 | familiaris | | | | |
| SEQ ID | Canis | IL-1ß | 4 | AGCUCUGUAACAAGAGUCUG | AGG |
| NO: 224 | familiaris | | | | |
| SEQ ID | Canis | IL-1ß | 4 | CGUGUCAGUCAUUGUAGCUU | UGG |
| NO: 225 | familiaris | | | | |
| SEQ ID | Canis | IL-1ß | 4 | UCCUGGAGGACCUGUGGGCA | GGG |
| NO: 226 | familiaris | | | | |
| SEQ ID | Canis | IL-1ß | 4 | GCUGAAGAAGCCCUGCCCAC | AGG |
| NO: 227 | familiaris | | | | |
| SEQ ID | Canis | IL-1ß | 4 | CAUCCUCCUGGAGGACCUGU | GGG |
| NO: 228 | familiaris | | | | |
| SEQ ID | Canis | IL-1ß | 4 | UCAUCCUCCUGGAGGACCUG | UGG |
| NO: 229 | familiaris | | | | |
| SEQ ID | Canis | IL-1ß | 4 | GCCCUGCCCACAGGUCCUCC | AGG |
| NO: 230 | familiaris | | | | |
| SEQ ID | Canis | IL-1ß | 4 | CUGCCCACAGGUCCUCCAGG | AGG |
| NO: 231 | familiaris | | | | |
| SEQ ID | Canis | IL-1ß | 4 | UCUUCAGGUCAUCCUCCUGG | AGG |
| NO: 232 | familiaris | | | | |
| SEQ ID | Canis | IL-1ß | 4 | UGCUCUUCAGGUCAUCCUCC | UGG |
| NO: 233 | familiaris | | | | |
| SEQ ID | Canis | IL-1ß | 4 | UGUAGCAAAAGAUGCUCUUC | AGG |
| NO: 234 | familiaris | | | | |
| SEQ ID | Canis | IL-1ß | 4 | UUUUGCUACAUCUUUGAAGA | AGG |
| NO: 235 | familiaris | | | | |
| SEQ ID | Equus | IL-1α | 4 | GUCAUAGCUUGCAUCAUAGA | AGG |
| NO: 236 | caballus | | | | |
| SEQ ID | Equus | IL-1α | 4 | CCAUGCAGUCCUCAGGAAGU | GGG |
| NO: 237 | caballus | | | | |
| SEQ ID | Equus | IL-1α | 4 | UCCAUGCAGUCCUCAGGAAG | UGG |
| NO: 238 | caballus | | | | |
| SEQ ID | Equus | IL-1α | 4 | AAGCUAUGACCCACUUCCUG | AGG |
| NO: 239 | caballus | | | | |
| SEQ ID | Equus | IL-1α | 4 | AAUGUAUCCAUGCAGUCCUC | AGG |
| NO: 240 | caballus | | | | |
| SEQ ID | Equus | IL-1α | 4 | CCCACUUCCUGAGGACUGCA | UGG |
| NO: 241 | caballus | | | | |
| SEQ ID | Equus | IL-1α | 4 | GGAUGUCUUAGAGGUUUCAG | AGG |
| NO: 242 | caballus | | | | |
| SEQ ID | Equus | IL-1α | 4 | GUUCAGCUUGGAUGUCUUAG | AGG |
| NO: 243 | caballus | | | | |
| SEQ ID | Equus | IL-1α | 4 | GCUCUCCUUGAAGUUCAGCU | UGG |
| NO: 244 | caballus | | | | |
| SEQ ID | Equus | IL-1α | 4 | GACAUCCAAGCUGAACUUCA | AGG |
| NO: 245 | caballus | | | | |
| SEQ ID | Equus | IL-1α | 4 | GCUGAACUUCAAGGAGAGCG | UGG |
| NO: 246 | caballus | | | | |
| SEQ ID | Equus | IL-1α | 4 | CUUCAAGGAGAGCGUGGUGC | UGG |
| NO: 247 | caballus | | | | |
| SEQ ID | Equus | IL-1α | 4 | CAAGGAGAGCGUGGUGCUGG | UGG |
| NO: 248 | caballus | | | | |
| SEQ ID | Equus | IL-1α | 4 | GUGGUGCUGGUGGCAGCCAA | CGG |
| NO: 249 | caballus | | | | |
| SEQ ID | Equus | IL-1α | 4 | UGGUGCUGGUGGCAGCCAAC | GGG |
| NO: 250 | caballus | | | | |
| SEQ ID | Equus | IL-1α | 4 | CUUCUUCAGAGUCUUCCCGU | UGG |
| NO: 251 | caballus | | | | |
| SEQ ID | Equus | IL-1α | 4 | GGAAGACUCUGAAGAAGAGA | CGG |
| NO: 252 | caballus | | | | |
| SEQ ID | Equus | IL-1α | 4 | AAUGGCUUCCAGGUCAUCAU | UGG |
| NO: 253 | caballus | | | | |
| SEQ ID | Equus | IL-1α | 4 | GUUCAUCACCAAUGAUGACC | UGG |
| NO: 254 | caballus | | | | |
| SEQ ID | Equus | IL-1α | 4 | UUCUUCUGGAUCAUUGGCAA | UGG |
| NO: 255 | caballus | | | | |
| SEQ ID | Equus | IL-1ß | 4 | GGUGGUGGGAGAUUUGCAAC | UGG |
| NO: 256 | caballus | | | | |
| SEQ ID | Equus | IL-1ß | 4 | AGUCUUGUUGUAGAGGUGGU | GGG |
| NO: 257 | caballus | | | | |
| SEQ ID | Equus | IL-1ß | 4 | AAGUCUUGUUGUAGAGGUGG | UGG |
| NO: 258 | caballus | | | | |
| SEQ ID | Equus | IL-1ß | 4 | UGAAAGUCUUGUUGUAGAGG | UGG |
| NO: 259 | caballus | | | | |
| SEQ ID | Equus | IL-1ß | 4 | GUUUGAAAGUCUUGUUGUAG | AGG |
| NO: 260 | caballus | | | | |
| SEQ ID | Equus | IL-1ß | 4 | ACAUGCCAUGUCAAUCAUUG | UGG |
| NO: 261 | caballus | | | | |
| SEQ ID | Equus | IL-1ß | 4 | CAUGUCAAUCAUUGUGGCUG | UGG |
| NO: 262 | caballus | | | | |
| SEQ ID | Mus | IL-1α | 4 | GCCAUAGCUUGCAUCAUAGA | AGG |
| NO: 263 | musculus | | | | |
| SEQ ID | Mus | IL-1α | 4 | UCCUUCUAUGAUGCAAGCUA | UGG |
| NO: 264 | musculus | | | | |
| SEQ ID | Mus | IL-1α | 4 | GGACAUCUUUGACGUUUCAG | AGG |
| NO: 265 | musculus | | | | |
| SEQ ID | Mus | IL-1α | 4 | GAUGUCCAACUUCACCUUCA | AGG |
| NO: 266 | musculus | | | | |
| SEQ ID | Mus | IL-1α | 4 | UGUCACCCGGCUCUCCUUGA | AGG |
| NO: 267 | musculus | | | | |
| SEQ ID | Mus | IL-1α | 4 | CUUCACCUUCAAGGAGAGCC | GGG |
| NO: 268 | musculus | | | | |
| SEQ ID | Mus | IL-1α | 4 | ACGUUGCUGAUACUGUCACC | CGG |
| NO: 269 | musculus | | | | |
| SEQ ID | Mus | IL-1α | 4 | GUAUCAGCAACGUCAAGCAA | CGG |
| NO: 270 | musculus | | | | |
| SEQ ID | Mus | IL-1α | 4 | UAUCAGCAACGUCAAGCAAC | GGG |
| NO: 271 | musculus | | | | |
| SEQ ID | Mus | IL-1α | 4 | GGAAGAUUCUGAAGAAGAGA | CGG |
| NO: 272 | musculus | | | | |
| SEQ ID | Mus | IL-1α | 4 | CUGCAGGUCAUCUUCAGUGA | AGG |
| NO: 273 | musculus | | | | |
| SEQ ID | Mus | IL-1α | 4 | ACCUUCCAGAUCAUGGGUUA | UGG |
| NO: 274 | musculus | | | | |
| SEQ ID | Mus | IL-1α | 4 | CUCCUUACCUUCCAGAUCAU | GGG |
| NO: 275 | musculus | | | | |
| SEQ ID | Mus | IL-1α | 4 | UCCAUAACCCAUGAUCUGGA | AGG |
| NO: 276 | musculus | | | | |
| SEQ ID | Mus | IL-1α | 4 | AACCCAUGAUCUGGAAGGUA | AGG |
| NO: 277 | musculus | | | | |
| SEQ ID | Mus | IL-1ß | 4 | GACAGCCCAGGUCAAAGGUU | UGG |
| NO: 278 | musculus | | | | |
| SEQ ID | Mus | IL-1ß | 4 | AUCAGGACAGCCCAGGUCAA | AGG |
| NO: 279 | musculus | | | | |
| SEQ ID | Mus | IL-1ß | 4 | UGCUUCCAAACCUUUGACCU | GGG |
| NO: 280 | musculus | | | | |
| SEQ ID | Mus | IL-1ß | 4 | UGCUCUCAUCAGGACAGCCC | AGG |
| NO: 281 | musculus | | | | |
| SEQ ID | Mus | IL-1ß | 4 | UGAAGCUGGAUGCUCUCAUC | AGG |
| NO: 282 | musculus | | | | |
| SEQ ID | Mus | IL-1ß | 4 | GCUGCUGCGAGAUUUGAAGC | UGG |
| NO: 283 | musculus | | | | |
| SEQ ID | Mus | IL-1ß | 4 | CAUCAACAAGAGCUUCAGGC | AGG |
| NO: 284 | musculus | | | | |
| SEQ ID | Mus | IL-1ß | 4 | GCAGGCAGUAUCACUCAUUG | UGG |
| NO: 285 | musculus | | | | |
| SEQ ID | Mus | IL-1ß | 4 | AGUAUCACUCAUUGUGGCUG | UGG |
| NO: 286 | musculus | | | | |
| SEQ ID | Mus | IL-1ß | 4 | UUGUGGCUGUGGAGAAGCUG | UGG |
| NO: 287 | musculus | | | | |
| SEQ ID | Mus | IL-1ß | 4 | AAGGUCCACGGGAAAGACAC | AGG |
| NO: 288 | musculus | | | | |
| SEQ ID | Mus | IL-1ß | 4 | AGCUACCUGUGUCUUUCCCG | UGG |
| NO: 289 | musculus | | | | |
| SEQ ID | Mus | IL-1ß | 4 | CCUCAUCCUGGAAGGUCCAC | GGG |
| NO: 290 | musculus | | | | |
| SEQ ID | Mus | IL-1ß | 4 | UCCUCAUCCUGGAAGGUCCA | CGG |
| NO: 291 | musculus | | | | |
| SEQ ID | Mus | IL-1ß | 4 | GCUCAUGUCCUCAUCCUGGA | AGG |
| NO: 292 | musculus | | | | |
| SEQ ID | Mus | IL-1ß | 4 | CCCGUGGACCUUCCAGGAUG | AGG |
| NO: 293 | musculus | | | | |
| SEQ ID | Mus | IL-1ß | 4 | AGGUGCUCAUGUCCUCAUCC | UGG |
| NO: 294 | musculus | | | | |
| SEQ ID | Mus | IL-1ß | 4 | UUCAAAGAUGAAGGAAAAGA | AGG |
| NO: 295 | musculus | | | | |
| SEQ ID | Mus | IL-1ß | 4 | AGUACCUUCUUCAAAGAUGA | AGG |
| NO: 296 | musculus | | | | |
| SEQ ID | Mus | IL-1ß | 4 | UUUUCCUUCAUCUUUGAAGA | AGG |
| NO: 297 | musculus | | | | |

In certain embodiments, the sequence of a guide RNA (e.g., a single guide RNA or sgRNA) may be modified to increase editing efficiency and/or reduce off-target effects. In certain embodiments, the sequence of a guide RNA may vary from the target sequence by about 1 base, about 2 bases, about 3 bases, about 4 bases, about 5 bases, about 5 bases, about 6 bases, about 7 bases, about 8 bases, about 9 bases, about 10 bases, about 15 bases, or greater than about 15 bases. In certain embodiments, the sequence of a guide RNA may vary from the target sequence by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or greater than about 20%. As used herein, variation form a target sequence may refer to the degree of complementarity.

In certain embodiments, a guide RNA used with a composition, method or system of the present disclosure is identical to a sequence as shown in any one of SEQ ID NO.: 21-34 and SEQ ID NO.: 168-297. In certain embodiments, a guide RNA used with a composition, method or system of the present disclosure is at least about 95% identical to a sequence as shown in any one of SEQ ID NO.: 21-34 and SEQ ID NO.: 168-297. In certain embodiments, a guide RNA used with a composition, method or system of the present disclosure is at least about 90% identical to a sequence as shown in any one of SEQ ID NO.: 21-34 and SEQ ID NO.: 168-297. In certain embodiments, a guide RNA used with a composition, method or system of the present disclosure is at least about 85% identical to a sequence as shown in any one of SEQ ID NO.: 21-34 and SEQ ID NO.: 168-297. In certain embodiments, a guide RNA used with a composition, method or system of the present disclosure is at least about 80% identical to a sequence as shown in any one of SEQ ID NO.: 21-34 and SEQ ID NO.: 168-297. In certain embodiments, a guide RNA used with a composition, method or system of the present disclosure is at least about 75% identical to a sequence as shown in any one of SEQ ID NO.: 21-34 and SEQ ID NO.: 168-297. In certain embodiments, a guide RNA used with a composition, method or system of the present disclosure is at least about 70% identical to a sequence as shown in any one of SEQ ID NO.: 21-34 and SEQ ID NO.: 168-297. In certain embodiments, a guide RNA used with a composition, method or system of the present disclosure is at least about 65% identical to a sequence as shown in any one of SEQ ID NO.: 21-34 and SEQ ID NO.: 168-297. In certain embodiments, a guide RNA used with a composition, method or system of the present disclosure is at least about 60% identical to a sequence as shown in any one of SEQ ID NO.: 21-34 and SEQ ID NO.: 168-297. In certain embodiments, a guide RNA used with a composition, method or system of the present disclosure is at least about 55% identical to a sequence as shown in any one of SEQ ID NO.: 21-34 and SEQ ID NO.: 168-297. In certain embodiments, a guide RNA used with a composition, method or system of the present disclosure is at least about 50% identical to a sequence as shown in any one of SEQ ID NO.: 21-34 and SEQ ID NO.: 168-297. In certain embodiments, a guide RNA used with a composition, method or system of the present disclosure is at least about 45% identical to a sequence as shown in any one of SEQ ID NO.: 21-34 and SEQ ID NO.: 168-297. In certain embodiments, a guide RNA used with a composition, method or system of the present disclosure is at least about 40% identical to a sequence as shown in any one of SEQ ID NO.: 21-34 and SEQ ID NO.: 168-297. In certain embodiments, a guide RNA used with a composition, method or system of the present disclosure is at least about 35% identical to a sequence as shown in any one of SEQ ID NO.: 21-34 and SEQ ID NO.: 168-297. In certain embodiments, a guide RNA used with a composition, method or system of the present disclosure is at least about 35% identical to a sequence as shown in any one of SEQ ID NO.: 21-34 and SEQ ID NO.: 168-297.

In certain embodiments, a guide RNA used with a composition, method or system of the present has 1 base substitution in a sequence as shown in any one of SEQ ID NO.: 21-34 and SEQ ID NO.: 168-297. In certain embodiments, a guide RNA used with a composition, method or system of the present has 2 base substitutions in a sequence as shown in any one of SEQ ID NO.: 21-34 and SEQ ID NO.: 168-297. In certain embodiments, a guide RNA used with a composition, method or system of the present has 3 base substitutions in a sequence as shown in any one of SEQ ID NO.: 21-34 and SEQ ID NO.: 168-297. In certain embodiments, a guide RNA used with a composition, method or system of the present has 4 base substitutions in a sequence as shown in any one of SEQ ID NO.: 21-34 and SEQ ID NO.: 168-297. In certain embodiments, a guide RNA used with a composition, method or system of the present has 4 base substitutions in a sequence as shown in any one of SEQ ID NO.: 21-34 and SEQ ID NO.: 168-297. In certain embodiments, a guide RNA used with a composition, method or system of the present has 6 base substitutions in a sequence as shown in any one of SEQ ID NO.: 21-34 and SEQ ID NO.: 168-297. In certain embodiments, a guide RNA used with a composition, method or system of the present has 7 base substitutions in a sequence as shown in any one of SEQ ID NO.: 21-34 and SEQ ID NO.: 168-297. In certain embodiments, a guide RNA used with a composition, method or system of the present has 8 base substitutions in a sequence as shown in any one of SEQ ID NO.: 21-34 and SEQ ID NO.: 168-297. In certain embodiments, a guide RNA used with a composition, method or system of the present has 9 base substitutions in a sequence as shown in any one of SEQ ID NO.: 21-34 and SEQ ID NO.: 168-297. In certain embodiments, a guide RNA used with a composition, method or system of the present has 10 base substitutions in a sequence as shown in any one of SEQ ID NO.: 21-34 and SEQ ID NO.: 168-297. In certain embodiments, a guide RNA used with a composition, method or system of the present has 11 base substitutions in a sequence as shown in any one of SEQ ID NO.: 21-34 and SEQ ID NO.: 168-297. In certain embodiments, a guide RNA used with a composition, method or system of the present has 12 base substitutions in a sequence as shown in any one of SEQ ID NO.: 21-34 and SEQ ID NO.: 168-297. In certain embodiments, a guide RNA used with a composition, method or system of the present has 13 base substitutions in a sequence as shown in any one of SEQ ID NO.: 21-34 and SEQ ID NO.: 168-297. In certain embodiments, a guide RNA used with a composition, method or system of the present has 14 base substitutions in a sequence as shown in any one of SEQ ID NO.: 21-34 and SEQ ID NO.: 168-297. In certain embodiments, a guide RNA used with a composition, method or system of the present has 15 base substitutions in a sequence as shown in any one of SEQ ID NO.: 21-34 and SEQ ID NO.: 168-297.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the human IL-1α gene by binding to at least a portion of Exon 1 of the human IL-1α gene. In certain embodiments, a guide RNA of the present disclosure is designed to or capable of knocking down the human IL-1α gene by binding to at least a portion of Exon 2 of the human IL-1α gene. In certain embodiments, a guide RNA of the present disclosure is designed to or capable of knocking down the human IL-1α gene by binding to at least a portion of Exon 3 of the human IL-1α gene. In certain embodiments, a guide RNA of the present disclosure is designed to or capable of knocking down the human IL-1α gene by binding to at least a portion of Exon 4 of the human IL-1α gene. In certain embodiments, a guide RNA of the present disclosure is designed to or capable of knocking down the human IL-1α gene by binding to at least a portion of Exon 5 of the human IL-1α gene. In certain embodiments, a guide RNA of the present disclosure is designed to or capable of knocking down the human IL-1α gene by binding to at least a portion of Exon 6 of the human IL-1α gene. In certain embodiments, a guide RNA of the present disclosure is designed to or capable of knocking down the human IL-1α gene by binding to at least a portion of Exon 7 of the human IL-1α gene. In certain embodiments, a guide RNA of the present disclosure is designed to or capable of knocking down the human IL-1α gene by binding to at least a portion of Exon 8 of the human IL-1α gene.

In certain embodiments, a guide RNA of the present disclosure is designed to or capable of knocking down the human IL-1β gene by binding to at least a portion of Exon 1 of the human IL-1β gene. In certain embodiments, a guide RNA of the present disclosure is designed to or capable of knocking down the human IL-1β gene by binding to at least a portion of Exon 2 of the human IL-1β gene. In certain embodiments, a guide RNA of the present disclosure is designed to or capable of knocking down the human IL-1β gene by binding to at least a portion of Exon 3 of the human IL-1β gene. In certain embodiments, a guide RNA of the present disclosure is designed to or capable of knocking down the human IL-1β gene by binding to at least a portion of Exon 4 of the human IL-1β gene. In certain embodiments, a guide RNA of the present disclosure is designed to or capable of knocking down the human IL-1β gene by binding to at least a portion of Exon 5 of the human IL-1β gene. In certain embodiments, a guide RNA of the present disclosure is designed to or capable of knocking down the human IL-1β gene by binding to at least a portion of Exon 6 of the human IL-1β gene. In certain embodiments, a guide RNA of the present disclosure is designed to or capable of knocking down the human IL-1β gene by binding to at least a portion of Exon 7 of the human IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Canis familiaris IL-1α gene by binding to at least a portion of Exon 1 of the Canis familiaris IL-1α gene. In certain embodiments, a guide RNA of the present disclosure is designed to or capable of knocking down the Canis familiaris IL-1α gene by binding to at least a portion of Exon 2 of the Canis familiaris IL-1α gene. In certain embodiments, a guide RNA of the present disclosure is designed to or capable of knocking down the Canis familiaris IL-1α gene by binding to at least a portion of Exon 3 of the Canis familiaris IL-1α gene. In certain embodiments, a guide RNA of the present disclosure is designed to or capable of knocking down the Canis familiaris IL-1α gene by binding to at least a portion of Exon 4 of the Canis familiaris IL-1α gene. In certain embodiments, a guide RNA of the present disclosure is designed to or capable of knocking down the Canis familiaris IL-1α gene by binding to at least a portion of Exon 5 of the Canis familiaris IL-1α gene. In certain embodiments, a guide RNA of the present disclosure is designed to or capable of knocking down the Canis familiaris IL-1α gene by binding to at least a portion of Exon 6 of the Canis familiaris IL-1α gene. In certain embodiments, a guide RNA of the present disclosure is designed to or capable of knocking down the Canis familiaris IL-1α gene by binding to at least a portion of Exon 7 of the Canis familiaris IL-1α gene.

In certain embodiments, a guide RNA of the present disclosure is designed to or capable of knocking down the Canis familiaris IL-1β gene by binding to at least a portion of Exon 1 of the Canis familiaris IL-1β gene. In certain embodiments, a guide RNA of the present disclosure is designed to or capable of knocking down the Canis familiaris IL-1β gene by binding to at least a portion of Exon 2 of the Canis familiaris IL-1β gene. In certain embodiments, a guide RNA of the present disclosure is designed to or capable of knocking down the Canis familiaris IL-1β gene by binding to at least a portion of Exon 3 of the Canis familiaris IL-1β gene. In certain embodiments, a guide RNA of the present disclosure is designed to or capable of knocking down the Canis familiaris IL-1β gene by binding to at least a portion of Exon 4 of the Canis familiaris IL-1β gene. In certain embodiments, a guide RNA of the present disclosure is designed to or capable of knocking down the Canis familiaris IL-1β gene by binding to at least a portion of Exon 5 of the Canis familiaris IL-1β gene. In certain embodiments, a guide RNA of the present disclosure is designed to or capable of knocking down the Canis familiaris IL-1β gene by binding to at least a portion of Exon 6 of the Canis familiaris IL-1β gene. In certain embodiments, a guide RNA of the present disclosure is designed to or capable of knocking down the Canis familiaris IL-1β gene by binding to at least a portion of Exon 7 of the Canis familiaris IL-1β gene. In certain embodiments, a guide RNA of the present disclosure is designed to or capable of knocking down the Canis familiaris IL-1β gene by binding to at least a portion of Exon 8 of the Canis familiaris IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Equus caballus IL-1α gene by binding to at least a portion of Exon 1 of the Equus caballus IL-1α gene. In certain embodiments, a guide RNA of the present disclosure is designed to or capable of knocking down the Equus caballus IL-1α gene by binding to at least a portion of Exon 2 of the Equus caballus IL-1α gene. In certain embodiments, a guide RNA of the present disclosure is designed to or capable of knocking down the Equus caballus IL-1α gene by binding to at least a portion of Exon 3 of the Equus caballus IL-1α gene. In certain embodiments, a guide RNA of the present disclosure is designed to or capable of knocking down the Equus caballus IL-1α gene by binding to at least a portion of Exon 4 of the Equus caballus IL-1α gene. In certain embodiments, a guide RNA of the present disclosure is designed to or capable of knocking down the Equus caballus IL-1α gene by binding to at least a portion of Exon 5 of the Equus caballus IL-1α gene. In certain embodiments, a guide RNA of the present disclosure is designed to or capable of knocking down the Equus caballus IL-1α gene by binding to at least a portion of Exon 6 of the Equus caballus IL-1α gene. In certain embodiments, a guide RNA of the present disclosure is designed to or capable of knocking down the Equus caballus IL-1α gene by binding to at least a portion of Exon 7 of the Equus caballus IL-1α gene.

In certain embodiments, a guide RNA of the present disclosure is designed to or capable of knocking down the Equus caballus IL-1β gene by binding to at least a portion of Exon 1 of the Equus caballus IL-1β gene. In certain embodiments, a guide RNA of the present disclosure is designed to or capable of knocking down the Equus caballus IL-1β gene by binding to at least a portion of Exon 2 of the Equus caballus IL-1β gene. In certain embodiments, a guide RNA of the present disclosure is designed to or capable of knocking down the Equus caballus IL-1β gene by binding to at least a portion of Exon 3 of the Equus caballus IL-1β gene. In certain embodiments, a guide RNA of the present disclosure is designed to or capable of knocking down the Equus caballus IL-1β gene by binding to at least a portion of Exon 4 of the Equus caballus IL-1β gene. In certain embodiments, a guide RNA of the present disclosure is designed to or capable of knocking down the Equus caballus IL-1β gene by binding to at least a portion of Exon 5 of the Equus caballus IL-1β gene. In certain embodiments, a guide RNA of the present disclosure is designed to or capable of knocking down the Equus caballus IL-1β gene by binding to at least a portion of Exon 6 of the Equus caballus IL-1β gene. In certain embodiments, a guide RNA of the present disclosure is designed to or capable of knocking down the Equus caballus IL-1β gene by binding to at least a portion of Exon 7 of the Equus caballus IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Mus musculus IL-1α gene by binding to at least a portion of Exon 1 of the Mus musculus IL-1α gene. In certain embodiments, a guide RNA of the present disclosure is designed to or capable of knocking down the Mus musculus IL-1α gene by binding to at least a portion of Exon 2 of the Mus musculus IL-1α gene. In certain embodiments, a guide RNA of the present disclosure is designed to or capable of knocking down the Mus musculus IL-1α gene by binding to at least a portion of Exon 3 of the Mus musculus IL-1α gene. In certain embodiments, a guide RNA of the present disclosure is designed to or capable of knocking down the Mus musculus IL-1α gene by binding to at least a portion of Exon 4 of the Mus musculus IL-1α gene. In certain embodiments, a guide RNA of the present disclosure is designed to or capable of knocking down the Mus musculus IL-1α gene by binding to at least a portion of Exon 5 of the Mus musculus IL-1α gene. In certain embodiments, a guide RNA of the present disclosure is designed to or capable of knocking down the Mus musculus IL-1α gene by binding to at least a portion of Exon 6 of the Mus musculus IL-1α gene. In certain embodiments, a guide RNA of the present disclosure is designed to or capable of knocking down the Mus musculus IL-1α gene by binding to at least a portion of Exon 7 of the Mus musculus IL-1α gene. In certain embodiments, a guide RNA of the present disclosure is designed to or capable of knocking down the Mus musculus IL-1α gene by binding to at least a portion of Exon 8 of the Mus musculus IL-1α gene.

In certain embodiments, a guide RNA of the present disclosure is designed to or capable of knocking down the Mus musculus IL-1β gene by binding to at least a portion of Exon 1 of the Mus musculus IL-1β gene. In certain embodiments, a guide RNA of the present disclosure is designed to or capable of knocking down the Mus musculus IL-1β gene by binding to at least a portion of Exon 2 of the Mus musculus IL-1β gene. In certain embodiments, a guide RNA of the present disclosure is designed to or capable of knocking down the Mus musculus IL-1β gene by binding to at least a portion of Exon 3 of the Mus musculus IL-1β gene. In certain embodiments, a guide RNA of the present disclosure is designed to or capable of knocking down the Mus musculus IL-1β gene by binding to at least a portion of Exon 4 of the Mus musculus IL-1β gene. In certain embodiments, a guide RNA of the present disclosure is designed to or capable of knocking down the Mus musculus IL-1β gene by binding to at least a portion of Exon 5 of the Mus musculus IL-1β gene. In certain embodiments, a guide RNA of the present disclosure is designed to or capable of knocking down the Mus musculus IL-1β gene by binding to at least a portion of Exon 6 of the Mus musculus IL-1β gene. In certain embodiments, a guide RNA of the present disclosure is designed to or capable of knocking down the Mus musculus IL-1β gene by binding to at least a portion of Exon 7 of the Mus musculus IL-1β gene.

In some instances, the sgRNA is introduced into a cell (e.g., an in vitro cell such as a primary cell for ex vivo therapy, or an in vivo cell such as in a patient) with a recombinant expression vector comprising a nucleotide sequence encoding a Cas nuclease (e.g., Cas9 polypeptide) or a variant or fragment thereof. In some embodiments, the sgRNA is complexed with a Cas nuclease (e.g., a Cas9 polypeptide) or a variant or fragment thereof to form a ribonucleoprotein (RNP)-based delivery system for introduction into a cell (e.g., an in vitro cell such as a primary cell for ex vivo therapy, or an in vivo cell such as in a patient). In other instances, the sgRNA is introduced into a cell (e.g., an in vitro cell such as a primary cell for ex vivo therapy, or an in vivo cell such as in a patient) with an mRNA encoding a Cas nuclease (e.g., Cas9 polypeptide) or a variant or fragment thereof.

Any heterologous or foreign nucleic acid (e.g., target locus-specific sgRNA and/or polynucleotide encoding a Cas9 polynucleotide) can be introduced into a cell using any method known to one skilled in the art. Such methods include, but are not limited to, electroporation, nucleofection, transfection, lipofection, transduction, microinjection, electroinjection, electrofusion, nanoparticle bombardment, transformation, conjugation, and the like.

The nucleic acid sequence of the sgRNA can be any polynucleotide sequence having sufficient complementarity with a target polynucleotide sequence (e.g., target DNA sequence) to hybridize with the target sequence and direct sequence-specific binding of a CRISPR complex to the target sequence. In some embodiments, the degree of complementarity between a guide sequence of the sgRNA and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 35 nucleotides, 40 nucleotides, 45 nucleotides, 50 nucleotides, 75 nucleotides, or more nucleotides in length. In some instances, a guide sequence is about 20 nucleotides in length. In other instances, a guide sequence is about 15 nucleotides in length. In other instances, a guide sequence is about 25 nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

The nucleic acid sequence of a sgRNA can be selected using any of the web-based software described above. Considerations for selecting a DNA-targeting RNA include the PAM sequence for the Cas nuclease (e.g., Cas9 polypeptide) to be used, and strategies for minimizing off-target modifications. Tools, such as the CRISPR Design Tool, can provide sequences for preparing the sgRNA, for assessing target modification efficiency, and/or assessing cleavage at off-target sites. Another consideration for selecting the sequence of a sgRNA includes reducing the degree of secondary structure within the guide sequence. Secondary structure may be determined by any suitable polynucleotide folding algorithm. Some programs are based on calculating the minimal Gibbs free energy. Examples of suitable algorithms include mFold (Zuker and Stiegler, Nucleic Acids Res, 9 (1981), 133-148), UNAFold package (Markham et al, Methods Mol Biol, 2008, 453:3-31) and RNAfold form the ViennaRNa Package.

The sgRNA can be about 10 to about 500 nucleotides, e.g., about 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, 280 nucleotides, 290 nucleotides, 300 nucleotides, 310 nucleotides, 320 nucleotides, 330 nucleotides, 340 nucleotides, 350 nucleotides, 360 nucleotides, 370 nucleotides, 380 nucleotides, 390 nucleotides, 400 nucleotides, 410 nucleotides, 420 nucleotides, 430 nucleotides, 440 nucleotides, 450 nucleotides, 460 nucleotides, 470 nucleotides, 480 nucleotides, 490 nucleotides, or about 500 nucleotides. In some embodiments, the sgRNA is about 20 to about 500 nucleotides, e.g., 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides 110 nucleotides, 115 nucleotides, 120 nucleotides, 125 nucleotides, 130 nucleotides, 135 nucleotides, 140 nucleotides, 145 nucleotides, 150 nucleotides, 155 nucleotides, 160 nucleotides, 165 nucleotides, 170 nucleotides, 175 nucleotides, 180 nucleotides, 185 nucleotides, 190 nucleotides, 195 nucleotides, 200 nucleotides, 205 nucleotides, 210 nucleotides, 215 nucleotides, 220 nucleotides, 225 nucleotides, 230 nucleotides, 235 nucleotides, 240 nucleotides, 245 nucleotides, 250 nucleotides, 255 nucleotides, 260 nucleotides, 265 nucleotides, 270 nucleotides, 275 nucleotides, 280 nucleotides, 285 nucleotides, 290 nucleotides, 295 nucleotides, 300 nucleotides, 305 nucleotides, 310 nucleotides, 315 nucleotides, 320 nucleotides, 325 nucleotides, 330 nucleotides, 335 nucleotides, 340 nucleotides, 345 nucleotides, 350 nucleotides, 355 nucleotides, 360 nucleotides, 365 nucleotides, 370 nucleotides, 375 nucleotides, 380 nucleotides, 385 nucleotides, 390 nucleotides, 395 nucleotides, 400 nucleotides, 405 nucleotides, 410 nucleotides, 415 nucleotides, 420 nucleotides, 425 nucleotides, 430 nucleotides, 435 nucleotides, 440 nucleotides, 445 nucleotides, 450 nucleotides, 455 nucleotides, 460 nucleotides, 465 nucleotides, 470 nucleotides, 475 nucleotides, 480 nucleotides, 485 nucleotides, 490 nucleotides, 495 nucleotides, or 500 nucleotides. In certain embodiments, the sgRNA is about 20 to about 100 nucleotides, e.g., about 20 nucleotides, e.g., 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, 80 nucleotides, 81 nucleotides, 82 nucleotides, 83 nucleotides, 84 nucleotides, 85 nucleotides, 86 nucleotides, 87 nucleotides, 88 nucleotides, 89 nucleotides, 90 nucleotides, 91 nucleotides, 92 nucleotides, 93 nucleotides, 94 nucleotides, 95 nucleotides, 96 nucleotides, 97 nucleotides, 98 nucleotides, 99 nucleotides, or about 100 nucleotides.

The scaffold sequence can be about 10 to about 500 nucleotides, e.g., about 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, 280 nucleotides, 290 nucleotides, 300 nucleotides, 310 nucleotides, 320 nucleotides, 330 nucleotides, 340 nucleotides, 350 nucleotides, 360 nucleotides, 370 nucleotides, 380 nucleotides, 390 nucleotides, 400 nucleotides, 410 nucleotides, 420 nucleotides, 430 nucleotides, 440 nucleotides, 450 nucleotides, 460 nucleotides, 470 nucleotides, 480 nucleotides, 490 nucleotides, or about 500 nucleotides. In some embodiments, the scaffold sequence is about 20 to about 500 nucleotides, e.g., 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides 110 nucleotides, 115 nucleotides, 120 nucleotides, 125 nucleotides, 130 nucleotides, 135 nucleotides, 140 nucleotides, 145 nucleotides, 150 nucleotides, 155 nucleotides, 160 nucleotides, 165 nucleotides, 170 nucleotides, 175 nucleotides, 180 nucleotides, 185 nucleotides, 190 nucleotides, 195 nucleotides, 200 nucleotides, 205 nucleotides, 210 nucleotides, 215 nucleotides, 220 nucleotides, 225 nucleotides, 230 nucleotides, 235 nucleotides, 240 nucleotides, 245 nucleotides, 250 nucleotides, 255 nucleotides, 260 nucleotides, 265 nucleotides, 270 nucleotides, 275 nucleotides, 280 nucleotides, 285 nucleotides, 290 nucleotides, 295 nucleotides, 300 nucleotides, 305 nucleotides, 310 nucleotides, 315 nucleotides, 320 nucleotides, 325 nucleotides, 330 nucleotides, 335 nucleotides, 340 nucleotides, 345 nucleotides, 350 nucleotides, 355 nucleotides, 360 nucleotides, 365 nucleotides, 370 nucleotides, 375 nucleotides, 380 nucleotides, 385 nucleotides, 390 nucleotides, 395 nucleotides, 400 nucleotides, 405 nucleotides, 410 nucleotides, 415 nucleotides, 420 nucleotides, 425 nucleotides, 430 nucleotides, 435 nucleotides, 440 nucleotides, 445 nucleotides, 450 nucleotides, 455 nucleotides, 460 nucleotides, 465 nucleotides, 470 nucleotides, 475 nucleotides, 480 nucleotides, 485 nucleotides, 490 nucleotides, 495 nucleotides, or 500 nucleotides. In certain embodiments, the scaffold sequence is about 20 to about 100 nucleotides, e.g., about 20 nucleotides, e.g., 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, 80 nucleotides, 81 nucleotides, 82 nucleotides, 83 nucleotides, 84 nucleotides, 85 nucleotides, 86 nucleotides, 87 nucleotides, 88 nucleotides, 89 nucleotides, 90 nucleotides, 91 nucleotides, 92 nucleotides, 93 nucleotides, 94 nucleotides, 95 nucleotides, 96 nucleotides, 97 nucleotides, 98 nucleotides, 99 nucleotides, or about 100 nucleotides.

The nucleotides of the sgRNA can include a modification in the ribose (e.g., sugar) group, phosphate group, nucleobase, or any combination thereof. In some embodiments, the modification in the ribose group comprises a modification at the 2′ position of the ribose.

In some embodiments, the nucleotide includes a 2′fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), a phosphodiamidate morpholino, or a combination thereof.

Modified nucleotides or nucleotide analogues can include sugar- and/or backbone-ribonucleotides (i.e., include modifications to the phosphate-sugar backbone). For example, the phosphodiester linkages of a native or natural RNA may be to include at least one of a nitrogen or sulfur heteroatom. In some backbone-ribonucleotides the phosphoester group connecting to adjacent ribonucleotides may be replaced by a group, e.g., of phosphothioate group. In some sugar-ribonucleotides, the 2′ moiety is a group selected from H, OR, R, halo, SH, SR, H2, HR, R₂ or ON, wherein R is C₁-C6 alkyl, alkenyl or alkynyl and halo is F, Cl, Br, or I.

In some embodiments, the nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the sgRNA contains one or more 2′-fluro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and nucleotides and nucleosides are well-known in the art and described in, e.g., U.S. Pat. Nos. 4,373,071, 4,458,066, 4,500,707, 4,668,777, 4,973,679, 5,047,524, 5,132,418, 5,153,319, 5,262,530, and 5,700,642. Numerous nucleosides and nucleotides that are suitable for use as described herein are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the sgRNA described herein includes a nucleobase-ribonucleotide, i.e., a ribonucleotide containing at least one non-naturally occurring nucleobase instead of a naturally occurring nucleobase. Non-limiting examples of nucleobases which can be incorporated into nucleosides and nucleotides include m5C (5-methylcytidine), m5U (5-methyluridine), m6A (N6-methyladenosine), s2U (2-thiouridine), Um (2′-O-methyluridine), mlA (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A(N6-hydroxynorvalylcarbamoyl adenosine), ms2hn6A (2-methylthio-N6-hydroxynorvalyl carbamoyladenosine), Ar(p) (2′-O-ribosyladenosine(phosphate)), I (inosine), mi1 (1-methylinosine), m′lm (1,2′-O-dimethylinosine), m3C (3-methylcytidine), Cm (2T-o-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), mlG (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (under hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-0-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), rn62Am (N6,N6,0-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), mlGm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxy cytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

The sgRNA can be synthesized by any method known by one of ordinary skill in the art. In some embodiments, the sgRNA is chemically synthesized. Modified sgRNAs can be synthesized using 2′-O-thionocarbamate-protected nucleoside phosphoramidites. Methods are described in, e.g., Dellinger et al., J. American Chemical Society, 133, 11540-11556 (2011); Threlfall et al., Organic & Biomolecular Chemistry, 10, 746-754 (2012); and Dellinger et al, J. American Chemical Society, 125, 940-950 (2003). Modified sgRNAs are commercially available from, e.g., TriLink BioTechnologies (San Diego, Calif.).

Additional detailed description of useful sgRNAs can be found in, e.g., Hendel et al., Nat Biotechnol, 2015, 33(9): 985-989 and Dever et al., Nature, 2016, 539: 384-389, the disclosures are herein incorporated by reference in their entirety for all purposes.

A person having skill in the art will appreciate that a guide RNA as disclosed in the present disclosure may be used in combination with any Cas protein known in the art (e.g., any Cas type, from any suitable organism or bacterial species.

The Cas protein may be a type I, type II, type III, type IV, type V, or type VI Cas protein. The Cas protein may comprise one or more domains. Non-limiting examples of domains include, a guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. The guide nucleic acid recognition and/or binding domain may interact with a guide nucleic acid. The nuclease domain may comprise catalytic activity for nucleic acid cleavage. The nuclease domain may lack catalytic activity to prevent nucleic acid cleavage. The Cas protein may be a chimeric Cas protein that is fused to other proteins or polypeptides. The Cas protein may be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas1O, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

The Cas protein may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

The Cas protein may be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, Proteobacterium, Legionella pneumophila, Parasutterella excrementihominis, Wolinella succinogenes, and Francisella novicida. The term, “derived,” in this instance, is defined as modified from the naturally-occurring variety of bacterial species to maintain a significant portion or significant homology to the naturally-occurring variety of bacterial species. A significant portion may be at least 10 consecutive nucleotides, at least 20 consecutive nucleotides, at least 30 consecutive nucleotides, at least 40 consecutive nucleotides, at least 50 consecutive nucleotides, at least 60 consecutive nucleotides, at least 70 consecutive nucleotides, at least 80 consecutive nucleotides, at least 90 consecutive nucleotides or at least 100 consecutive nucleotides. Significant homology may be at least 50% homologous, at last 60% homologous, at least 70% homologous, at least 80% homologous, at least 90% homologous, or at least 95% homologous. The derived species may be modified while retaining an activity of the naturally-occurring variety.

### Gene Editing Methods

As discussed above, embodiments of the present disclosure provide compositions and methods to treat joint disorders, wherein a portion of the joint cells are genetically modified via gene-editing to treat a joint disorder. Embodiments of the present disclosure embrace genetic editing through nucleotide insertion (RNA or DNA), or recombinant protein insertion, into a population of synoviocytes for both promotion of the expression of one or more proteins and inhibition of the expression of one or more proteins, as well as combinations thereof. Embodiments of the present disclosure also provide methods for delivering gene-editing compositions to joint cells, and in particular delivering gene-editing compositions to synoviocytes. There are several gene-editing technologies that may be used to genetically modify joint cells, which are suitable for use in accordance with the present disclosure.

In some embodiments, a method of genetically modifying joint cells includes the step of stable incorporation of genes for production of one or more proteins. In an embodiment, a method of genetically modifying a portion of a joint's synoviocytes includes the step of retroviral transduction. In an embodiment, a method of genetically modifying a portion of a joint's synoviocytes includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a portion of a joint's synoviocytes includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a portion of a joint's synoviocytes includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some aspects, viral vectors or systems are used to introduce a gene-editing system into cells comprising a joint. In some aspects, the cells are synovial fibroblasts. In some aspects, the viral vectors are an AAV vector. In some aspects, the AAV vector comprises a serotype selected from the group consisting of: AAV1, AAV1(Y705+731F+T492V), AAV2(Y444+500+730F+T491V), AAV3(Y705+731F), AAV4, AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

In an embodiment, a method of genetically modifying a portion of a joint's synoviocytes includes the step of stable incorporation of genes for production or inhibition (e.g., silencing) of one or more proteins. In an embodiment, a method of genetically modifying a portion of a joint's synoviocytes includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Feigner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a portion of a joint's synoviocytes includes the step of transfection using methods described in U.S. Pat. Nos. 5,766,902; 6,025,337; 6,410,517; 6,475,994; and 7,189,705; the disclosures of each of which are incorporated by reference herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR-Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with the methods of the present disclosure include CRISPR methods, TALE methods, and ZFN methods, which are described in more detail below.

### CRISPR Methods

A pharmaceutical composition for the treatment or prevention of a joint disease or condition comprising a gene-editing system, wherein said gene-editing system targets at least one locus related to joint function, wherein the gene-editing at least a portion of a joint's synoviocytes by a CRISPR method (e.g., CRISPR-Cas9, CRISPR-Cas13a, or CRISPR/Cpf1 (also known as CRISPR-Cas12a). According to particular embodiments, the use of a CRISPR method to gene-edit joint synoviocytes causes expression of one or more immune checkpoint genes to be silenced or reduced in at least a portion of the joint's synoviocytes.

CRISPR stands for “Clustered Regularly Interspaced Short Palindromic Repeats.” A method of using a CRISPR system for gene editing is also referred to herein as a CRISPR method. There are three types of CRISPR systems which incorporate RNAs and Cas proteins, and which may be used in accordance with the present disclosure: Types II, V, and VI. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA, or RNA, of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR-Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR-Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR-Cas system is directly portable to human cells by co-delivery of plasmids expressing the Cas9 endo-nuclease and the necessary crRNA and tracrRNA (or sgRNA)components. Different variants of Cas proteins may be used to reduce targeting limitations (e.g., orthologs of Cas9, such as Cpf1).

### CRSIPR-Cas Mediated Homologous Recombination

The CRISPR-Cas system for homologous recombination (HR) includes a Cas nuclease (e.g., Cas9 nuclease) or a variant or fragment thereof, a DNA-targeting RNA (e.g., single guide RNA (sgRNA)) containing a guide sequence that targets the Cas nuclease to the target genomic DNA and a scaffold sequence that interacts with the Cas nuclease, and a donor template. The CRISPR-Cas system can be utilized to create a double-strand break at a desired target gene locus in the genome of a cell, and harness the cell's endogenous mechanisms to repair the induced break by homology-directed repair (HDR).

The CRISPR-Cas9 nuclease can facilitate locus-specific chromosomal integration of exogenous DNA delivered by AAV vectors. Typically, the size of the exogenous DNA (e.g., transgene, expression cassette, and the like) that can be integrated is limited by the DNA packaging capacity of an AAV vector which is about 4.0 kb. With the inclusion of two homology arms that are necessary for homologous recombination, a single AAV vector can only deliver less than about 3.7 kb of exogenous DNA. The method described herein allows for the delivery of exogenous DNA that is 4 kb or longer by splitting the nucleotide sequence between two different AAV vectors. The donor templates are designed for sequential homologous recombination events that can integrate and fuse the two parts of the nucleotide sequence.

Homologous recombination of the present disclosure can be performed using an engineered nuclease system for genome editing such as, but not limited to, CRISPR-Cas nucleases, zinc finger nucleases (ZFNs), transcription activator-like effector nucleases (TALENs), engineered mega-nucleases. In one aspect, a CRISPR-Cas-based nuclease system is used. Detailed descriptions of useful nuclease system can be found, e.g., in Gaj et al., Trends Biotechnol, 2013, July: 31(7):397-405.

Any suitable CRISPR/Cas system may be used for the methods and compositions disclosed herein. The CRISPR/Cas system may be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. The CRISPR/Cas system may be a type I, a type II, a type III, a type IV, a type V, a type VI system, or any other suitable CRISPR/Cas system. The CRISPR/Cas system as used herein may be a Class 1, Class 2, or any other suitably classified CRISPR/Cas system. The Class 1 CRISPR/Cas system may use a complex of multiple Cas proteins to effect regulation. The Class 1 CRISPR/Cas system may comprise, for example, type I (e.g., I, IA, IB, IC, ID, IE, IF, IU), type III (e.g., III, IIIA, IIIB, IIIC, HID), and type IV (e.g., IV, IVA, IVB) CRISPR/Cas type. The Class 2 CRISPR/Cas system may use a single large Cas protein to effect regulation. The Class 2 CRISPR/Cas systems may comprise, for example, type II (e.g., II, IIA, IIB) and type V CRISPR/Cas type. CRISPR systems may be complementary to each other, and/or can lend functional units in trans to facilitate CRISPR locus targeting.

In some embodiments, a nucleotide sequence encoding the Cas nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXTl, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Host cells are necessary for generating infectious AAV vectors as well as for generating AAV virions based on the disclosed AAV vectors. Various host cells are known in the art and find use in the methods of the present disclosure. Any host cells described herein or known in the art can be employed with the compositions and methods described herein.

In some embodiments, the host cell for use in generating infectious virions can be selected from any biological organism, including prokaryotic (e.g., bacterial) cells, and eukaryotic cells, including, insect cells, yeast cells and mammalian cells. A variety of cells, e.g., mammalian cells, including, e.g., murine cells, and primate cells (e.g., human cells) can be used. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, CHO, 293, Vero, NIH 3T3, PC12, Huh-7 Saos, C2C12, RAT1, Sf9, L cells, HT1080, human embryonic kidney (HEK), human embryonic stem cells, human adult tissue stem cells, pluripotent stem cells, induced pluripotent stem cells, reprogrammed stem cells, organoid stem cells, bone marrow stem cells, HLHepG2, HepG2 and primary fibroblast, hepatocyte and myoblast cells derived from mammals including human, monkey, mouse, rat, rabbit, and hamster. The requirement for the cell used is it is capable of infection or transfection by an AAV vector. In some embodiments, the host cell is one that has rep and cap stably transfected in the cell.

In some embodiments, the preparation of a host cell according to the disclosure involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods for providing the desired nucleotide sequence.

In addition to the AAV vector, the host cell can contain sequences to drive expression of the AAV capsid polypeptide (in the host cell and rep (replication) sequences of the same serotype as the serotype of the AAV Inverted Terminal Repeats (ITRs) found in the AAV vector, or a cross-complementing serotype. The AAV capsid and rep (replication) sequences may be independently obtained from an AAV source and may be introduced into the host cell in any manner known to one of skill in the art or as described herein. Additionally, when pseudotyping an AAV vector in an AAV8 capsid for example, the sequences encoding each of the essential rep (replication) proteins may be supplied by AAV8, or the sequences encoding the rep (replication) proteins may be supplied by different AAV serotypes (e.g., AAV1, AAV2, AAV3, AAV4, AAV5, AAV6, AAV7, and/or AAV9).

In some embodiments, the host cell stably contains the capsid protein under the control of a suitable promoter. In some embodiments, the capsid protein is supplied to the host cell in trans. When delivered to the host cell in trans, the capsid protein may be delivered via a plasmid containing the sequences necessary to direct expression of the selected capsid protein in the host cell. In some embodiments, when delivered to the host cell in trans, the vector encoding the capsid protein also carries other sequences required for packaging the AAV, e.g., the rep (replication) sequences.

In some embodiments, the host cell stably contains the rep (replication) sequences under the control of a suitable promoter. In another embodiment, the rep (replication) proteins are supplied to the host cell in trans. When delivered to the host cell in trans, the rep (replication) proteins may be delivered via a plasmid containing the sequences necessary to direct expression of the selected rep (replication) proteins in the host cell. In some embodiments, when delivered to the host cell in trans, the vector encoding the capsid protein (also carries other sequences required for packaging the AAV vector, e.g., the rep (replication) sequences.

In some embodiments, the rep (replication) and capsid sequences may be transfected into the host cell on a single nucleic acid molecule and exist stably in the cell as an unintegrated episome. In another embodiment, the rep (replication) and capsid sequences are stably integrated into the chromosome of the cell. Another embodiment has the rep (replication) and capsid sequences transiently expressed in the host cell. For example, a useful nucleic acid molecule for such transfection comprises, from 5′ to 3′, a promoter, an optional spacer interposed between the promoter and the start site of the rep (replication) gene sequence, an AAV rep (replication) gene sequence, and an AAV capsid gene sequence.

Although the molecule(s) providing rep (replication) and capsid can exist in the host cell transiently (i.e., through transfection), in some embodiments, one or both of the rep (replication) and capsid proteins and the promoter(s) controlling their expression be stably expressed in the host cell, e.g., as an episome or by integration into the chromosome of the host cell. The methods employed for constructing embodiments of the disclosure are conventional genetic engineering or recombinant engineering techniques such as those described in the references above.

A variety of methods of generating AAV virions are known in the art and can be used to generate AAV virions comprising the AAV vectors described herein. Generally, the methods involved inserting or transducing an AAV vector of the disclosure into a host cell capable of packaging the AAV vector into and AAV virion. Exemplary methods are described and referenced below; however, any method known to one of skill in the art can be employed to generate the AAV virions of the disclosure.

An AAV vector comprising a heterologous nucleic acid (e.g., a donor template) and used to generate an AAV virion can be constructed using methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Depending on the expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter, adenovirus major late promoter (Ad MLP), a herpes simplex virus (HSV) promoter, a cytomegalovirus (CMV) promoter (such as the CMV immediate early promoter region; CMVIE), a rous sarcoma virus (RSV) promoter, a human U6 small nuclear promoter (U6), an enhanced U6 promoter, and a human HI promoter (HI), etc.

In some embodiments, polynucleotide encoding a Cas nuclease can be used in the present disclosure. Such a polynucleotide (e.g., mRNA) can be commercially obtained from, for example, TriLink BioTechnologies, GE Dharmacon, ThermoFisher, and the like.

In certain embodiments, a Cas nuclease (e.g., Cas9 polypeptide) can be used in the present disclosure. Detailed description of useful Cas9 polypeptides can be found in, e.g., Hendel et al., Nat Biotechnol, 2015, 33(9): 985-989 and Dever et al., Nature, 2016, 539: 384-389, the disclosures are herein incorporated by reference in their entirety for all purposes.

In some embodiments, a Cas nuclease (e.g., Cas9 polypeptide) is complexed with a sgRNA to form a Cas ribonucleoprotein (e.g., Cas9 ribonucleoprotein). The molar ratio of Cas nuclease to sgRNA can be any range that facilitates sequential homologous recombination of the targeting AAV vectors and target genetic locus. In some embodiments, the molar ratio of Cas9 polypeptide to sgRNA is about 1:5; 1:4; 1:3; 1:2.5; 1:2; or 1:1. In other embodiments, the molar ratio of Cas9 polypeptide to sgRNA is about 1:2 to about 1:3. In certain embodiments, the molar ratio of Cas9 polypeptide to sgRNA is about 1:2.5.

The Cas nuclease and variants or fragments thereof can be introduced into a cell (e.g., a cell isolated from a subject, or an in vivo cell such as in a subject) as a Cas polypeptide or a variant or fragment thereof, an mRNA encoding a Cas polypeptide or a variant or fragment thereof, a recombinant expression vector comprising a nucleotide sequence encoding a Cas polypeptide or a variant or fragment thereof, or a Cas ribonucleoprotein. One skilled in the art would recognize that any method of delivering an exogenous polynucleotide, polypeptide, or a ribonucleoprotein can be used. Non-limiting examples of such methods include electroporation, nucleofection, transfection, lipofection, transduction, microinjection, electroinjection, electrofusion, nanoparticle bombardment, transformation, conjugation, and the like.

Non-limiting examples of genes that may be silenced or inhibited by permanently gene-editing synoviocytes via a CRISPR method include IL-1α, IL-1β, IL-4, IL-9, IL-10, IL-13, and TNF-α.

Non-limiting examples of genes that may be enhanced by permanently gene-editing synoviocytes via a CRISPR method include IL-1α, IL-1β, IL-4, IL-9, IL-10, IL-13, and TNF-α.

Examples of systems, methods, and compositions for altering the expression of a target gene sequence by a CRISPR method, and which may be used in accordance with embodiments of the present disclosure, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, which are incorporated by reference herein. Resources for carrying out CRISPR methods, such as plasmids for expressing CRISPR-Cas9 and CRISPR-Cpf1, are commercially available from companies such as GenScript.

In an embodiment, genetic modifications of at least a portion of a joint's synoviocytes, as described herein, may be performed using the CRISPR-Cpf1 system as described in U.S. Pat. No. 9,790,490, the disclosure of which is incorporated by reference herein.

In an embodiment, genetic modifications of at least a portion of a joint's synoviocytes, as described herein, may be performed using a CRISPR-Cas system comprising single vector systems as described in U.S. Pat. No. 9,907,863, the disclosure of which is incorporated by reference herein. TALE Methods

A pharmaceutical composition for the treatment or prevention of a joint disease or condition comprising a gene-editing system, wherein said gene-editing system targets at least one locus related to joint function, wherein the method further comprises gene-editing at least a portion of joint synoviocytes by a TALE method. According to particular embodiments, the use of a TALE method to target at least one locus related to joint function, wherein the gene-editing at least a portion of a joint's synoviocytes. Alternatively, the use of a TALE method during to target at least one locus related to joint function, wherein the gene-editing at least a portion of a joint's synoviocytes to cause expression of at least one locus related to joint function genes to be enhanced in at least a portion of the joint synoviocytes.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, Ky., USA), and Life Technologies (Grand Island, N.Y., USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing synoviocytes via a TALE method include IL-1α, IL-1β, IL-4, IL-9, IL-10, IL-13, and TNF-α.

Non-limiting examples of genes that may be enhanced by permanently gene-editing synoviocytes via a TALE method include IL-1α, IL-1β, IL-4, IL-9, IL-10, IL-13, and TNF-α.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### Zinc Finger Methods

A pharmaceutical composition for the treatment or prevention of a joint disease or condition comprising a gene-editing system, wherein said gene-editing system targets at least one locus related to joint function, wherein the method further comprises gene-editing at least a portion of joint synoviocytes by a zinc finger or zinc finger nuclease method. According to particular embodiments, the use of a zinc finger method to target at least one locus related to joint function, wherein the gene-editing at least a portion of a joint's synoviocytes. Alternatively, the use of a zinc finger method during to target at least one locus related to joint function, wherein the gene-editing at least a portion of a joint's synoviocytes to cause expression of at least one locus related to joint function genes to be enhanced in at least a portion of the joint synoviocytes.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, Calif., USA) has developed a propriety platform (CompoZr®) for zinc-finger construction in partnership with Sigma-Aldrich (St. Louis, Mo., USA).

Non-limiting examples of genes that may be silenced or inhibited by permanently gene-editing synoviocytes via a zinc finger method include IL-1α, IL-1β, IL-4, IL-9, IL-10, IL-13, TNF-α. IL-6, IL-8, IL-18, a matrix metalloproteinase (MMP), or a component of the NLRP3 inflammasome. In some embodiments, the component of the NLRP3 inflammasome comprises NLRP3, ASC (apoptosis-associated speck-like protein containing a CARD), caspase-1, and combinations thereof.

Non-limiting examples of genes that may be enhanced by permanently gene-editing synoviocytes via a zinc finger method include group comprising IL-1Ra, TIMP-1, TIMP-2, TIMP-3, TIMP-4, and combinations thereof. In an aspect, the disclosure provides compositions for up-regulation of anti-inflammatory cytokines.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

In some aspects, cells may be gene-edited ex vivo, wherein the gene-editing targets one or more anti-inflammatory cytokine locus. In some aspects, the cells are non-synovial cells. In some aspects, the cells are mesenchymal stem cells. In some aspect, the cells are macrophages. In some aspects, the present disclosure provides for a pharmaceutical composition for the treatment or prevention of a joint disease or condition comprising a population of gene-edited cells, wherein said gene-edited cells are edited by a gene-editing system targeting at least one locus related to joint function. In an aspect, the population of gene-edited cells are injected into a synovial joint.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### Example Embodiments

In some embodiments, the present disclosure provides a pharmaceutical composition for the treatment or prevention of a joint disease or condition, the composition including a therapeutically effective amount of one or more nucleic acids encoding a Clustered Regularly Interspaced Short Palindromic Repeats (CRISPR) gene-editing system. The system includes a CRISPR Associated Protein 9 (Cas9) protein, and at least one guide RNA targeting an IL-1α or IL-1β gene, wherein the target sequence is adjacent to a protospacer adjacent motif (PAM) sequence for the Cas9 protein.

In some embodiments, the at least one guide RNA targets a human IL-1α gene, and includes a crRNA sequence that is complementary to a target sequence in exon 2 of the IL-1α gene. In some embodiments, the crRNA sequence forms no more than 5 mismatches with the target sequence in exon 2 of the human IL-1α gene. In some embodiments, the crRNA sequence forms no more than 4 mismatches with the target sequence in exon 2 of the human IL-1α gene. In some embodiments, the crRNA sequence forms no more than 3 mismatches with the target sequence in exon 2 of the human IL-1α gene. In some embodiments, the crRNA sequence forms no more than 2 mismatches with the target sequence in exon 2 of the human IL-1α gene. In some embodiments, the crRNA sequence forms no more than 1 mismatch with the target sequence in exon 2 of the human IL-1α gene. In some embodiments, the crRNA sequence forms no mismatches with the target sequence in exon 2 of the human IL-1α gene.

In some embodiments, the at least one guide RNA targets a human IL-1α gene, and includes a crRNA sequence that is complementary to a target sequence in exon 3 of the IL-1α gene. In some embodiments, the crRNA sequence forms no more than 5 mismatches with the target sequence in exon 3 of the human IL-1α gene. In some embodiments, the crRNA sequence forms no more than 4 mismatches with the target sequence in exon 3 of the human IL-1α gene. In some embodiments, the crRNA sequence forms no more than 3 mismatches with the target sequence in exon 3 of the human IL-1α gene. In some embodiments, the crRNA sequence forms no more than 2 mismatches with the target sequence in exon 3 of the human IL-1α gene. In some embodiments, the crRNA sequence forms no more than 1 mismatch with the target sequence in exon 3 of the human IL-1α gene. In some embodiments, the crRNA sequence forms no mismatches with the target sequence in exon 3 of the human IL-1α gene.

In some embodiments, the at least one guide RNA targets a human IL-1α gene, and includes a crRNA sequence that is complementary to a target sequence in exon 4 of the IL-1α gene. In some embodiments, the crRNA sequence forms no more than 5 mismatches with the target sequence in exon 4 of the human IL-1α gene. In some embodiments, the crRNA sequence forms no more than 4 mismatches with the target sequence in exon 4 of the human IL-1α gene. In some embodiments, the crRNA sequence forms no more than 3 mismatches with the target sequence in exon 4 of the human IL-1α gene. In some embodiments, the crRNA sequence forms no more than 2 mismatches with the target sequence in exon 4 of the human IL-1α gene. In some embodiments, the crRNA sequence forms no more than 1 mismatch with the target sequence in exon 4 of the human IL-1α gene. In some embodiments, the crRNA sequence forms no mismatches with the target sequence in exon 4 of the human IL-1α gene.

In some embodiments, the at least one guide RNA targets a human IL-1α gene, and includes a crRNA sequence that is complementary to a target sequence in exon 5 of the IL-1α gene. In some embodiments, the crRNA sequence forms no more than 5 mismatches with the target sequence in exon 5 of the human IL-1α gene. In some embodiments, the crRNA sequence forms no more than 4 mismatches with the target sequence in exon 5 of the human IL-1α gene. In some embodiments, the crRNA sequence forms no more than 3 mismatches with the target sequence in exon 5 of the human IL-1α gene. In some embodiments, the crRNA sequence forms no more than 2 mismatches with the target sequence in exon 5 of the human IL-1α gene. In some embodiments, the crRNA sequence forms no more than 1 mismatch with the target sequence in exon 5 of the human IL-1α gene. In some embodiments, the crRNA sequence forms no mismatches with the target sequence in exon 5 of the human IL-1α gene.

In some embodiments, the at least one guide RNA targets a human IL-1α gene, and includes a crRNA sequence that is complementary to a target sequence in exon 6 of the IL-1α gene. In some embodiments, the crRNA sequence forms no more than 5 mismatches with the target sequence in exon 6 of the human IL-1α gene. In some embodiments, the crRNA sequence forms no more than 4 mismatches with the target sequence in exon 6 of the human IL-1α gene. In some embodiments, the crRNA sequence forms no more than 3 mismatches with the target sequence in exon 6 of the human IL-1α gene. In some embodiments, the crRNA sequence forms no more than 2 mismatches with the target sequence in exon 6 of the human IL-1α gene. In some embodiments, the crRNA sequence forms no more than 1 mismatch with the target sequence in exon 6 of the human IL-1α gene. In some embodiments, the crRNA sequence forms no mismatches with the target sequence in exon 6 of the human IL-1α gene.

In some embodiments, the at least one guide RNA targets a human IL-1α gene, and includes a crRNA sequence that is complementary to a target sequence in exon 7 of the IL-1α gene. In some embodiments, the crRNA sequence forms no more than 5 mismatches with the target sequence in exon 7 of the human IL-1α gene. In some embodiments, the crRNA sequence forms no more than 4 mismatches with the target sequence in exon 7 of the human IL-1α gene. In some embodiments, the crRNA sequence forms no more than 3 mismatches with the target sequence in exon 7 of the human IL-1α gene. In some embodiments, the crRNA sequence forms no more than 2 mismatches with the target sequence in exon 7 of the human IL-1α gene. In some embodiments, the crRNA sequence forms no more than 1 mismatch with the target sequence in exon 7 of the human IL-1α gene. In some embodiments, the crRNA sequence forms no mismatches with the target sequence in exon 7 of the human IL-1α gene.

In some embodiments, the at least one guide RNA targets a human IL-1α gene, and includes a crRNA sequence that is complementary to a target sequence in exon 2 or exon 3 of the IL-1α gene. In some embodiments, the at least one guide RNA targets a human IL-1α gene, and includes a crRNA sequence that is complementary to a target sequence in exon 2 or exon 4 of the IL-1α gene. In some embodiments, the at least one guide RNA targets a human IL-1α gene, and includes a crRNA sequence that is complementary to a target sequence in exon 2 or exon 5 of the IL-1α gene. In some embodiments, the at least one guide RNA targets a human IL-1α gene, and includes a crRNA sequence that is complementary to a target sequence in exon 2 or exon 6 of the IL-1α gene. In some embodiments, the at least one guide RNA targets a human IL-1α gene, and includes a crRNA sequence that is complementary to a target sequence in exon 2 or exon 7 of the IL-1α gene.

In some embodiments, the at least one guide RNA targets a human IL-1α gene, and includes a crRNA sequence that is complementary to a target sequence in exon 3 or exon 4 of the IL-1α gene. In some embodiments, the at least one guide RNA targets a human IL-1α gene, and includes a crRNA sequence that is complementary to a target sequence in exon 3 or exon 5 of the IL-1α gene. In some embodiments, the at least one guide RNA targets a human IL-1α gene, and includes a crRNA sequence that is complementary to a target sequence in exon 3 or exon 6 of the IL-1α gene. In some embodiments, the at least one guide RNA targets a human IL-1α gene, and includes a crRNA sequence that is complementary to a target sequence in exon 3 or exon 7 of the IL-1α gene.

In some embodiments, the at least one guide RNA targets a human IL-1α gene, and includes a crRNA sequence that is complementary to a target sequence in exon 4 or exon 5 of the IL-1α gene. In some embodiments, the at least one guide RNA targets a human IL-1α gene, and includes a crRNA sequence that is complementary to a target sequence in exon 4 or exon 6 of the IL-1α gene. In some embodiments, the at least one guide RNA targets a human IL-1α gene, and includes a crRNA sequence that is complementary to a target sequence in exon 4 or exon 7 of the IL-1α gene.

In some embodiments, the at least one guide RNA targets a human IL-1α gene, and includes a crRNA sequence that is complementary to a target sequence in exon 5 or exon 6 of the IL-1α gene. In some embodiments, the at least one guide RNA targets a human IL-1α gene, and includes a crRNA sequence that is complementary to a target sequence in exon 5 or exon 7 of the IL-1α gene. In some embodiments, the at least one guide RNA targets a human IL-1α gene, and includes a crRNA sequence that is complementary to a target sequence in exon 6 or exon 7 of the IL-1α gene.

In some embodiments, the at least one guide RNA targets a human IL-1α gene, and includes a crRNA sequence that is complementary to a target sequence in exon 2, exon 3, or exon 4 of the IL-1α gene. In some embodiments, the at least one guide RNA targets a human IL-1α gene, and includes a crRNA sequence that is complementary to a target sequence in exon 2, exon 3, or exon 5 of the IL-1α gene. In some embodiments, the at least one guide RNA targets a human IL-1α gene, and includes a crRNA sequence that is complementary to a target sequence in exon 2, exon 3, or exon 6 of the IL-1α gene. In some embodiments, the at least one guide RNA targets a human IL-1α gene, and includes a crRNA sequence that is complementary to a target sequence in exon 2, exon 3, or exon 7 of the IL-1α gene. In some embodiments, the at least one guide RNA targets a human IL-1α gene, and includes a crRNA sequence that is complementary to a target sequence in exon 2, exon 4, or exon 5 of the IL-1α gene. In some embodiments, the at least one guide RNA targets a human IL-1α gene, and includes a crRNA sequence that is complementary to a target sequence in exon 2, exon 4, or exon 6 of the IL-1α gene. In some embodiments, the at least one guide RNA targets a human IL-1α gene, and includes a crRNA sequence that is complementary to a target sequence in exon 2, exon 4, or exon 7 of the IL-1α gene. In some embodiments, the at least one guide RNA targets a human IL-1α gene, and includes a crRNA sequence that is complementary to a target sequence in exon 2, exon 5, or exon 6 of the IL-1α gene. In some embodiments, the at least one guide RNA targets a human IL-1α gene, and includes a crRNA sequence that is complementary to a target sequence in exon 2, exon 5, or exon 7 of the IL-1α gene. In some embodiments, the at least one guide RNA targets a human IL-1α gene, and includes a crRNA sequence that is complementary to a target sequence in exon 2, exon 6, or exon 7 of the IL-1α gene.

In some embodiments, the at least one guide RNA targets a human IL-1α gene, and includes a crRNA sequence that is complementary to a target sequence in exon 3, exon 4, or exon 5 of the IL-1α gene. In some embodiments, the at least one guide RNA targets a human IL-1α gene, and includes a crRNA sequence that is complementary to a target sequence in exon 3, exon 4, or exon 6 of the IL-1α gene. In some embodiments, the at least one guide RNA targets a human IL-1α gene, and includes a crRNA sequence that is complementary to a target sequence in exon 3, exon 4, or exon 7 of the IL-1α gene. In some embodiments, the at least one guide RNA targets a human IL-1α gene, and includes a crRNA sequence that is complementary to a target sequence in exon 3, exon 5, or exon 6 of the IL-1α gene. In some embodiments, the at least one guide RNA targets a human IL-1α gene, and includes a crRNA sequence that is complementary to a target sequence in exon 3, exon 5, or exon 7 of the IL-1α gene. In some embodiments, the at least one guide RNA targets a human IL-1α gene, and includes a crRNA sequence that is complementary to a target sequence in exon 3, exon 6, or exon 7 of the IL-1α gene.

In some embodiments, the at least one guide RNA targets a human IL-1α gene, and includes a crRNA sequence that is complementary to a target sequence in exon 4, exon 5, or exon 6 of the IL-1α gene. In some embodiments, the at least one guide RNA targets a human IL-1α gene, and includes a crRNA sequence that is complementary to a target sequence in exon 4, exon 5, or exon 7 of the IL-1α gene. In some embodiments, the at least one guide RNA targets a human IL-1α gene, and includes a crRNA sequence that is complementary to a target sequence in exon 4, exon 6, or exon 7 of the IL-1α gene. In some embodiments, the at least one guide RNA targets a human IL-1α gene, and includes a crRNA sequence that is complementary to a target sequence in exon 5, exon 6, or exon 7 of the IL-1α gene.

In some embodiments, the at least one guide RNA targets a human IL-1α gene, and includes a crRNA sequence that is complementary to a target sequence in exon 2, exon 3, exon 4, or exon 5 of the IL-1α gene. In some embodiments, the at least one guide RNA targets a human IL-1α gene, and includes a crRNA sequence that is complementary to a target sequence in exon 2, exon 3, exon 4, or exon 6 of the IL-1α gene. In some embodiments, the at least one guide RNA targets a human IL-1α gene, and includes a crRNA sequence that is complementary to a target sequence in exon 2, exon 3, exon 4, or exon 7 of the IL-1α gene. In some embodiments, the at least one guide RNA targets a human IL-1α gene, and includes a crRNA sequence that is complementary to a target sequence in exon 2, exon 4, exon 5, or exon 6 of the IL-1α gene. In some embodiments, the at least one guide RNA targets a human IL-1α gene, and includes a crRNA sequence that is complementary to a target sequence in exon 2, exon 4, exon 5, or exon 7 of the IL-1α gene. In some embodiments, the at least one guide RNA targets a human IL-1α gene, and includes a crRNA sequence that is complementary to a target sequence in exon 2, exon 4, exon 6, or exon 7 of the IL-1α gene. In some embodiments, the at least one guide RNA targets a human IL-1α gene, and includes a crRNA sequence that is complementary to a target sequence in exon 2, exon 5, exon 6, or exon 7 of the IL-1α gene.

In some embodiments, the at least one guide RNA targets a human IL-1α gene, and includes a crRNA sequence that is complementary to a target sequence in exon 3, exon 4, exon 5, or exon 6 of the IL-1α gene. In some embodiments, the at least one guide RNA targets a human IL-1α gene, and includes a crRNA sequence that is complementary to a target sequence in exon 3, exon 4, exon 5, or exon 7 of the IL-1α gene. In some embodiments, the at least one guide RNA targets a human IL-1α gene, and includes a crRNA sequence that is complementary to a target sequence in exon 3, exon 4, exon 6, or exon 7 of the IL-1α gene. In some embodiments, the at least one guide RNA targets a human IL-1α gene, and includes a crRNA sequence that is complementary to a target sequence in exon 3, exon 5, exon 6, or exon 7 of the IL-1α gene. In some embodiments, the at least one guide RNA targets a human IL-1α gene, and includes a crRNA sequence that is complementary to a target sequence in exon 4, exon 5, exon 6, or exon 7 of the IL-1α gene.

In some embodiments, the at least one guide RNA targets a human IL-1α gene, and includes a crRNA sequence that is complementary to a target sequence in exon 2, exon 3, exon 4, exon 5, or exon 6 of the IL-1α gene. In some embodiments, the at least one guide RNA targets a human IL-1α gene, and includes a crRNA sequence that is complementary to a target sequence in exon 2, exon 3, exon 4, exon 5, or exon 7 of the IL-1α gene. In some embodiments, the at least one guide RNA targets a human IL-1α gene, and includes a crRNA sequence that is complementary to a target sequence in exon 3, exon 4, exon 5, exon 6, or exon 7 of the IL-1α gene. In some embodiments, the at least one guide RNA targets a human IL-1α gene, and includes a crRNA sequence that is complementary to a target sequence in exon 2, exon 4, exon 5, exon 6, or exon 7 of the IL-1α gene. In some embodiments, the at least one guide RNA targets a human IL-1α gene, and includes a crRNA sequence that is complementary to a target sequence in exon 2, exon 3, exon 5, exon 6, or exon 6 of the IL-1α gene. In some embodiments, the at least one guide RNA targets a human IL-1α gene, and includes a crRNA sequence that is complementary to a target sequence in exon 2, exon 3, exon 4, exon 6, or exon 7 of the IL-1α gene. In some embodiments, the at least one guide RNA targets a human IL-1α gene, and includes a crRNA sequence that is complementary to a target sequence in exon 2, exon 3, exon 4, exon 5, exon 6, or exon 7 of the IL-1α gene.

In some embodiments, the at least one guide RNA targets a human IL-1α gene, and includes a crRNA sequence having at least 75% identity to a sequence selected from the group consisting of SEQ ID NOs: 298-387. In some embodiments, the crRNA sequence has at least 80% identity to a sequence selected from the group consisting of SEQ ID NOs: 298-387. In some embodiments, the crRNA sequence has at least 85% identity to a sequence selected from the group consisting of SEQ ID NOs: 298-387. In some embodiments, the crRNA sequence has at least 90% identity to a sequence selected from the group consisting of SEQ ID NOs: 298-387. In some embodiments, the crRNA sequence has at least 95% identity to a sequence selected from the group consisting of SEQ ID NOs: 298-387. In some embodiments, the crRNA sequence is selected from the group consisting of SEQ ID NOs: 298-387.

In some embodiments, the at least one guide RNA targets a human IL-1α gene, and includes a crRNA sequence having at least 75% identity to SEQ ID NO:301. In some embodiments, the crRNA sequence has at least 80% identity to SEQ ID NO:301. In some embodiments, the crRNA sequence has at least 85% identity to SEQ ID NO:301. In some embodiments, the crRNA sequence has at least 90% identity to SEQ ID NO:301. In some embodiments, the crRNA sequence has at least 95% identity to SEQ ID NO:301. In some embodiments, the crRNA sequence is SEQ ID NO:301.

In some embodiments, the at least one guide RNA targets a human IL-1α gene, and includes a crRNA sequence having at least 75% identity to SEQ ID NO:309. In some embodiments, the crRNA sequence has at least 80% identity to SEQ ID NO:309. In some embodiments, the crRNA sequence has at least 85% identity to SEQ ID NO:309. In some embodiments, the crRNA sequence has at least 90% identity to SEQ ID NO:309. In some embodiments, the crRNA sequence has at least 95% identity to SEQ ID NO:309. In some embodiments, the crRNA sequence is SEQ ID NO:309.

In some embodiments, the at least one guide RNA targets a human IL-1β gene, and includes a crRNA sequence having at least 75% identity to a sequence selected from the group consisting of SEQ ID NOs: 388-496. In some embodiments, the crRNA sequence has at least 80% identity to a sequence selected from the group consisting of SEQ ID NOs: 388-496. In some embodiments, the crRNA sequence has at least 85% identity to a sequence selected from the group consisting of SEQ ID NOs: 388-496. In some embodiments, the crRNA sequence has at least 90% identity to a sequence selected from the group consisting of SEQ ID NOs: 388-496. In some embodiments, the crRNA sequence has at least 95% identity to a sequence selected from the group consisting of SEQ ID NOs: 388-496. In some embodiments, the crRNA sequence is selected from the group consisting of SEQ ID NOs: 388-496.

In some embodiments, the at least one guide RNA targets a human IL-1β gene, and includes a crRNA sequence that is complementary to a target sequence in exon 2 of the IL-1β gene. In some embodiments, the crRNA sequence forms no more than 5 mismatches with the target sequence in exon 2 of the human IL-1β gene. In some embodiments, the crRNA sequence forms no more than 4 mismatches with the target sequence in exon 2 of the human IL-1β gene. In some embodiments, the crRNA sequence forms no more than 3 mismatches with the target sequence in exon 2 of the human IL-1β gene. In some embodiments, the crRNA sequence forms no more than 2 mismatches with the target sequence in exon 2 of the human IL-1β gene. In some embodiments, the crRNA sequence forms no more than 1 mismatch with the target sequence in exon 2 of the human IL-1β gene. In some embodiments, the crRNA sequence forms no mismatches with the target sequence in exon 2 of the human IL-1β gene.

In some embodiments, the at least one guide RNA targets a human IL-1β gene, and includes a crRNA sequence that is complementary to a target sequence in exon 3 of the IL-1β gene. In some embodiments, the crRNA sequence forms no more than 5 mismatches with the target sequence in exon 3 of the human IL-1β gene. In some embodiments, the crRNA sequence forms no more than 4 mismatches with the target sequence in exon 3 of the human IL-1β gene. In some embodiments, the crRNA sequence forms no more than 3 mismatches with the target sequence in exon 3 of the human IL-1β gene. In some embodiments, the crRNA sequence forms no more than 2 mismatches with the target sequence in exon 3 of the human IL-1β gene. In some embodiments, the crRNA sequence forms no more than 1 mismatch with the target sequence in exon 3 of the human IL-1β gene. In some embodiments, the crRNA sequence forms no mismatches with the target sequence in exon 3 of the human IL-1β gene.

In some embodiments, the at least one guide RNA targets a human IL-1β gene, and includes a crRNA sequence that is complementary to a target sequence in exon 4 of the IL-1β gene. In some embodiments, the crRNA sequence forms no more than 5 mismatches with the target sequence in exon 4 of the human IL-1β gene. In some embodiments, the crRNA sequence forms no more than 4 mismatches with the target sequence in exon 4 of the human IL-1β gene. In some embodiments, the crRNA sequence forms no more than 3 mismatches with the target sequence in exon 4 of the human IL-1β gene. In some embodiments, the crRNA sequence forms no more than 2 mismatches with the target sequence in exon 4 of the human IL-1β gene. In some embodiments, the crRNA sequence forms no more than 1 mismatch with the target sequence in exon 4 of the human IL-1β gene. In some embodiments, the crRNA sequence forms no mismatches with the target sequence in exon 4 of the human IL-1β gene.

In some embodiments, the at least one guide RNA targets a human IL-1β gene, and includes a crRNA sequence that is complementary to a target sequence in exon 5 of the IL-1β gene. In some embodiments, the crRNA sequence forms no more than 5 mismatches with the target sequence in exon 5 of the human IL-1β gene. In some embodiments, the crRNA sequence forms no more than 4 mismatches with the target sequence in exon 5 of the human IL-1β gene. In some embodiments, the crRNA sequence forms no more than 3 mismatches with the target sequence in exon 5 of the human IL-1β gene. In some embodiments, the crRNA sequence forms no more than 2 mismatches with the target sequence in exon 5 of the human IL-1β gene. In some embodiments, the crRNA sequence forms no more than 1 mismatch with the target sequence in exon 5 of the human IL-1β gene. In some embodiments, the crRNA sequence forms no mismatches with the target sequence in exon 5 of the human IL-1β gene.

In some embodiments, the at least one guide RNA targets a human IL-1β gene, and includes a crRNA sequence that is complementary to a target sequence in exon 6 of the IL-1β gene. In some embodiments, the crRNA sequence forms no more than 5 mismatches with the target sequence in exon 6 of the human IL-1β gene. In some embodiments, the crRNA sequence forms no more than 4 mismatches with the target sequence in exon 6 of the human IL-1β gene. In some embodiments, the crRNA sequence forms no more than 3 mismatches with the target sequence in exon 6 of the human IL-1β gene. In some embodiments, the crRNA sequence forms no more than 2 mismatches with the target sequence in exon 6 of the human IL-1β gene. In some embodiments, the crRNA sequence forms no more than 1 mismatch with the target sequence in exon 6 of the human IL-1β gene. In some embodiments, the crRNA sequence forms no mismatches with the target sequence in exon 6 of the human IL-1β gene.

In some embodiments, the at least one guide RNA targets a human IL-1β gene, and includes a crRNA sequence that is complementary to a target sequence in exon 7 of the IL-1β gene. In some embodiments, the crRNA sequence forms no more than 5 mismatches with the target sequence in exon 7 of the human IL-1β gene. In some embodiments, the crRNA sequence forms no more than 4 mismatches with the target sequence in exon 7 of the human IL-1β gene. In some embodiments, the crRNA sequence forms no more than 3 mismatches with the target sequence in exon 7 of the human IL-1β gene. In some embodiments, the crRNA sequence forms no more than 2 mismatches with the target sequence in exon 7 of the human IL-1β gene. In some embodiments, the crRNA sequence forms no more than 1 mismatch with the target sequence in exon 7 of the human IL-1β gene. In some embodiments, the crRNA sequence forms no mismatches with the target sequence in exon 7 of the human IL-1β gene.

In some embodiments, the at least one guide RNA targets a human IL-1β gene, and includes a crRNA sequence that is complementary to a target sequence in exon 2 or exon 3 of the IL-1β gene. In some embodiments, the at least one guide RNA targets a human IL-1β gene, and includes a crRNA sequence that is complementary to a target sequence in exon 2 or exon 4 of the IL-1β gene. In some embodiments, the at least one guide RNA targets a human IL-1β gene, and includes a crRNA sequence that is complementary to a target sequence in exon 2 or exon 5 of the IL-1β gene. In some embodiments, the at least one guide RNA targets a human IL-1β gene, and includes a crRNA sequence that is complementary to a target sequence in exon 2 or exon 6 of the IL-1β gene. In some embodiments, the at least one guide RNA targets a human IL-1β gene, and includes a crRNA sequence that is complementary to a target sequence in exon 2 or exon 7 of the IL-1β gene.

In some embodiments, the at least one guide RNA targets a human IL-1β gene, and includes a crRNA sequence that is complementary to a target sequence in exon 3 or exon 4 of the IL-1β gene. In some embodiments, the at least one guide RNA targets a human IL-1β gene, and includes a crRNA sequence that is complementary to a target sequence in exon 3 or exon 5 of the IL-1β gene. In some embodiments, the at least one guide RNA targets a human IL-1β gene, and includes a crRNA sequence that is complementary to a target sequence in exon 3 or exon 6 of the IL-1β gene. In some embodiments, the at least one guide RNA targets a human IL-1β gene, and includes a crRNA sequence that is complementary to a target sequence in exon 3 or exon 7 of the IL-1β gene.

In some embodiments, the at least one guide RNA targets a human IL-1β gene, and includes a crRNA sequence that is complementary to a target sequence in exon 4 or exon 5 of the IL-1β gene. In some embodiments, the at least one guide RNA targets a human IL-1β gene, and includes a crRNA sequence that is complementary to a target sequence in exon 4 or exon 6 of the IL-1β gene. In some embodiments, the at least one guide RNA targets a human IL-1β gene, and includes a crRNA sequence that is complementary to a target sequence in exon 4 or exon 7 of the IL-1β gene.

In some embodiments, the at least one guide RNA targets a human IL-1β gene, and includes a crRNA sequence that is complementary to a target sequence in exon 5 or exon 6 of the IL-1β gene. In some embodiments, the at least one guide RNA targets a human IL-1β gene, and includes a crRNA sequence that is complementary to a target sequence in exon 5 or exon 7 of the IL-1β gene. In some embodiments, the at least one guide RNA targets a human IL-1β gene, and includes a crRNA sequence that is complementary to a target sequence in exon 6 or exon 7 of the IL-1β gene.

In some embodiments, the at least one guide RNA targets a human IL-1β gene, and includes a crRNA sequence that is complementary to a target sequence in exon 2, exon 3, or exon 4 of the IL-1β gene. In some embodiments, the at least one guide RNA targets a human IL-1β gene, and includes a crRNA sequence that is complementary to a target sequence in exon 2, exon 3, or exon 5 of the IL-1β gene. In some embodiments, the at least one guide RNA targets a human IL-1β gene, and includes a crRNA sequence that is complementary to a target sequence in exon 2, exon 3, or exon 6 of the IL-1β gene. In some embodiments, the at least one guide RNA targets a human IL-1β gene, and includes a crRNA sequence that is complementary to a target sequence in exon 2, exon 3, or exon 7 of the IL-1β gene. In some embodiments, the at least one guide RNA targets a human IL-1β gene, and includes a crRNA sequence that is complementary to a target sequence in exon 2, exon 4, or exon 5 of the IL-1β gene. In some embodiments, the at least one guide RNA targets a human IL-1β gene, and includes a crRNA sequence that is complementary to a target sequence in exon 2, exon 4, or exon 6 of the IL-1β gene. In some embodiments, the at least one guide RNA targets a human IL-1β gene, and includes a crRNA sequence that is complementary to a target sequence in exon 2, exon 4, or exon 7 of the IL-1β gene. In some embodiments, the at least one guide RNA targets a human IL-1β gene, and includes a crRNA sequence that is complementary to a target sequence in exon 2, exon 5, or exon 6 of the IL-1β gene. In some embodiments, the at least one guide RNA targets a human IL-1β gene, and includes a crRNA sequence that is complementary to a target sequence in exon 2, exon 5, or exon 7 of the IL-1β gene. In some embodiments, the at least one guide RNA targets a human IL-1β gene, and includes a crRNA sequence that is complementary to a target sequence in exon 2, exon 6, or exon 7 of the IL-1β gene.

In some embodiments, the at least one guide RNA targets a human IL-1β gene, and includes a crRNA sequence that is complementary to a target sequence in exon 3, exon 4, or exon 5 of the IL-1β gene. In some embodiments, the at least one guide RNA targets a human IL-1β gene, and includes a crRNA sequence that is complementary to a target sequence in exon 3, exon 4, or exon 6 of the IL-1β gene. In some embodiments, the at least one guide RNA targets a human IL-1β gene, and includes a crRNA sequence that is complementary to a target sequence in exon 3, exon 4, or exon 7 of the IL-1β gene. In some embodiments, the at least one guide RNA targets a human IL-1β gene, and includes a crRNA sequence that is complementary to a target sequence in exon 3, exon 5, or exon 6 of the IL-1β gene. In some embodiments, the at least one guide RNA targets a human IL-1β gene, and includes a crRNA sequence that is complementary to a target sequence in exon 3, exon 5, or exon 7 of the IL-1β gene. In some embodiments, the at least one guide RNA targets a human IL-1β gene, and includes a crRNA sequence that is complementary to a target sequence in exon 3, exon 6, or exon 7 of the IL-1β gene.

In some embodiments, the at least one guide RNA targets a human IL-1β gene, and includes a crRNA sequence that is complementary to a target sequence in exon 4, exon 5, or exon 6 of the IL-1β gene. In some embodiments, the at least one guide RNA targets a human IL-1β gene, and includes a crRNA sequence that is complementary to a target sequence in exon 4, exon 5, or exon 7 of the IL-1β gene. In some embodiments, the at least one guide RNA targets a human IL-1β gene, and includes a crRNA sequence that is complementary to a target sequence in exon 4, exon 6, or exon 7 of the IL-1β gene. In some embodiments, the at least one guide RNA targets a human IL-1β gene, and includes a crRNA sequence that is complementary to a target sequence in exon 5, exon 6, or exon 7 of the IL-1β gene.

In some embodiments, the at least one guide RNA targets a human IL-1β gene, and includes a crRNA sequence that is complementary to a target sequence in exon 2, exon 3, exon 4, or exon 5 of the IL-1β gene. In some embodiments, the at least one guide RNA targets a human IL-1β gene, and includes a crRNA sequence that is complementary to a target sequence in exon 2, exon 3, exon 4, or exon 6 of the IL-1β gene. In some embodiments, the at least one guide RNA targets a human IL-1β gene, and includes a crRNA sequence that is complementary to a target sequence in exon 2, exon 3, exon 4, or exon 7 of the IL-1β gene. In some embodiments, the at least one guide RNA targets a human IL-1β gene, and includes a crRNA sequence that is complementary to a target sequence in exon 2, exon 4, exon 5, or exon 6 of the IL-1β gene. In some embodiments, the at least one guide RNA targets a human IL-1β gene, and includes a crRNA sequence that is complementary to a target sequence in exon 2, exon 4, exon 5, or exon 7 of the IL-1β gene. In some embodiments, the at least one guide RNA targets a human IL-1β gene, and includes a crRNA sequence that is complementary to a target sequence in exon 2, exon 4, exon 6, or exon 7 of the IL-1β gene. In some embodiments, the at least one guide RNA targets a human IL-1β gene, and includes a crRNA sequence that is complementary to a target sequence in exon 2, exon 5, exon 6, or exon 7 of the IL-1β gene.

In some embodiments, the at least one guide RNA targets a human IL-1β gene, and includes a crRNA sequence that is complementary to a target sequence in exon 3, exon 4, exon 5, or exon 6 of the IL-1β gene. In some embodiments, the at least one guide RNA targets a human IL-1β gene, and includes a crRNA sequence that is complementary to a target sequence in exon 3, exon 4, exon 5, or exon 7 of the IL-1β gene. In some embodiments, the at least one guide RNA targets a human IL-1β gene, and includes a crRNA sequence that is complementary to a target sequence in exon 3, exon 4, exon 6, or exon 7 of the IL-1β gene. In some embodiments, the at least one guide RNA targets a human IL-1β gene, and includes a crRNA sequence that is complementary to a target sequence in exon 3, exon 5, exon 6, or exon 7 of the IL-1β gene. In some embodiments, the at least one guide RNA targets a human IL-1β gene, and includes a crRNA sequence that is complementary to a target sequence in exon 4, exon 5, exon 6, or exon 7 of the IL-1β gene.

In some embodiments, the at least one guide RNA targets a human IL-1β gene, and includes a crRNA sequence that is complementary to a target sequence in exon 2, exon 3, exon 4, exon 5, or exon 6 of the IL-1β gene. In some embodiments, the at least one guide RNA targets a human IL-1β gene, and includes a crRNA sequence that is complementary to a target sequence in exon 2, exon 3, exon 4, exon 5, or exon 7 of the IL-1β gene. In some embodiments, the at least one guide RNA targets a human IL-1β gene, and includes a crRNA sequence that is complementary to a target sequence in exon 3, exon 4, exon 5, exon 6, or exon 7 of the IL-1β gene. In some embodiments, the at least one guide RNA targets a human IL-1β gene, and includes a crRNA sequence that is complementary to a target sequence in exon 2, exon 4, exon 5, exon 6, or exon 7 of the IL-1β gene. In some embodiments, the at least one guide RNA targets a human IL-1β gene, and includes a crRNA sequence that is complementary to a target sequence in exon 2, exon 3, exon 5, exon 6, or exon 6 of the IL-1β gene. In some embodiments, the at least one guide RNA targets a human IL-1β gene, and includes a crRNA sequence that is complementary to a target sequence in exon 2, exon 3, exon 4, exon 6, or exon 7 of the IL-1β gene. In some embodiments, the at least one guide RNA targets a human IL-1β gene, and includes a crRNA sequence that is complementary to a target sequence in exon 2, exon 3, exon 4, exon 5, exon 6, or exon 7 of the IL-1β gene.

In some embodiments, the at least one guide RNA targets a human IL-1β gene, and includes a crRNA sequence having at least 75% identity to SEQ ID NO:462. In some embodiments, the crRNA sequence has at least 80% identity to SEQ ID NO:462. In some embodiments, the crRNA sequence has at least 85% identity to SEQ ID NO:462. In some embodiments, the crRNA sequence has at least 90% identity to SEQ ID NO:462. In some embodiments, the crRNA sequence has at least 95% identity to SEQ ID NO:462. In some embodiments, the crRNA sequence is SEQ ID NO:462.

In some embodiments, the at least one guide RNA targets a human IL-1β gene, and includes a crRNA sequence having at least 75% identity to SEQ ID NO:391. In some embodiments, the crRNA sequence has at least 80% identity to SEQ ID NO:391. In some embodiments, the crRNA sequence has at least 85% identity to SEQ ID NO:391. In some embodiments, the crRNA sequence has at least 90% identity to SEQ ID NO:391. In some embodiments, the crRNA sequence has at least 95% identity to SEQ ID NO:391. In some embodiments, the crRNA sequence is SEQ ID NO:391.

In some embodiments, the at least one guide RNA targets a human IL-1β gene, and includes a crRNA sequence having at least 75% identity to SEQ ID NO:393. In some embodiments, the crRNA sequence has at least 80% identity to SEQ ID NO:393. In some embodiments, the crRNA sequence has at least 85% identity to SEQ ID NO:393. In some embodiments, the crRNA sequence has at least 90% identity to SEQ ID NO:393. In some embodiments, the crRNA sequence has at least 95% identity to SEQ ID NO:393. In some embodiments, the crRNA sequence is SEQ ID NO:393.

In some embodiments, the at least one guide RNA targets a human IL-1β gene, and includes a crRNA sequence having at least 75% identity to SEQ ID NO:388. In some embodiments, the crRNA sequence has at least 80% identity to SEQ ID NO:388. In some embodiments, the crRNA sequence has at least 85% identity to SEQ ID NO:388. In some embodiments, the crRNA sequence has at least 90% identity to SEQ ID NO:388. In some embodiments, the crRNA sequence has at least 95% identity to SEQ ID NO:388. In some embodiments, the crRNA sequence is SEQ ID NO:388.

In some embodiments, the at least one guide RNA targets a human IL-1β gene, and includes a crRNA sequence having at least 75% identity to SEQ ID NO:389. In some embodiments, the crRNA sequence has at least 80% identity to SEQ ID NO:389. In some embodiments, the crRNA sequence has at least 85% identity to SEQ ID NO:389. In some embodiments, the crRNA sequence has at least 90% identity to SEQ ID NO:389. In some embodiments, the crRNA sequence has at least 95% identity to SEQ ID NO:389. In some embodiments, the crRNA sequence is SEQ ID NO:389.

In some embodiments, the at least one guide RNA targets a canine IL-1α gene, and includes a crRNA sequence having at least 75% identity to a sequence selected from the group consisting of SEQ ID NOs: 522-590. In some embodiments, the crRNA sequence has at least 80% identity to a sequence selected from the group consisting of SEQ ID NOs: 522-590. In some embodiments, the crRNA sequence has at least 85% identity to a sequence selected from the group consisting of SEQ ID NOs: 522-590. In some embodiments, the crRNA sequence has at least 90% identity to a sequence selected from the group consisting of SEQ ID NOs: 522-590. In some embodiments, the crRNA sequence has at least 95% identity to a sequence selected from the group consisting of SEQ ID NOs: 522-590. In some embodiments, the crRNA sequence is selected from the group consisting of SEQ ID NOs: 522-590.

In some embodiments, the at least one guide RNA targets a canine IL-1α gene, and includes a crRNA sequence having at least 75% identity to SEQ ID NO:552. In some embodiments, the crRNA sequence has at least 80% identity to SEQ ID NO:552. In some embodiments, the crRNA sequence has at least 85% identity to SEQ ID NO:552. In some embodiments, the crRNA sequence has at least 90% identity to SEQ ID NO:552. In some embodiments, the crRNA sequence has at least 95% identity to SEQ ID NO:552. In some embodiments, the crRNA sequence is SEQ ID NO:552.

In some embodiments, the at least one guide RNA targets a canine IL-1α gene, and includes a crRNA sequence having at least 75% identity to SEQ ID NO:554. In some embodiments, the crRNA sequence has at least 80% identity to SEQ ID NO:554. In some embodiments, the crRNA sequence has at least 85% identity to SEQ ID NO:554. In some embodiments, the crRNA sequence has at least 90% identity to SEQ ID NO:554. In some embodiments, the crRNA sequence has at least 95% identity to SEQ ID NO:554. In some embodiments, the crRNA sequence is SEQ ID NO:554.

In some embodiments, the at least one guide RNA targets a canine IL-1α gene, and includes a crRNA sequence having at least 75% identity to SEQ ID NO:578. In some embodiments, the crRNA sequence has at least 80% identity to SEQ ID NO:578. In some embodiments, the crRNA sequence has at least 85% identity to SEQ ID NO:578. In some embodiments, the crRNA sequence has at least 90% identity to SEQ ID NO:578. In some embodiments, the crRNA sequence has at least 95% identity to SEQ ID NO:578. In some embodiments, the crRNA sequence is SEQ ID NO:578.

In some embodiments, the at least one guide RNA targets a canine IL-1α gene, and includes a crRNA sequence having at least 75% identity to SEQ ID NO:579. In some embodiments, the crRNA sequence has at least 80% identity to SEQ ID NO:579. In some embodiments, the crRNA sequence has at least 85% identity to SEQ ID NO:579. In some embodiments, the crRNA sequence has at least 90% identity to SEQ ID NO:579. In some embodiments, the crRNA sequence has at least 95% identity to SEQ ID NO:579. In some embodiments, the crRNA sequence is SEQ ID NO:579.

In some embodiments, the at least one guide RNA targets a canine IL-1β gene, and includes a crRNA sequence having at least 75% identity to a sequence selected from the group consisting of SEQ ID NOs: 497-551. In some embodiments, the crRNA sequence has at least 80% identity to a sequence selected from the group consisting of SEQ ID NOs: 497-551. In some embodiments, the crRNA sequence has at least 85% identity to a sequence selected from the group consisting of SEQ ID NOs: 497-551. In some embodiments, the crRNA sequence has at least 90% identity to a sequence selected from the group consisting of SEQ ID NOs: 497-551. In some embodiments, the crRNA sequence has at least 95% identity to a sequence selected from the group consisting of SEQ ID NOs: 497-551. In some embodiments, the crRNA sequence is selected from the group consisting of SEQ ID NOs: 497-551.

In some embodiments, the at least one guide RNA targets a canine IL-1β gene, and includes a crRNA sequence having at least 75% identity to SEQ ID NO:498. In some embodiments, the crRNA sequence has at least 80% identity to SEQ ID NO:498. In some embodiments, the crRNA sequence has at least 85% identity to SEQ ID NO:498. In some embodiments, the crRNA sequence has at least 90% identity to SEQ ID NO:498. In some embodiments, the crRNA sequence has at least 95% identity to SEQ ID NO:498. In some embodiments, the crRNA sequence is SEQ ID NO:498.

In some embodiments, the at least one guide RNA targets a canine IL-1β gene, and includes a crRNA sequence having at least 75% identity to SEQ ID NO:506. In some embodiments, the crRNA sequence has at least 80% identity to SEQ ID NO:506. In some embodiments, the crRNA sequence has at least 85% identity to SEQ ID NO:506. In some embodiments, the crRNA sequence has at least 90% identity to SEQ ID NO:506. In some embodiments, the crRNA sequence has at least 95% identity to SEQ ID NO:506. In some embodiments, the crRNA sequence is SEQ ID NO:506.

In some embodiments, the pharmaceutical composition includes one or more viral vectors, as described herein, collectively comprising the one or more nucleic acids. In some embodiments, the one or more viral vectors include a recombinant virus selected from a retrovirus, an adenovirus, an adeno-associated virus, a lentivirus, and a herpes simplex virus-1. In some embodiments, the one of more viral vectors include a recombinant adeno-associated virus (AAV). In some embodiments, the recombinant AAV is of serotype 5 (AAV5). In some embodiments, the recombinant AAV is of serotype 6 (AAV6).

In some embodiments, the one of more viral vectors include a first viral vector comprising a first nucleic acid, in the one or more nucleic acids, encoding the Cas9 protein, and a second viral vector comprising a second nucleic acid, in the one or more nucleic acids, encoding the at least one guide RNA. In some embodiments, the one of more viral vectors comprise a viral vector comprising a single nucleic acid, wherein the single nucleic acid encodes the Cas9 protein and the at least one guide RNA.

In some embodiments, the composition includes one or more liposomes collectively comprising the one or more nucleic acids. In some embodiments, the one or more nucleic acids are present in a naked state.

In some embodiments, the Cas9 protein is an S. pyogenes Cas9 polypeptide. In some embodiments, the Cas9 protein is an S. aureus Cas9 polypeptide.

In some embodiments, the composition is formulated for parenteral administration. In some embodiments, the composition is formulated for intra-articular injection within a joint of a subject.

In another aspect, the disclosure provides a method for the treatment or prevention of a joint disease or condition in a subject in need thereof. The method includes administering, to a joint of the subject, a pharmaceutical composition comprising a pharmaceutically effective amount of a composition comprising one or more nucleic acids encoding a Clustered Regularly Interspaced Short Palindromic Repeats (CRISPR) gene-editing system. The system includes a CRISPR Associated Protein 9 (Cas9) protein, and at least one guide RNA targeting an IL-1α or IL-1β gene, wherein the target sequence is adjacent to a protospacer adjacent motif (PAM) sequence for the Cas9 protein.

In some embodiments, the at least one guide RNA targets a human IL-1α gene, and includes a crRNA sequence having at least 75% identity to a sequence selected from the group consisting of SEQ ID NOs: 298-387. In some embodiments, the crRNA sequence has at least 80% identity to a sequence selected from the group consisting of SEQ ID NOs: 298-387. In some embodiments, the crRNA sequence has at least 85% identity to a sequence selected from the group consisting of SEQ ID NOs: 298-387. In some embodiments, the crRNA sequence has at least 90% identity to a sequence selected from the group consisting of SEQ ID NOs: 298-387. In some embodiments, the crRNA sequence has at least 95% identity to a sequence selected from the group consisting of SEQ ID NOs: 298-387. In some embodiments, the crRNA sequence is selected from the group consisting of SEQ ID NOs: 298-387.

In some embodiments, the at least one guide RNA targets a human IL-1α gene, and includes a crRNA sequence having at least 75% identity to SEQ ID NO:301. In some embodiments, the crRNA sequence has at least 80% identity to SEQ ID NO:301. In some embodiments, the crRNA sequence has at least 85% identity to SEQ ID NO:301. In some embodiments, the crRNA sequence has at least 90% identity to SEQ ID NO:301. In some embodiments, the crRNA sequence has at least 95% identity to SEQ ID NO:301. In some embodiments, the crRNA sequence is SEQ ID NO:301.

In some embodiments, the at least one guide RNA targets a human IL-1α gene, and includes a crRNA sequence having at least 75% identity to SEQ ID NO:309. In some embodiments, the crRNA sequence has at least 80% identity to SEQ ID NO:309. In some embodiments, the crRNA sequence has at least 85% identity to SEQ ID NO:309. In some embodiments, the crRNA sequence has at least 90% identity to SEQ ID NO:309. In some embodiments, the crRNA sequence has at least 95% identity to SEQ ID NO:309. In some embodiments, the crRNA sequence is SEQ ID NO:309.

In some embodiments, the at least one guide RNA targets a human IL-1β gene, and includes a crRNA sequence having at least 75% identity to a sequence selected from the group consisting of SEQ ID NOs: 388-496. In some embodiments, the crRNA sequence has at least 80% identity to a sequence selected from the group consisting of SEQ ID NOs: 388-496. In some embodiments, the crRNA sequence has at least 85% identity to a sequence selected from the group consisting of SEQ ID NOs: 388-496. In some embodiments, the crRNA sequence has at least 90% identity to a sequence selected from the group consisting of SEQ ID NOs: 388-496. In some embodiments, the crRNA sequence has at least 95% identity to a sequence selected from the group consisting of SEQ ID NOs: 388-496. In some embodiments, the crRNA sequence is selected from the group consisting of SEQ ID NOs: 388-496.

In some embodiments, the at least one guide RNA targets a human IL-1β gene, and includes a crRNA sequence having at least 75% identity to SEQ ID NO:462. In some embodiments, the crRNA sequence has at least 80% identity to SEQ ID NO:462. In some embodiments, the crRNA sequence has at least 85% identity to SEQ ID NO:462. In some embodiments, the crRNA sequence has at least 90% identity to SEQ ID NO:462. In some embodiments, the crRNA sequence has at least 95% identity to SEQ ID NO:462. In some embodiments, the crRNA sequence is SEQ ID NO:462.

In some embodiments, the at least one guide RNA targets a human IL-1β gene, and includes a crRNA sequence having at least 75% identity to SEQ ID NO:391. In some embodiments, the crRNA sequence has at least 80% identity to SEQ ID NO:391. In some embodiments, the crRNA sequence has at least 85% identity to SEQ ID NO:391. In some embodiments, the crRNA sequence has at least 90% identity to SEQ ID NO:391. In some embodiments, the crRNA sequence has at least 95% identity to SEQ ID NO:391. In some embodiments, the crRNA sequence is SEQ ID NO:391.

In some embodiments, the at least one guide RNA targets a human IL-1β gene, and includes a crRNA sequence having at least 75% identity to SEQ ID NO:393. In some embodiments, the crRNA sequence has at least 80% identity to SEQ ID NO:393. In some embodiments, the crRNA sequence has at least 85% identity to SEQ ID NO:393. In some embodiments, the crRNA sequence has at least 90% identity to SEQ ID NO:393. In some embodiments, the crRNA sequence has at least 95% identity to SEQ ID NO:393. In some embodiments, the crRNA sequence is SEQ ID NO:393.

In some embodiments, the at least one guide RNA targets a human IL-1β gene, and includes a crRNA sequence having at least 75% identity to SEQ ID NO:388. In some embodiments, the crRNA sequence has at least 80% identity to SEQ ID NO:388. In some embodiments, the crRNA sequence has at least 85% identity to SEQ ID NO:388. In some embodiments, the crRNA sequence has at least 90% identity to SEQ ID NO:388. In some embodiments, the crRNA sequence has at least 95% identity to SEQ ID NO:388. In some embodiments, the crRNA sequence is SEQ ID NO:388.

In some embodiments, the at least one guide RNA targets a human IL-1β gene, and includes a crRNA sequence having at least 75% identity to SEQ ID NO:389. In some embodiments, the crRNA sequence has at least 80% identity to SEQ ID NO:389. In some embodiments, the crRNA sequence has at least 85% identity to SEQ ID NO:389. In some embodiments, the crRNA sequence has at least 90% identity to SEQ ID NO:389. In some embodiments, the crRNA sequence has at least 95% identity to SEQ ID NO:389. In some embodiments, the crRNA sequence is SEQ ID NO:389.

In some embodiments, the at least one guide RNA targets a canine IL-1α gene, and includes a crRNA sequence having at least 75% identity to a sequence selected from the group consisting of SEQ ID NOs: 522-590. In some embodiments, the crRNA sequence has at least 80% identity to a sequence selected from the group consisting of SEQ ID NOs: 522-590. In some embodiments, the crRNA sequence has at least 85% identity to a sequence selected from the group consisting of SEQ ID NOs: 522-590. In some embodiments, the crRNA sequence has at least 90% identity to a sequence selected from the group consisting of SEQ ID NOs: 522-590. In some embodiments, the crRNA sequence has at least 95% identity to a sequence selected from the group consisting of SEQ ID NOs: 522-590. In some embodiments, the crRNA sequence is selected from the group consisting of SEQ ID NOs: 522-590.

In some embodiments, the at least one guide RNA targets a canine IL-1α gene, and includes a crRNA sequence having at least 75% identity to SEQ ID NO:552. In some embodiments, the crRNA sequence has at least 80% identity to SEQ ID NO:552. In some embodiments, the crRNA sequence has at least 85% identity to SEQ ID NO:552. In some embodiments, the crRNA sequence has at least 90% identity to SEQ ID NO:552. In some embodiments, the crRNA sequence has at least 95% identity to SEQ ID NO:552. In some embodiments, the crRNA sequence is SEQ ID NO:552.

In some embodiments, the at least one guide RNA targets a canine IL-1α gene, and includes a crRNA sequence having at least 75% identity to SEQ ID NO:554. In some embodiments, the crRNA sequence has at least 80% identity to SEQ ID NO:554. In some embodiments, the crRNA sequence has at least 85% identity to SEQ ID NO:554. In some embodiments, the crRNA sequence has at least 90% identity to SEQ ID NO:554. In some embodiments, the crRNA sequence has at least 95% identity to SEQ ID NO:554. In some embodiments, the crRNA sequence is SEQ ID NO:554.

In some embodiments, the at least one guide RNA targets a canine IL-1α gene, and includes a crRNA sequence having at least 75% identity to SEQ ID NO:578. In some embodiments, the crRNA sequence has at least 80% identity to SEQ ID NO:578. In some embodiments, the crRNA sequence has at least 85% identity to SEQ ID NO:578. In some embodiments, the crRNA sequence has at least 90% identity to SEQ ID NO:578. In some embodiments, the crRNA sequence has at least 95% identity to SEQ ID NO:578. In some embodiments, the crRNA sequence is SEQ ID NO:578.

In some embodiments, the at least one guide RNA targets a canine IL-1α gene, and includes a crRNA sequence having at least 75% identity to SEQ ID NO:579. In some embodiments, the crRNA sequence has at least 80% identity to SEQ ID NO:579. In some embodiments, the crRNA sequence has at least 85% identity to SEQ ID NO:579. In some embodiments, the crRNA sequence has at least 90% identity to SEQ ID NO:579. In some embodiments, the crRNA sequence has at least 95% identity to SEQ ID NO:579. In some embodiments, the crRNA sequence is SEQ ID NO:579.

In some embodiments, the at least one guide RNA targets a canine IL-1α gene, and includes a crRNA sequence that is complementary to a target sequence in exon 2 of the IL-1α gene. In some embodiments, the crRNA sequence forms no more than 5 mismatches with the target sequence in exon 2 of the canine IL-1α gene. In some embodiments, the crRNA sequence forms no more than 4 mismatches with the target sequence in exon 2 of the canine IL-1α gene. In some embodiments, the crRNA sequence forms no more than 3 mismatches with the target sequence in exon 2 of the canine IL-1α gene. In some embodiments, the crRNA sequence forms no more than 2 mismatches with the target sequence in exon 2 of the canine IL-1α gene. In some embodiments, the crRNA sequence forms no more than 1 mismatch with the target sequence in exon 2 of the canine IL-1α gene. In some embodiments, the crRNA sequence forms no mismatches with the target sequence in exon 2 of the canine IL-1α gene.

In some embodiments, the at least one guide RNA targets a canine IL-1α gene, and includes a crRNA sequence that is complementary to a target sequence in exon 3 of the IL-1α gene. In some embodiments, the crRNA sequence forms no more than 5 mismatches with the target sequence in exon 3 of the canine IL-1α gene. In some embodiments, the crRNA sequence forms no more than 4 mismatches with the target sequence in exon 3 of the canine IL-1α gene. In some embodiments, the crRNA sequence forms no more than 3 mismatches with the target sequence in exon 3 of the canine IL-1α gene. In some embodiments, the crRNA sequence forms no more than 2 mismatches with the target sequence in exon 3 of the canine IL-1α gene. In some embodiments, the crRNA sequence forms no more than 1 mismatch with the target sequence in exon 3 of the canine IL-1α gene. In some embodiments, the crRNA sequence forms no mismatches with the target sequence in exon 3 of the canine IL-1α gene.

In some embodiments, the at least one guide RNA targets a canine IL-1α gene, and includes a crRNA sequence that is complementary to a target sequence in exon 4 of the IL-1α gene. In some embodiments, the crRNA sequence forms no more than 5 mismatches with the target sequence in exon 4 of the canine IL-1α gene. In some embodiments, the crRNA sequence forms no more than 4 mismatches with the target sequence in exon 4 of the canine IL-1α gene. In some embodiments, the crRNA sequence forms no more than 3 mismatches with the target sequence in exon 4 of the canine IL-1α gene. In some embodiments, the crRNA sequence forms no more than 2 mismatches with the target sequence in exon 4 of the canine IL-1α gene. In some embodiments, the crRNA sequence forms no more than 1 mismatch with the target sequence in exon 4 of the canine IL-1α gene. In some embodiments, the crRNA sequence forms no mismatches with the target sequence in exon 4 of the canine IL-1α gene.

In some embodiments, the at least one guide RNA targets a canine IL-1α gene, and includes a crRNA sequence that is complementary to a target sequence in exon 5 of the IL-1α gene. In some embodiments, the crRNA sequence forms no more than 5 mismatches with the target sequence in exon 5 of the canine IL-1α gene. In some embodiments, the crRNA sequence forms no more than 4 mismatches with the target sequence in exon 5 of the canine IL-1α gene. In some embodiments, the crRNA sequence forms no more than 3 mismatches with the target sequence in exon 5 of the canine IL-1α gene. In some embodiments, the crRNA sequence forms no more than 2 mismatches with the target sequence in exon 5 of the canine IL-1α gene. In some embodiments, the crRNA sequence forms no more than 1 mismatch with the target sequence in exon 5 of the canine IL-1α gene. In some embodiments, the crRNA sequence forms no mismatches with the target sequence in exon 5 of the canine IL-1α gene.

In some embodiments, the at least one guide RNA targets a canine IL-1α gene, and includes a crRNA sequence that is complementary to a target sequence in exon 6 of the IL-1α gene. In some embodiments, the crRNA sequence forms no more than 5 mismatches with the target sequence in exon 6 of the canine IL-1α gene. In some embodiments, the crRNA sequence forms no more than 4 mismatches with the target sequence in exon 6 of the canine IL-1α gene. In some embodiments, the crRNA sequence forms no more than 3 mismatches with the target sequence in exon 6 of the canine IL-1α gene. In some embodiments, the crRNA sequence forms no more than 2 mismatches with the target sequence in exon 6 of the canine IL-1α gene. In some embodiments, the crRNA sequence forms no more than 1 mismatch with the target sequence in exon 6 of the canine IL-1α gene. In some embodiments, the crRNA sequence forms no mismatches with the target sequence in exon 6 of the canine IL-1α gene.

In some embodiments, the at least one guide RNA targets a canine IL-1α gene, and includes a crRNA sequence that is complementary to a target sequence in exon 7 of the IL-1α gene. In some embodiments, the crRNA sequence forms no more than 5 mismatches with the target sequence in exon 7 of the canine IL-1α gene. In some embodiments, the crRNA sequence forms no more than 4 mismatches with the target sequence in exon 7 of the canine IL-1α gene. In some embodiments, the crRNA sequence forms no more than 3 mismatches with the target sequence in exon 7 of the canine IL-1α gene. In some embodiments, the crRNA sequence forms no more than 2 mismatches with the target sequence in exon 7 of the canine IL-1α gene. In some embodiments, the crRNA sequence forms no more than 1 mismatch with the target sequence in exon 7 of the canine IL-1α gene. In some embodiments, the crRNA sequence forms no mismatches with the target sequence in exon 7 of the canine IL-1α gene.

In some embodiments, the at least one guide RNA targets a canine IL-1α gene, and includes a crRNA sequence that is complementary to a target sequence in exon 2 or exon 3 of the IL-1α gene. In some embodiments, the at least one guide RNA targets a canine IL-1α gene, and includes a crRNA sequence that is complementary to a target sequence in exon 2 or exon 4 of the IL-1α gene. In some embodiments, the at least one guide RNA targets a canine IL-1α gene, and includes a crRNA sequence that is complementary to a target sequence in exon 2 or exon 5 of the IL-1α gene. In some embodiments, the at least one guide RNA targets a canine IL-1α gene, and includes a crRNA sequence that is complementary to a target sequence in exon 2 or exon 6 of the IL-1α gene. In some embodiments, the at least one guide RNA targets a canine IL-1α gene, and includes a crRNA sequence that is complementary to a target sequence in exon 2 or exon 7 of the IL-1α gene.

In some embodiments, the at least one guide RNA targets a canine IL-1α gene, and includes a crRNA sequence that is complementary to a target sequence in exon 3 or exon 4 of the IL-1α gene. In some embodiments, the at least one guide RNA targets a canine IL-1α gene, and includes a crRNA sequence that is complementary to a target sequence in exon 3 or exon 5 of the IL-1α gene. In some embodiments, the at least one guide RNA targets a canine IL-1α gene, and includes a crRNA sequence that is complementary to a target sequence in exon 3 or exon 6 of the IL-1α gene. In some embodiments, the at least one guide RNA targets a canine IL-1α gene, and includes a crRNA sequence that is complementary to a target sequence in exon 3 or exon 7 of the IL-1α gene.

In some embodiments, the at least one guide RNA targets a canine IL-1α gene, and includes a crRNA sequence that is complementary to a target sequence in exon 4 or exon 5 of the IL-1α gene. In some embodiments, the at least one guide RNA targets a canine IL-1α gene, and includes a crRNA sequence that is complementary to a target sequence in exon 4 or exon 6 of the IL-1α gene. In some embodiments, the at least one guide RNA targets a canine IL-1α gene, and includes a crRNA sequence that is complementary to a target sequence in exon 4 or exon 7 of the IL-1α gene.

In some embodiments, the at least one guide RNA targets a canine IL-1α gene, and includes a crRNA sequence that is complementary to a target sequence in exon 5 or exon 6 of the IL-1α gene. In some embodiments, the at least one guide RNA targets a canine IL-1α gene, and includes a crRNA sequence that is complementary to a target sequence in exon 5 or exon 7 of the IL-1α gene. In some embodiments, the at least one guide RNA targets a canine IL-1α gene, and includes a crRNA sequence that is complementary to a target sequence in exon 6 or exon 7 of the IL-1α gene.

In some embodiments, the at least one guide RNA targets a canine IL-1α gene, and includes a crRNA sequence that is complementary to a target sequence in exon 2, exon 3, or exon 4 of the IL-1α gene. In some embodiments, the at least one guide RNA targets a canine IL-1α gene, and includes a crRNA sequence that is complementary to a target sequence in exon 2, exon 3, or exon 5 of the IL-1α gene. In some embodiments, the at least one guide RNA targets a canine IL-1α gene, and includes a crRNA sequence that is complementary to a target sequence in exon 2, exon 3, or exon 6 of the IL-1α gene. In some embodiments, the at least one guide RNA targets a canine IL-1α gene, and includes a crRNA sequence that is complementary to a target sequence in exon 2, exon 3, or exon 7 of the IL-1α gene. In some embodiments, the at least one guide RNA targets a canine IL-1α gene, and includes a crRNA sequence that is complementary to a target sequence in exon 2, exon 4, or exon 5 of the IL-1α gene. In some embodiments, the at least one guide RNA targets a canine IL-1α gene, and includes a crRNA sequence that is complementary to a target sequence in exon 2, exon 4, or exon 6 of the IL-1α gene. In some embodiments, the at least one guide RNA targets a canine IL-1α gene, and includes a crRNA sequence that is complementary to a target sequence in exon 2, exon 4, or exon 7 of the IL-1α gene. In some embodiments, the at least one guide RNA targets a canine IL-1α gene, and includes a crRNA sequence that is complementary to a target sequence in exon 2, exon 5, or exon 6 of the IL-1α gene. In some embodiments, the at least one guide RNA targets a canine IL-1α gene, and includes a crRNA sequence that is complementary to a target sequence in exon 2, exon 5, or exon 7 of the IL-1α gene. In some embodiments, the at least one guide RNA targets a canine IL-1α gene, and includes a crRNA sequence that is complementary to a target sequence in exon 2, exon 6, or exon 7 of the IL-1α gene.

In some embodiments, the at least one guide RNA targets a canine IL-1α gene, and includes a crRNA sequence that is complementary to a target sequence in exon 3, exon 4, or exon 5 of the IL-1α gene. In some embodiments, the at least one guide RNA targets a canine IL-1α gene, and includes a crRNA sequence that is complementary to a target sequence in exon 3, exon 4, or exon 6 of the IL-1α gene. In some embodiments, the at least one guide RNA targets a canine IL-1α gene, and includes a crRNA sequence that is complementary to a target sequence in exon 3, exon 4, or exon 7 of the IL-1α gene. In some embodiments, the at least one guide RNA targets a canine IL-1α gene, and includes a crRNA sequence that is complementary to a target sequence in exon 3, exon 5, or exon 6 of the IL-1α gene. In some embodiments, the at least one guide RNA targets a canine IL-1α gene, and includes a crRNA sequence that is complementary to a target sequence in exon 3, exon 5, or exon 7 of the IL-1α gene. In some embodiments, the at least one guide RNA targets a canine IL-1α gene, and includes a crRNA sequence that is complementary to a target sequence in exon 3, exon 6, or exon 7 of the IL-1α gene.

In some embodiments, the at least one guide RNA targets a canine IL-1α gene, and includes a crRNA sequence that is complementary to a target sequence in exon 4, exon 5, or exon 6 of the IL-1α gene. In some embodiments, the at least one guide RNA targets a canine IL-1α gene, and includes a crRNA sequence that is complementary to a target sequence in exon 4, exon 5, or exon 7 of the IL-1α gene. In some embodiments, the at least one guide RNA targets a canine IL-1α gene, and includes a crRNA sequence that is complementary to a target sequence in exon 4, exon 6, or exon 7 of the IL-1α gene. In some embodiments, the at least one guide RNA targets a canine IL-1α gene, and includes a crRNA sequence that is complementary to a target sequence in exon 5, exon 6, or exon 7 of the IL-1α gene.

In some embodiments, the at least one guide RNA targets a canine IL-1α gene, and includes a crRNA sequence that is complementary to a target sequence in exon 2, exon 3, exon 4, or exon 5 of the IL-1α gene. In some embodiments, the at least one guide RNA targets a canine IL-1α gene, and includes a crRNA sequence that is complementary to a target sequence in exon 2, exon 3, exon 4, or exon 6 of the IL-1α gene. In some embodiments, the at least one guide RNA targets a canine IL-1α gene, and includes a crRNA sequence that is complementary to a target sequence in exon 2, exon 3, exon 4, or exon 7 of the IL-1α gene. In some embodiments, the at least one guide RNA targets a canine IL-1α gene, and includes a crRNA sequence that is complementary to a target sequence in exon 2, exon 4, exon 5, or exon 6 of the IL-1α gene. In some embodiments, the at least one guide RNA targets a canine IL-1α gene, and includes a crRNA sequence that is complementary to a target sequence in exon 2, exon 4, exon 5, or exon 7 of the IL-1α gene. In some embodiments, the at least one guide RNA targets a canine IL-1α gene, and includes a crRNA sequence that is complementary to a target sequence in exon 2, exon 4, exon 6, or exon 7 of the IL-1α gene. In some embodiments, the at least one guide RNA targets a canine IL-1α gene, and includes a crRNA sequence that is complementary to a target sequence in exon 2, exon 5, exon 6, or exon 7 of the IL-1α gene.

In some embodiments, the at least one guide RNA targets a canine IL-1α gene, and includes a crRNA sequence that is complementary to a target sequence in exon 3, exon 4, exon 5, or exon 6 of the IL-1α gene. In some embodiments, the at least one guide RNA targets a canine IL-1α gene, and includes a crRNA sequence that is complementary to a target sequence in exon 3, exon 4, exon 5, or exon 7 of the IL-1α gene. In some embodiments, the at least one guide RNA targets a canine IL-1α gene, and includes a crRNA sequence that is complementary to a target sequence in exon 3, exon 4, exon 6, or exon 7 of the IL-1α gene. In some embodiments, the at least one guide RNA targets a canine IL-1α gene, and includes a crRNA sequence that is complementary to a target sequence in exon 3, exon 5, exon 6, or exon 7 of the IL-1α gene. In some embodiments, the at least one guide RNA targets a canine IL-1α gene, and includes a crRNA sequence that is complementary to a target sequence in exon 4, exon 5, exon 6, or exon 7 of the IL-1α gene.

In some embodiments, the at least one guide RNA targets a canine IL-1α gene, and includes a crRNA sequence that is complementary to a target sequence in exon 2, exon 3, exon 4, exon 5, or exon 6 of the IL-1α gene. In some embodiments, the at least one guide RNA targets a canine IL-1α gene, and includes a crRNA sequence that is complementary to a target sequence in exon 2, exon 3, exon 4, exon 5, or exon 7 of the IL-1α gene. In some embodiments, the at least one guide RNA targets a canine IL-1α gene, and includes a crRNA sequence that is complementary to a target sequence in exon 3, exon 4, exon 5, exon 6, or exon 7 of the IL-1α gene. In some embodiments, the at least one guide RNA targets a canine IL-1α gene, and includes a crRNA sequence that is complementary to a target sequence in exon 2, exon 4, exon 5, exon 6, or exon 7 of the IL-1α gene. In some embodiments, the at least one guide RNA targets a canine IL-1α gene, and includes a crRNA sequence that is complementary to a target sequence in exon 2, exon 3, exon 5, exon 6, or exon 6 of the IL-1α gene. In some embodiments, the at least one guide RNA targets a canine IL-1α gene, and includes a crRNA sequence that is complementary to a target sequence in exon 2, exon 3, exon 4, exon 6, or exon 7 of the IL-1α gene. In some embodiments, the at least one guide RNA targets a canine IL-1α gene, and includes a crRNA sequence that is complementary to a target sequence in exon 2, exon 3, exon 4, exon 5, exon 6, or exon 7 of the IL-1α gene.

In some embodiments, the at least one guide RNA targets a canine IL-1β gene, and includes a crRNA sequence having at least 75% identity to a sequence selected from the group consisting of SEQ ID NOs: 497-551. In some embodiments, the crRNA sequence has at least 80% identity to a sequence selected from the group consisting of SEQ ID NOs: 497-551. In some embodiments, the crRNA sequence has at least 85% identity to a sequence selected from the group consisting of SEQ ID NOs: 497-551. In some embodiments, the crRNA sequence has at least 90% identity to a sequence selected from the group consisting of SEQ ID NOs: 497-551. In some embodiments, the crRNA sequence has at least 95% identity to a sequence selected from the group consisting of SEQ ID NOs: 497-551. In some embodiments, the crRNA sequence is selected from the group consisting of SEQ ID NOs: 497-551.

In some embodiments, the at least one guide RNA targets a canine IL-1β gene, and includes a crRNA sequence having at least 75% identity to SEQ ID NO:498. In some embodiments, the crRNA sequence has at least 80% identity to SEQ ID NO:498. In some embodiments, the crRNA sequence has at least 85% identity to SEQ ID NO:498. In some embodiments, the crRNA sequence has at least 90% identity to SEQ ID NO:498. In some embodiments, the crRNA sequence has at least 95% identity to SEQ ID NO:498. In some embodiments, the crRNA sequence is SEQ ID NO:498.

In some embodiments, the at least one guide RNA targets a canine IL-1β gene, and includes a crRNA sequence having at least 75% identity to SEQ ID NO:506. In some embodiments, the crRNA sequence has at least 80% identity to SEQ ID NO:506. In some embodiments, the crRNA sequence has at least 85% identity to SEQ ID NO:506. In some embodiments, the crRNA sequence has at least 90% identity to SEQ ID NO:506. In some embodiments, the crRNA sequence has at least 95% identity to SEQ ID NO:506. In some embodiments, the crRNA sequence is SEQ ID NO:506.

In some embodiments, the at least one guide RNA targets a canine IL-1β gene, and includes a crRNA sequence that is complementary to a target sequence in exon 2 of the IL-1β gene. In some embodiments, the crRNA sequence forms no more than 5 mismatches with the target sequence in exon 2 of the canine IL-1β gene. In some embodiments, the crRNA sequence forms no more than 4 mismatches with the target sequence in exon 2 of the canine IL-1β gene. In some embodiments, the crRNA sequence forms no more than 3 mismatches with the target sequence in exon 2 of the canine IL-1β gene. In some embodiments, the crRNA sequence forms no more than 2 mismatches with the target sequence in exon 2 of the canine IL-1β gene. In some embodiments, the crRNA sequence forms no more than 1 mismatch with the target sequence in exon 2 of the canine IL-1β gene. In some embodiments, the crRNA sequence forms no mismatches with the target sequence in exon 2 of the canine IL-1β gene.

In some embodiments, the at least one guide RNA targets a canine IL-1β gene, and includes a crRNA sequence that is complementary to a target sequence in exon 3 of the IL-1β gene. In some embodiments, the crRNA sequence forms no more than 5 mismatches with the target sequence in exon 3 of the canine IL-1β gene. In some embodiments, the crRNA sequence forms no more than 4 mismatches with the target sequence in exon 3 of the canine IL-1β gene. In some embodiments, the crRNA sequence forms no more than 3 mismatches with the target sequence in exon 3 of the canine IL-1β gene. In some embodiments, the crRNA sequence forms no more than 2 mismatches with the target sequence in exon 3 of the canine IL-1β gene. In some embodiments, the crRNA sequence forms no more than 1 mismatch with the target sequence in exon 3 of the canine IL-1β gene. In some embodiments, the crRNA sequence forms no mismatches with the target sequence in exon 3 of the canine IL-1β gene.

In some embodiments, the at least one guide RNA targets a canine IL-1β gene, and includes a crRNA sequence that is complementary to a target sequence in exon 4 of the IL-1β gene. In some embodiments, the crRNA sequence forms no more than 5 mismatches with the target sequence in exon 4 of the canine IL-1β gene. In some embodiments, the crRNA sequence forms no more than 4 mismatches with the target sequence in exon 4 of the canine IL-1β gene. In some embodiments, the crRNA sequence forms no more than 3 mismatches with the target sequence in exon 4 of the canine IL-1β gene. In some embodiments, the crRNA sequence forms no more than 2 mismatches with the target sequence in exon 4 of the canine IL-1β gene. In some embodiments, the crRNA sequence forms no more than 1 mismatch with the target sequence in exon 4 of the canine IL-1β gene. In some embodiments, the crRNA sequence forms no mismatches with the target sequence in exon 4 of the canine IL-1β gene.

In some embodiments, the at least one guide RNA targets a canine IL-1β gene, and includes a crRNA sequence that is complementary to a target sequence in exon 5 of the IL-1β gene. In some embodiments, the crRNA sequence forms no more than 5 mismatches with the target sequence in exon 5 of the canine IL-1β gene. In some embodiments, the crRNA sequence forms no more than 4 mismatches with the target sequence in exon 5 of the canine IL-1β gene. In some embodiments, the crRNA sequence forms no more than 3 mismatches with the target sequence in exon 5 of the canine IL-1β gene. In some embodiments, the crRNA sequence forms no more than 2 mismatches with the target sequence in exon 5 of the canine IL-1β gene. In some embodiments, the crRNA sequence forms no more than 1 mismatch with the target sequence in exon 5 of the canine IL-1β gene. In some embodiments, the crRNA sequence forms no mismatches with the target sequence in exon 5 of the canine IL-1β gene.

In some embodiments, the at least one guide RNA targets a canine IL-1β gene, and includes a crRNA sequence that is complementary to a target sequence in exon 6 of the IL-1β gene. In some embodiments, the crRNA sequence forms no more than 5 mismatches with the target sequence in exon 6 of the canine IL-1β gene. In some embodiments, the crRNA sequence forms no more than 4 mismatches with the target sequence in exon 6 of the canine IL-1β gene. In some embodiments, the crRNA sequence forms no more than 3 mismatches with the target sequence in exon 6 of the canine IL-1β gene. In some embodiments, the crRNA sequence forms no more than 2 mismatches with the target sequence in exon 6 of the canine IL-1β gene. In some embodiments, the crRNA sequence forms no more than 1 mismatch with the target sequence in exon 6 of the canine IL-1β gene. In some embodiments, the crRNA sequence forms no mismatches with the target sequence in exon 6 of the canine IL-1β gene.

In some embodiments, the at least one guide RNA targets a canine IL-1β gene, and includes a crRNA sequence that is complementary to a target sequence in exon 7 of the IL-1β gene. In some embodiments, the crRNA sequence forms no more than 5 mismatches with the target sequence in exon 7 of the canine IL-1β gene. In some embodiments, the crRNA sequence forms no more than 4 mismatches with the target sequence in exon 7 of the canine IL-1β gene. In some embodiments, the crRNA sequence forms no more than 3 mismatches with the target sequence in exon 7 of the canine IL-1β gene. In some embodiments, the crRNA sequence forms no more than 2 mismatches with the target sequence in exon 7 of the canine IL-1β gene. In some embodiments, the crRNA sequence forms no more than 1 mismatch with the target sequence in exon 7 of the canine IL-1β gene. In some embodiments, the crRNA sequence forms no mismatches with the target sequence in exon 7 of the canine IL-1β gene.

In some embodiments, the at least one guide RNA targets a canine IL-1β gene, and includes a crRNA sequence that is complementary to a target sequence in exon 2 or exon 3 of the IL-1β gene. In some embodiments, the at least one guide RNA targets a canine IL-1β gene, and includes a crRNA sequence that is complementary to a target sequence in exon 2 or exon 4 of the IL-1β gene. In some embodiments, the at least one guide RNA targets a canine IL-1β gene, and includes a crRNA sequence that is complementary to a target sequence in exon 2 or exon 5 of the IL-1β gene. In some embodiments, the at least one guide RNA targets a canine IL-1β gene, and includes a crRNA sequence that is complementary to a target sequence in exon 2 or exon 6 of the IL-1β gene. In some embodiments, the at least one guide RNA targets a canine IL-1β gene, and includes a crRNA sequence that is complementary to a target sequence in exon 2 or exon 7 of the IL-1β gene.

In some embodiments, the at least one guide RNA targets a canine IL-1β gene, and includes a crRNA sequence that is complementary to a target sequence in exon 3 or exon 4 of the IL-1β gene. In some embodiments, the at least one guide RNA targets a canine IL-1β gene, and includes a crRNA sequence that is complementary to a target sequence in exon 3 or exon 5 of the IL-1β gene. In some embodiments, the at least one guide RNA targets a canine IL-1β gene, and includes a crRNA sequence that is complementary to a target sequence in exon 3 or exon 6 of the IL-1β gene. In some embodiments, the at least one guide RNA targets a canine IL-1β gene, and includes a crRNA sequence that is complementary to a target sequence in exon 3 or exon 7 of the IL-1β gene.

In some embodiments, the at least one guide RNA targets a canine IL-1β gene, and includes a crRNA sequence that is complementary to a target sequence in exon 4 or exon 5 of the IL-1β gene. In some embodiments, the at least one guide RNA targets a canine IL-1β gene, and includes a crRNA sequence that is complementary to a target sequence in exon 4 or exon 6 of the IL-1β gene. In some embodiments, the at least one guide RNA targets a canine IL-1β gene, and includes a crRNA sequence that is complementary to a target sequence in exon 4 or exon 7 of the IL-1β gene.

In some embodiments, the at least one guide RNA targets a canine IL-1β gene, and includes a crRNA sequence that is complementary to a target sequence in exon 5 or exon 6 of the IL-1β gene. In some embodiments, the at least one guide RNA targets a canine IL-1β gene, and includes a crRNA sequence that is complementary to a target sequence in exon 5 or exon 7 of the IL-1β gene. In some embodiments, the at least one guide RNA targets a canine IL-1β gene, and includes a crRNA sequence that is complementary to a target sequence in exon 6 or exon 7 of the IL-1β gene.

In some embodiments, the at least one guide RNA targets a canine IL-1β gene, and includes a crRNA sequence that is complementary to a target sequence in exon 2, exon 3, or exon 4 of the IL-1β gene. In some embodiments, the at least one guide RNA targets a canine IL-1β gene, and includes a crRNA sequence that is complementary to a target sequence in exon 2, exon 3, or exon 5 of the IL-1β gene. In some embodiments, the at least one guide RNA targets a canine IL-1β gene, and includes a crRNA sequence that is complementary to a target sequence in exon 2, exon 3, or exon 6 of the IL-1β gene. In some embodiments, the at least one guide RNA targets a canine IL-1β gene, and includes a crRNA sequence that is complementary to a target sequence in exon 2, exon 3, or exon 7 of the IL-1β gene. In some embodiments, the at least one guide RNA targets a canine IL-1β gene, and includes a crRNA sequence that is complementary to a target sequence in exon 2, exon 4, or exon 5 of the IL-1β gene. In some embodiments, the at least one guide RNA targets a canine IL-1β gene, and includes a crRNA sequence that is complementary to a target sequence in exon 2, exon 4, or exon 6 of the IL-1β gene. In some embodiments, the at least one guide RNA targets a canine IL-1β gene, and includes a crRNA sequence that is complementary to a target sequence in exon 2, exon 4, or exon 7 of the IL-1β gene. In some embodiments, the at least one guide RNA targets a canine IL-1β gene, and includes a crRNA sequence that is complementary to a target sequence in exon 2, exon 5, or exon 6 of the IL-1β gene. In some embodiments, the at least one guide RNA targets a canine IL-1β gene, and includes a crRNA sequence that is complementary to a target sequence in exon 2, exon 5, or exon 7 of the IL-1β gene. In some embodiments, the at least one guide RNA targets a canine IL-1β gene, and includes a crRNA sequence that is complementary to a target sequence in exon 2, exon 6, or exon 7 of the IL-1β gene.

In some embodiments, the at least one guide RNA targets a canine IL-1β gene, and includes a crRNA sequence that is complementary to a target sequence in exon 3, exon 4, or exon 5 of the IL-1β gene. In some embodiments, the at least one guide RNA targets a canine IL-1β gene, and includes a crRNA sequence that is complementary to a target sequence in exon 3, exon 4, or exon 6 of the IL-1β gene. In some embodiments, the at least one guide RNA targets a canine IL-1β gene, and includes a crRNA sequence that is complementary to a target sequence in exon 3, exon 4, or exon 7 of the IL-1β gene. In some embodiments, the at least one guide RNA targets a canine IL-1β gene, and includes a crRNA sequence that is complementary to a target sequence in exon 3, exon 5, or exon 6 of the IL-1β gene. In some embodiments, the at least one guide RNA targets a canine IL-1β gene, and includes a crRNA sequence that is complementary to a target sequence in exon 3, exon 5, or exon 7 of the IL-1β gene. In some embodiments, the at least one guide RNA targets a canine IL-1β gene, and includes a crRNA sequence that is complementary to a target sequence in exon 3, exon 6, or exon 7 of the IL-1β gene.

In some embodiments, the at least one guide RNA targets a canine IL-1β gene, and includes a crRNA sequence that is complementary to a target sequence in exon 4, exon 5, or exon 6 of the IL-1β gene. In some embodiments, the at least one guide RNA targets a canine IL-1β gene, and includes a crRNA sequence that is complementary to a target sequence in exon 4, exon 5, or exon 7 of the IL-1β gene. In some embodiments, the at least one guide RNA targets a canine IL-1β gene, and includes a crRNA sequence that is complementary to a target sequence in exon 4, exon 6, or exon 7 of the IL-1β gene. In some embodiments, the at least one guide RNA targets a canine IL-1β gene, and includes a crRNA sequence that is complementary to a target sequence in exon 5, exon 6, or exon 7 of the IL-1β gene.

In some embodiments, the at least one guide RNA targets a canine IL-1β gene, and includes a crRNA sequence that is complementary to a target sequence in exon 2, exon 3, exon 4, or exon 5 of the IL-1β gene. In some embodiments, the at least one guide RNA targets a canine IL-1β gene, and includes a crRNA sequence that is complementary to a target sequence in exon 2, exon 3, exon 4, or exon 6 of the IL-1β gene. In some embodiments, the at least one guide RNA targets a canine IL-1β gene, and includes a crRNA sequence that is complementary to a target sequence in exon 2, exon 3, exon 4, or exon 7 of the IL-1β gene. In some embodiments, the at least one guide RNA targets a canine IL-1β gene, and includes a crRNA sequence that is complementary to a target sequence in exon 2, exon 4, exon 5, or exon 6 of the IL-1β gene. In some embodiments, the at least one guide RNA targets a canine IL-1β gene, and includes a crRNA sequence that is complementary to a target sequence in exon 2, exon 4, exon 5, or exon 7 of the IL-1β gene. In some embodiments, the at least one guide RNA targets a canine IL-1β gene, and includes a crRNA sequence that is complementary to a target sequence in exon 2, exon 4, exon 6, or exon 7 of the IL-1β gene. In some embodiments, the at least one guide RNA targets a canine IL-1β gene, and includes a crRNA sequence that is complementary to a target sequence in exon 2, exon 5, exon 6, or exon 7 of the IL-1β gene.

In some embodiments, the at least one guide RNA targets a canine IL-1β gene, and includes a crRNA sequence that is complementary to a target sequence in exon 3, exon 4, exon 5, or exon 6 of the IL-1β gene. In some embodiments, the at least one guide RNA targets a canine IL-1β gene, and includes a crRNA sequence that is complementary to a target sequence in exon 3, exon 4, exon 5, or exon 7 of the IL-1β gene. In some embodiments, the at least one guide RNA targets a canine IL-1β gene, and includes a crRNA sequence that is complementary to a target sequence in exon 3, exon 4, exon 6, or exon 7 of the IL-1β gene. In some embodiments, the at least one guide RNA targets a canine IL-1β gene, and includes a crRNA sequence that is complementary to a target sequence in exon 3, exon 5, exon 6, or exon 7 of the IL-1β gene. In some embodiments, the at least one guide RNA targets a canine IL-1β gene, and includes a crRNA sequence that is complementary to a target sequence in exon 4, exon 5, exon 6, or exon 7 of the IL-1β gene.

In some embodiments, the at least one guide RNA targets a canine IL-1β gene, and includes a crRNA sequence that is complementary to a target sequence in exon 2, exon 3, exon 4, exon 5, or exon 6 of the IL-1β gene. In some embodiments, the at least one guide RNA targets a canine IL-1β gene, and includes a crRNA sequence that is complementary to a target sequence in exon 2, exon 3, exon 4, exon 5, or exon 7 of the IL-1β gene. In some embodiments, the at least one guide RNA targets a canine IL-1β gene, and includes a crRNA sequence that is complementary to a target sequence in exon 3, exon 4, exon 5, exon 6, or exon 7 of the IL-1β gene. In some embodiments, the at least one guide RNA targets a canine IL-1β gene, and includes a crRNA sequence that is complementary to a target sequence in exon 2, exon 4, exon 5, exon 6, or exon 7 of the IL-1β gene. In some embodiments, the at least one guide RNA targets a canine IL-1β gene, and includes a crRNA sequence that is complementary to a target sequence in exon 2, exon 3, exon 5, exon 6, or exon 6 of the IL-1β gene. In some embodiments, the at least one guide RNA targets a canine IL-1β gene, and includes a crRNA sequence that is complementary to a target sequence in exon 2, exon 3, exon 4, exon 6, or exon 7 of the IL-1β gene. In some embodiments, the at least one guide RNA targets a canine IL-1β gene, and includes a crRNA sequence that is complementary to a target sequence in exon 2, exon 3, exon 4, exon 5, exon 6, or exon 7 of the IL-1β gene.

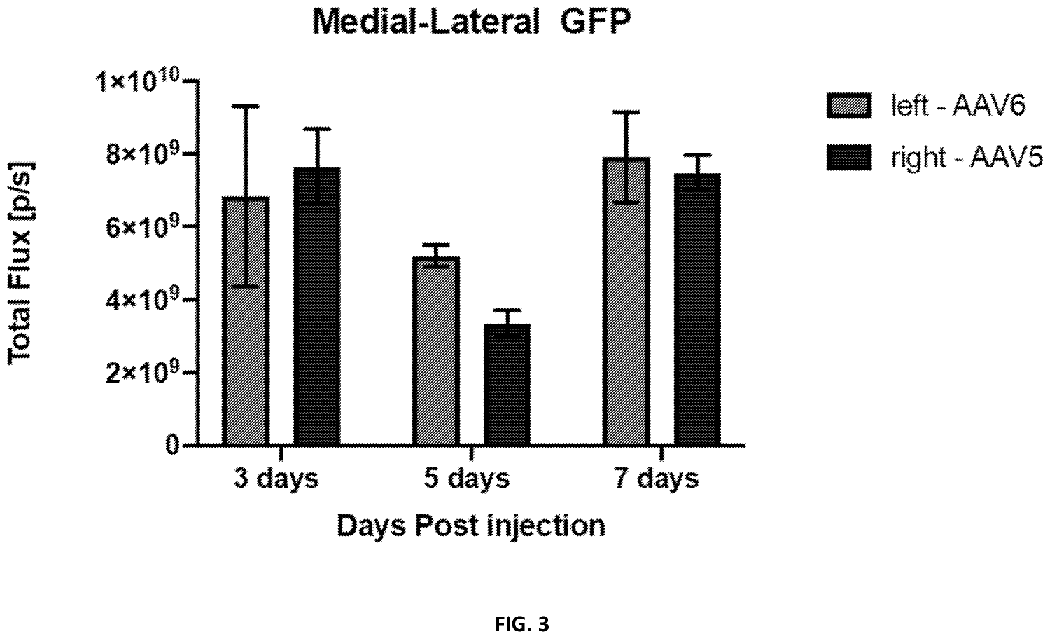

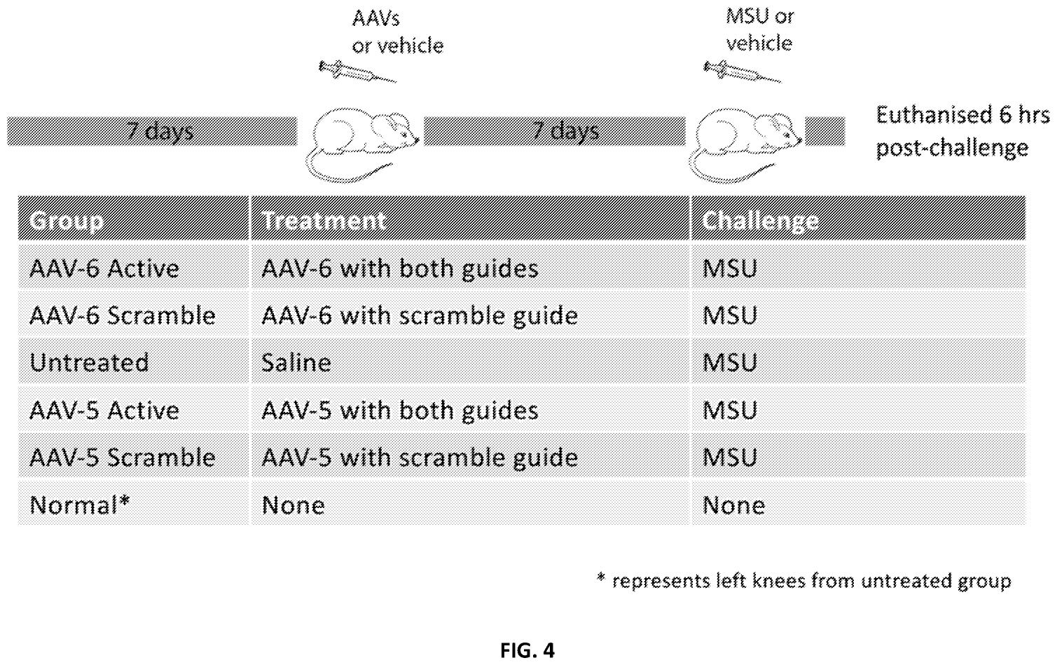

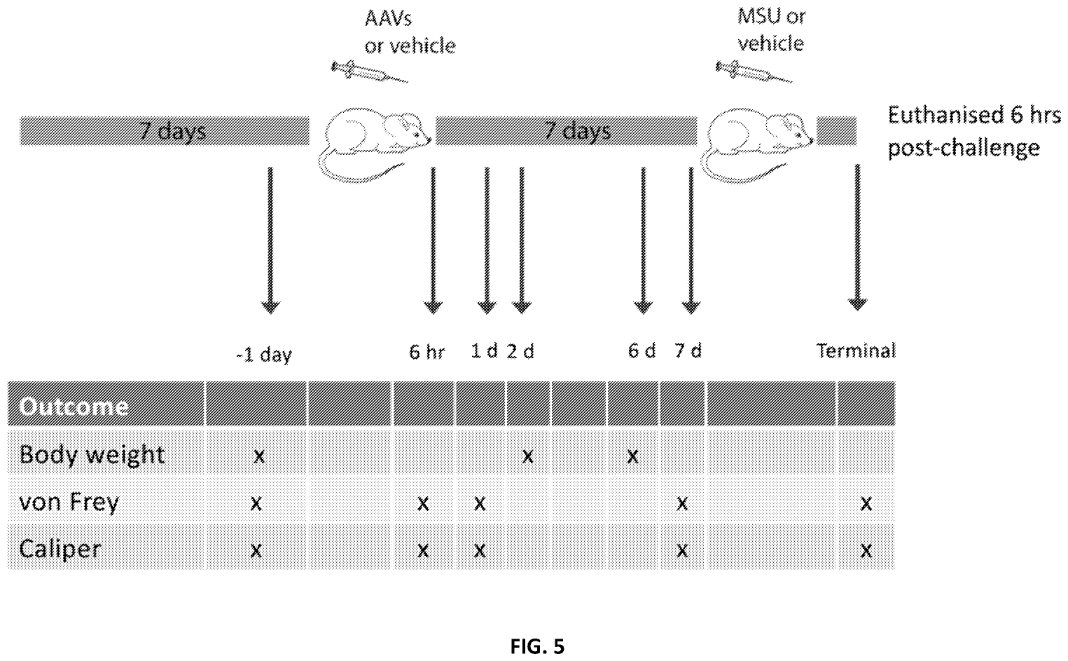

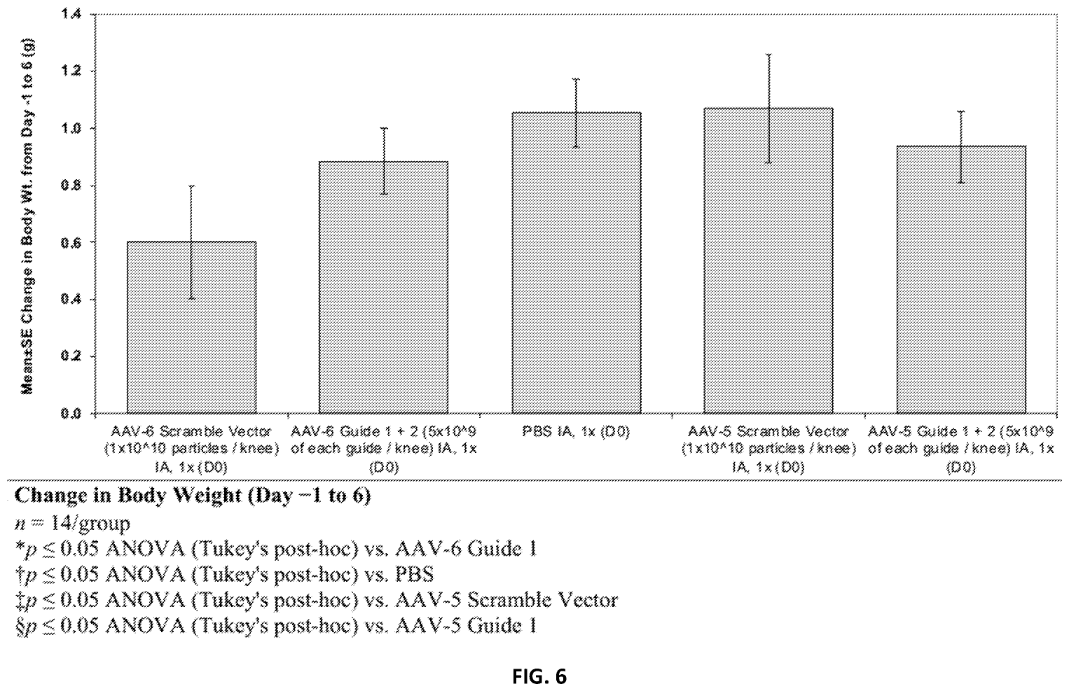

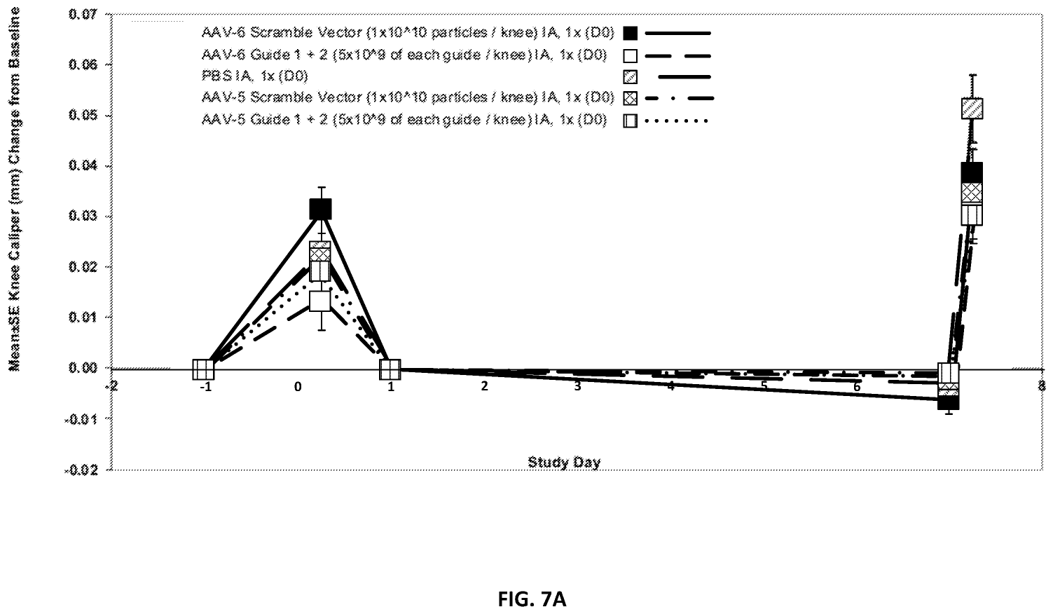

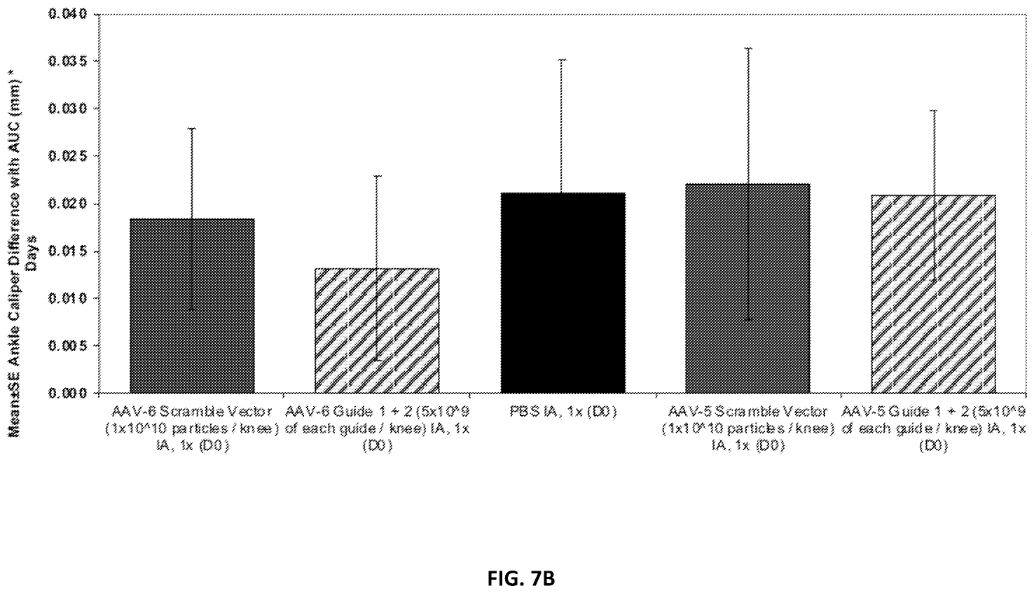

Generally, the crRNA sequences described herein may include one or more nucleotide substitutions, e.g., relative to the reverse complement of the target sequence. Guidance for making nucleotide substitutions can be found, for example, in Jiang et al. and Doudna (Jiang and Doudna, Annu. Rev. Biophys., 46:505-29 (2017)), the content of which is incorporated herein by reference, in its entirety, for all purposes. Specifically, Jiang and Doudna considers molecular structures generated for many different confirmations of the CRISPR/Cas9 system, ranging from apo Cas9 protein (FIG. 3) to Cas9-sgRNA complexes bound to the target strand of an invaded double-stranded DNA molecule (FIGS. 5 and 7), to arrive at a detailed molecule model of CRISPR/Cas9 binding and cleavage in FIG. 6. From these molecular models, the person of ordinary skill in the art would know which nucleotide positions in the crRNA sequence would be more tolerant of mismatches with the target sequence.

For instance, Jiang teaches that the PAM-proximal 10-12 nucleotides, also known as the ‘seed region’ of the crRNA targeting sequence, are most critical for robust CRISPR/Cas9 binding. Specifically, Jiang discloses that mismatches in the seed region “severely impair or completely abrogate target DNA binding and cleavage, whereas close homology in the seed region often leads to off-target binding events even with many mismatches elsewhere,” i.e., in the PAM-distal 8-10 nucleotides. Jiang at 512. Similarly, Jiang teaches that “[p]erfect complementarity between the seed region of sgRNA and target DNA is necessary for Cas9-mediated DNA targeting and cleavage, whereas imperfect base pairing at the nonseed region is much more tolerated for target binding specificity. Id., citations omitted.

Accordingly, in some embodiments, a crRNA sequence used in the compositions and/or methods of the disclosure include one or more nucleotide substitutions, e.g., relative to any of SEQ ID NOs: 298-590, within the PAM-distal 8-10 nucleotides. In some embodiments, a crRNA sequence includes one nucleotide substitution, e.g., relative to any of SEQ ID NOs: 298-590, within the PAM-distal 8-10 nucleotides. In some embodiments, a crRNA sequence includes two nucleotide substitutions, e.g., relative to any of SEQ ID NOs: 298-590, within the PAM-distal 8-10 nucleotides. In some embodiments, a crRNA sequence includes three nucleotide substitutions, e.g., relative to any of SEQ ID NOs: 298-590, within the PAM-distal 8-10 nucleotides. In some embodiments, a crRNA sequence includes four nucleotide substitutions, e.g., relative to any of SEQ ID NOs: 298-590, within the PAM-distal 8-10 nucleotides. In some embodiments, a crRNA sequence includes five nucleotide substitutions, e.g., relative to any of SEQ ID NOs: 298-590, within the PAM-distal 8-10 nucleotides.

Accordingly, in some embodiments, a crRNA sequence used in the compositions and/or methods of the disclosure include one or more nucleotide substitutions, e.g., relative to any of SEQ ID NOs: 298-590, within the first 8 positions of the crRNA sequence. In some embodiments, a crRNA sequence includes one nucleotide substitution, e.g., relative to any of SEQ ID NOs: 298-590, within the first 8 positions of the crRNA sequence. In some embodiments, a crRNA sequence includes two nucleotide substitutions, e.g., relative to any of SEQ ID NOs: 298-590, within the first 8 positions of the crRNA sequence. In some embodiments, a crRNA sequence includes three nucleotide substitutions, e.g., relative to any of SEQ ID NOs: 298-590, within the first 8 positions of the crRNA sequence. In some embodiments, a crRNA sequence includes four nucleotide substitutions, e.g., relative to any of SEQ ID NOs: 298-590, within the first 8 positions of the crRNA sequence. In some embodiments, a crRNA sequence includes five nucleotide substitutions, e.g., relative to any of SEQ ID NOs: 298-590, within the first 8 positions of the crRNA sequence.

Similarly, in some embodiments, a crRNA sequence used in the compositions and/or methods of the disclosure include one or more nucleotide substitutions, e.g., relative to any of SEQ ID NOs: 298-590, within the first 10 positions of the crRNA sequence. In some embodiments, a crRNA sequence includes one nucleotide substitution, e.g., relative to any of SEQ ID NOs: 298-590, within the first 10 positions of the crRNA sequence. In some embodiments, a crRNA sequence includes two nucleotide substitutions, e.g., relative to any of SEQ ID NOs: 298-590, within the first 10 positions of the crRNA sequence. In some embodiments, a crRNA sequence includes three nucleotide substitutions, e.g., relative to any of SEQ ID NOs: 298-590, within the first 10 positions of the crRNA sequence. In some embodiments, a crRNA sequence includes four nucleotide substitutions, e.g., relative to any of SEQ ID NOs: 298-590, within the first 10 positions of the crRNA sequence. In some embodiments, a crRNA sequence includes five nucleotide substitutions, e.g., relative to any of SEQ ID NOs: 298-590, within the first 10 positions of the crRNA sequence.

Further, Jiang and Doudna postulates that base pairing of PAM-distal nucleotides at positions 14-17 of the crRNA targeting sequence are important for cleavage activity, following binding to the target sequence.

Accordingly, in some embodiments, a crRNA sequence used in the compositions and/or methods of the disclosure include one or more nucleotide substitutions, e.g., relative to any of SEQ ID NOs: 298-590, within nucleotide positions 1-3 and 8-10 of the crRNA sequence. In some embodiments, a crRNA sequence includes one nucleotide substitution, e.g., relative to any of SEQ ID NOs: 298-590, within nucleotide positions 1-3 and 8-10 of the crRNA sequence. In some embodiments, a crRNA sequence includes two nucleotide substitutions, e.g., relative to any of SEQ ID NOs: 298-590, within nucleotide positions 1-3 and 8-10 of the crRNA sequence. In some embodiments, a crRNA sequence includes three nucleotide substitutions, e.g., relative to any of SEQ ID NOs: 298-590, within nucleotide positions 1-3 and 8-10 of the crRNA sequence. In some embodiments, a crRNA sequence includes four nucleotide substitutions, e.g., relative to any of SEQ ID NOs: 298-590, within nucleotide positions 1-3 and 8-10 of the crRNA sequence. In some embodiments, a crRNA sequence includes five nucleotide substitutions, e.g., relative to any of SEQ ID NOs: 298-590, within nucleotide positions 1-3 and 8-10 of the crRNA sequence.

Similarly, in some embodiments, a crRNA sequence used in the compositions and/or methods of the disclosure includes one or more nucleotide substitutions, e.g., relative to any of SEQ ID NOs: 298-590, within nucleotide positions 1-3 and 8 of the crRNA sequence. In some embodiments, a crRNA sequence includes one nucleotide substitution, e.g., relative to any of SEQ ID NOs: 298-590, within nucleotide positions 1-3 and 8 of the crRNA sequence. In some embodiments, a crRNA. In some embodiments, a crRNA sequence includes two nucleotide substitutions, e.g., relative to any of SEQ ID NOs: 298-590, within nucleotide positions 1-3 and 8 of the crRNA sequence. In some embodiments, a crRNA. In some embodiments, a crRNA sequence includes three nucleotide substitutions, e.g., relative to any of SEQ ID NOs: 298-590, within nucleotide positions 1-3 and 8 of the crRNA sequence. In some embodiments, a crRNA. In some embodiments, a crRNA sequence includes four nucleotide substitutions, e.g., relative to any of SEQ ID NOs: 298-590, within nucleotide positions 1-3 and 8 of the crRNA sequence. In some embodiments, a crRNA.

In yet other embodiments, a crRNA sequence used in the compositions and/or methods of the disclosure include one or more nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes one nucleotide substitution, e.g., relative to any of SEQ ID NOs: 298-590, throughout the entire sequence of the crRNA, e.g., as determined through experimentation. In some embodiments, a crRNA sequence used in the compositions and/or methods of the disclosure includes two nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes three nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes four nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes five nucleotide substitutions, e.g., relative to any of SEQ ID NOs: 298-590, throughout the entire sequence of the crRNA, e.g., as determined through experimentation.

In some embodiments, the joint disease or condition is arthritis. In some embodiments, the arthritis is osteoarthritis.

In some embodiments, the administering includes intra-articular injection of the pharmaceutical composition into the joint of the subject. In some embodiments, the pharmaceutical composition is administered during surgery. In some embodiments, the pharmaceutical composition is administered after surgery. In some embodiments, the pharmaceutical composition is a controlled release pharmaceutical composition.

In some embodiments, the pharmaceutical composition includes one or more viral vectors, as described herein, collectively comprising the one or more nucleic acids. In some embodiments, the one or more viral vectors include a recombinant virus selected from a retrovirus, an adenovirus, an adeno-associated virus, a lentivirus, and a herpes simplex virus-1. In some embodiments, the one of more viral vectors include a recombinant adeno-associated virus (AAV). In some embodiments, the recombinant AAV is of serotype 5 (AAV5). In some embodiments, the recombinant AAV is of serotype 6 (AAV6).

In some embodiments, the one of more viral vectors include a first viral vector comprising a first nucleic acid, in the one or more nucleic acids, encoding the Cas9 protein, and a second viral vector comprising a second nucleic acid, in the one or more nucleic acids, encoding the at least one guide RNA. In some embodiments, the one of more viral vectors comprise a viral vector comprising a single nucleic acid, wherein the single nucleic acid encodes the Cas9 protein and the at least one guide RNA.

In some embodiments, the composition includes one or more liposomes collectively comprising the one or more nucleic acids. In some embodiments, the one or more nucleic acids are present in a naked state.

In some embodiments, the Cas9 protein is an S. pyogenes Cas9 polypeptide. In some embodiments, the Cas9 protein is an S. aureus Cas9 polypeptide.

### Methods of Treating Osteoarthritis and Other Diseases

The compositions and methods described herein can be used in a method for treating diseases. In an embodiment, they are for use in treating inflammatory joint disorders. They may also be used in treating other disorders as described herein and in the following paragraphs. In an aspect, the compositions and methods are used to treat osteoarthritis (OA).

In some embodiments, the present disclosure provides a method for the treatment or prevention of a joint disease or condition the method comprising introducing a gene-editing system, wherein the gene-editing system targets at least one locus related to joint function. In some embodiments, the joint disease is osteoarthritis. In an aspect, the method is used to treat a canine with osteoarthritis. In another aspect, the method is used to treat a mammal with degenerative joint disease. In some aspects, the method is used to treat a canine or an equine with a joint disease. In some aspects, the method is used to treat osteoarthritis, post-traumatic arthritis, post-infectious arthritis, rheumatoid arthritis, gout, pseudogout, auto-immune mediated arthritides, inflammatory arthritides, inflammation-mediated and immune-mediated diseases of joints.

In some embodiments, the method further comprises gene-editing a portion of a the joint synoviocytes to reduce or silence the expression of one or more of IL-1α, IL-1β, IL-4, IL-9, IL-10, IL-13, and TNF-α. In an aspect, the method further comprises gene-editing a portion of a the joint synoviocytes to reduce or silence the expression of one or more of IL-1α, IL-1β.

In an aspect, the method further comprises gene-editing, wherein the gene-editing comprises one or more methods selected from a CRISPR method, a TALE method, a zinc finger method, and a combination thereof.

In some aspects, the method further comprises delivering the gene-editing using an AAV vector, a lentiviral vector, or a retroviral vector. In a preferred embodiment, the method further comprises delivering the gene-editing using AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10. In some aspects, the AAV vector comprises a serotype selected from the group consisting of: AAV1, AAV5, AAV6, AAV6 (Y705F/Y731F/T492V), AAV8, AAV9, and AAV9 (Y731F).

### Pharmaceutical Compositions and Methods of Administration

The methods described herein include the use of pharmaceutical compositions comprising CRISPR gene (e.g., IL-1α and/or IL-1β) editing complexes as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

In one embodiment, a composition of the present disclosure can be delivered to a subject subcutaneously (e.g., intra-articular injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implants, subcutaneous implants, and transdermal implants.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79(3-4): 141-152.

The pharmaceutical compositions can be included in a container, kit, pack, or dispenser together with instructions for administration.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1. Reducing IL-1 Expression by CRISPR Gene-Engineering in a Mouse Model of Osteoarthritis

Sixty C57B mice are selected and distributed into four groups of fifteen mice each. The DMM surgical method is used to induce OA in each of the mice. Once the mice have developed OA, the mice are treated as follows:

Group 1: Direct injection into the OA joint a CRISPR AAV vector engineered to target IL-1α and IL-1β, and silence or reduce the expression of IL-1 protein.

Group 2: Direct injection into the OA joint a CRISPR AAV vector engineered with a “nonsense” payload that will not affect an IL-1 production; a negative control.

Group 3: Direct injection into the OA joint a CRISPR AAV vector engineered to target IL-1Ra, and silence or reduce the expression of IL-1Ra protein.

Group 4: Direct injection into the OA joint sterile buffered saline; a control for the injection process.

The mice are monitored before and after treatment to assess effects on their locomotion, and exploratory activities. Mechanical sensitivity and changes to the gait are also monitored. Allodynia and hind limb grip force may also be monitored.

After about eight weeks, the animals are sacrificed and the OA joint tissue assessed for gross histopathology, and IL-1 expression by IHC. Biomarkers of inflammation are also assessed, for example, MMP-3 expression in the OA joint.

Group 1 mice, treated with a CRISPR AAV vector engineered to target IL-1α and IL-1β, and silence or reduce the expression of IL-1 protein, will show reduced levels of IL-1 by IHC, tissue regeneration by histopathology, and lower levels of inflammation biomarkers than any of the three other Groups. Group 3 mice will show relatively higher levels of inflammation biomarker than any of the other three groups.

### Example 2. Assessing Guide Cutting Efficiency Against Mouse ILIA and IL1B

In Vitro Cleavage Assay

CRISPR guide RNA's (Phosphorothionate-modified sgRNA, Table 3) were designed against Exon 4 of Il1a and Exon 4 of Il1b (Il1a-201 ENSMUST00000028882.1 and Il1b-201 ENSMUST00000028881.13; see Table 2 for target sequences on Exon 4 of Il1a and Exon 4 of Il1b). C57BL/6 mouse genomic DNA was used to amplify Exon 4 of Il1a and Il1b by PCR (Phusion High-Fidelity DNA polymerase, NEB cat #M0530S) Il1a primer fwd: CATTGGGAGGATGCTTAGGA (SEQ ID NO:620), Il1a primer rev: GGCTGCTTTCTCTCCAACAG (SEQ ID NO:621), Il1b primer fwd: AGGAAGCCTGTGTCTGGTTG (SEQ ID NO:622), Il1b primer rev: TGGCATCGTGAGATAAGCTG (SEQ ID NO:623). Amplicons were PCR purified (QiaQuick PCR purification kit cat #28106). Guide cutting efficiency was determined using an in vitro cleavage assay using 100 ng purified PCR product, 200 ng modified guide RNA (Sigma Aldrich) and 0.5 μg TrueCut Spy Cas9 protein V2 (Invitrogen A36498) or 0.5 μg Gene Snipper NLS Sau Cas9 (BioVision Cat #M1281-50-1). The two types of Cas9, S. pyogenes Cas9 and S. aureus Cas9, were compared for their editing capabilities. A 2% agarose gel was used for a qualitative readout of the cleavage assay.

Editing Cell Lines

CRISPR guide RNA's (Phosphorothionate-modified sgRNA, Table 2) were designed against Exon 4 of Il1a and Exon 4 of Il1b (Il1a-201 ENSMUST00000028882.1 and Il1b-201 ENSMUST00000028881.13). Guide RNA cutting efficiency was determined in a pool of J774.2 and NIH3T3 cells using Sanger sequencing and Synthego ICE (see, e.g., Inference of CRISPR Edits from Sanger Trace Data, Hsiau T, Maures T, Waite K, Yang J et al. biorxiv. 2018, which is incorporated by reference herein for all purposes), or TIDE (see, e.g., Easy quantitative assessment of genome editing by sequence trace decomposition, Brinkman E, Chen T, Amendola M and Van Steensel B. Nucleic Acids res 2014, which is incorporated by reference herein for all purposes) web tools to calculate percent editing. The experiment also compared the efficiency of S. pyogenes Cas9 and S. aureus Cas9. The cells were electroporated (Amaxa 4D Nucleofector unit, Lonza) with 5 μg TrueCut Spy Cas9 protein V2 (Invitrogen A36498) or 5 μg EnGen Sau Cas9 protein (NEB M0654T) with 100 pmol modified guide RNA (Sigma Aldrich). SF nucleofector solution and programme CM139 was used for J774.2 cells and SG nucleofector solution and programme EN158 was used for NIH3T3 cells. A cell pellet was taken 3 days' post electroporation and gDNA was extracted from each pool (Qiagen, DNeasy blood and tissue kit, 69506). Exon 4 of Il1a or Il1b was amplified in the appropriate pool by PCR (Phusion High-Fidelity DNA polymerase, NEB, cat #M0530S). Ilia primer fwd: TGGTTTCAGGAAAACCCAAG (SEQ ID NO:624), Il1a primer rev: GCAGTATGGCCAAGAAAGGA (SEQ ID NO:625), Il1b primer fwd: AGGAAGCCTGTGTCTGGTTG (SEQ ID NO:622), Il1b primer rev: CTGGGCAAGAACATTGGATT (SEQ ID NO:626). Amplicons were subjected to Sanger sequencing, and analyzed using either the Synthego ICE or TIDE web tools to determine the absence of wild type sequence in each clone and the presence of indels resulting in a frameshift in the cDNA sequence.

TABLE 2
Target Il1a and Il1b Sequences

| Identifier | Guide ID | Gene | Exon | Cas9 | Target Sequence 5′-3′ | PAM |
|---|---|---|---|---|---|---|
| SEQ ID NO: 7 | sg43 | Il1a | 4 | S. pyogenes | GTATCAGCAACGTCAAGCAA | CGG |
| SEQ ID NO: 8 | sg44 | Il1a | 4 | S. pyogenes | CTGCAGGTCATCTTCAGTGA | AGG |
| SEQ ID NO: 9 | sg45 | Il1a | 4 | S. pyogenes | TATCAGCAACGTCAAGCAAC | GGG |
| SEQ ID NO: 10 | sg46 | Il1a | 4 | S. pyogenes | GCCATAGCTTGCATCATAGA | AGG |
| SEQ ID NO: 11 | sg47 | Il1b | 4 | S. pyogenes | CATCAACAAGAGCTTCAGGC | AGG |
| SEQ ID NO: 12 | sg48 | Il1b | 4 | S. pyogenes | TGCTCTCATCAGGACAGCCC | AGG |
| SEQ ID NO: 13 | sg49 | Il1b | 4 | S. pyogenes | GCTCATGTCCTCATCCTGGA | AGG |
| SEQ ID NO: 14 | sg50 | Il1b | 4 | S. pyogenes | CCTCATCCTGGAAGGTCCAC | GGG |
| SEQ ID NO: 15 | sg51 | Il1a | 4 | S. aureus | TTACTCCTTACCTTCCAGATC | ATGGGT |
| SEQ ID NO: 16 | sg52 | Il1a | 4 | S. aureus | GAAACTCAGCCGTCTCTTCTT | CAGAAT |
| SEQ ID NO: 17 | sg53 | Il1a | 4 | S. aureus | CAACTTCACCTTCAAGGAGAG | CCGGGT |
| SEQ ID NO: 18 | sg54 | Il1b | 4 | S. aureus | GTGTCTTTCCCGTGGACCTTC | CAGGAT |
| SEQ ID NO: 19 | sg55 | Il1b | 4 | S. aureus | CACAGCTTCTCCACAGCCACA | AGTAGT |
| SEQ ID NO: 20 | sg56 | Il1b | 4 | S. aureus | GTGCTGCTGCGAGATTTGAAG | CTGGAT |

TABLE 3
CRISPR Guide RNA′s.

| Identifier | Guide ID | Gene | Exon | Cas9 | CRNA Sequence 5′-3′ | PAM |
|---|---|---|---|---|---|---|
| SEQ ID NO: 21 | sg43 | Il1a | 4 | S. pyogenes | GUAUCAGCAACGUCAAGCAA | CGG |
| SEQ ID NO: 22 | sg44 | Il1a | 4 | S. pyogenes | CUGCAGGUCAUCUUCAGUGA | AGG |
| SEQ ID NO: 23 | sg45 | Il1a | 4 | S. pyogenes | UAUCAGCAACGUCAAGCAAC | GGG |
| SEQ ID NO: 24 | sg46 | Il1a | 4 | S. pyogenes | GCCAUAGCUUGCAUCAUAGA | AGG |
| SEQ ID NO: 25 | sg47 | Il1b | 4 | S. pyogenes | CAUCAACAAGAGCUUCAGGC | AGG |
| SEQ ID NO: 26 | sg48 | Il1b | 4 | S. pyogenes | UGCUCUCAUCAGGACAGCCC | AGG |
| SEQ ID NO: 27 | sg49 | Il1b | 4 | S. pyogenes | GCUCAUGUCCUCAUCCUGGA | AGG |
| SEQ ID NO: 28 | sg50 | Il1b | 4 | S. pyogenes | CCUCAUCCUGGAAGGUCCAC | GGG |
| SEQ ID NO: 29 | sg51 | Il1a | 4 | S. aureus | UUACUCCUUACCUUCCAGAUC | ATGGGT |
| SEQ ID NO: 30 | sg52 | Il1a | 4 | S. aureus | GAAACUCAGCCGUCUCUUCUU | CAGAAT |
| SEQ ID NO: 31 | sg53 | Il1a | 4 | S. aureus | CAACUUCACCUUCAAGGAGAG | CCGGGT |
| SEQ ID NO: 32 | sg54 | Il1b | 4 | S. aureus | GUGUCUUUCCCGUGGACCUUC | CAGGAT |
| SEQ ID NO: 33 | sg55 | Il1b | 4 | S. aureus | CACAGCUUCUCCACAGCCACA | AGTAGT |
| SEQ ID NO: 34 | sg56 | Il1b | 4 | S. aureus | GUGCUGCUGCGAGAUUUGAAG | CTGGAT |

Each cRNA (see, e.g., Table 3) was synthesized as a single guide RNA consisting of the cRNA sequences above fused to the tracrRNA sequences below (see, e.g., SEQ ID Nos: 35-36). In certain embodiments, an A< >U flip is used to increase guide RNA activity.

Sau Cas9:
(SEQ ID NO: 35)
GUUAUAGUACUCUGGAAACAGAAUCUACUAAAACAAGGCAAAAUGCCGU
GUUUAUCUCGUCAACUUGUUGGCGAGAUUUUU
Spy Cas9:
(SEQ ID NO: 36)
GUUAUAGAGCUAGAAAUAGCAAGUUAAAAUAAGGCUAGUCCGUUAUCAA
CUUGAAAAAGUGGCACCGAGUCGGUGCUUUUUU

In Vitro Cleavage Assay

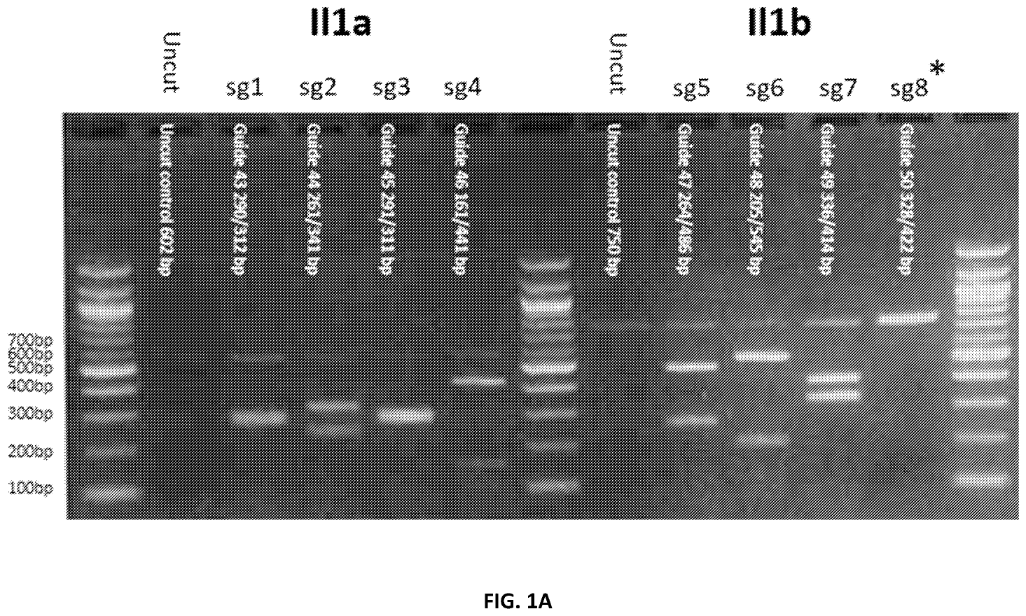

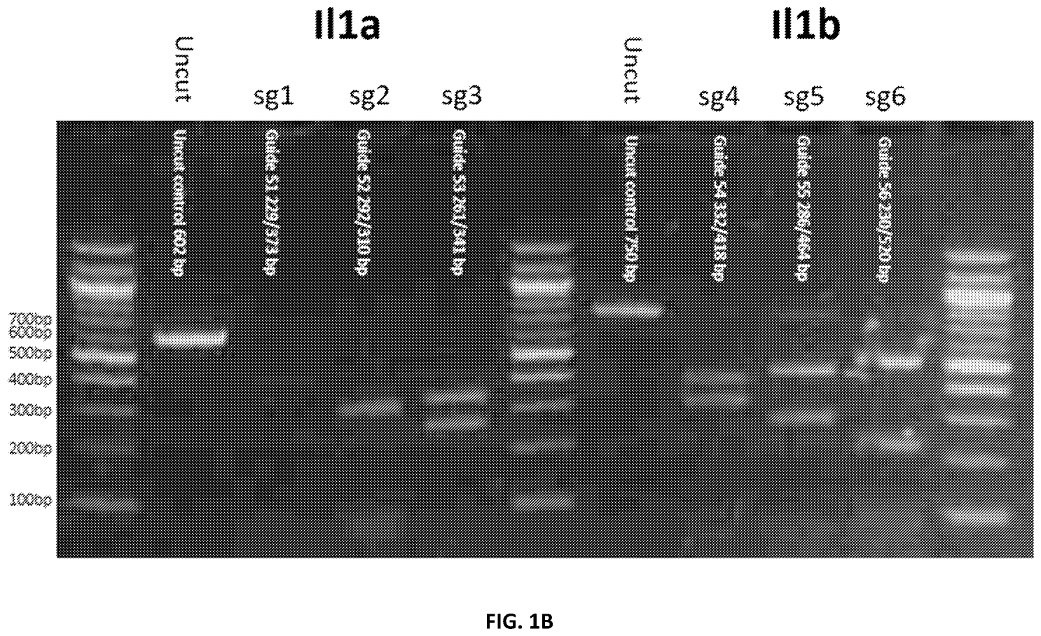

FIG. 1A illustrates agarose gel electrophoresis analysis of 100 ng mouse DNA, cleaved by 0.5 μg Spy Cas9 and 200 ng modified guide RNA's 43-46 for Il1a gene and 47-50 for IL1B. DNA is cut at a specific site by the cas9 using the guide RNA to create a predictable band pattern on the agarose gel compared to the uncut control (without wishing to be bound by any particular theory, the agarose gel electrophoresis for sg8* appears to show a failed synthesis).

FIG. 1B illustrates agarose gel electrophoresis analysis of 100 ng mouse DNA, cleaved by 0.5 μg Sau Cas9 and 200 ng modified guide RNA's 51-53 for Il1a gene and 54-56 for Il1b. DNA is cut at a specific site by the Cas9 using the guide RNA to create a predictable band pattern on the agarose gel compared to the uncut control.

Editing Cell Lines

Genomic DNA was extracted from the edited pools and the Il1a or Il1b exon 4 was PCR amplified in the appropriate pools. The PCR products were sent for sanger sequencing and then deconvoluted using TIDE or Synthego ICE software. Synthego ICE was used to deconvolute the Spy Cas9 pools. The software can determine the patterns of editing in each pool based on the guide RNA sequence and PAM site. It can distinguish between editing which has caused an in frame deletion that could lead to a truncated functional protein, and editing which has causes a frameshift mutation which will lead to a true knockout. The SauCas9 pools were analysed with TIDE because Synthego ICE software cannot deconvolute SauCas9 editing. TIDE analysis works in a similar way to ICE by determining patterns of editing in a pool based on the guide RNA and PAM site. However, rather than giving a true knockout score, it gives an editing efficiency score, which cannot distinguish between in frame and frameshift editing patterns. Therefore, editing efficiency scores may over represent the guide RNA's ability to knockout a protein. SpyCas9 is the standard protein used in CRISPR gene editing. However, it is 4101 bp compared to Sau Cas9 which is 3156 bp. Due to the size limitations of packaging some viruses, such as AAV, it was decided to compare the editing capabilities of SauCas9 and SpyCas9 to see whether the smaller Sau Cas9 could be used in the vector being designed for this project.

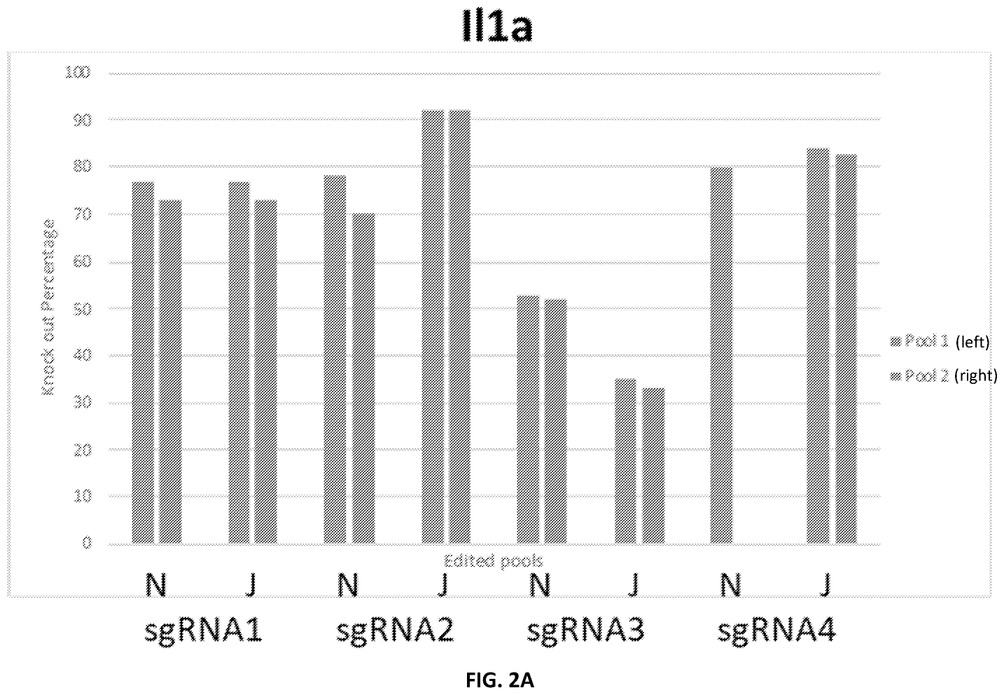

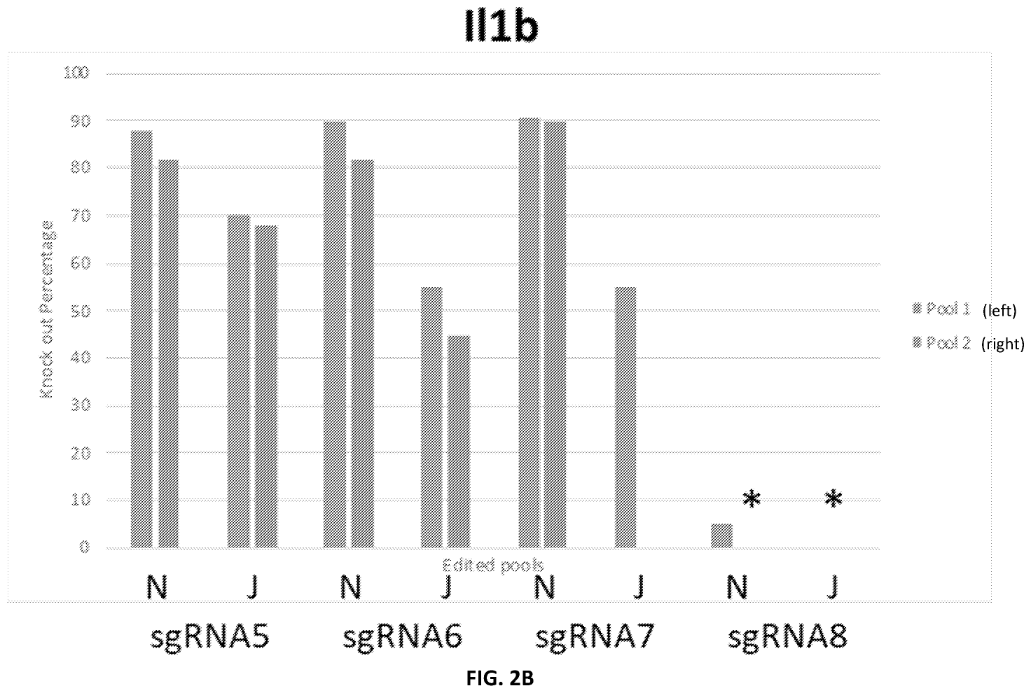

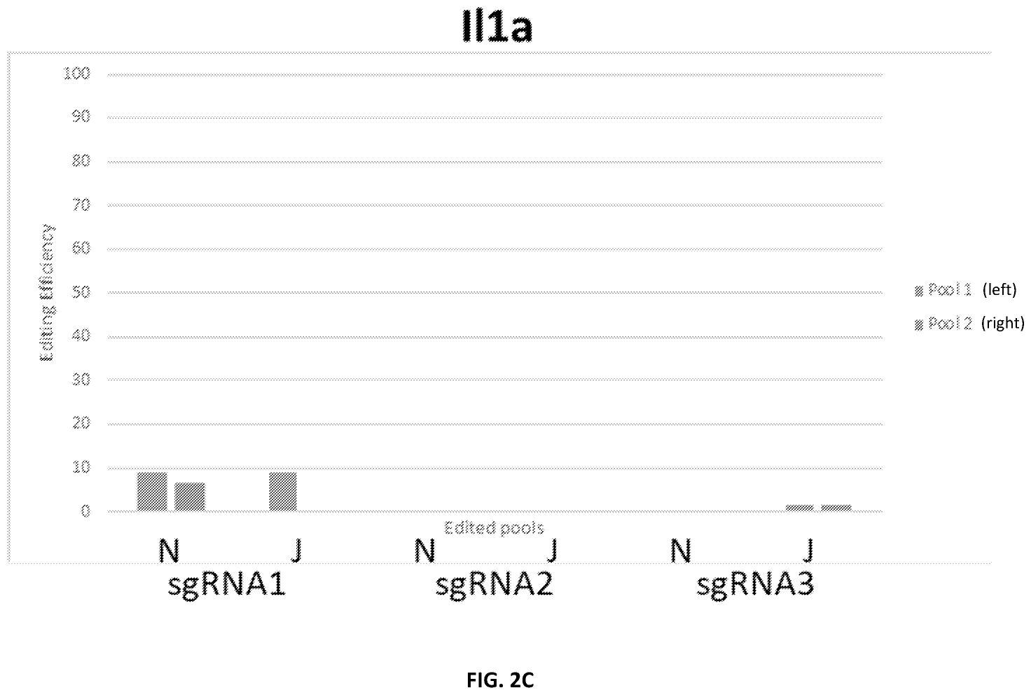

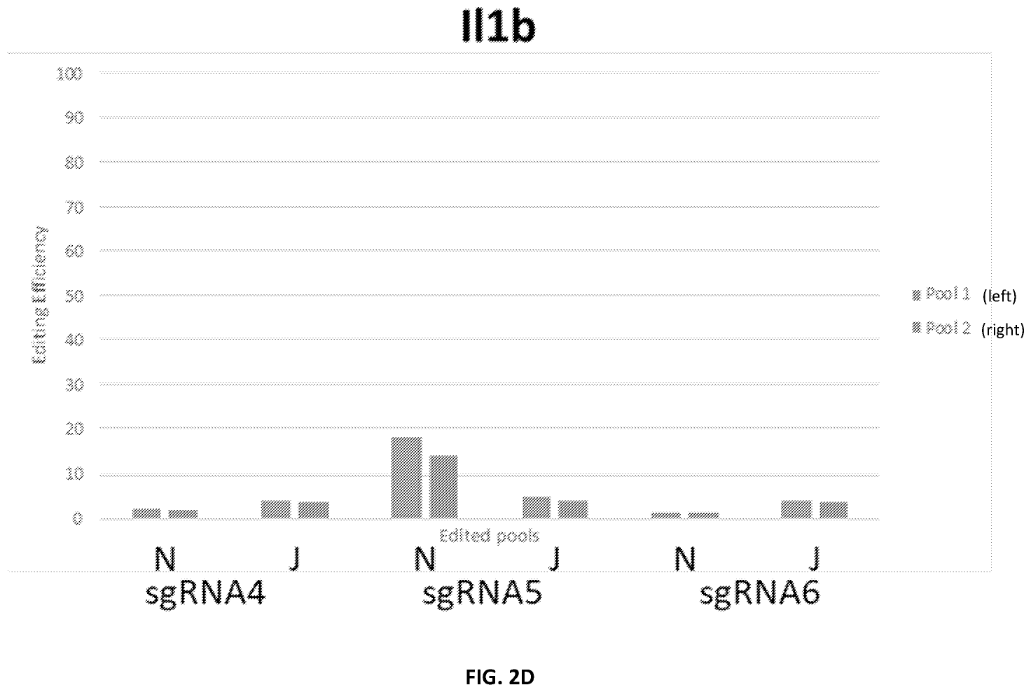

FIGS. 2A-2D illustrate graphs displaying editing efficiencies of Spy Cas9 (FIGS. 2A and 2B) and SauCas9 (FIGS. 2C and 2D) used with a range of guide RNA's in J774.2 (“J”) and NIH3T3 (“N”) cells. Editing efficiencies were determined using Synthego ICE or TIDE sanger deconvolution software. FIG. 2A: knock out efficiency of Il1a using guide RNA 43-46 with SpyCas9 in J774.2 and NIH3T3. Synthego ICE was used to deconvolute the sanger sequence trace and determine knock out efficiency. FIG. 2B: knock out efficiency of Il1b using guide RNA 47-50 with SpyCas9 in J774.2 and NIH3T3; without wishing to be bound by any particular theory, the data for sgRNA8 appears to show a failed synthesis. Synthego ICE was used to deconvolute the sanger sequence trace and determine knock out efficiency. FIG. 2C: knock out efficiency of Il1a using guide RNA 51-53 with saCas9 in J774.2 and NIH3T3. TIDE was used to deconvolute the sanger sequence trace and determine the editing efficiency. FIG. 2D: knock out efficiency of Il1b using guide RNA 54-56 with Sau Cas9 in J774.2 and NIH3T3. TIDE was used to deconvolute the sanger sequence trace and determine the editing efficiency.

### Example 3. Reducing IL-1β Expression by CRISPR Gene-Engineering in a Mouse Uric Acid Model

Time Course Experiment to Determine Optimal Pre-Treatment Time

A pilot experiment is performed to determine optimal pre-treatment time of mice with virus prior to challenging the mice with uric acid. Mice are injected with GFP-labeled AAV5 vector into the knee joint. Viral load is then quantified by PCR and location of viral infection is quantified by histology at 3, 5, and 7 days after infection. A treatment time that yields robust expression of virus inside the joint is selected as the optimal lead time for injecting viral vectors into the mice for the experiments to determine the reduction of IL-1b in a mouse uric acid model by a CRISPR AAV vector engineered to target IL-1b and silence or reduce expression of IL-1b.

Experiment to Confirm CRISPR AAV (AAV-spCas9) Knock Down of IL-1b Expression and Treatment Effect in Uric Acid Model

Mice are selected and distributed into three groups:

Group 1: mice injected with a CRISPR AAV vector (AAV-spCas9) engineered to target IL-1b, and silence or reduce expression of IL-1 protein,

Group 2: mice injected with “scrambled” guide RNA/Cas9 (AAV-spCas9), a CRISPR AAV vector engineered with a payload that will not affect IL-1 production, and

Group 3: mice injected with saline.

The mice are then challenged with uric acid after an optimal pre-treatment time. Within 24 hours of injection with uric acid, the animals are sacrificed and the joint tissue is analyzed for cytokine expression (e.g., assessed for IL-1 expression by IHC). The joint tissue may also be assessed for gross histopathology and for expression of biomarkers of inflammation.

Group 1 mice treated with a CRISPR AAV vector engineered to target IL-1b, and silence or reduce the expression of IL-1 protein, will show reduced levels of IL-1 by IHC and lower levels of inflammation biomarkers than any of the two other groups.

### Example 4. Time Course Study of Intra-Articular Injection of AAV in Mice

A study was conducted to evaluate the time course for injecting AAV into the joint of male C57BL/6 mice.

Materials & Methods

Test Article Identification and Preparation—The eGFP AAVPrime™ Purified Adeno-associated Viral Particles: GFP-tagged AAV5 GeneCopoeia™, catalogue No. AB201, lot No. GC08222K1902, 1.18×10¹³ Genome Copies/mL) and AAV6 (GeneCopoeia™, catalogue No. AB401, lot No. GC09242K1905, 5.47×10¹² Genome Copies/mL) we supplied. AAV-particles were shipped on dry ice and were stored at −80° C. immediately upon receipt. Just prior to dosing, the AAV-particles were reconstituted in phosphate buffered saline (PBS without calcium and magnesium: Corning, lot No. 11419005) for IA dosing at 10 μL per knee. See the study protocol (Appendix A) for additional details of test article preparation, storage, and handling.

Test System Identification—Male C57BL/6 mice (N=30) that were 8 to 10 weeks old were obtained from The Jackson Laboratory (Bar Harbor, Me.). The mice weighed approximately 24 to 29 grams (mean of 26 g) at enrollment on study day 0. The animals were identified by a distinct mark at the base of the tail delineating group and animal number. After randomization, all cages were labeled with protocol number, group numbers, and animal numbers with appropriate color-coding (Appendix A).

Environment & Husbandry—Upon arrival, the animals were housed 3 to 5 per cage in polycarbonate cages with wood chip bedding and suspended food and water bottles. The mice were housed either in shoebox cages (static airflow, approximately 70 in2 floor space) with filter tops or in individually ventilated pie cages (passive airflow, approximately 70-75 in2 floor space). Animal care including room, cage, and equipment sanitation conformed to the guidelines cited in the Guide for the Care and Use of Laboratory Animals (8th Edition). National Research Council, National Academy of Sciences, Washington, D.C., 2011, which is incorporated by reference herein in its entirety for all purposes.

The animals were acclimated for 4 days prior to being paced in the study. An attending veterinarian was on site or on call during the live phase of the study. No concurrent medications were given.

During the acclimation and study periods, the animals were housed in a laboratory environment with temperatures ranging 19° C. to 25° C. and relative humidity of 30% to 70%. Automatic timers provided 12 hours of light and 12 hours of dark. The animals were allowed access ad libitum to Envigo Teklad 8640 diet and fresh municipal tap water.

Experimental Design—On study day 0, the mice were randomized by body weight into treatment groups. Following randomization, the animals were dosed by intra-articular (IA) injection as indicated in Table 4. Animal body weights were measured as described in section 8.5.1. The mice were euthanized for necropsy and tissue collection at 3 time points (days 3, 5, and 7) as described below in the section titled ‘Necropsy Specimens’.

TABLE 4
Group and Treatment Information

| | | | Dose | | | | |
|---|---|---|---|---|---|---|---|
| | | | Level | Dose | Dose Conc. | Dose | |
| Group | N | Treatment | (particles) | Vol. | (particles/ml) | Route | Regimen |
| 1 | 30 | GFP- | 5 × 10⁹ | 10 μL | 5 × 10₁₁/mL | IA | 1× |
| | | tagged | | | | (right | (Day 0) |
| | | AAV5 | | | | knee) | |
| | | GFP- | 5 × 10⁹ | 10 μL | 5 × 10¹¹/mL | IA | 1× |
| | | tagged | | | | (left | (Day 0) |
| | | AAV6 | | | | knee) | |

Observations, Measurements, and Specimens

Body Weight Measurements—The mice were weighed for randomization on study day 0 and again on days 1, 3, 5, and 7. Body weight measurements can be found in Table 6.

Necropsy Specimens—The mice were necropsied on study days 3, 5, and 7 as indicated in Table 5.

TABLE 5
Necropsy Schedule

| Group | Animal No. | Time-point(s) |
|---|---|---|
| 1 | 1-10 | Day 3 |
| 1 | 11-20 | Day 5 |
| 1 | 21-30 | Day 7 |

At necropsy, the mice were bled to exsanguination via cardiac puncture followed by cervical dislocation. Right and left knees were harvested from all animals. The skin and muscle were removed from the joints while keeping the joint capsule intact. Joints were flash-frozen separately in 15-mL conical tubes labeled with only mouse number, day of collection, and right or left leg. Knee joints were stored frozen at −80° C. for shipment.

Animal Disposition—Animal carcasses were disposed of according to BBP SOPS.

Specimen and Raw Data Storage—Specimens (right and left knee joints), study data, and reports were delivered during or at the completion of the study.

Statement of Effect of Deviations on the Quality and Integrity of the Study—There were no deviations from the study protocol.

Results/Conclusions

On study day 0, male C57BL/6 mice received IA injections of GFP-tagged AAV5 (5×10⁹ particles, 10 μL) into right knees and IA injections of GFP-tagged AAV6 (5×10⁹ particles, 10 μL) into left knees. The animals were weighed on study days 0, 1, 3, 5, and 7. Necropsies were performed on study day 3 (animals 1-10), day 5 (animals 11-20), and day 7 (animals 21-30), and right and left knee joints were collected for shipment. The live portion of this study was completed successfully including animal weighing, dosing, and biological sample collection. All animals survived to study termination.

### References

Guide for the Care and Use of Laboratory Animals (8th Edition). National Research Council, National Academy of Sciences, Washington, D.C., 2011, which is incorporated by reference herein in its entirety for all purposes.

TABLE 6
Body Weight and Dose Calculation Data (MTC-UCM-1)

| Treatment | | | | | | |
|---|---|---|---|---|---|---|
| Group | | Day 0 | Day 1 | | Day 3 | |
| Group 1 | Day 0 | Dose | | Body | | Body |
| C57Bl/6 | Body | Vol. | Body | Wt. % | Body | Wt. % |
| IA [symbol] | Wt. | 10 μl | Wt. | [symbol] | Wt. | [symbol] |
| 1× (D0) | (g) | (ml) | (g) | Baseline | (g) | Baseline |
| 1 | 26.68 | 0.01 | 28.53 | −0.6% | 26.22 | −1.7% |
| 2 | 27.63 | 0.01 | 27.55 | −0.4% | 20.54 | −4.0% |
| 3 | 28.64 | 0.01 | 27.97 | −2.3% | 28.57 | −0.2% |
| 4 | 28.13 | 0.01 | 27.63 | −1.9% | 27.63 | −1.8% |
| 5 | 26.18 | 0.01 | 26.07 | −0.4% | 25.87 | −1.2% |
| 6 | 26.38 | 0.01 | 26.02 | −1.4% | 26.30 | −0.3% |
| 7 | 29.13 | 0.01 | 29.21 | −0.1% | 28.83 | −1.0% |
| 8 | 24.22 | 0.01 | 23.90 | −1.3% | 23.51 | −2.9% |
| 9 | 25.40 | 0.01 | 24.97 | −1.7% | 24.49 | −3.6% |
| 10 | 24.85 | 0.01 | 24.21 | −2.6% | 23.86 | −4.0% |
| 11 | 27.76 | 0.01 | 27.26 | −1.8% | 28.02 | 0.9% |
| 12 | 25.23 | 0.01 | 24.77 | −1.8% | 24.90 | −1.3% |
| 13 | 24.92 | 0.01 | 24,35 | −1.9% | 24.59 | −1.3% |
| 14 | 24.33 | 0.01 | 28.19 | −0.6% | 23.86 | −1.9% |
| 15 | 23.83 | 0.01 | 23.83 | 0.0% | 23.26 | −2.4% |
| 16 | 24.83 | 0.01 | 24.39 | −1.8% | 24.15 | −2.7% |
| 17 | 25.94 | 0.01 | 25.93 | 0.0% | 26.28 | 1.3% |
| 18 | 27.21 | 0.01 | 37.44 | 0.8% | 27.60 | 1.4% |
| 19 | 25.61 | 0.01 | 25.17 | −1.7% | 25.22 | −1.5% |
| 20 | 27.81 | 0.01 | 27.26 | −2.0% | 26.99 | −2.9% |
| 21 | 26.63 | 0.01 | 26.63 | 0.0% | 26.73 | 0.3% |
| 22 | 26.96 | 0.01 | 27.40 | 1.6% | 25.35 | −4.3% |
| 23 | 27.89 | 0.01 | 27.21 | −1.7% | 27.22 | −1.7% |
| 24 | 25.90 | 0.01 | 23.71 | −0.7% | 25.45 | −1.7% |
| 25 | 24.03 | 0.01 | 28.11 | 0.3% | 23.40 | −2.6% |
| 26 | 27.60 | 0.01 | 26.67 | −3.4% | 27.00 | −2.2% |
| 27 | 27.87 | 0.01 | 27.59 | −1.0% | 27.22 | −2.3% |
| 28 | 24.43 | 0.01 | 24.25 | −0.7% | 24.14 | −1.2% |
| 29 | 26.75 | 0.01 | 26.02 | −2.7% | 26.46 | −1.1% |
| 30 | 25.93 | 0.01 | 23.99 | 0.2% | 25.80 | −0.5% |
| Mean | 26.28 | | 26.01 | −1.0% | 25.86 | −1.6% |
| SE | 0.27 | | 0.27 | 0.2% | 0.29 | 0.3% |
| Treatment | | | | | | |

| Group | Day 5 | | Day 7 | | |
|---|---|---|---|---|---|
| Group 1 | | Body | | Body | Change in |
| C57Bl/6 | Body | Wt. % | Body | Wt. % | Bodyweight |
| IA [symbol] | Wt. | [symbol] | Wt. | [symbol] | from |
| 1× (D0) | (g) | Baseline | (g) | Baseline | Baseline (g) |
| 1 | | | | | −0.46 |
| 2 | | | | | −1.11 |
| 3 | | | | | −0.07 |
| 4 | | | | | −0.50 |
| 5 | | | | | −0.31 |
| 6 | | | | | −0.08 |
| 7 | | | | | −0.30 |
| 8 | | | | | −0.71 |
| 9 | | | | | −0.91 |
| 10 | | | | | −0.99 |
| 11 | 28.62 | 3.1% | | | 0.86 |
| 12 | 25.04 | −0.8% | | | −0.19 |
| 13 | 24.94 | 0.1% | | | 0.02 |
| 14 | 23.68 | −2.7% | | | −0.65 |
| 15 | 23.71 | −0.5% | | | −0.11 |
| 16 | 24.26 | −2.3% | | | −0.57 |
| 17 | 26.15 | 0.8% | | | 0.21 |
| 18 | 27.88 | 2.3% | | | 0.67 |
| 19 | 25.64 | 0.1% | | | 0.03 |
| 20 | 27.71 | −0.4% | | | −0.10 |
| 21 | 26.44 | −0.7% | 26.70 | 0.3% | 0.07 |
| 22 | 25.76 | −4.5% | 25.75 | −1.5% | −1.21 |
| 23 | 23.53 | −0.6% | 27.33 | −1.3% | −0.36 |
| 24 | 25.61 | −1.1% | 26.00 | 0.4% | 0.10 |
| 25 | 23.76 | −1.1% | 24.10 | 0.3% | 0.07 |
| 26 | 23.44 | −0.6% | 27.18 | −1.5% | −0.42 |
| 27 | 27.08 | −2.8% | 27.30 | −2.0% | −0.57 |
| 28 | 34.50 | 0.3% | 24.26 | 1.4% | 0.33 |
| 29 | 26.48 | −1.0% | 26.48 | −1.0% | −0.27 |
| 30 | 25.93 | −0.8% | 25.89 | −0.2% | −0.04 |
| Mean | 25.90 | −0.6% | 26.15 | −0.8% | −0.25 |
| SE | 0.33 | 0.4% | 0.34 | 0.5% | 0.09 |

[symbol]

indicates data missing or illegible when filed

Protocol

Test System

Number of animals: 33 (30+3 extra)

### Species/Strain or Breed: C57BL/6

### Vendor: Jackson

Age/Wt at Arrival: 8-10 weeks old (˜20 grams)

### Gender: Male

Age/Wt Range at Study Initiation: At least 9 weeks by study initiation
Acclimation: Will be acclimated for at least 3 days after arrival at BBP
Housing: 3-5 animals/cage

| Mon | Tue | Wed | Thu | Fri | Sat | Sun |
|---|---|---|---|---|---|---|
| Week 1 | Week 1 | Week 1 | Week 1 | Week 1 | Week 1 | Week 1 |
| Day −4 | Day −3 | Day −2 | Day −1 | Day 0 | Day 1 | Day 2 |
| Distribute | | | | Weigh & Randomize. | Weigh | |
| animals on | | | | IA Injections | | |
| arrival into | | | | | | |
| groups for | | | | | | |
| acclimation | | | | | | |
| Week 2 | Week 2 | Week 2 | Week 2 | Week 2 | Week 2 | Week 2 |
| Day 3 | Day 4 | Day 5 | Day 6 | Day 7 | Day 8 | Day 9 |
| Weigh, Necropsy | | Weigh, Necropsy | | Weigh, Necropsy | | |
| Animals 1-10 | | Animals 11-20 | | Animals 21-30 | | |

Materials

| Name | Supplier | Cat #* |
|---|---|---|
| Isoflurance | VetOne | 502017 |
| Syringes & Needle | BD | As needed |
| Serum Separator Tubes | Greiner | #450472 (via Fisher) |
| (if needed) | Bio-One | |
| Li Hep Mini-Collect (if needed) | Greiner | #450480 (via Fisher) |
| | Bio-One | |
| EDTA Mini-Collect (if needed) | Greiner | #450477 (via Fisher) |
| | Bio-One | |
| K3EDTA (if needed) | Covidien | #8881311149 (via Fisher) |
| K3EDTA Vacutainer (if needed) | BD | #367856 (via Fisher) |
| Na Hep Vacutainer (if needed) | BD | #367871 (via Fisher) |
| Li Hep Vacutainer (if needed) | BD | #367960 (via Fisher) |

Study Calendar

Test Article and Vehicle Information

Unformulated Test Article Storage Conditions—GFP-tagged AAV5 (Group 1): −80 C; GFP-tagged AAV6 (Group 1): −80° C.

Vehicle Information—GFP-tagged AAV5 (Group 1): PBS (w/o Ca & Mg); GFP-tagged AAV6 (Group 1): PBS (w/o Ca & Mg).

Test Article Formulation Instructions & Calculations—GFP-tagged AAV5 (Group 1): Dilute stock to appropriate concentration using PBS; GFP-tagged AAV6 (Group 1): Dilute stock to appropriate concentration using PBS.

Dosing Formulations and Vehicle Storage & Stability—GFP-tagged AAV5 (Group 1): Dilute just prior to injecting; GFP-tagged AAV6 (Group 1): Dilute just prior to injecting.

Disposition of Test Articles Following Dosing—GFP-tagged AAV5 (Group 1): Discard formulations, retain stock solution for future studies; GFP-tagged AAV6 (Group 1): Discard formulations, retain stock solution for future studies.

Live Phase Deliverables

Live Phase Data Collection

| | | | |
|---|---|---|---|
| Type | Study Day | Grp (An) | Details |

| | | |
|---|---|---|
| Body Weight | Day 0, 1, 3, 5, 7 | All (Remaining) |

Necropsy Information

### Sacrifice Schedule:

Group 1 An 1-10: Day 3
Group 1 An 11-20: Day 5
Group 1 An 21-30: Day 7
   Method of Euthanasia: Bleed by cardiac puncture to exsanguinate followed by cervical dislocation.

### Time Points: Not Timed

Necropsy Tissue Sample Collection:

| Type | Gr/An | Details | Storage Condition | Disposition |
|---|---|---|---|---|
| Right | All | Remove skin and | Flash freeze | Ship |
| Injected | | muscle keeping joint | (15 ml conical | |
| Knee | | capsule intact | vial*) | |
| Left | All | Remove skin and | Flash freeze | Ship |
| Injected | | muscle keeping joint | (15 ml conical | |
| Knee | | capsule intact | vial*) | |

*Label tubes with only mouse number, day of collection, and left or right leg. Samples will be tested without reference to whether they are AAV-2 or AAV-5 injected. Key to be provided only after PCR completion.

Sample Analysis

Tissue Specimens—Hind limbs from AAV-injected mice were snap-frozen and shipped. On arrival, specimens were transferred to the −80° C. freezer for storage.

GFP Expression in Target Tissues—Hind limbs (paired) were thawed at room temperature and imaged in an IVIS bioluminescence imaging system (Lumina III; Perkin Elmer). GFP fluorescence was quantified using excitation at 488 nm and measuring emission at 510 nm. A total of 4 mice were evaluated at each time point (3 days, 5 days and 7 days). Tissues from the remaining 6 animals at each time point were retained for subsequent confirmation of viral burden using real-time PCR.

Results—As can be seen in FIG. 3, there was high-level expression of GFP within injected knee joints at 3 days post-injection. Viral loads decreased slightly at 5 days, then rose again to 7 days. With the limited sample size in this pilot study there was no significant difference between the behaviours of AAV-5 and AAV-6.

Discussion—The data from this study support the use of either AAV-5 or AAV-6 for intra-articular delivery of CRISPR-Cas9 into the mouse knee joint. The levels of both viral serotypes increased from 5 to 7 day, leaving open the possibility that they may have increased further if the follow-up had been extended to 2 or maybe 3 weeks. Additional work would be needed to confirm this, but the data thus far would suggest that there should be an interval of at least one week before the injection of the vector and challenge with intra-articular monoiodoacetate (MIA) crystals.

Background & Rationale—The monoiodoacetate (MIA)-induced OA model is used in this work for two reasons. First, natural (spontaneous) OA is extremely uncommon in mice, whereas the injection of MIA results in an induced model of OA that is relatively fast in onset, predictable and that provides good clinical correlation to the disease phenotype see in human OA patients, including intra-articular inflammation, pain and cartilage degeneration. Second, in contrast with surgical models such as destabilization of the medial meniscus (DMM) and transection of the anterior cruciate ligament (ACLT), the MIA model does not involve surgical incision of the joint capsule, making it much more relevant to the capsules of human patients with OA.

Injection of MIA crystals in rodents reproduces OA-like lesions and functional impairment that can be analyzed and quantified by techniques such as behavioral testing and objective lameness assessment. MIA is an inhibitor of glyceraldehyde-3-phosphatase and the resulting alterations in cellular glycolysis eventual cause the death of cells within the joint, including chondrocytes. Chondrocyte death manifests as cartilage degeneration and alterations in proteoglycan staining. Mice injected with MIA usually exhibit pain-like behavior within 72 hours, and cartilage loss by around 4 weeks post-injection. Increases in IL-1 expression have been documented within 2-3 days of injection in rats and in mice.

Study Design—Mice are injected unilaterally with either MIA or the saline vehicle control (one joint per animal). Within each group, half of the animals are pre-treated with the AAV-CRISPR-Cas9 vector targeting the mouse IL-1 beta gene, and the other half are injected with an AAV-CRISPR-Cas9 scrambled control. Animals from both groups will be taken off study at one of two time points: an early time point of 48 hours, to allow for assessment of the impact of therapy on the levels of IL-1 within the synovial fluid, and a late time point of 4 weeks to allow for assessment of the impact of therapy on cartilage breakdown and histological evidence of osteoarthritis.

Methods

Experimental Animals—A total of 80 mice are used in this study. The experimental procedures are reviewed and approved by the local IACUC. Mice are housed in micro-isolator cages, fed a standard laboratory animal diet, and allowed access to water ad libitum.

MIA Model & Anti-IL1 Therapy—Mice are acclimated for a period of 7 days ahead of the study. On the first day of the study, mice are anaesthetized with an inhaled mixture of isoflurane in oxygen. Once a surgical plane of anesthesia has been confirmed, the right hind limb is clipped and the skin scrubbed with a surgical antiseptic. 40 mice (Treated) receive an intra-articular injection of the AAV-CRISPR-Cas9 vector targeting IL-1, and the remaining 40 animals (Control) are injected intra-articularly with the AAV-CRISPR-Cas9 scrambled control. Seven days later, half of the animals in each group are injected in the same joint with MIA and half with the saline vehicle. This leads to the establishment of four study groups:

Group 1: Treated-MIA (20 mice)

Group 2: Control-MIA (20 mice)

Group 3: Treated-Vehicle (20 mice)

Group 4: Control-Vehicle (20 mice)

Ten mice per group are euthanised 48 hours after the MIA challenge in order to document IL-1 levels in the knee joint. The remaining animals will be housed for 4 weeks in order to evaluate the effects of therapy on pain behavior (behavioral testing, including von Frey testing), lameness (limb use), joint swelling (caliper measurement) and joint pathology (histopathology).

Euthanasia & Tissue Collection—Mice are killed by exsanguination, followed by cervical dislocation. Joints are opened and either flushed for IL-1 measurement (48-hour group) or immersion fixed in 10% formalin for decalcified histopathology (4-week group).

### Example 5. Efficacy of AAV-6 and AAV-5 Mediated CRISPR Treatment in MSU-Crystal Induced Joint Arthritis in Mice

Introduction and Objectives

The objective of these studies is to identify compounds/proteins that inhibit the inflammation induced by monosodium urate (MSU) crystal induced release of interleukin 1J3 (IL-1J3). This is a simple prescreen that identifies anti-inflammatory activity of various types of anti-inflammatory agents, especially IL-1 pathway blockers like interleukin receptor antagonists or antibodies that block IL-1 or IL1R1 (Torres R, et al. Hyperalgesia, synovitis and multiple biomarkers of inflammation are suppressed by interleukin 1 inhibition in a novel animal model of gouty arthritis. Ann Rheym Dis. 2009; 68(10):1602-1608, which is incorporated by reference herein in its entirety for all purposes). Gout is the most common form of inflammatory arthritis and is increasing in prevalence worldwide (Roddy E and Doherty M. Epidemiology of Gout. Arthritis Research & Therapy. 2010; 12(6):223, which is incorporated by reference herein in its entirety for all purposes). Gouty arthritis is characterized by increased serum urate concentration and deposits of monosodium urate crystals (MSU) in and around the joints, leading to swollen joints and severe pain (Sabina E P, Chandel S, and Rasool M K. Inhibition of monosodium urate crystal-induced inflammation by withaferin A. J Pharm Pharmaceut Sci. 2008; 11(4):46-55, which is incorporated by reference herein in its entirety for all purposes). Current treatments include nonsteroidal anti-inflammatory drugs (NSAIDs), steroids, or colchicine. For some patients these treatments may not be effective in treating gout or have adverse side effects (Sabina, 2008; Getting S J, et al. Activation of melanocortin type 3 receptor as a molecular mechanism for adrenocorticotropic hormone efficacy in gouty arthritis. Arthritis & Rheumatism. 2002; 46(10):2765-2775, which is incorporated by reference herein in its entirety for all purposes). The MSU-induced inflammation model provides a good, simple screening tool for identifying compounds that may have activity in the more complex disease process, such as systemic arthritis and more complex IL-1 driven diseases.

A study was conducted to evaluate the efficacy of adeno-associated virus (AAV)-mediated CRISPR therapy in monosodium urate (MSU) crystal induced inflammation in mice. On study day 0, male C57BL/6 mice were dosed into the right knee with a single (lx) intra-articular (IA) injection of placebo control (diluent, phosphate buffered saline [PBS]), a mixture of two variants of AAV-6 (one carrying Guide RNA 1 and the other carrying Guide RNA 2, 5×109 virus genome [vg] copies per mL), a mixture of two variants of AAV-5 (Guide 1+Guide 2, 5×109 vg/mL), the scrambled AAV-6 control (carrying non-targeting guide RNA, 1×1010 vg/mL), or the scrambled AAV-5 control (1×1010 vg/mL). On study day 7, the mice were given injections into right knee (same joint as treatment) with MSU crystals (25 mg/mL: 250 μg in 10 μL PBS). The mice were euthanized for necropsy approximately 6 hours post-MSU injection on study day 7. Efficacy evaluation was based on animal body weights, von Frey testing, and knee caliper measurements.

Mice treated IA (lx on day 0) with AAV-6 (Guide 1+2: 5×109 vg/guide per knee) showed a statistically significant reduction in referred pain, as measured by von Frey testing, 6 hours after MSU injection on day 7 as compared to mice injected IA with AAV-5 scramble vector (p=0.025) with results being nearly significant as compared to the AAV-6 scramble vector and PBS control groups (p=0.051 and p=0.075, respectively). Area under the curve (AUC) calculations for von Frey assessments did not differ statistically across groups. Animal body weight gain and knee swelling did not differ statistically across groups (Table 7). All animals survived to study termination.

TABLE 7
Summary of Data

| | | von Frey Absolute | Knee Caliper Change |
|---|---|---|---|
| | | Threshold AUC | from Baseline AUC |
| | | (day −1 | (day −1 |
| Group Treatment | | to 7) - Right Foot | to 7) - Right Knee |
| 1 | AAV-6 Scramble Vector (1 × 10¹⁰ | 5.92 (0.39) | 0.02 (0.01) |
| | particles/knee), IA, 1× (d0) | | |
| 2 | AAV-6 Guide 1 + 2 (5 × 10⁹ | 6.01 (0.36) | 0.01 (0.01) |
| | of each guide/knee), IA, 1× (d0) | | |
| 3 | PBS, IA (d0) | 6.12 (0.33) | 0.02 (0.01) |
| 4 | AAV-5 Scramble Vector (1 × 10¹⁰ | 5.09 (0.31) | 0.02 (0.01) |
| | particles/knee), IA, 1× (d0) | | |
| 5 | AAV-5 Guide 1 + 2 (5 × 10⁹ | 5 73 (0.39) | 0 02 (0.01) |
| | of each guide/knee), IA, 1× (d0) | | |

Values represent group means and standard errors (SE)
PBS = Phosphate buffered saline control,
AAV = adeno-associated virus,
AUC = Area under the curve
*p < 0.05 ANOVA (Tukey's post-hoc) vs. AAV-6 Guide 1
†p < 0.05 ANOVA (Tukey's post-hoc) vs. PBS
‡p < 0.05 ANOVA (Tukey's post-hoc) vs. AAV-5 Scramble Vector
§p < 0.05 ANOVA (Tukey's post-hoc) vs. AAV-5 Guide 1

Summary of Clinical Outcomes—No significant differences were observed between groups over time. No clinical evidence that virus injection provoked a response greater than that seen in the vehicle group. No clinical evidence that virus injection altered the effects of MSU on joint swelling. Specific role of IL-1 in MSU-induced inflammation is unclear, so lack of clinical effect may not be unexpected.

Summary of qPCR—qPCR data confirm that CRISPR editing with AAV-6 or AAV-5 is effective in restoring IL-1 beta mRNA expression to normal levels. Statistical significance is hard to demonstrate given the sample size. Confirmation of this effect can be obtained through IHC analysis of synovial tissues.

Regulatory Compliance

This study was conducted in accordance with the test facility standard operating procedures (SOPs), the World Health Organization Quality Practices in Basic Biomedical Research guidelines, and in compliance with all state and federal regulations, including USDA Animal Welfare Act 9 CFR Parts 1-3. Federal Register 39129, Jul. 22, 1993.

Institutional Animal Care and Use

This study was conducted in accordance with The Guide for the Care & Use of Laboratory Animals (8^{th} Edition). No acceptable alternative test systems were identified for the animals used in this study.

Materials and Methods

Test Article Identification and Preparation

AAV vectors were pre-formulated as a viral particle suspension (>5×10¹² virus genome [vg] copies per mL) in frozen aliquots. The aliquots were stored at −80° C. and reconstituted in diluent (sterile filtered PBS [Corning, lot No. 01420007]) immediately before use. Standard biosafety level 2 (BSL-2) handling was used by personnel handling the AAV vectors prior to injection. The AAV scramble controls were prepared in sterile PBS to form working stocks containing 1×10¹² vg/mL for IA injection at 10 μL/knee to deliver 1×10¹⁰ vg of the scramble control into the knee joint. The active AAV vectors were prepared by mixing equal parts of each of the two active AAV-5 or AAV-6 constructs with sterile PBS to form working stocks containing 5×10¹¹ vg/mL for each of the two guides. The active AAV formulations were injected IA at 10 μL/knee to deliver 5×10⁹ vg of each of the two guides into the knee joint. See the study protocol (Appendix B) for further details of test article preparation, storage, and handling.

The AAV vectors were identified as follows:

AAVPrimeTM Adeno-Associated Virus - Serotype6 (AAV-6)
Particles for sgRNA (GeneCopoeia ™, catalog
No. AA06-MCP001682-AD01-2-200-a, lot No. GC03182K2001)
AAVPrimeTM Adeno-Associated Virus - Serotype6 (AAV-6)
Particles for sgRNA (GeneCopoeia ™, catalog
No. AA06-MCP001682-AD01-2-200-b, lot No. GC03202K2001)
AAVPrimeTM Adeno-Associated Virus - Serotype6 (AAV-6)
Particles for sgRNA (GeneCopoeia ™, catalog
No. AA06-CCPCTR01-AD01-200, lot No. GC03112K2002)
AAVPrimeTM Adeno-Associated Virus - Serotype5 (AAV-5)
Particles for sgRNA (GeneCopoeia ™, catalogue
No. AA05-MCP001682-AD01-2-200A, lot No. GC03182K2002)
AAVPrimeTM Adeno-Associated Virus - Serotype5 (AAV-5)
Particles for sgRNA (GeneCopoeia ™, catalogue
No. AA05-MCP001682-AD01-2-200B, lot No. GC03182K2003)
AAVPrimeTM Adeno-Associated Virus - Serotype5 (AAV-5)
Particles for sgRNA (GeneCopoeiaTM, catalogue
No. AA05-CCPCTR01-AD01-200, lot No. GC03032K2003)

Monosodium urate (MSU) crystals were obtained from Invivogen (catalogue No. Tlrl-25-MSU, lot No. MSU-42-01). MSU crystals were prepared at 25 mg/mL in PBS (without Ca or Mg: Corning, catalogue No. 21-031-CV, lot No. 31719003) in a plastic tube, vortexed for approximately 1 minute, sonicated for approximately 15 to 20 minutes, and vortexed before pipetting and use.

Test System

| | |
|---|---|
| Number of animals: | 75 (70 + 5 extra) |
| Species/Strain or Breed: | C57BL/6 |
| Vendor: | Jackson |
| Age/Wt at Arrival: | 8-10 weeks old (~20 grams) |
| Gender: | Male |
| Age/Wt Range at Study Initiation: | At least 9 weeks by study initiation |
| Acclimation: | Will be acclimated for at least 3 |
| | days after arrival at BBP |
| Housing: | 3-5 animals/cage |

Male C57BL/6 mice (N=70+4 extra) that were 8 to 10 weeks of age were obtained from The Jackson Laboratory (Bar Harbor, Me.). The mice weighed approximately 20 to 29 grams (mean of approx. 25 g) at enrollment on study day −1.

Animals were identified by color-coded dots at the base of the tail delineating animal number. After enrollment, all cages were labeled with protocol number, group number, and animal numbers.

Environment and Husbandry

Upon arrival, the animals were housed 3 to 5 per cage in polycarbonate cages with corncob bedding and suspended food and water bottles. The mice were housed in individually ventilated pie cages (passive airflow, approximately 0.045-0.048 m2 floor space). Animal care including room, cage, and equipment sanitation conformed to the guidelines cited in the Guide for the Care and Use of Laboratory Animals (Guide, 2011) and the applicable BBP SOPs.

The animals were acclimated for 9 days prior to being paced in the study. An attending veterinarian was on site or on call during the live phase of the study. No concurrent medications were given.

During the acclimation and study periods, the animals were housed in a laboratory environment with temperatures ranging 19° C. to 25° C. and relative humidity of 30% to 70%. Automatic timers provided 12 hours of light and 12 hours of dark. The animals were allowed access ad libitum to Envigo Teklad 8640 diet fresh municipal tap water.

Study Design

On study day −1, the animals were randomized by body weight into treatment groups, knees were shaved, and baseline knee caliper measurements were taken. On study day 0, the animals were dosed with treatments (IA into the right knee) as indicated in Table 8. On study day 7, the animals were given IA injections of MSU crystals (a total of 10 μL, 250 μg of MSU) into right knees (same knee as treatments). Body weight measurements were taken as described. Referred pain was measured by von Frey testing at 5 time points as described. Caliper measurements of right knees were taken at 5 time points as described. The animals were euthanized for necropsy following the final behavioral testing on day 7, as described.

TABLE 8
Group and Treatment Information

| | | | | Dose Level | | Dose Conc. | | |
|---|---|---|---|---|---|---|---|---|
| Grp | N | MSU | Treatment | (particles/knee) | Dose Vol. | (particles/mL) | Dose Route | Regimen |
| 1 | 14 | Yes | AAV-6 | 1 × 10¹⁰ | 10 μL | 1 × 10¹² | IA | 1× (Day 0) |
| | | | Scramble Vector | | | | (right knee) | |
| 2 | 14 | Yes | AAV-6 | 5 × 10⁹ | 10 μL | 1 × 10¹² | IA | 1× (Day 0) |
| | | | Guide 1 + 2 | (of each guide) | | | (right knee) | |
| 3 | 14 | Yes | PBS | — | 10 μL | — | IA | 1× (Day 0) |
| | | | | | | | (right knee) | |
| 4 | 14 | Yes | AAV-5 | 1 × 10¹⁰ | 10 μL | 1 × 10¹² | IA | 1× (Day 0) |
| | | | Scramble Vector | | | | (right knee) | |
| 5 | 14 | Yes | AAV-5 | 5 × 10⁹ | 10 μL | 1 × 10¹² | IA | 1× (Day 0) |
| | | | Guide 1 + 2 | (of each guide) | | | (right knee) | |

Disease Induction

MSU crystals were prepared at a concentration of 25 mg/mL in sterile PBS. Crystals did solubilize, and injection preparation was carefully mixed prior to use. 10 uL of MSU crystal solution was injected into the right knee joint.

Unformulated MSU Storage Conditions: Room temperature, protected
from light
Vehicle: PBS
Dose Route: IA into the right knee
Dose Volume: 10 uL per knee
MSU Formulation Instructions & Calculations: 0.01 ml/knee * 75
animals = 0.72 ml of 25 mg/ml MSU needed, minimum
MSU Storage & Stability: Stored in glass. Prepared just prior to dosing.
Vortexed before injecting
MSU Disposition Following Dosing: Formulations discarded after dosing.
Unformulated stock retained.

Body Weight Measurements and Live Phase Sampling

The mice were weighed on study day −1 (pre-injection) for randomization, and body weights were measured again on study days 2 and 6. Animal body weight measurements can be found in Table 9.

TABLE 9
Body Weight and Dose Calculation Data (MGA-UCM-2)

| Treatment Group | | | | | | | |
|---|---|---|---|---|---|---|---|
| Group 2 | | | | | | | |
| AAV-6 Scramble | Day −1 | Day 8 | Day 2 | | Day 6 | | Change in |
| Vector (1 × 10\hat{ }10 particles / knee) | Body Wt. | Dose Vol. | Body Wt. | Body Wt. % Δ | Body Wt. | Body Wt. % Δ | Body Weight |
| IA, 1× (D0) | (g) | 10 μl (ml) | (g) | from Baseline | (g) | from Baseline | Day −1 to 6 |
| 1 | 25.99 | 0.01 | 24.19 | −6.9% | 24.67 | −5.1% | −1 |
| 2 | 25.00 | 0.01 | 24.64 | −1.4% | 25.32 | 1.3% | 0 |
| 3 | 23.50 | 0.01 | 24.61 | 4.7% | 24.95 | 6.2% | 1 |
| 4 | 23.92 | 0.01 | 23.14 | −3.3% | 23.83 | −0.5% | 0 |
| 5 | 23.30 | 0.01 | 23.34 | −0.7% | 23.78 | 2.3% | 0 |
| 6 | 23.84 | 0.01 | 23.97 | 4.0% | 24.51 | 6.4% | 3 |
| 7 | 27.44 | 0.01 | 28.10 | 2.4% | 27.90 | 1.7% | 0 |
| 8 | 22.28 | 0.01 | 22.83 | 0.2% | 23.59 | 3.5% | 1 |
| 9 | 24.80 | 0.01 | 24.24 | −0.2% | 25.73 | 3.7% | 1 |
| 10 | 27.46 | 0.01 | 27.97 | 1.9% | 28.66 | 4.4% | 3 |
| 11 | 26.50 | 0.01 | 26.57 | 0.3% | 26.88 | 1.4% | 0 |
| 12 | 26.51 | 0.01 | 26.48 | −0.1% | 27.78 | 4.8% | 1 |
| 13 | 22.80 | 0.01 | 23.38 | 2.5% | 23.77 | 4.3% | 1 |
| 14 | 24.27 | 0.01 | 24.68 | −0.7% | 24.90 | 0.5% | 0 |
| Extra | 21.95 | 0.01 | 22.56 | 2.8% | 23.34 | 6.3% | 1 |
| Mean | 24.84 | | 24.88 | 0.19% | 25.4 | 2.47% | 0.6 |
| SE | 0.45 | | 0.5 | 0.80% | 0.5 | 0.80% | 0.2 |
| ANOVA (Tukey's post-hoc) vs. Group 2 | | | | | | | 0.584 |
| ANOVA (Tukey's post-hoc) vs. Group 3 | | | | | | | 0.243 |
| ANOVA (Tukey's post-hoc) vs. Group 4 | | | | | | | 0.212 |
| ANOVA (Tukey's post-hoc) vs. Group 5 | | | | | | | 0.543 |
| Group 2 | | | | | | | |
| AAV-6 Guide 1 + 2 | Day −1 | Day −1 | Day 2 | | Day 6 | | Change in |
| ( [symbol] × 10\hat{ }9 of each guide / knee) | Body Wt. | Dose Vol. | Body Wt. | Body Wt. % Δ | Body Wt. | Body Wt. % Δ | Body Weight |
| IA, 1× (D0) | (g) | 10 μl (ml) | Wt. (g) | Baseline | Wt. (g) | Baseline | Day −1 to 6 |
| 1 | 22.56 | 0.01 | 22.47 | −0.4% | 22.92 | 1.6% | 0 |
| 2 | 28.49 | 0.01 | 29.00 | 1.8% | 29.08 | 2.1% | 1 |
| 3 | 24.65 | 0.01 | 24.65 | 0.0% | 25.06 | 1.7% | 0 |
| 4 | 23.63 | 0.01 | 23.29 | −1.4% | 24.05 | 1.8% | 0 |
| 5 | 22.78 | 0.01 | 23.19 | 1.8% | 24.14 | 6.0% | 1 |
| 6 | 27.32 | 0.01 | 27.60 | 1.0% | 28.54 | 4.3% | 1 |
| 7 | 26.83 | 0.01 | 27.49 | 2.5% | 28.00 | 4.4% | 1 |
| 8 | 26.96 | 0.01 | 26.84 | −0.4% | 28.70 | 6.5% | 2 |
| 9 | 22.80 | 0.01 | 22.88 | 0.4% | 23.66 | 3.8% | 1 |
| 10 | 22.45 | 0.01 | 23.12 | 3.0% | 23.86 | 6.3% | 1 |
| 11 | 24.83 | 0.01 | 24.53 | −1.2% | 25.42 | 2.4% | 1 |
| 12 | 25.80 | 0.01 | 26.11 | 1.2% | 26.56 | 2.9% | 1 |
| 13 | 25.30 | 0.01 | 29.58 | 3.3% | 26.14 | 3.3% | 1 |
| 14 | 23.45 | 0.01 | 23.88 | 1.8% | 24.14 | 2.9% | 1 |
| Extra | 25.84 | 0.01 | 26.43 | 2.3% | 26.29 | 1.7% | 0 |
| Mean | 24.85 | | 25.05 | 0.79% | 25.7 | 3.57% | 0.9 |
| SE | 0.53 | | 0.56 | 0.36% | 0.6 | 0.48% | 0.1 |
| ANOVA (Tukey's post-hoc) vs. Group 3 | | | | | | | 0.940 |
| ANOVA (Tukey's post-hoc) vs. Group 4 | | | | | | | 0.919 |
| ANOVA (Tukey's post-hoc) vs. Group 5 | | | | | | | 0.999 |
| Group 3 | Day −1 | Day −1 | Day 2 | | Day 6 | | Change in |
| PBS | Body Wt. | Dose Vol. | Body | Body Wt. % Δ | Body Wt. | Body Wt. % Δ | Body Weight |
| IA, 1× (D0) | (g) | 10 μl (ml) | Wt. (g) | Baseline | (g) | Baseline | Day −1 to 6 |
| 1 | 23.14 | 0.01 | 28.19 | 3.0% | 29.19 | −1.5% | 2 |
| 2 | 25.35 | 0.01 | 25.11 | −0.9% | 26.13 | −4.1% | 1 |
| 3 | 24.72 | 0.01 | 25.03 | 1.3% | 25.41 | −3.8% | 1 |
| 4 | 24.53 | 0.01 | 29.04 | 1.3% | 26.63 | −3.8% | 1 |
| 5 | 24.50 | 0.01 | 25.00 | 1.9% | 26.16 | −2.6% | 1 |
| 6 | 23.25 | 0.01 | 25.30 | 0.9% | 29.96 | −2.1% | 1 |
| 7 | 25.45 | 0.01 | 25.01 | 1.4% | 36.42 | −2.0% | 1 |
| 8 | 25.50 | 0.01 | 26.48 | 2.6% | 36.12 | −4.0% | 1 |
| 9 | 24.77 | 0.01 | 24.33 | 2.3% | 26.24 | −5.0% | 1 |
| 10 | 19.83 | 0.01 | 20.15 | 3.0% | 21.23 | −7.1% | 1 |
| 11 | 25.66 | 0.01 | 26.30 | 2.5% | 27.14 | −5.0% | 1 |
| 12 | 24.27 | 0.01 | 24.58 | 1.3% | 25.63 | −5.1% | 1 |
| 13 | 20.95 | 0.01 | 26.01 | 4.5% | 21.23 | −5.9% | 1 |
| 14 | 34.74 | 0.01 | 24.58 | 0.6% | 34.92 | −0.7% | 0 |
| Mean | 24.79 | | 28.22 | 1.77% | 23.5 | 4.36% | 1.1 |
| SE | 8.62 | | 0.03 | 0.36% | 0.6 | 0.51% | 0.1 |
| ANOVA (Tukey's post-hoc) vs. Group 4 | | | 28.33 | | | | >0.9999 |
| ANOVA (Tukey's post-hoc) vs. Group 5 | | | 0.03 | | | | 0.983 |
| Group 4 | | | | | | | |
| AAV-6 Scramble | Day −1 | Day −1 | Day 2 | | Day 6 | | Change in |
| Vector (1 × 10\hat{ }10 particles / knee) | Body Wt. | Dose Vol. | Body | Body Wt. % Δ | Body Wt. | Body Wt. % Δ | Body Weight |
| IA, 1× (D0) | (g) | 10 μl (ml) | Wt. (g) | Baseline | (g) | Baseline | Day −1 to 6 |
| 1 | 25.60 | 0.01 | 25.68 | −2.7% | 26.51 | −5.0% | 2 |
| 2 | 24.60 | 0.01 | 26.68 | −2.5% | 30.85 | −6.0% | 2 |
| 3 | 23.37 | 0.01 | 25.41 | −0.2% | 28.32 | −6.6% | 0 |
| 4 | 23.46 | 0.01 | 27.43 | −2.0% | 34.73 | −5.4% | 1 |
| 5 | 24.56 | 0.01 | 24.37 | −1.0% | 36.16 | −4.8% | 1 |
| 6 | 26.95 | 0.01 | 27.27 | −1.2% | 27.89 | −3.3% | 1 |
| 7 | 24.79 | 0.01 | 25.07 | −0.3% | 25.63 | −2.8% | 1 |
| 8 | 24.52 | 0.01 | 24.60 | −0.3% | 25.38 | −3.5% | 1 |
| 9 | 24.66 | 0.01 | 27.00 | −0.9% | 27.21 | 10.3% | 3 |
| 10 | 24.62 | 0.01 | 26.66 | −5.8% | 26.11 | −6.1% | 1 |
| 11 | 23.73 | 0.01 | 24.66 | −1.4% | 33.38 | −6.6% | 0 |
| 12 | 23.42 | 0.01 | 24.04 | −2.0% | 34.58 | −6.2% | 1 |
| 13 | 22.83 | 0.01 | 22.85 | −0.3% | 23.22 | −2.8% | 0 |
| 14 | 25.50 | 0.01 | 25.86 | −0.9% | 25.82 | −1.3% | 0 |
| Extra | [symbol] | 0.01 | [symbol] | [symbol] | [symbol] | [symbol] | [symbol] |
| Mean | 24.93 | | 35.46 | 2.33% | 26.8 | 4.27% | 1.1 |
| SE | 0.42 | | 0.43 | 8.73% | 0.5 | 0.74% | 8.2 |
| ANOVA (Tukey's post-hoc) vs. Group 5 | | | | | | | 0.073 |
| Group 5 | Day −1 | Day −1 | Day 2 | | Day 3 | | Change in |
| AAV-6 Guide 1 + 2 [symbol] of each guide / bones | Body Wt. | Dose Vol. | Body Wt. | Body Wt. % Δ | Body Wt. | Body Wt. % Δ | Body Weight |
| IA, 1× (D0) | (g) | 10 μl (ml) | (g) | Baseline | (g) | Baseline | Day −1 to 6 |
| 1 | 25.57 | 0.01 | 26.47 | −2.5% | 20.21 | −2.2% | 1 |
| 2 | 23.49 | 0.01 | 23.37 | −3.3% | 24.14 | −2.1% | 1 |
| 3 | 24.28 | 0.01 | 24.42 | −0.6% | 24.71 | −1.8% | 0 |
| 4 | 23.69 | 0.01 | 23.31 | −3.0% | 24.18 | −0.7% | 1 |
| 5 | 24.53 | 0.01 | 25.34 | −3.3% | 24.87 | −1.8% | 0 |
| 6 | 25.60 | 0.01 | 26.60 | −2.7% | 27.25 | −5.2% | 1 |
| 7 | 25.93 | 0.01 | 26.68 | −6.6% | 26.28 | −1.4% | 0 |
| 8 | 29.32 | 0.01 | 25.48 | −0.8% | 26.46 | −4.0% | 1 |
| 9 | 25.32 | 0.01 | 17.51 | −3.8% | 37.38 | −5.4% | 1 |
| 10 | 27.50 | 0.01 | 27.83 | −0.5% | 28.82 | −4.8% | 1 |
| 11 | 24.69 | 0.01 | 25.34 | −3.4% | 25.85 | −4.7% | 1 |
| 12 | 24.13 | 0.01 | 24.97 | −3.4% | 26.01 | −2.3% | 2 |
| 13 | 22.60 | 0.01 | 23.59 | −1.7% | 22.59 | −1.7% | 0 |
| 14 | 23.96 | 0.01 | 24.32 | 1.5% | 24.93 | −4.0% | 1 |
| Extra | [symbol] | 0.01 | [symbol] | [symbol] | [symbol] | [symbol] | 1 |
| Mean | 24.90 | | 25.38 | 1.56% | 25.7 | 3.75% | 0.8 |
| SE | 8.35 | | 0.46 | 0.49% | 6.4 | 0.56% | 0.1 |

[symbol]

indicates data missing or illegible when filed

Von Frey Methods

Von Frey analysis was performed on right hind paws at 5 time points: baseline (day −1), 6 hours post-dose (day 0), 24 hours post-dose (day 1), pre-MSU injection (0 h, day 7), and 6 hours post-MSU injection (day 7). The groups were blinded to the researcher during von Frey testing.

The von Frey method evaluates mechanical allodynia (pain due to a stimulus that does not normally provoke pain) based on the response of animals to the application of calibrated filaments (Bioseb, Vitrolles, France) to the foot. The filaments are identified by a number representing log 10 of the force in milligrams×10. The animals are habituated to the testing rack three times (45 to 60 minutes) prior to baseline evaluation. When testing, the von Frey hair is placed on the surface of the hind paw and pushed smoothly until the hair has a significant bend in it; the hair is pressed against the paw for six seconds. Responses are recoded as either a 0 (no response) or a 1 (response). A response is defined as lifting the hind paw away from the hair, jerking the leg away, walking away from the hair, etc. The starting hair is 3.22, if the animal responds the tester moves down to 2.83, if there is no response to the 3.22 hair then the tester moves up to 3.61; the tester continues to test hairs based on the response and moves up or down, as appropriate. The hair increments are as follows: 1.65, 2.36, 2.44, 2.83, 3.22, 3.61, 3.84, 4.08, 4.17. Each paw is tested 5 times, moving up and down between hairs until the final filament is reached. Data is entered into a spreadsheet and used to translate the response rate into a paw withdraw threshold. Results of testing are converted to an absolute threshold (50% response rate) in grams, using the formula 10(x+yz)/10000, where x equals the log unit value of the final tested filament, y equals the tabular value for the response pattern from Dixon's up-and-down method for small samples (Dixon, 1965), and z equals the average interval between filament values. Testing is done on the hind portions of the hind paw as the heel tends to give a more reliable and sensitive response. The testers monitor the animals for hyper-responding or freezing, in which case the animals are left alone until calm. Von Frey data can be found in Table 10.

TABLE 10
von Frey Data (MGA-UCM-2)

| Treatment Group | | | | | | | | | | | |
|---|---|---|---|---|---|---|---|---|---|---|---|
| Group 3 | | | | | | | | | | | |
| AAV-3 Scramble | Day −1 | | Day 0.25 | | Day 1 | | Day 7 | | Day 7.25 | | |
| Vector (1 × 10\hat{ }10 particles / knee) | Absolute Threshold | | Absolute Threshold | | Absolute Threshold | | Absolute Threshold | | Absolute Threshold | | Day 4-7 |
| IA, 1× (D0) | Log units | (g) | Log units | (g) | Log units | (g) | Log units | (g) | Log units | (g) | AUC |
| 1 | 3.72500 | 0.53 | 3.96000 | 0.91 | 3.96000 | 0.93 | 3.72500 | 0.53 | 3.72500 | 0.53 | 6.05 |
| 2 | 3.96000 | 0.53 | 3.72500 | 0.53 | 3.72500 | 0.53 | 4.12500 | 1.33 | 3.41500 | 0.26 | 7.09 |
| 3 | 3.72500 | 0.53 | 3.82500 | 0.11 | 3.02500 | 0.11 | 3.96000 | 0.91 | 3.41500 | 0.26 | 3.68 |
| 4 | 3.96000 | 0.91 | 3.96000 | 0.91 | 3.96000 | 0.91 | 3.96000 | 0.91 | 2.63500 | 0.04 | 7.42 |
| 5 | 3.72500 | 0.53 | 3.02500 | 0.53 | 3.02500 | 0.11 | 3.72500 | 0.53 | 3.41500 | 0.26 | 2.91 |
| 6 | 3.96000 | 0.91 | 3.72500 | 0.53 | 3.96000 | 0.91 | 3.96000 | 0.91 | 3.02500 | 0.11 | 7.04 |
| 7 | 4.12500 | 1.33 | 3.72500 | 0.53 | 3.41500 | 0.26 | 3.72500 | 0.53 | 3.43500 | 0.26 | 3.93 |
| 8 | 3.96000 | 0.91 | 3.96000 | 0.91 | 3.96000 | 0.91 | 3.96000 | 0.91 | 3.41500 | 0.26 | 7.44 |
| 9 | 3.72500 | 0.53 | 3.72500 | 0.53 | 3.90000 | 0.91 | 3.72500 | 0.53 | 3.41500 | 0.26 | 5.63 |
| 10 | 3.96000 | 0.91 | 3.96000 | 0.91 | 3.96000 | 0.91 | 3.96000 | 0.91 | 2.63500 | 0.04 | 7.42 |
| 11 | 3.96000 | 0.91 | 3.56000 | 0.91 | 3.72500 | 0.53 | 3.96000 | 0.91 | 3.02500 | 0.11 | 6.34 |
| 12 | 4.12500 | 1.53 | 3.72500 | 0.53 | 3.72500 | 0.53 | 3.96000 | 0.91 | 3.41500 | 0.26 | 6.04 |
| 13 | 3.96000 | 0.91 | 3.72500 | 0.53 | 3.72500 | 0.53 | 3.96000 | 0.91 | 2.63500 | 0.04 | 5.75 |
| 14 | 3.96000 | 0.91 | 3.56000 | 0.91 | 3.96000 | 0.91 | 3.72300 | 0.53 | 3.72500 | 0.53 | 6.29 |
| Extra | 4.12500 | 1.33 | 3.41500 | 0.26 | 3.41300 | 0.26 | 4.12500 | 1.33 | 3.02500 | 0.11 | 6.35 |
| Mean | | 0.86 | | 0.66 | | 0.64 | | 0.81 | | 0.23 | 5.92 |
| SE | | 0.07 | | 0.07 | | 0.08 | | 0.06 | | 0.04 | 0.39 |
| ANOVA (Tukey's post-hoc) vs. Group 2 | | 0.979 | | 0.987 | | 1.000 | | 0.974 | | 0.631 | 1.000 |
| ANOVA (Tukey's post-hoc) vs. Group 3 | | 0.998 | | >0.9999 | | >0.9949 | | 0.907 | | >0.9999 | 0.994 |
| ANOVA (Tukey's post-hoc) vs. Group 4 | | 0.999 | | 0.943 | | 0.495 | | 0.936 | | 0.987 | 0.478 |
| ANOVA (Tukey's post-hoc) vs. Group 5 | | 0.984 | | 0.873 | | 0.995 | | 0.998 | | 0.599 | 0.996 |
| Group 2 | | | | | | | | | | | |
| AAV-3 Guide 1 + 2 | Day −1 | | Day 0.25 | | Day 1 | | Day 7 | | Day 7.25 | | |
| (1 × 10\hat{ }9 of each guide / knee) | Absolute Threshold | | Absolute Threshold | | Absolute Threshold | | Absolute Threshold | | Absolute Threshold | | Day 4-7 |
| IA, 1× (D0) | Log units | (g) | Log units | (g) | Log units | (g) | Log units | (g) | Log units | (g) | AUC |
| 1 | 3.96000 | 0.91 | 3.41500 | 0.26 | 3.72500 | 0.53 | 4.12500 | 1.53 | 3.43500 | 0.26 | 6.82 |
| 2 | 3.96000 | 0.91 | 3.02500 | 0.11 | 3.96000 | 0.91 | 3.72500 | 0.53 | 3.41500 | 0.26 | 5.45 |
| 3 | 3.72500 | 0.53 | 3.72300 | 0.53 | 3.41500 | 0.26 | 3.96000 | 0.91 | 3.72500 | 0.53 | 4.66 |
| 4 | 3.72500 | 0.53 | 3.96000 | 0.91 | 3.96000 | 0.91 | 3.96000 | 0.93 | 3.43500 | 0.26 | 7.20 |
| 5 | 3.96000 | 0.91 | 3.96000 | 0.91 | 3.96000 | 0.91 | 3.72500 | 0.53 | 3.72500 | 0.53 | 6.20 |
| 6 | 3.96000 | 0.93 | 3.96000 | 0.91 | 3.96000 | 0.91 | 4.12500 | 1.33 | 3.72500 | 0.53 | 8.79 |
| 7 | 4.12500 | 1.33 | 3.72500 | 0.53 | 3.72500 | 0.53 | 3.96000 | 0.93 | 3.96000 | 0.91 | 6.12 |
| 8 | 3.72500 | 0.53 | 3.41500 | 0.26 | 3.02500 | 0.11 | 3.96000 | 0.91 | 3.96000 | 0.93 | 3.91 |
| 9 | 4.12500 | 1.33 | 3.41500 | 0.26 | 3.96000 | 0.91 | 3.72500 | 0.53 | 3.72500 | 0.53 | 5.90 |
| 10 | 3.96000 | 0.91 | 3.96000 | 0.91 | 3.02500 | 0.31 | 3.96000 | 0.91 | 3.41500 | 0.26 | 4.72 |
| 11 | 3.96000 | 0.91 | 3.72500 | 0.53 | 3.72500 | 0.53 | 3.72500 | 0.53 | 3.72500 | 0.53 | 4.62 |
| 12 | 3.96000 | 0.93 | 3.96000 | 0.91 | 3.41500 | 0.26 | 3.96000 | 0.91 | 3.41500 | 0.26 | 5.24 |
| 13 | 3.96000 | 0.91 | 3.72500 | 0.53 | 3.96000 | 0.91 | 3.96000 | 0.91 | 3.02500 | 0.11 | 7.04 |
| 14 | 4.12500 | 1.33 | 3.72500 | 0.53 | 3.96000 | 0.91 | 3.96000 | 0.91 | 3.72500 | 0.53 | 7.36 |
| Extra | 3.72500 | 0.53 | 3.96000 | 0.91 | 3.72500 | 0.53 | 3.32300 | 0.53 | 3.41500 | 0.26 | 4.73 |
| Mean | | 0.92 | | 0.58 | | 0.62 | | 0.86 | | 0.46 | 6.01 |
| SE | | 0.07 | | 0.08 | | 0.09 | | 0.07 | | 0.46 | 6.36 |
| ANOVA (Tukey's post-hoc) vs. Group 3 | | 0.886 | | 0.941 | | >4.9999 | | 0.998 | | 0.075 | 1.000 |
| ANOVA (Tukey's post-hoc) vs. Group 4 | | 0.924 | | >0.9999 | | 0.639 | | 0.690 | | 0.025 | 0.369 |
| ANOVA (Tukey's post-hoc) vs. Group 5 | | 0.828 | | 1.000 | | 0.999 | | 0.999 | | 0.835 | 0.981 |
| Group 3 | Day −1 | | Day 0.25 | | Day 1 | | Day 7 | | Day 7.25 | | |
| PBS | Absolute Threshold | | Absolute Threshold | | Absolute Threshold | | Absolute Threshold | | Absolute Threshold | | Day −1-7 |
| IA, 1× (D0) | Log units | (g) | Log units | (g) | Log units | (g) | Log units | (g) | Log units | (g) | AUC |
| 1 | 3.96000 | 0.93 | 3.41500 | 0.26 | 2.96000 | 0.91 | 4.32500 | 1.33 | 3.72500 | 0.53 | 5.34 |
| 2 | 3.96000 | 0.91 | 3.96000 | 0.91 | 3.96000 | 0.91 | 3.96000 | 0.91 | 2.63500 | 0.04 | 7.42 |
| 3 | 3.96000 | 0.91 | 3.72500 | 0.53 | 3.96000 | 0.91 | 3.96000 | 0.91 | 3.41500 | 0.26 | 7.06 |
| 4 | 3.72500 | 0.53 | 3.96000 | 0.91 | 3.96000 | 0.91 | 3.32500 | 0.53 | 2.63500 | 0.04 | 5.99 |
| 5 | 3.72500 | 0.53 | 3.96000 | 0.91 | 3.72500 | 0.53 | 3.96000 | 0.91 | 2.63500 | 0.04 | 5.89 |
| 6 | 3.96000 | 0.91 | 3.96000 | 0.91 | 3.96000 | 0.91 | 3.72500 | 0.53 | 3.41300 | 0.26 | 6.25 |
| 7 | 3.96000 | 0.91 | 3.72500 | 0.53 | 3.72500 | 0.53 | 3.96000 | 0.91 | 3.72500 | 0.53 | 5.81 |
| 8 | 4.12500 | 1.33 | 3.72500 | 0.53 | 3.41500 | 0.26 | 3.72500 | 0.53 | 3.41500 | 0.26 | 3.93 |
| 9 | 3.72300 | 0.53 | 3.41500 | 0.26 | 3.72500 | 0.53 | 3.96000 | 0.91 | 3.41300 | 0.26 | 5.27 |
| 10 | 3.96000 | 0.91 | 3.96000 | 0.91 | 3.72500 | 0.53 | 3.96000 | 0.91 | 3.72500 | 0.53 | 6.39 |
| 11 | 3.72500 | 0.53 | 3.72500 | 0.53 | 3.72500 | 0.53 | 3.72500 | 0.53 | 2.63500 | 0.04 | 4.32 |
| 12 | 2.96000 | 0.91 | 3.96000 | 0.91 | 3.72500 | 0.53 | 4.12500 | 1.33 | 2.02500 | 0.11 | 7.45 |
| 13 | 3.96000 | 0.93 | 3.72500 | 0.53 | 3.72500 | 0.53 | 4.32500 | 1.33 | 3.02500 | 0.11 | 7.07 |
| 14 | 3.96000 | 0.91 | 3.72500 | 0.53 | 3.41500 | 0.26 | 3.96000 | 0.91 | 3.41500 | 0.26 | 4.86 |
| Mean | | 0.83 | | 0.66 | | 0.63 | | 0.89 | | 0.23 | 6.12 |
| SE | | 0.06 | | 0.87 | | 0.06 | | 0.08 | | 0.05 | 0.33 |
| ANOVA (Tukey's post-hoc) vs. Group 4 | | >0.9999 | | 0.962 | | 0.400 | | 0.548 | | 0.985 | 0.259 |
| ANOVA (Tukey's post-hoc) vs. Group 5 | | 0.999 | | 0.901 | | 0.998 | | 0.983 | | 0.660 | 0.937 |
| Group 4 | | | | | | | | | | | |
| AAV-3 Scramble | Day −1 | | Day 0.25 | | Day 1 | | Day 7 | | Day 7.25 | | |
| Vector (1 × 10\hat{ }10 particles / knee) | Absolute Threshold | | Absolute Threshold | | Absolute Threshold | | Absolute Threshold | | Absolute Threshold | | Day −1-7 |
| IA, 1× (D0) | Log units | (g) | Log units | (g) | Log units | (g) | Log units | (g) | Log units | (g) | AUC |
| 1 | 3.72500 | 0.53 | 3.96000 | 0.91 | 3.72500 | 0.53 | 3.96000 | 0.93 | 3.41500 | 0.26 | 5.92 |
| 2 | 3.96000 | 0.91 | 3.72500 | 0.53 | 3.41500 | 0.26 | 3.72500 | 0.53 | 2.63500 | 0.04 | 3.64 |
| 3 | 3.72500 | 0.53 | 3.02500 | 0.13 | 3.72500 | 0.53 | 1.96000 | 0.93 | 3.41500 | 0.26 | 5.31 |
| 4 | 3.72500 | 0.53 | 3.72500 | 0.53 | 3.72500 | 0.53 | 3.72500 | 0.53 | 2.63500 | 0.04 | 4.32 |
| 5 | 4.12500 | 1.33 | 3.96000 | 0.91 | 3.72500 | 0.53 | 3.96000 | 0.91 | 3.41300 | 0.26 | 6.42 |
| 6 | 3.96000 | 0.91 | 3.96000 | 0.91 | 3.72500 | 0.53 | 3.72500 | 0.53 | 3.41500 | 0.26 | 4.97 |
| 7 | 3.96000 | 0.91 | 3.96000 | 0.91 | 3.72500 | 0.53 | 3.96000 | 0.93 | 2.63500 | 0.04 | 6.33 |
| 8 | 3.96000 | 0.91 | 3.96000 | 0.91 | 3.62500 | 0.11 | 3.72500 | 0.53 | 3.02300 | 0.11 | 3.51 |
| 9 | 3.96000 | 0.93 | 3.72500 | 0.53 | 3.96000 | 0.91 | 3.96000 | 0.93 | 2.63500 | 0.04 | 7.03 |
| 10 | 3.96000 | 0.93 | 3.72500 | 0.53 | 3.02500 | 0.11 | 3.72500 | 0.53 | 3.41500 | 0.26 | 3.15 |
| 11 | 4.12500 | 1.33 | 3.02500 | 0.11 | 3.72500 | 0.53 | 3.96000 | 0.91 | 2.72500 | 0.53 | 5.65 |
| 12 | 3.72500 | 0.53 | 3.41500 | 0.26 | 3.72500 | 0.53 | 3.96000 | 0.91 | 3.02500 | 0.11 | 5.25 |
| 13 | 3.72500 | 0.53 | 3.72500 | 0.53 | 3.72500 | 0.53 | 3.72500 | 0.53 | 3.72500 | 0.53 | 4.38 |
| 14 | 3.96000 | 0.91 | 3.72500 | 0.53 | 3.72500 | 0.53 | 3.96000 | 0.91 | 2.63500 | 0.04 | 5.75 |
| Extra | 3.96000 | 0.91 | 3.96000 | 0.91 | [symbol] | 0.53 | 3.72500 | 0.53 | 3.48500 | 0.26 | 4.97 |
| Mean | | 0.84 | | 0.59 | | 0.48 | | 0.75 | | 0.20 | 5.09 |
| SE | | 0.07 | | 0.08 | | 0.05 | | 0.05 | | 0.05 | 0.31 |
| ANOVA (Tukey's post-hoc) vs. Group 5 | | 0.999 | | 0.999 | | 0.776 | | 0.869 | | 0.411 | 0.713 |
| Group 3 | Day −1 | | Day 0.25 | | Day 1 | | Day 7 | | Day 7.25 | | |
| AAV-3 Guide 1 + 2 (5 × 30\hat{ }9 of each | Absolute Threshold | | Absolute Threshold | | Absolute Threshold | | Absolute Threshold | | Absolute Threshold | | Day −1-7 |
| guides knee) IA, 1× (D0) | Log units | (g) | Log units | (g) | Log units | (g) | Log units | (g) | Log units | (g) | AUC |
| 1 | 3.72500 | 0.53 | 3.96300 | 0.91 | 3.95000 | 0.91 | 3.96000 | 0.91 | 3.41500 | 0.26 | 7.20 |
| 2 | 3.72500 | 0.53 | 3.41500 | 0.26 | 3.98048 | 0.91 | 3.96000 | 0.91 | 3.72500 | 0.53 | 6.39 |
| 3 | 4.12500 | 1.33 | 3.96000 | 0.91 | 3.72500 | 0.53 | 3.96000 | 0.91 | 3.96000 | 0.91 | 6.50 |
| 4 | 3.72500 | 0.53 | 3.42500 | 0.11 | 3.96000 | 0.91 | 3.72500 | 0.53 | 3.81300 | 0.28 | 5.21 |
| 5 | 3.72500 | 0.91 | 3.96000 | 0.91 | 3.95000 | 0.91 | 4.17500 | 1.33 | 3.96000 | 0.81 | 5.60 |
| 6 | 3.96000 | 0.53 | 3.96000 | 0.91 | 3.82500 | 0.11 | 3.72300 | 0.30 | 2.63500 | 0.04 | 3.30 |
| 7 | 3.72500 | 0.91 | 3.72500 | 0.53 | 3.72500 | 0.53 | 3.12500 | 1.33 | 3.42500 | 0.11 | 8.83 |
| 8 | 3.96000 | 0.91 | 3.41500 | 0.26 | 3.41500 | 0.26 | 3.96000 | 0.91 | 3.72500 | 0.53 | 4.62 |
| 9 | 3.96000 | 0.91 | 3.72540 | 0.53 | 3.96000 | 0.91 | 3.96000 | 0.91 | 3.81300 | 0.26 | 7.06 |
| 10 | 3.96000 | 0.53 | 3.92500 | 0.11 | 3.72500 | 0.53 | 3.72500 | 0.33 | 3.41500 | 0.26 | 4.16 |
| 11 | 3.72500 | 0.91 | 3.96000 | 0.91 | 3.62580 | 0.11 | 3.96000 | 0.91 | 3.41500 | 0.26 | 4.48 |
| 12 | 3.96000 | 1.33 | 3.41500 | 0.26 | 3.96000 | 0.91 | 3.72500 | 0.53 | 2.83500 | 0.54 | 8.57 |
| 13 | 4.12500 | 0.91 | 3.96000 | 0.91 | 3.31500 | 0.26 | 3.98000 | 0.91 | 2.72300 | 0.53 | 2.54 |
| 14 | 3.96000 | 0.91 | 3.41500 | 0.28 | 3.72500 | 0.53 | 3.72500 | 0.53 | 3.82500 | 0.11 | 4.29 |
| Extra | 3.96000 | 0.91 | 8.82580 | 0.11 | 3.72500 | 0.53 | [symbol] | 0.41 | 3.82500 | 0.11 | 5.33 |
| Mean | | 0.81 | | 0.56 | | 0.59 | | 0.84 | | 0.36 | 5.73 |
| SE | | 0.08 | | 0.89 | | 0.09 | | 0.87 | | 0.08 | 0.39 |

[symbol]

indicates data missing or illegible when filed

Caliper Methods

Caliper measurements of right knees were taken at 5 time points: baseline (day −1), 6 hours post-dose (day 0), 24 hours post-dose (day 1), pre-MSU injection (0 h, day 7), and 6 hours post-MSU injection (day 7). Knee caliper measurements were made using a spring-loaded micrometer caliper (Mitutuyo). Knee caliper measurements can be found in Table 11.

TABLE 11
Knee Caliper Data (MGA-UCM-2)

| Treatment Group | | | | | | | | | | | |
|---|---|---|---|---|---|---|---|---|---|---|---|
| | Day −1 | | Day 0.25 | | Day 1 | | Day 7 | | Day 7.25 | | |
| Group 1 | Caliper | | Caliper | | Caliper | | Caliper | | Caliper | | |
| AAV-4 Scramble | Measurement ( [symbol] ) | | Measurement ( [symbol] ) | | Measurement ( [symbol] ) | | Measurement ( [symbol] ) | | Measurement ( [symbol] ) | | Day 4-7 |
| Vector ( [symbol] × [symbol] particles / | | A from | | A from | | A from | | A from | | A from | A from Baseline |
| knee) IA, 1× (D0) | Right | Baseline (D-1) | Right | Baseline (D-1) | Right | Baseline (D-1) | Right | Baseline (D-1) | Right | Baseline (D-1) | AEC |
| 1 | 3.63 | 0.00 | 3.65 | 0.02 | 3.63 | 0.00 | 3.63 | 0.00 | 3.68 | 0.05 | 0.03 |
| 2 | 3.58 | 0.00 | 3.02 | 0.04 | 3.88 | 0.00 | 3.89 | −0.03 | 3.40 | 0.02 | −0.05 |
| 3 | 3.67 | 0.00 | 3.72 | 0.05 | 3.65 | 0.00 | 3.67 | 0.00 | 3.70 | 0.03 | 0.05 |
| 4 | 3.65 | 0.00 | 3.30 | 0.05 | 3.65 | 0.00 | 3.84 | −0.01 | 3.68 | 0.03 | 0.02 |
| 5 | 3.58 | 0.00 | 3.62 | 0.02 | 3.58 | 0.00 | 3.58 | 0.03 | 3.62 | 0.04 | 0.05 |
| 6 | 3.60 | 0.00 | 3.61 | 0.01 | 3.65 | 0.00 | 3.69 | 0.00 | 3.62 | 0.02 | 0.61 |
| 7 | 3.89 | 0.00 | 3.94 | 0.05 | 3.89 | 0.00 | 3.87 | −0.02 | 3.91 | 0.02 | −0.91 |
| 8 | 3.73 | 0.00 | 3.74 | 0.01 | 3.73 | 0.00 | 3.73 | 0.03 | 3.78 | 0.05 | 0.82 |
| 9 | 3.76 | 0.00 | 3.75 | 0.03 | 3.76 | 0.00 | 3.69 | −0.01 | 3.74 | 0.04 | 0.60 |
| 10 | 3.86 | 0.00 | 3.89 | 0.03 | 3.86 | 0.00 | 3.87 | 0.01 | 3.75 | 0.09 | 0.67 |
| 11 | 3.65 | 0.00 | 3.69 | 0.04 | 3.63 | 0.00 | 3.63 | 0.00 | 3.79 | 0.85 | 0.05 |
| 12 | 3.61 | 0.00 | 3.68 | 0.05 | 3.63 | 0.00 | 3.63 | 0.00 | 3.67 | 0.04 | 0.06 |
| 13 | 1.72 | 0.00 | 3.72 | 0.00 | 3.72 | 0.00 | 3.72 | 0.00 | 3.75 | 0.03 | 0.60 |
| 14 | 3.78 | 0.00 | 3.80 | 0.02 | 3.78 | 0.00 | 3.78 | −0.02 | 3.81 | 0.81 | −0.01 |
| Extra | 3.34 | 0.00 | 3.37 | 0.03 | 3.34 | 0.00 | 3.34 | 0.00 | 3.66 | 0.62 | 0.03 |
| Mean | 3.70 | 0.40 | 3.75 | 0.03 | 3.76 | 0.00 | 3.69 | −0.01 | 3.74 | 0.84 | 0.02 |
| SE | 0.43 | 0.00 | 0.03 | 0.00 | 0.03 | 0.00 | 0.03 | 0.00 | 0.03 | 0.80 | 0.01 |
| ANOVA (Tukey's post-hoc) vs. Group 2 | | | | 0.129 | | | | 0.719 | | 0.923 | 0.998 |
| ANOVA (Tukey's post-hoc) vs. Group 3 | | | | 0.813 | | | | 0.959 | | 0.543 | 1.000 |
| ANOVA (Tukey's post-hoc) vs. Group 4 | | | | 0.854 | | | | 0.836 | | 0.971 | 0.999 |
| ANOVA (Tukey's post-hoc) vs. Group 5 | | | | 0.378 | | | | 0.618 | | 0.833 | 1.080 |
| | Day −1 | | Day 0.25 | | Day 1 | | Day 7 | | Day 7.25 | | |
| Group 3 | Caliper | | Caliper | | Caliper | | Caliper | | Caliper | | |
| AAV-3 Guide 1 + 2 | Measurement ( [symbol] ) | | Measurement ( [symbol] ) | | Measurement ( [symbol] ) | | Measurement ( [symbol] ) | | Measurement ( [symbol] ) | | |
| ( [symbol] × 10\hat{ }9 of each guide / | | A from | | A from | | A from | | A from | | A from | Day 4-7 |
| knee) IA, 1× (D0) | Right | Baseline (D-1) | Right | Baseline (D-1) | Right | Baseline (D-1) | Right | Baseline (D-1) | Right | Baseline (D-1) | AUC |
| 1 | 3.48 | 0.00 | 3.48 | 0.08 | 3.48 | 0.00 | 3.48 | 0.00 | 3.51 | 0.03 | 0.00 |
| 2 | 4.04 | 0.00 | 4.04 | 0.00 | 4.04 | 0.00 | 4.02 | −0.02 | 4.04 | 0.00 | −0.06 |
| 3 | 3.67 | 0.00 | 3.67 | 0.00 | 3.67 | 0.00 | 3.67 | 0.00 | 3.71 | 0.84 | 0.01 |
| 4 | 3.62 | 0.00 | 3.62 | 0.00 | 3.62 | 0.00 | 3.62 | 0.00 | 3.67 | 0.05 | 0.01 |
| 5 | 3.58 | 0.00 | 3.58 | 0.00 | 3.58 | 0.00 | 3.58 | 0.00 | 3.62 | 0.08 | 0.01 |
| 6 | 3.80 | 0.00 | 3.68 | 0.05 | 3.80 | 0.00 | 3.81 | 0.01 | 3.84 | 0.04 | 0.09 |
| 7 | 3.88 | 0.00 | 3.89 | 0.01 | 3.88 | 0.00 | 3.88 | 0.00 | 3.80 | 0.02 | 0.01 |
| 8 | 3.75 | 0.00 | 3.75 | 0.00 | 3.75 | 0.00 | 3.75 | 0.00 | 3.79 | 0.04 | 0.01 |
| 9 | 3.56 | 0.00 | 3.60 | 0.04 | 3.56 | 0.00 | 3.56 | 0.00 | 3.68 | 0.08 | 0.03 |
| 10 | 3.64 | 0.00 | 3.64 | 0.08 | 3.64 | 0.00 | 3.64 | 0.00 | 3.69 | 0.03 | 0.03 |
| 11 | 3.65 | 0.00 | 3.78 | 0.05 | 3.65 | 0.00 | 3.65 | 0.00 | 3.89 | 0.04 | 0.06 |
| 12 | 3.75 | 0.00 | 3.55 | 0.00 | 3.55 | 0.00 | 3.54 | −0.01 | 3.78 | 0.03 | 0.03 |
| 13 | 3.68 | 0.00 | 3.68 | 0.08 | 3.68 | 0.00 | 3.68 | 0.00 | 3.68 | 0.08 | 0.03 |
| 14 | 3.65 | 0.00 | 3.69 | 0.04 | 3.61 | 0.00 | 3.65 | 0.00 | 3.65 | 0.00 | 0.04 |
| Extra | 3.63 | 0.00 | 3.64 | 0.02 | 3.63 | 0.00 | 3.63 | 0.00 | 3.63 | 0.01 | 0.62 |
| Mean | 3.78 | 0.00 | 3.71 | 0.81 | 3.78 | 0.80 | 3.78 | 0.00 | 3.73 | 0.03 | 0.81 |
| SE | 0.84 | 0.00 | 0.94 | 0.81 | 0.94 | 0.80 | 0.94 | 0.80 | 0.04 | 0.01 | 0.81 |
| ANOVA (Tukey's post-hoc) vs. Group 3 | | | | 0.741 | | | | 0.394 | | 0.352 | 0.989 |
| ANOVA (Tukey's post-hoc) vs. Group 4 | | | | 0.986 | | | | >0.9899 | | 0.984 | 0.982 |
| ANOVA (Tukey's post-hoc) vs. Group 5 | | | | 0.935 | | | | 0.999 | | 1.880 | 0.989 |
| | Day −1 | | Day 0.25 | | Day 1 | | Day 7 | | Day 7.25 | | |
| Group 3 | Caliper | | Caliper | | Caliper | | Caliper | | Caliper | | |
| AAV-3 Guide 1 + 2 | Measurement ( [symbol] ) | | Measurement ( [symbol] ) | | Measurement ( [symbol] ) | | Measurement ( [symbol] ) | | Measurement ( [symbol] ) | | |
| ( [symbol] × 10\hat{ }9 of each guide / | | A from | | A from | | A from | | A from | | A from | Day −1-7 |
| knee) IA, 1× (D0) | Right | Baseline (D-1) | Right | Baseline (D-1) | Right | Baseline (D-1) | Right | Baseline (D-1) | Right | Baseline (D-1) | AUC |
| 1 | 3.68 | 0.00 | 3.72 | 0.04 | 3.68 | 0.00 | 3.68 | 0.00 | 3.70 | 0.02 | 0.04 |
| 2 | 3.68 | 0.00 | 3.68 | 0.00 | 3.68 | 0.00 | 3.67 | −0.01 | 3.79 | 0.02 | −0.03 |
| 3 | 3.71 | 0.00 | 3.71 | 0.00 | 3.71 | 0.00 | 3.70 | −0.01 | 3.72 | 0.01 | −0.03 |
| 4 | 3.68 | 0.00 | 3.69 | 0.03 | 3.86 | 0.00 | 3.85 | −0.01 | 3.73 | 0.07 | 0.01 |
| 5 | 3.65 | 0.00 | 3.66 | 0.01 | 3.65 | 0.00 | 3.65 | 0.00 | 3.68 | 0.03 | 0.01 |
| 6 | 3.68 | 0.00 | 3.70 | 0.02 | 3.68 | 0.00 | 3.69 | 0.01 | 3.70 | 0.02 | 0.03 |
| 7 | 3.74 | 0.00 | 3.77 | 0.83 | 3.74 | 0.00 | 3.74 | 0.00 | 3.75 | 0.01 | 0.03 |
| 8 | 3.68 | 0.00 | 3.68 | 0.02 | 3.68 | 0.00 | 3.69 | 0.00 | 3.71 | 0.02 | 0.00 |
| 9 | 3.72 | 0.00 | 3.75 | 0.03 | 3.72 | 0.00 | 3.73 | 0.01 | 3.75 | 0.03 | 0.06 |
| 10 | 3.74 | 0.00 | 3.74 | 0.00 | 3.74 | 0.00 | 3.73 | −0.01 | 3.76 | 0.02 | −0.01 |
| 11 | 3.62 | 0.00 | 3.67 | 0.05 | 3.62 | 0.00 | 3.62 | 0.00 | 3.67 | 0.05 | 0.06 |
| 12 | 3.79 | 0.00 | 3.79 | 0.08 | 3.79 | 0.00 | 3.79 | 0.00 | 3.87 | 0.08 | 0.01 |
| 13 | 3.67 | 0.00 | 3.73 | 0.04 | 3.67 | 0.00 | 3.67 | 0.00 | 3.70 | 0.03 | 0.04 |
| 14 | 3.68 | 0.00 | 3.70 | 0.02 | 3.68 | 0.00 | 3.69 | 0.01 | 3.79 | 0.02 | 0.05 |
| Extra | 3.72 | 0.00 | 3.32 | 0.00 | 3.72 | 0.00 | 3.72 | 0.00 | 3.72 | 0.03 | 0.03 |
| Mean | 3.69 | 0.00 | 3.71 | 0.03 | 3.69 | 0.00 | 3.68 | 0.00 | 3.72 | 0.03 | 0.02 |
| SE | 0.41 | 0.00 | 0.41 | 0.00 | 0.01 | 0.00 | 0.03 | 0.00 | 0.01 | 0.01 | 0.01 |
| | Day −1 | | Day 0.25 | | Day 1 | | Day 7 | | Day 7.25 | | |
| | Caliper | | Caliper | | Caliper | | Caliper | | Caliper | | |
| Group 3 | Measurement ( [symbol] ) | | Measurement ( [symbol] ) | | Measurement ( [symbol] ) | | Measurement ( [symbol] ) | | Measurement ( [symbol] ) | | Day −1-7 |
| PBS | | A from | | A from | | A from | | A from | | A from | A from Baseline |
| IA, 1× (D0) | Right | Baseline (D-1) | Right | Baseline (D-1) | Right | Baseline (D-1) | Right | Baseline (D-1) | Right | Baseline (D-1) | AUC |
| 1 | 3.85 | 0.00 | 3.59 | 0.04 | 3.55 | 0.00 | 3.85 | 0.00 | 3.99 | 0.05 | 0.05 |
| 2 | 3.78 | 0.00 | 3.78 | 0.00 | 3.78 | 0.00 | 3.76 | −0.02 | 3.75 | 0.01 | −0.06 |
| 3 | 3.68 | 0.00 | 3.72 | 0.04 | 3.68 | 0.00 | 3.68 | 0.00 | 3.73 | 0.07 | 0.05 |
| 4 | 3.72 | 0.00 | 3.77 | 0.05 | 3.72 | 0.00 | 3.74 | 0.02 | 3.79 | 0.07 | 0.12 |
| 5 | 3.62 | 0.00 | 3.62 | 0.00 | 3.62 | 0.00 | 3.62 | 0.00 | 3.78 | 0.08 | 0.01 |
| 6 | 3.68 | 0.00 | 3.68 | 0.00 | 3.68 | 0.00 | 3.68 | −0.02 | 3.74 | 0.06 | −0.06 |
| 7 | 3.78 | 0.00 | 3.78 | 0.00 | 3.78 | 0.00 | 3.78 | 0.00 | 3.85 | 0.08 | 0.01 |
| 8 | 3.72 | 0.00 | 3.76 | 0.04 | 3.72 | 0.00 | 3.73 | 0.01 | 3.80 | 0.08 | 0.08 |
| 9 | 3.63 | 0.00 | 3.69 | 0.04 | 3.63 | 0.00 | 3.69 | 0.00 | 3.71 | 0.06 | 0.05 |
| 10 | 3.58 | 0.00 | 3.58 | 0.08 | 3.58 | 0.00 | 3.58 | 0.08 | 3.59 | 0.01 | 0.00 |
| 11 | 3.61 | 0.00 | 3.68 | 0.03 | 3.63 | 0.00 | 3.63 | 0.00 | 3.68 | 0.03 | 0.06 |
| 12 | 3.79 | 0.00 | 3.79 | 0.00 | 3.79 | 0.00 | 3.73 | −0.02 | 3.82 | 0.03 | −0.06 |
| 13 | 3.58 | 0.00 | 3.58 | 0.02 | 3.58 | 0.00 | 0.03 | 3.56 | 3.61 | 0.05 | 0.03 |
| 14 | 3.75 | 0.00 | 3.78 | 0.03 | 3.73 | 0.00 | 3.72 | −0.01 | 3.75 | 0.02 | 0.02 |
| Mean | 3.70 | 0.00 | 3.72 | 0.02 | 3.70 | 0.00 | 3.70 | 0.09 | 3.75 | 0.05 | 0.02 |
| SE | 0.02 | 0.00 | 0.02 | 0.01 | 0.02 | 0.00 | 0.02 | 0.00 | 0.02 | 0.05 | 0.05 |
| ANOVA (Tukey's post-hoc) vs. Group 4 | | | | >0.9999 | | | 0.997 | 0.997 | | 0.214 | >0.9999 |
| ANOVA (Tukey's post-hoc) vs. Group 5 | | | | 0.980 | | | 0.975 | 0.978 | | 0.360 | >0.9999 |
| | Day −1 | | Day 0.25 | | Day 1 | | Day 7 | | Day 7.25 | | |
| Group 4 | Caliper | | Caliper | | Caliper | | Caliper | | Caliper | | |
| AAV-6 Serendite Value | Measurement ( [symbol] ) | | Measurement ( [symbol] ) | | Measurement ( [symbol] ) | | Measurement ( [symbol] ) | | Measurement ( [symbol] ) | | |
| 0 × 10\hat{ }10 particles / | | A from | | A from | | A from | | A from | | A from | Day −1-7 |
| knee) IA, 1× (D0) | Right | Baseline (D-1) | Right | Baseline (D-1) | Right | Baseline (D-1) | Right | Baseline (D-1) | Right | Baseline (D-1) | AUC |
| 1 | 3.67 | 0.00 | 3.70 | 0.03 | 3.67 | 0.00 | 3.88 | 0.01 | 3.70 | 0.03 | 0.07 |
| 2 | 3.77 | 0.00 | 3.50 | 0.01 | 3.77 | 0.00 | 3.73 | −0.02 | 3.81 | 0.04 | −0.01 |
| 3 | 3.72 | 0.00 | 3.74 | 0.02 | 3.72 | 0.00 | 3.73 | 0.01 | 3.75 | 0.03 | 0.05 |
| 4 | 3.61 | 0.00 | 3.63 | 0.02 | 3.51 | 0.00 | 3.61 | 0.03 | 3.66 | 0.05 | 0.03 |
| 5 | 3.68 | 0.00 | 3.71 | 0.01 | 3.68 | 0.00 | 3.68 | 0.00 | 3.714 | 0.03 | 0.03 |
| 6 | 3.84 | 0.00 | 3.84 | 0.00 | 3.54 | 0.00 | 3.82 | −0.03 | 3.87 | 0.03 | −0.05 |
| 7 | 3.72 | 0.00 | 3.74 | 0.02 | 3.72 | 0.00 | 3.72 | 0.00 | 3.77 | 0.05 | 0.03 |
| 8 | 3.70 | 0.00 | 3.82 | 0.12 | 3.79 | 0.00 | 3.71 | 0.01 | 3.72 | 0.02 | 0.15 |
| 9 | 3.50 | 0.00 | 3.81 | 0.01 | 3.60 | 0.00 | 3.81 | 0.01 | 3.83 | 0.03 | 0.05 |
| 10 | 3.72 | 0.00 | 3.72 | 0.60 | 3.72 | 0.00 | 3.72 | 0.00 | 3.76 | 0.04 | 0.00 |
| 11 | 3.60 | 0.00 | 3.60 | 0.00 | 3.50 | 0.00 | 3.68 | 0.00 | 3.62 | 0.02 | 0.00 |
| 12 | 3.63 | 0.00 | 3.64 | 0.01 | 3.63 | 0.00 | 3.63 | 0.00 | 3.69 | 0.08 | 0.02 |
| 13 | 3.59 | 0.00 | 3.58 | 0.50 | 3.58 | 0.00 | 3.57 | −0.02 | 3.42 | 0.01 | −0.08 |
| 14 | 3.68 | 0.00 | 3.70 | 0.02 | 3.88 | 0.00 | 3.58 | 0.00 | 3.71 | 0.03 | 0.02 |
| Extra | 3.73 | 0.00 | 3.79 | 0.01 | 3.74 | 0.00 | 3.22 | −0.02 | 3.73 | 0.01 | 0.05 |
| Mean | 3.78 | 0.00 | 3.72 | 0.02 | 3.70 | 0.00 | 3.69 | 0.88 | 3.73 | 0.84 | 0.82 |
| SE | 0.82 | 0.00 | 0.03 | 0.01 | 0.02 | 8.00 | 0.02 | 0.00 | 0.02 | 0.00 | 0.01 |
| ANOVA (Tukey's pass-hoc) vs. Group 3 | | 0.00 | | 0.998 | | | 1.000 | | | 0.962 | >0. |

[symbol]

indicates data missing or illegible when filed

Moribund or Found Dead Animals

If animals were found dead no samples were taken. For animals needing to be euthanized, regardless of reason, samples were taken as they would at necropsy (see Necropsy Information section). Animals on health assessment may be given SC fluids as well as hydrogel and food on the bottom of the cage.

Study Group Designations

| | | | | Dose Level | | Dose Conc. | | |
|---|---|---|---|---|---|---|---|---|
| Grp | N | MSU | Treatment | (particles/knee) | Dose Vol. | (particles/ml) | Dose Route | Regimen |
| 1 | 14 | Yes | AAV-6 | 1 × 10¹⁰ | 10 μL | 1 × 10¹² | IA | 1× (Day 0) |
| | | | Scramble Vector | | | | (right knee) | |
| 2 | 14 | Yes | AAV-6 | 5 × 10⁹ | 10 μL | 1 × 10¹² | IA | 1× (Day 0) |
| | | | Guide 1 + 2 | (of each guide) | | | (right knee) | |
| 3 | 14 | Yes | PBS | — | 10 μL | — | IA | 1× (Day 0) |
| | | | | | | | (right knee) | |
| 4 | 14 | Yes | AAV-5 | 1 × 10¹⁰ | 10 μL | 1 × 10¹² | IA | 1× (Day 0) |
| | | | Scramble Vector | | | | (right knee) | |
| 5 | 14 | Yes | AAV-5 | 5 × 10⁹ | 10 μL | 1 × 10¹² | IA | 1× (Day 0) |
| | | | Guide 1 + 2 | (of each guide) | | | (right knee) | |

Study Calendar

| Mon | Tue | Wed | Thu | Fri | Sat | Sun |
|---|---|---|---|---|---|---|
| May 4, 2020 | May 5, 2020 | May 6, 2020 | May 7, 2020 | May 8, 2020 | May 9, 2020 | May 10, 2020 |
| Day −10 | Day −9 | Day −8 | Day −7 | Day −6 | Day −5 | Day −4 |
| Distribute | | | | Acclimate to VF | | |
| animals on arrival | | | | | | |
| into groups for | | | | | | |
| acclimation | | | | | | |
| May 11, 2020 | May 12, 2020 | May 13, 2020 | May 14, 2020 | May 15, 2020 | May 16, 2020 | May 17, 2020 |
| Day −3 | Day −2 | Day −1 | Day 0 | Day 1 | Day 2 | Day 3 |
| Acclimate to VF | Acclimate to VF | Weigh & | Timed Dose, | Timed Caliper, | Weigh | |
| | | Randomize, | Timed Caliper, | Timed Behavior | | |
| | | Baseline Caliper, | Timed Behavior | | | |
| | | Baseline | | | | |
| | | Behavior. Shave | | | | |
| | | Knees | | | | |
| May 18, 2020 | May 19, 2020 | May 20, 2020 | May 21, 2020 | May 22, 2020 | May 23, 2020 | May 24, 2020 |
| Day 4 | Day 5 | Day 6 | Day 7 | Day 8 | Day 9 | Day 10 |
| | | Weigh, Shave | Timed MSU, | | | |
| | | Knees? | Timed Caliper, | | | |
| | | | Timed Behavior, | | | |
| | | | Timed Necropsy | | | |

Materials

| Name | Supplier | Cat #* |
|---|---|---|
| MSU crystals | Invivogen | Tlrl-msu-25 |
| Vehicle (PBS) | Corning | 21-031-CV |
| Gene | SgRNA | AA06-CCPCTRO1-AD01-200 |
| Gene | SgRNA | AA06-MCP001682-AD01-2-200A |
| Gene | SgRNA | AA06-MCP001682-AD01-2-200B |
| Gene | SgRNA | AA05 -CCPCTROI-AD01-200 |
| Gene | SgRNA | AA05-MCP001682-AD01-2-200-9 |
| Gene | SgRNA | AA05-MCP001682-AD01-2-200-6 |
| Isoflurane | VetOne | 502017 |
| Syringes & Needles | BD | As needed |
| Serum Separator Tubes | Greiner Bio-One | #450472 (via Fisher) |
| (if needed) | | |
| Li Hep Mini-Collect | Greiner Bio-One | #450480 (via Fisher) |
| (if needed) | | |
| EDTA Mini-Collect | Greiner Bio-One | #450477 (via Fisher) |
| (if needed) | | |
| K3EDTA (if needed) | Covidien | #8881311149 (via Fisher) |

Test Article and Vehicle Information

| | | | | | | Com- | | |
|---|---|---|---|---|---|---|---|---|
| | | | | Days | Doses | pound | 25% | TOTAL |
| | | | ml/ | of | Per | Needed | Over- | Needed |
| Grp | Cpmd | N | animal | Dosing | Day | (ml) | age | (ml) |
| 1 | AAV-6 | 14 | 0.01 | 1 | 1 | 0.14 | 0.04 | 0.18 |
| | Scramble | | | | | | | |
| | Vector | | | | | | | |
| 2 | AAV-6 | 14 | 0.01 | 1 | 1 | 0.14 | 0.04 | 0.18 |
| | Guide | | | | | | | |
| | 1 + 2 | | | | | | | |
| 3 | PBS | 14 | 0.01 | 1 | 1 | 0.14 | 0.04 | 0.18 |
| 4 | AAV-5 | 14 | 0.01 | 1 | 1 | 0.14 | 0.04 | 0.18 |
| | Scramble | | | | | | | |
| | Vector | | | | | | | |
| 5 | AAV-5 | 14 | 0.01 | 1 | 1 | 0.14 | 0.04 | 0.18 |
| | Guide | | | | | | | |
| | 1 + 2 | | | | | | | |

Unformulated Test Article Storage Conditions:

AAV-6 Scrambled Vector (Group 1): −80 C.
AAV-6 Guide 1 + 2 (Group 2): −80 C.
PBS (Group 3): 4 C.
AAV-5 Scrambled Vector (Group 4): −80 C.
AAV-5 Guide 1 + 2 (Group 5): −80 C.

Vehicle Information:

AAV-6 Scrambled Vector (Group 1): Sterile PBS (w/o Ca & Mg)
AAV-6 Guide 1 + 2 (Group 2): Sterile PBS (w/o Ca & Mg)
PBS (Group 3): Sterile PBS (w/o Ca & Mg)
AAV-5 Scrambled Vector (Group 4): Sterile PBS (w/o Ca & Mg)
AAV-5 Guide 1 + 2 (Group 5): Sterile PBS (w/o Ca & Mg)

Test Article Formulation Instructions & Calculations:

AAV-6 Scrambled Vector (Group 1): Dilute stock to appropriate
concentration using sterile PBS (See AAV Preparation section)
AAV-6 Guide 1 + 2 (Group 2): Dilute stock to appropriate
concentration using sterile PBS (See AAV Preparation section)
PBS (Group 3): Use sterile PBS (See AAV Preparation section)
AAV-5 Scrambled Vector (Group 4): Dilute stock to appropriate
concentration using sterile PBS (See AAV Preparation section)
AAV-5 Guide 1 + 2 (Group 5): Dilute stock to appropriate
concentration using sterile PBS (See AAV Preparation section)

Dosing Formulations and Vehicle Storage & Stability:

AAV-6 Scrambled Vector (Group 1): Dilute just prior to injecting
AAV-6 Guide 1 + 2 (Group 2): Dilute just prior to injecting
AAV-5 Scrambled Vector (Group 4): Dilute just prior to injecting
AAV-5 Guide 1 + 2 (Group 5): Dilute just prior to injecting

Disposition of Test Articles Following Dosing:

AAV-6 Scrambled Vector (Group 1): Discard formulations, retain stock
solution at −80 C. for future studies
AAV-6 Guide 1 + 2 (Group 2): Discard formulations, retain stock solution
at −80 C. for future studies
AAV-5 Scrambled Vector (Group 4): Discard formulations, retain stock
solution at −80 C. for future studies
AAV-5 Guide 1 + 2 (Group 5): Discard formulations, retain stock solution
at −80 C. for future studies

Live Phase

Live Phase Data Collection

| Type | Study Day | Grp (An) | Details |
|---|---|---|---|
| Body Weight | Day −1, 2, 6 | All | |
| Knee Caliper | Day −1 (Baseline) | All | Right Knee |
| | Day 0 (6 hr) | | Change from |
| | Day 1 (24 hour) | | baseline |
| | Day 7 (0 hr, 6 hour) | | |
| Von Frey | Day −1 (Baseline) | All | Right foot only |
| | Day 0 (6 hr) | | |
| | Day 1 (24 hour) | | |
| | Day 7 (0 hr, 6 hour) | | |

Necropsy Information

The animals were necropsied after the final behavioral testing on study day 7 (approx. 6 h post-MSU). At necropsy, the animals were bled by cardiac puncture to exsanguination and euthanized by cervical dislocation for tissue collection. Whole blood was processed for serum (≥200 ?IL/mouse), which was stored frozen at −80° C. for shipment to the study sponsor. Right (injected) and left (normal) knees from all animals were collected (skin, muscle, and feet were removed while keeping the knee joint intact). The joints were flash-frozen straight in 15-mL conical tubes for shipment to the sponsor.

| | |
|---|---|
| Sacrifice Schedule: | Study Day 7 (6 hours post MSU): |
| | ALL ANIMALS |
| Method of Euthanasia: | Bleed by cardiac puncture to exsanguinate |
| | followed by cervical dislocation. |
| Time Points: | After final behavior time point (6 hours |
| | post MSU) |

Necropsy Sample Collection:

| | Process | Anti- | | Final | Storage | |
|---|---|---|---|---|---|---|
| Type | For | Coag | Gr/An | Volume | Condition | Disposition |
| Cardiac | Serum | N/A | All | ≥200 μl | −80 C., | Ship to |
| Puncture Whole | | | | | Epps | Sponsor |
| Blood | | | | | | |

Necropsy Tissue Sample Collection:

| Type | Gr/An | Details | Storage Condition | Disposition |
|---|---|---|---|---|
| Right | All | Remove skin & | Flash freeze | Ship to |
| (Injected) | | muscle keeping | straight, place | Sponsor |
| Knee | | knee joint intact, | in 15 ml conical | |
| | | remove foot | | |
| Left | All | Remove skin & | Flash freeze | Ship to |
| (Normal) | | muscle keeping | straight, place | Sponsor |
| Knee | | knee joint intact, | in 15 ml conical | |
| | | remove foot | | |

Statistical Analysis

Data were entered into Microsoft Excel and means and standard errors (SE) for each group were determined. The groups were compared using a one-way analysis of variance (ANOVA) or a repeated measures (RM) ANOVA with a Tukey's post-hoc analysis. ANOVA were performed using Prism v8.0.2 software (GraphPad). Unless indicated, BBP performs statistical analysis on raw (untransformed) data only. Statistical tests make certain assumptions regarding the data's normality and homogeneity of variance, and further analysis may be required if testing resulted in violations of these assumptions. Significance for all tests was set at p<0.050 with p values rounded to the third decimal place.

AAV Preparation

Standard Operating Procedure for Preparing Virus for Injection

Create a working stock that contains 5×10\hat{ }11 vg per ml for each construct (Guide 1, Guide 2). Note that for equivalence, the scramble control group needs to be injected with a total of 1×10\hat{ }10 copies of the scrambled vector. Diluent (PBS) will be used in the vehicle control group.

Materials

AA06-CCPCTR01-AD01-200: Scramble control AAV-6 particles,
100 microliter solution containing 5 × 10\hat{ }12 vg per ml.
AA06-MCP001682-AD01-2-200-Guide 1: AAVPrimeTMparticles,
AAV-6 containing Guide 1, 100 microliter solution containing
5 × 10\hat{ }12 vg per ml.
AA06-MCP001682-AD01-2-200-Guide 2: AAVPrimeTMparticles,
AAV-6 containing Guide 2, 100 microliter solution containing
5 × 10\hat{ }12 vg per ml.
AA05-CCPCTR01-AD01-200: Scramble control AAV-5 particles,
100 microliter solution containing >5 × 10\hat{ }12 vg per ml.
AA05-MCP001682-AD01-2-200-Guide 1: AAVPrimeTMparticles,
AAV-5 containing Guide 1, 100 microliter solution
containing >5 × 10\hat{ }12 vg per ml.
AA05-MCP001682-AD01-2-200-Guide 2: AAVPrimeTMparticles,
AAV-5 containing Guide 2, 100 microliter solution
containing >5 × 10\hat{ }12 vg per ml.

Procedure

Group 1: AAV-6 Scramble Control—Aliquot 400 microliters of sterile-filtered Ca- and Mg-free PBS into a sterile Eppendorf tube. Add 100 microliters (equivalent to 5×10\hat{ }11 vg) of the stock AA06-CCPCTR01-AD01-200. This results in a 0.5 ml volume of working stock containing 1×10\hat{ }12 vg/ml of the AAV-5 scramble control. Injection of 10 microliters of this solution into the knee joint delivers 1×10\hat{ }10 vg of the AAV-6 scramble control.

Group 2: Active AAV-6 Guide 1+2—Aliquot 800 microliters of sterile-filtered Ca- and Mg-free PBS into a sterile Eppendorf tube. Add 100 microliters (equivalent to 5×10\hat{ }11 vg) of each of the two active AAV-6 constructs—this means 100 microliters (equivalent to 5×10\hat{ }11 vg) of AA06-MCP001682-AD01-2-200-a and 100 microliters (equivalent to 5×10\hat{ }11 vg) of the stock AA06-MCP001682-AD01-2-200-b. This results in a 1 ml volume of working stock containing 5×10\hat{ }11 vg/ml for each of the two guides. Injection of 10 microliters of this solution into the knee joint delivers 5×10\hat{ }9 vg for each of the two AAV-6 guides.

Group 3: PBS—The sterile-filtered Ca- and Mg-free PBS used to dilute the virus stock served as the vehicle control for this study. It was dosed at 10 microliters per knee joint.

Group 4: AAV-5 Scramble Control—Aliquot 400 microliters of sterile-filtered Ca- and Mg-free PBS into a sterile Eppendorf tube. Add 100 microliters (equivalent to 5×10\hat{ }11 vg) of the stock AA05-CCPCTR01-AD01-200. This results in a 0.5 ml volume of working stock containing 1×10\hat{ }12 vg/ml of the AAV-5 scramble control. Injection of 10 microliters of this solution into the knee joint delivered 1×10\hat{ }10 vg of the AAV-5 scramble control.

Group 5: Active AAV-6 Guide 1+2—Aliquot 800 microliters of sterile-filtered Ca- and Mg-free PBS into a sterile Eppendorf tube. Add 100 microliters (equivalent to 5×10\hat{ }11 vg) of each of the two active AAV-6 constructs—this means 100 microliters (equivalent to 5×10\hat{ }11 vg) of AA05-MCP001682-AD01-2-200-a and 100 microliters (equivalent to 5×10\hat{ }11 vg) of the stock AA05-MCP001682-AD01-2-200-b. This results in a 1 ml volume of working stock containing 5×10\hat{ }11 vg/ml for each of the two guides. Injection of 10 microliters of this solution into the knee joint delivered 5×10\hat{ }9 vg for each of the two AAV-6 guides.

qPCR

Snap-frozen synovial tissues resected en bloc (including distal femur and proximal tibia) and placed in RLT buffer. Homogenised using Cyrolys Evolution tissue homogenizer (“HARD” programme cycle). RNA extracted using RNeasy or RNeasy Plus kits followed by QIAshredder (from QIAGEN). RNA quantified using Nanonstring. cDNA reverse transcribed and qPCR performed using mouse-specific primers for IL-1 beta, beta-actin and RPL13.

Results

As shown in FIG. 6, PBS control mice had mean body weight gain of 4.3% (1.1 g) over the course of the study. Body weight gain did not differ statistically across groups (Table 7, Table 9).

As shown in FIG. 7A, knee caliper measurements in all groups peaked 6 hours post-dose on study day 0, returned to baseline by 24 hours post-dose, and then peaked again 6 hours post-MSU on study day 7. Knee caliper change from baseline did not differ statistically across groups over time (Table 11). As shown in FIG. 7B, knee caliper change AUC for days −1 through 7 did not differ statistically across groups (Table 7, Table 11).

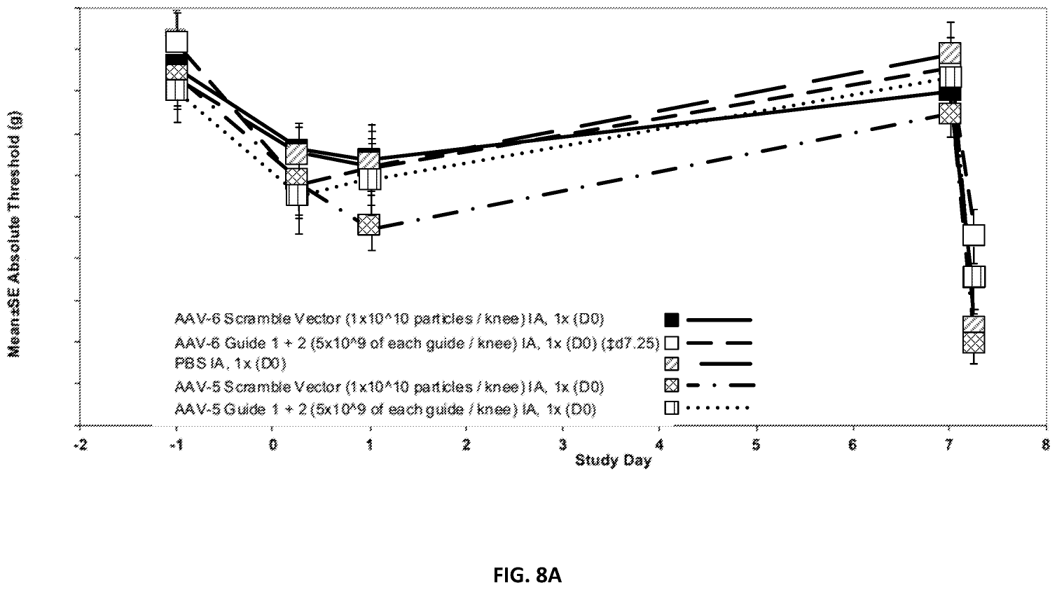

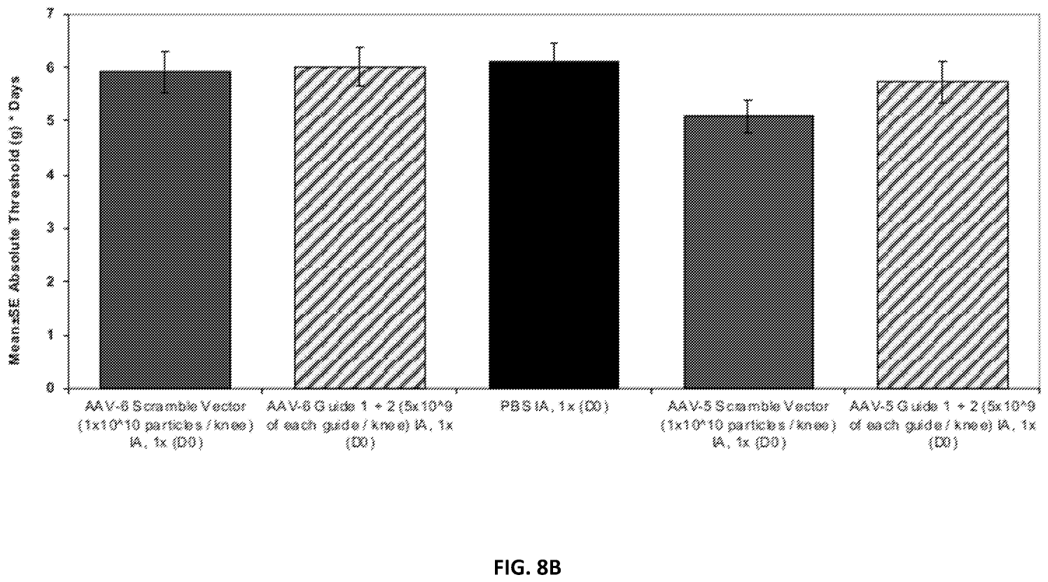

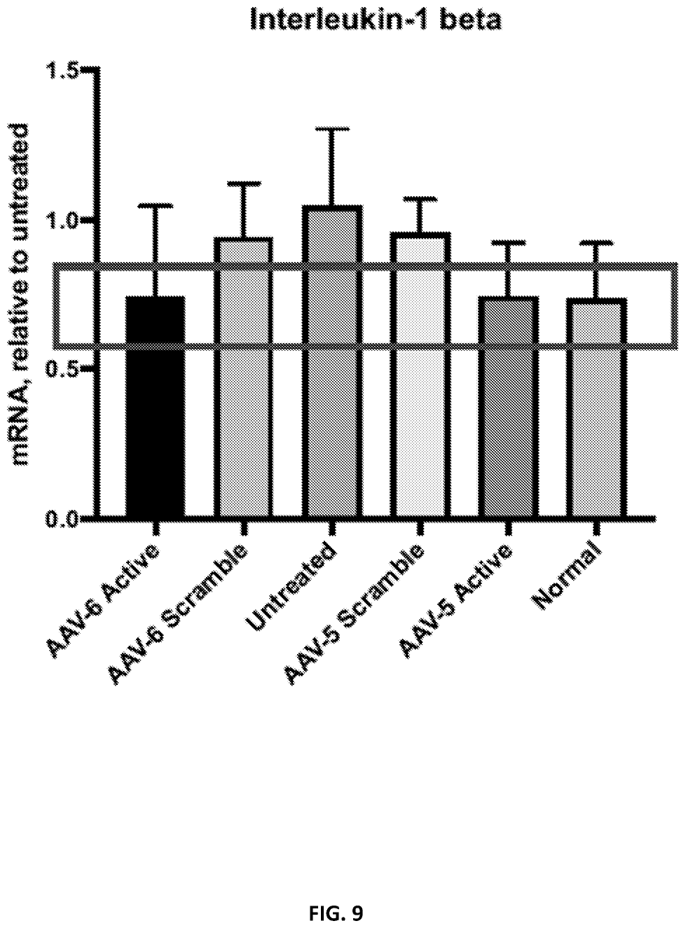

As shown in FIG. 8A, von Frey absolute thresholds decreased slightly in all groups following IA dosing on study day 0 and then trended toward baseline on study day 7 before decreasing sharply following IA injection of MSU. There were no statistical differences over time between the AAV scramble vector control groups and the PBS control group. Mice treated with AAV-6 (Guide 1+2) had von Frey absolute thresholds that were increased 6 hours post-MSU on day 7 as compared to the control groups; the increase in von Frey absolute thresholds at this time point nearly reached statistical significance as compared to the AAV-6 scramble vector and PBS control groups (p=0.051 and p=0.075, respectively) and was statistically significant as compared to the AAV-5 scramble vector control group (p=0.025). Mice treated with AAV-5 (Guide 1+2) had von Frey absolute thresholds that did not differ statistically from the control groups over time (Table 10). As shown in FIG. 8B, von Frey absolute threshold AUC for days −1 through 7 did not differ statistically across groups (Table 7, Table 10).

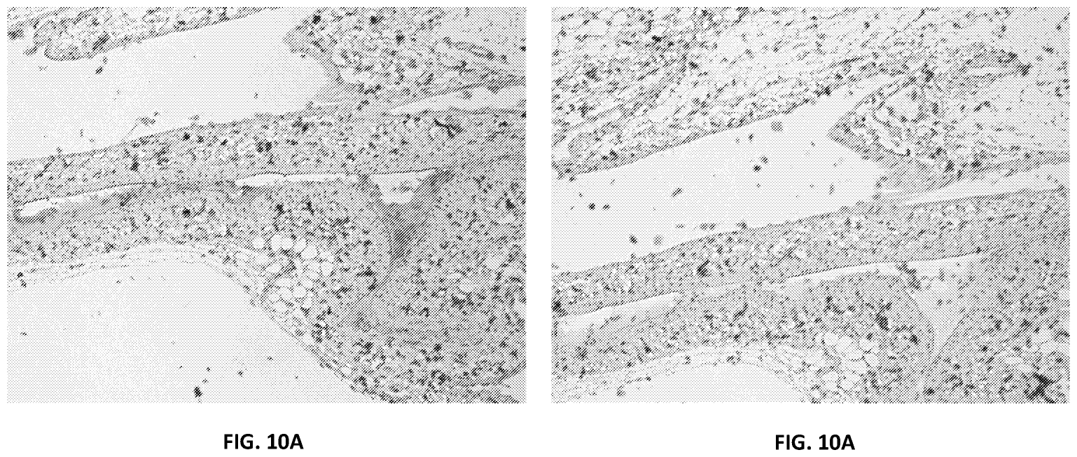

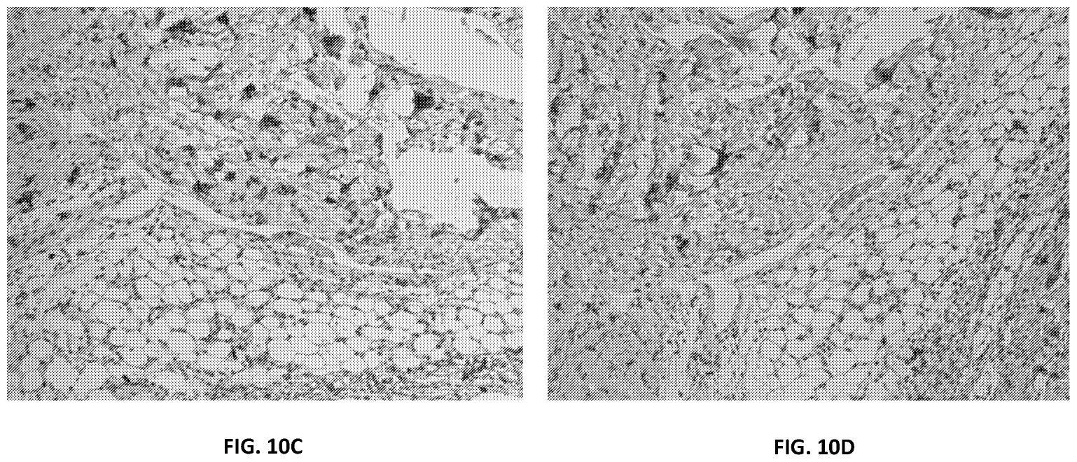

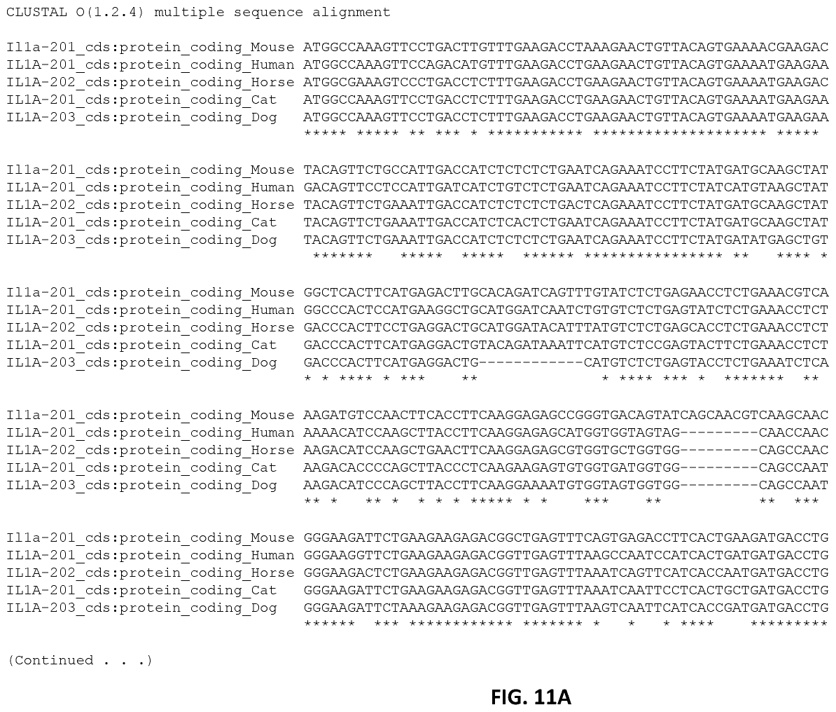

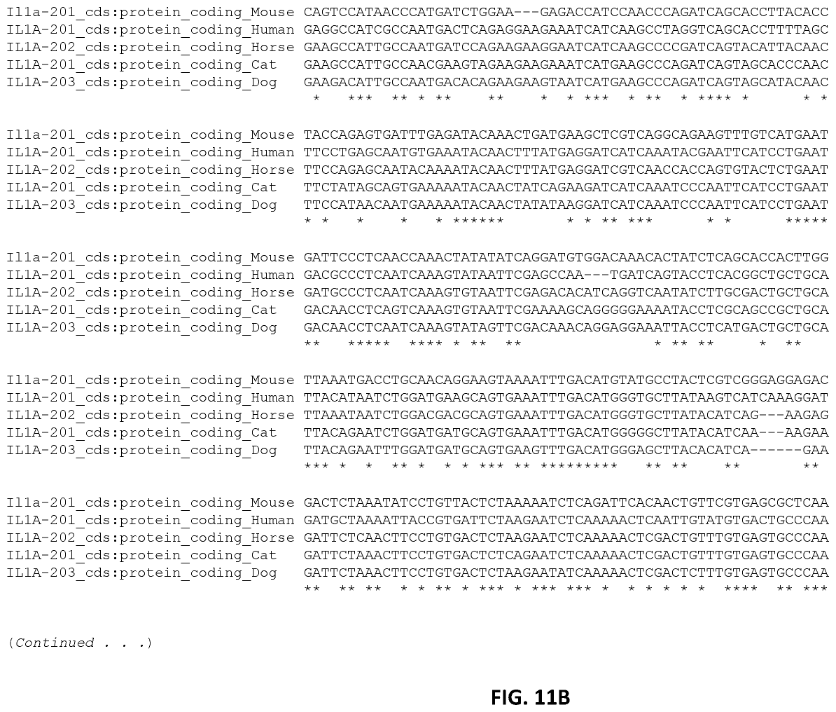

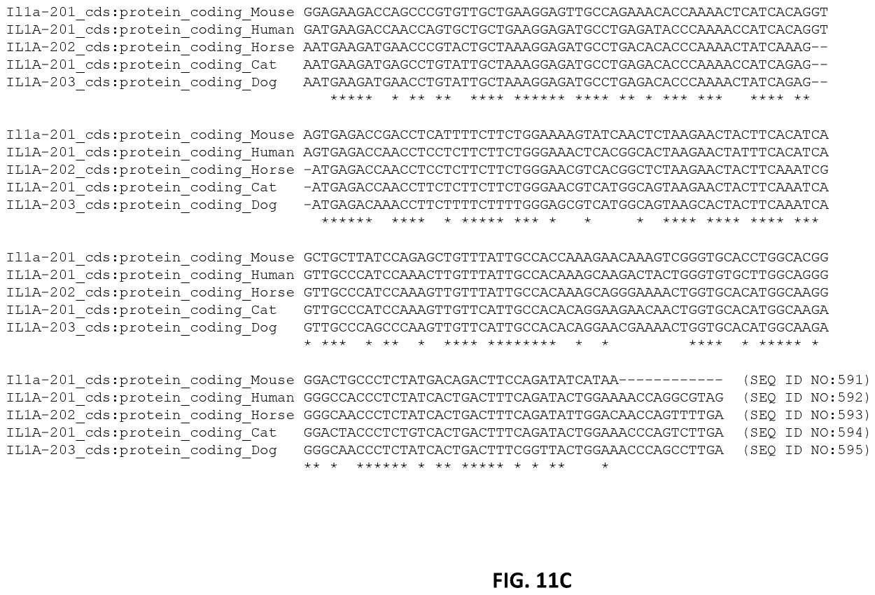

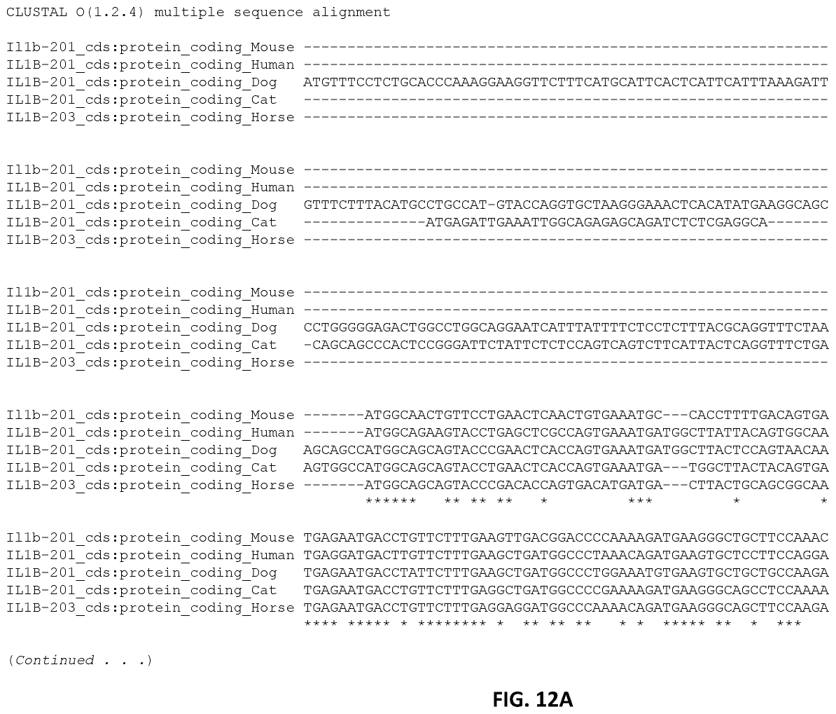

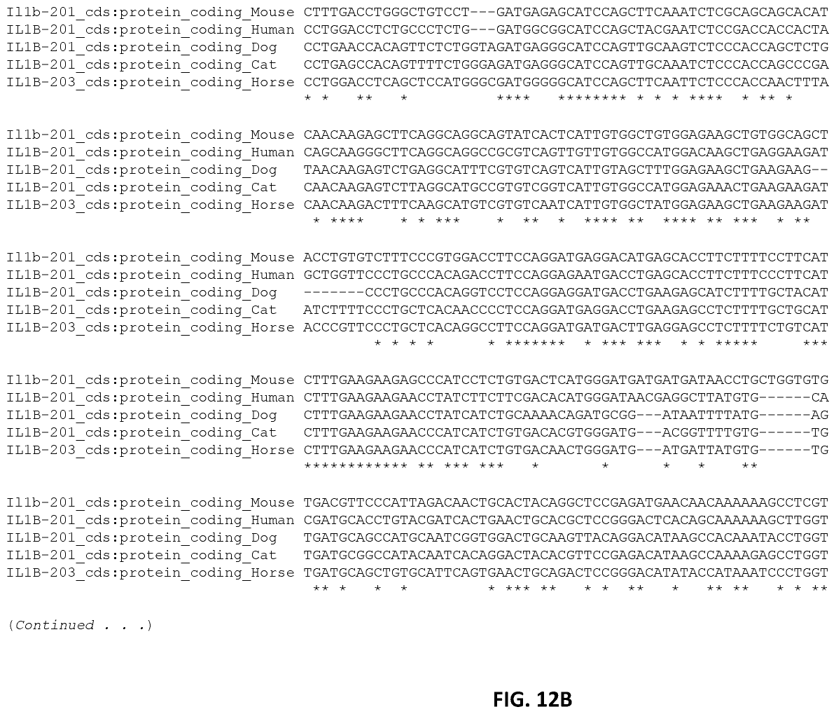

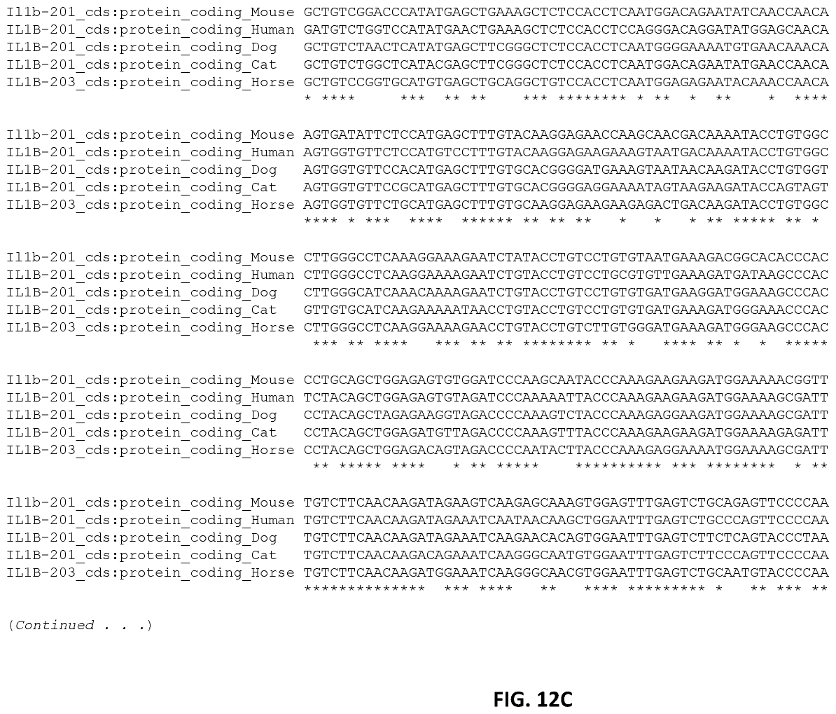

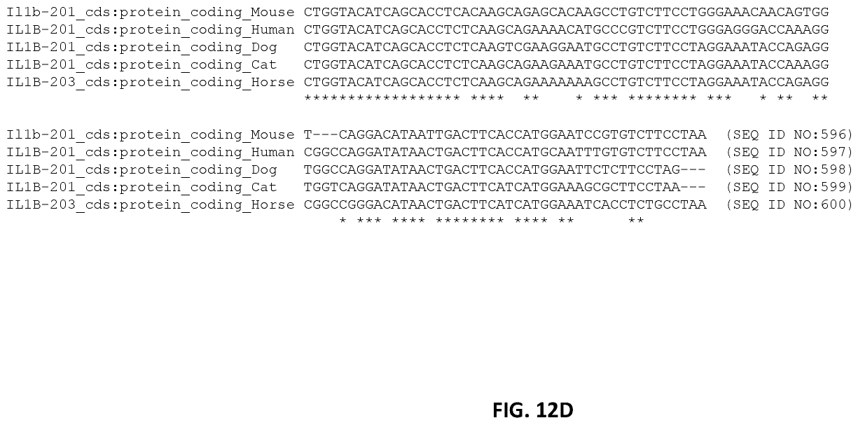

As shown in FIG. 10, immunohistochemistry data for murine IL-1β in synovial tissue showed reduced IL-1β expression in CRISPR-treated animals. (A) In MSU injected animals pre-treated with PBS there is robust expression of IL-1β (brown staining). This effect is not seen in CRISPR-treated animals (panel C). The absence of IL-1β (brown staining) in CRISPR-treated animals resembles negative antibody controls (panels B & D). All images are 10×original magnification.

Discussion & Conclusions

Mice treated IA (1× on day 0) with AAV-6 (Guide 1+2: 5×10⁹ vg/guide per knee) showed a statistically significant reduction in referred pain, as measured by von Frey testing, 6 hours after MSU injection on day 7 as compared to mice injected IA with AAV-5 scramble vector (p=0.025) with results being nearly significant as compared to the AAV-6 scramble vector and PBS control groups (p=0.051 and p=0.075, respectively). AUC calculations for von Frey assessments did not differ statistically across groups. Animal body weight gain and knee swelling did not differ statistically across groups. All animals survived to study termination.

### Example 6. Guide RNA Design

Guide RNAs targeting human IL-1α and IL-1β (Table 14) were designed according to the following procedure:

1. Identify appropriate genome assembly and gene model (Tools: Ensemble, UCSC Genome Browser);

2. Identify key functional domains to map out targeting window (Tools: Ensemble; Literature);

3. Generate list of all possible guide RNAs across key exons (Tools: Ensemble, UCSC, InDelphi);

4. Rank guides based on ML-predicted frameshifting score and exclude poor performers;

5. Exclude guides <5 bp from intron: exon boundaries and with homopolynucleotide tracts of 6×T's or greater;

6. Determine on-target (Doench 2016) and off-target (Hsu 2013) metrics for each guide (Tools: UCSC, Deskgen);

7. Filter out guides with poor on- and off-target scores to generate final list; and

8. Rank based on frameshift index.

Guide RNAs targeting cat, dog, or horse IL-1α and IL-1β (Table 15) were designed according to the following procedure:
1. Identify appropriate genome assembly and gene model (Tools: Ensemble, UCSC Genome Browser)
2. Identify key functional domains to map out targeting window (Tools: Ensemble; Literature)
3. Retrieve coding sequence from appropriate exons and relevant flanking intronic sequences (Tools: Ensemble. APE)
4. Generate list of all possible guide RNAs across key exons (Tools: Ensemble, InDelphi)
5. Rank guides based on ML-predicted frameshifting score and exclude poor performers
6. Exclude guides <5 bp from intron:exon boundaries and with homopolynucleotide tracts of 6×T's or greater
7. Determine off-target metrics for each guide (Tools: Cas Off-Finder, Excel)
8. Filter out guides with poor off-target scores to generate final list
9. Rank based on frameshift index.

Gene transcript information for all species considered are included in Table 16.

TABLE 14

| Gene | SEQ | | | | Frameshift | Doench | Hsu | | |
|---|---|---|---|---|---|---|---|---|---|
| symbol | ID NO: | Species | Chromosome | Genome | (%) | 2016 | 2013 | gRNA | PAM |
| IL1B | 627 | Human | chr2 | hg38 | 80.1 | 55 | 76 | AGCTGGATGCCGCCATCCAG | AGG |
| IL1B | 628 | Human | chr2 | hg38 | 78.5 | 44 | 71 | ACCACTACAGCAAGGGCTTC | AGG |
| IL1B | 629 | Human | chr2 | hg38 | 75.4 | 63 | 92 | CATGGCCACAACAACTGACG | CGG |
| IL1B | 630 | Human | chr2 | hg38 | 74 | 60 | 97 | GGTGGTCGGAGATTCGTAGC | TGG |
| IL1B | 631 | Human | chr2 | hg38 | 87.6 | 55 | 61 | CTACAGCAAGGGCTTCAGGC | AGG |
| IL1B | 632 | Human | chr2 | hg38 | 85.6 | 61 | 58 | GACCTCTGCCCTCTGGATGG | CGG |
| IL1B | 633 | Human | chr2 | hg38 | 85.2 | 35 | 62 | CTCTCCGCAGTGCTCCTTCC | AGG |
| IL1B | 634 | Human | chr2 | hg38 | 78.5 | 50 | 62 | CATTCTCCTGGAAGGTCTGT | GGG |
| IL1A | 635 | Human | chr2 | hg38 | 92.1 | 70 | 87 | ATGGTGGTAGTAGCAACCAA | CGG |
| IL1A | 636 | Human | chr2 | hg38 | 88.3 | 51 | 88 | GCCATAGCTTACATGATAGA | AGG |
| IL1A | 637 | Human | chr2 | hg38 | 87 | 68 | 88 | CAGAGACAGATGATCAATGG | AGG |
| IL1A | 638 | Human | chr2 | hg38 | 87 | 50 | 74 | ATTCAGAGACAGATGATCAA | TGG |
| IL1A | 639 | Human | chr2 | hg38 | 83.1 | 53 | 91 | TGGTGGTAGTAGCAACCAAC | GGG |
| IL1A | 640 | Human | chr2 | hg38 | 85.7 | 60 | 67 | CTCCAGGTCATCATCAGTGA | TGG |

TABLE 15

| Gene | SEQ | | | | | Frameshift | CasOffFinder | | |
|---|---|---|---|---|---|---|---|---|---|
| symbol | ID NO: | Species | Chromosome | Exon | Genome | (%) | total | gRNA | PAM |
| ILIA | 641 | Horse | chr15 | 4 | EquCab3.0 | 80.9 | 21 | TGGTGCTGGTGGCAGCCAAC | GGG |
| IL1A | 642 | Horse | chr15 | 4 | EquCab3.0 | 79.2 | 14 | CTTCTTCAGAGTCTTCCCGT | TGG |
| ILIA | 643 | Horse | chr15 | 3 | EquCab3.0 | 74.9 | 37 | TTACCTGAGTCAGAGAGAGA | TGG |
| ILIA | 644 | Horse | chr15 | 4 | EquCab3.0 | 74.6 | 11 | AAGCTATGACCCACTTCCTG | AGG |
| IL1A | 645 | Horse | chr15 | 4 | EquCab3.0 | 71.5 | 14 | CCATGCAGTCCTCAGGAAGT | GGG |
| IL1A | 646 | Dog | chr17 | 3 | canFam4 | 75.7 | 7 | ACATGCAGTCCTCATGAAGT | GGG |
| IL1A | 647 | Dog | chr17 | 3 | canFam4 | 73.8 | 10 | GACATCCCAGCTTACCTTCA | AGG |
| ILIA | 648 | Dog | chr17 | 3 | canFam4 | 72.6 | 10 | GAGCTGTGACCCACTTCATG | AGG |
| ILIA | 649 | Dog | chr17 | 3 | canFam4 | 79.2 | 14 | CATTTTCCTTGAAGGTAAGC | TGG |
| ILIA | 650 | Dog | chr17 | 3 | canFam4 | 87 | 16 | TGGTAGTGGTGGCAGCCAAT | GGG |
| IL1A | 651 | Dog | chr17 | 3 | canFam4 | 76.8 | 18 | ATTTTCCTTGAAGGTAAGCT | GGG |
| ILIA | 652 | Dog | chr17 | 3 | canFam4 | 79.1 | 23 | CTTCTTTAGAATCTTCCCAT | TGG |
| IL1A | 653 | Dog | chr17 | 3 | canFam4 | 71.4 | 23 | GGATGTCTTTGAGATTTCAG | AGG |
| IL1A | 654 | Dog | chr17 | 3 | canFam4 | 88.5 | 28 | GTGGTAGTGGTGGCAGCCAA | TGG |
| ILIA | 655 | Dog | chr17 | 3 | canFam4 | 78.7 | 31 | CACTACCACATTTTCCTTGA | AGG |
| IL1A | 656 | Cat | chrA3 | 3 | Fc9.0 | 73.3 | 12 | GGTAAGCTGGGGTGTCTTAG | AGG |
| IL1A | 657 | Cat | chrA3 | 3 | Fc9.0 | 74.3 | 13 | ATTCCTCACTGCTGATGACC | TGG |
| ILIA | 658 | Cat | chrA3 | 3 | Fc9.0 | 86.7 | 15 | TGGTGATGGTGGCAGCCAAT | GGG |
| IL1A | 659 | Cat | chrA3 | 3 | Fc9.0 | 79.3 | 20 | CTTCCAGGTCATCAGCAGTG | AGG |
| IL1B | 660 | Horse | chr15 | 4 | EquCab3.0 | 87.6 | 27 | TGAAAGTCTTGTTGTAAAGT | TGG |
| IL1B | 661 | Horse | chr15 | 4 | EquCab3.0 | 79.6 | 15 | GACCTCAGCTCCATGGGCGA | TGG |
| IL1B | 662 | Horse | chr15 | 4 | EquCab3.0 | 78.5 | 13 | CTGGATGCCCCCATCGCCCA | TGG |
| IL1B | 663 | Horse | chr15 | 4 | EquCab3.0 | 75.4 | 17 | CCCCATCGCCCATGGAGCTG | AGG |
| IL1B | 664 | Horse | chr15 | 4 | EquCab3.0 | 75.2 | 12 | AAGTCTTGTTGTAAAGTTGG | TGG |
| IL1B | 665 | Dog | chr17 | 3 | canFam4 | 82.1 | 2 | CGTGTCAGTCATTGTAGCTT | TGG |
| IL1B | 666 | Dog | chr17 | 3 | canFam4 | 74.2 | 11 | CCACAGTTCTCTGGTAGATG | AGG |
| IL1B | 667 | Dog | chr17 | 3 | canFam4 | 80 | 13 | AGACCTGAACCACAGTTCTC | TGG |
| IL1B | 668 | Dog | chr17 | 3 | canFam4 | 80 | 14 | CCTCATCTACCAGAGAACTG | TGG |
| IL1B | 669 | Dog | chr17 | 3 | canFam4 | 80 | 15 | TCAGACTCTTGTTACAGAGC | TGG |
| IL1B | 670 | Cat | chrA3 | 2 | Fc9.0 | 82.4 | 8 | GTAGTAAGCCATCATTTCAC | TGG |
| IL1B | 671 | Cat | chrA3 | 4 | Fc9.0 | 8 | 4 | GAGTCTTAGGCATGCCGTGT | CGG |
| IL1B | 672 | Cat | chrA3 | 4 | Fc9.0 | 81.8 | 14 | AAACCTGAGCCACAGTTTTC | TGG |
| IL1B | 673 | Cat | chrA3 | 2 | Fc9.0 | 80.7 | 17 | ATCATTTCACTGGTGAGTTC | AGG |
| IL1B | 674 | Cat | chrA3 | 4 | Fc9.0 | 80.4 | 18 | ACTCTTGTTGTCGGGCTGGT | GGG |
| IL1B | 675 | Cat | chrA3 | 4 | Fc9.0 | 80.1 | 14 | GACTCTTGTTGTCGGGCTGG | TGG |
| IL1B | 676 | Cat | chrA3 | 4 | Fc9.0 | 76.7 | 24 | CCTCATCTCCCAGAAAACTG | TGG |
| IL1B | 677 | Cat | chrA3 | 3 | Fc9.0 | 76 | 23 | TGAGAATGACCTGTTCTTTG | AGG |
| IL1B | 678 | Cat | chrA3 | 4 | Fc9.0 | 75.7 | 5 | TAAGACTCTTGTTGTCGGGC | TGG |
| IL1B | 679 | Cat | chrA3 | 4 | Fc9.0 | 74.3 | 18 | AACCTGAGCCACAGTTTTCT | GGG |

TABLE 16

| Gene | Species | Ensemble | Transcript | Transcript_ID | Location |
|---|---|---|---|---|---|
| IL1A | Human | ENSG00000115008 | IL1A-201 | ENST00000263339.4 | Chromosome 2: 112,773,925-112,784,493 |
| IL1A | Mouse | ENSMUSG00000027399 | Il1a-201 | ENSMUST00000028882.1 | Chromosome 2: 129,299,610-129,309,972 |
| IL1A | Cat | ENSFCAG00000008095 | IL1A-201 | ENSFCAT00000008097.5 | Chromosome A3: 105,113,193-105,121,209 |
| IL1A | Dog | ENSCAFG00000007245 | IL1A-201 | ENSCAFT00000045928.3 | Chromosome 17: 36,970,573-36,978,736 |
| IL1A | Horse | ENSECAG00000023727 | IL1A-202 | ENSECAT00000025409.2 | Primary_assembly 15: 16,360,700-16,369,812 |
| IL1B | Human | ENSG00000125538 | IL1B-201 | ENST00000263341.7 | Chromosome 2: 112,829,751-112,836,779 |
| IL1B | Mouse | ENSMUSG00000027398 | Il1b-201 | ENSMUST00000028881.13 | Chromosome 2: 129,364,570-129,371,139 |
| IL1B | Cat | ENSFCAG00000005899 | IL1B-201 | ENSFCAT00000005904.5 | Chromosome A3: 105,049,334-105,056,407 |
| IL1B | Dog | ENSCAFG00000007249 | IL1B-201 | ENSCAFT00000011613.4 | Chromosome 17: 37,019,329-37,024,176 |
| IL1B | Horse | ENSECAG00000000051 | IL1B-202 | ENSECAT00000061518.2 | Primary_assembly 15: 16,418,738-16,444,697 |

### Example 7. Immunohistochemistry for Murine IL-1β

Immunohistochemistry was performed on Murine synovial tissue to detect IL-1β according to the following protocol:

Reagents & Preparation

1. 10×PBS with 0.5% (v/v) Tween-20

2. Sterile PBS

3. IHC buffer

4. Primary antibody (goat anti-mouse IL-1; AF-401-NA, R&D Systems, Inc.) reconstituted to a final concentration of 0.2 mg/ml in sterile PBS.
a. Short-term storage at +4 C
b. Long-term storage at −20 to −70 C

5. Reconstitute the control antibody (normal goat IgG; AB-108-C, R&D Systems Inc.) at a final concentration of 1 mg/ml in sterile PBS.
a. Short-term storage at +4 C
b. Long-term storage at −20 to −70 C

6. Secondary antibody (HRP conjugated donkey anti-goat IgG; ab6885; Abcam) comes as reconstituted product
a. Short-term storage at +4 C
b. Long-term storage at −20 to −70 C

7. Peroxidase block (BLOXALL reagent, Vector Laboratories)

8. Normal horse serum, diluted to 2.5% (v/v) (Impress Polymer Kit; Vector Laboratories)

9. DAB chromogen

Method

Antigen retrieval was performed for 1 hour (manual or automated, user preference), and then samples were transferred to PBS for short-term storage. Peroxidase block was performed for 10 minutes at room temperature, and samples were subsequently washed in IHC buffer for 5 mins. Samples were blocked with control horse serum for 60 minutes at room temperature, and exposed to primary antibody (1:100 or 1:200 diluted in 1×PBS-Tween) for 2 hours at room temperature. Samples were washed in IHC buffer twice for 5 mins (each wash). Samples were then exposed to secondary antibody (1:500 diluted in 1×PBS-Tween) for 1 hour, and washed in IHC buffer twice for 5 mins (each wash). Detection was performed with DAB chromogen for 30 seconds. Counterstaining was performed with Mayer's haematoxylin (6 minutes), and samples were returned through graded alcohol series to xylene. DPX mountant was applied, and a coverslip was attached.

As shown in FIG. 10, immunohistochemistry data for murine IL-1β in synovial tissue showed reduced IL-1β expression in CRISPR-treated animals. (A) In MSU injected animals pre-treated with PBS there is robust expression of IL-1β (brown staining). This effect is not seen in CRISPR-treated animals (panel C). The absence of IL-1β (brown staining) in CRISPR-treated animals resembles negative antibody controls (panels B & D). All images are 10×original magnification. (A) and (B) are adjacent sections taken from the same joint in the same animal, with (A) showing tissue labeled specifically for IL-1 beta, and (B) showing tissue labeled with the negative (isotope) control antibody. Differences in staining reflect demonstrable IL-1 beta expression in MSU injected animals pre-treated with PBS in this animal (e.g., a positive control animal pre-treated with PBS, then challenged with MSU crystal). (C) and (D) are similarly adjacent sections, but from an animal that was pre-treated with the CRISPR editing virus prior to MSU injection. (C) There is no obvious staining for IL-1 beta in the section treated with IL-1 antibody, and (D) the same negative pattern is seen in the section treated with the negative (isotope) control antibody. Without wishing to be bound by any particular theory, this confirms that there is no detectable IL-1 beta expression in the synovium of CRISPR-treated animals.

### Example 8. Design and Validation of CRISPR/Cas9 RNA Guides Against Canine and Human Interleukin-1 Alpha (IL-1α) and Interleukin-1 Beta (IL-1β)

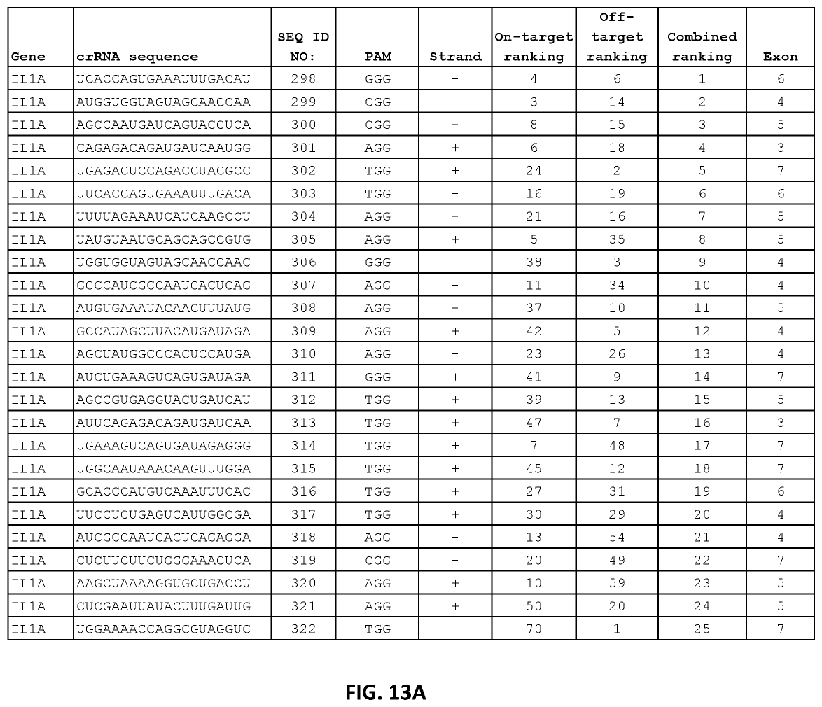

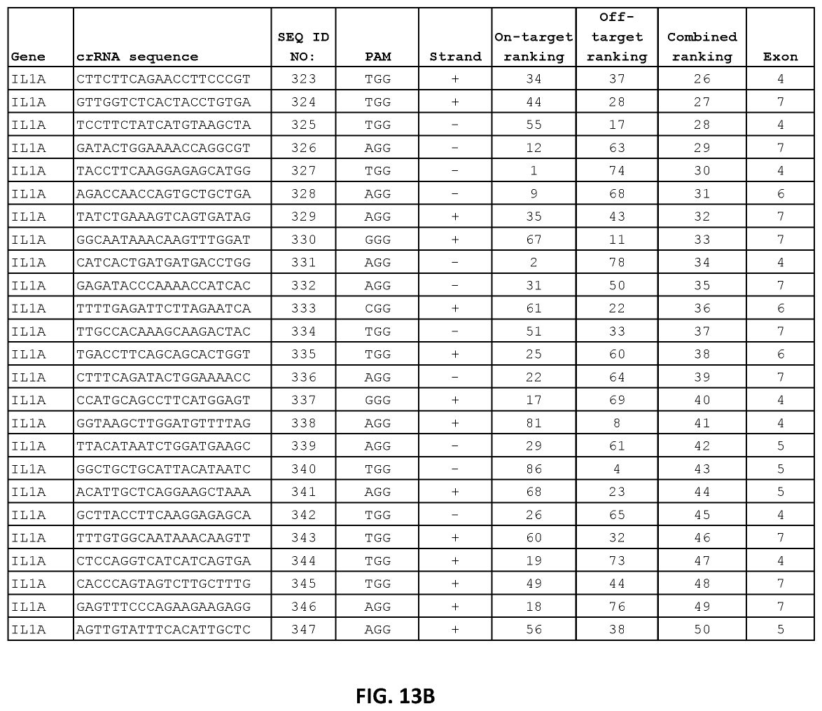

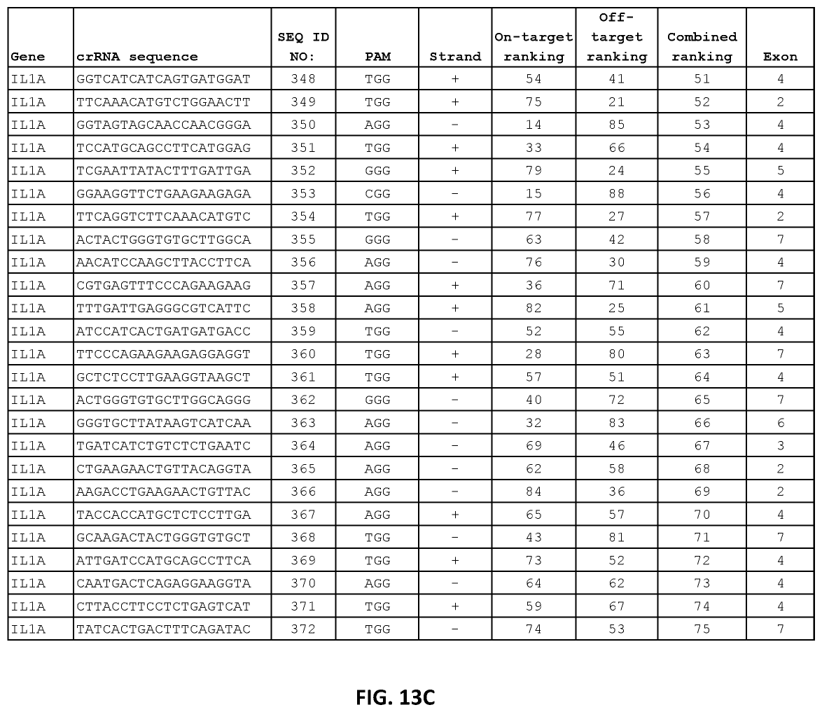

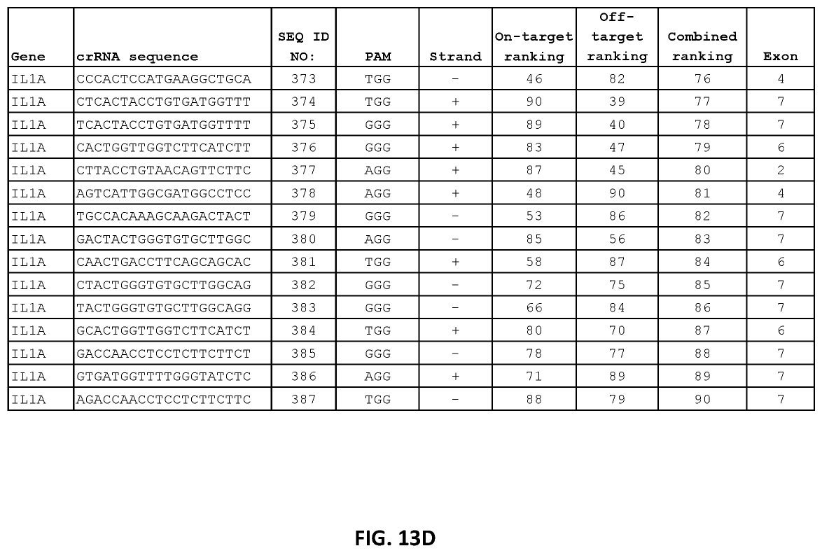

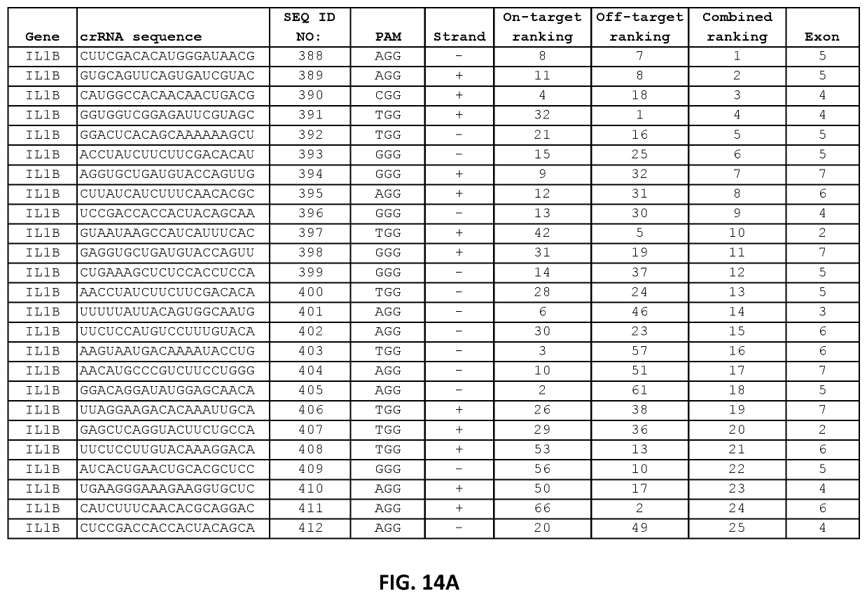

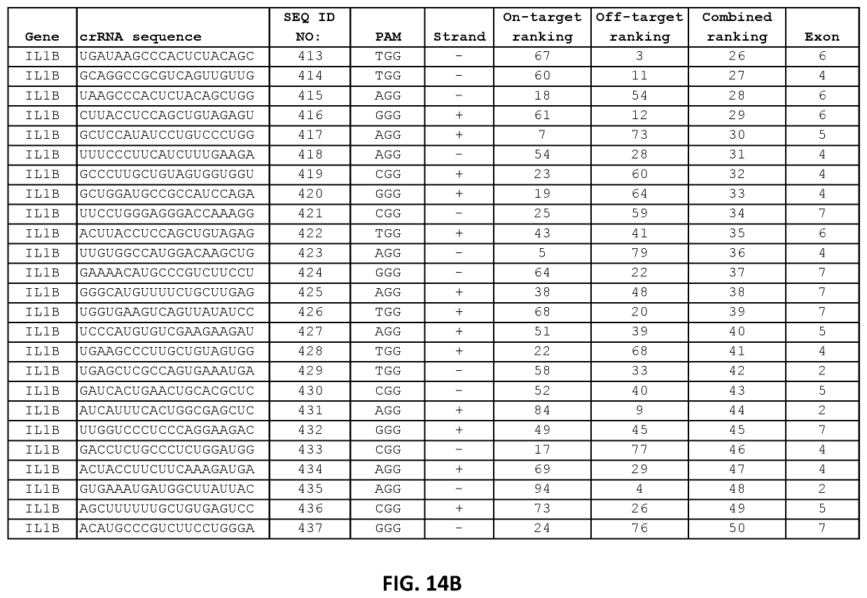

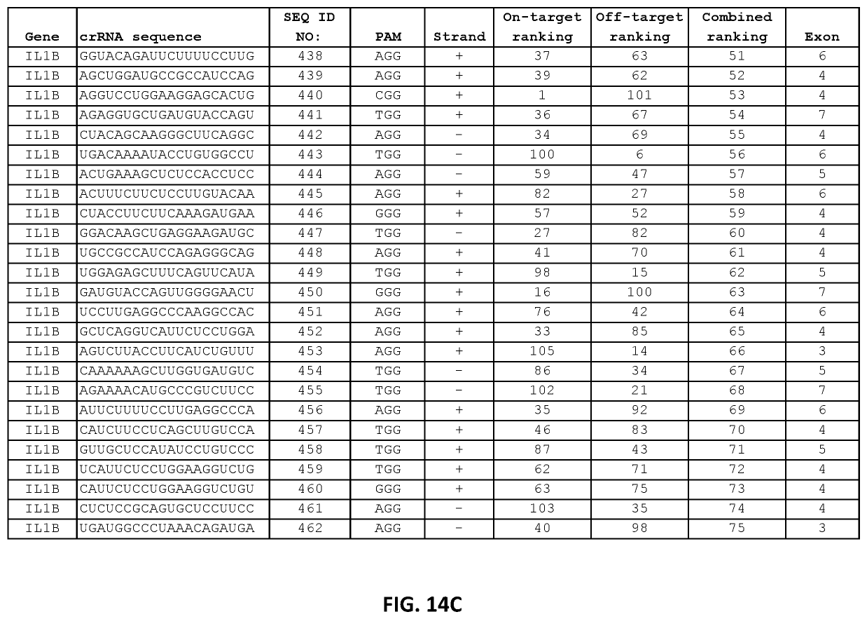

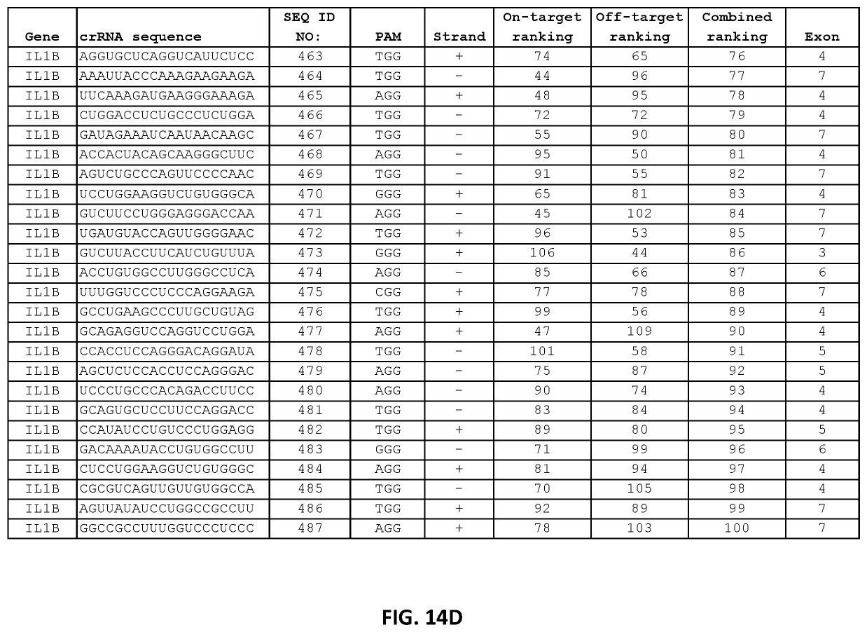

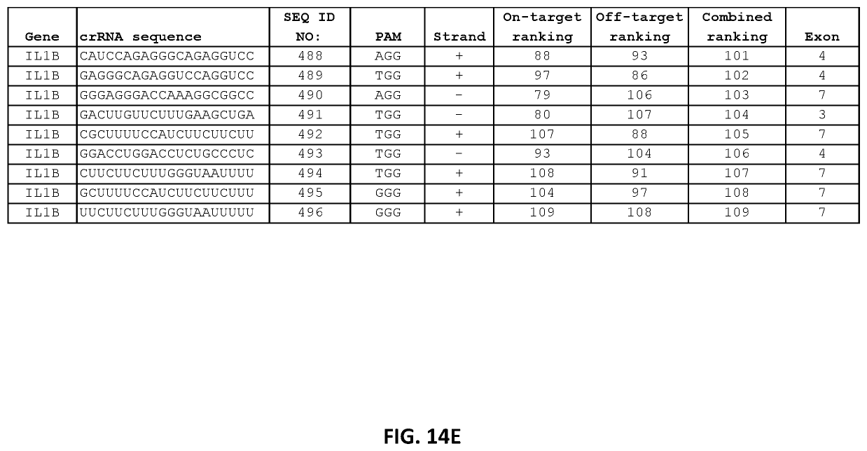

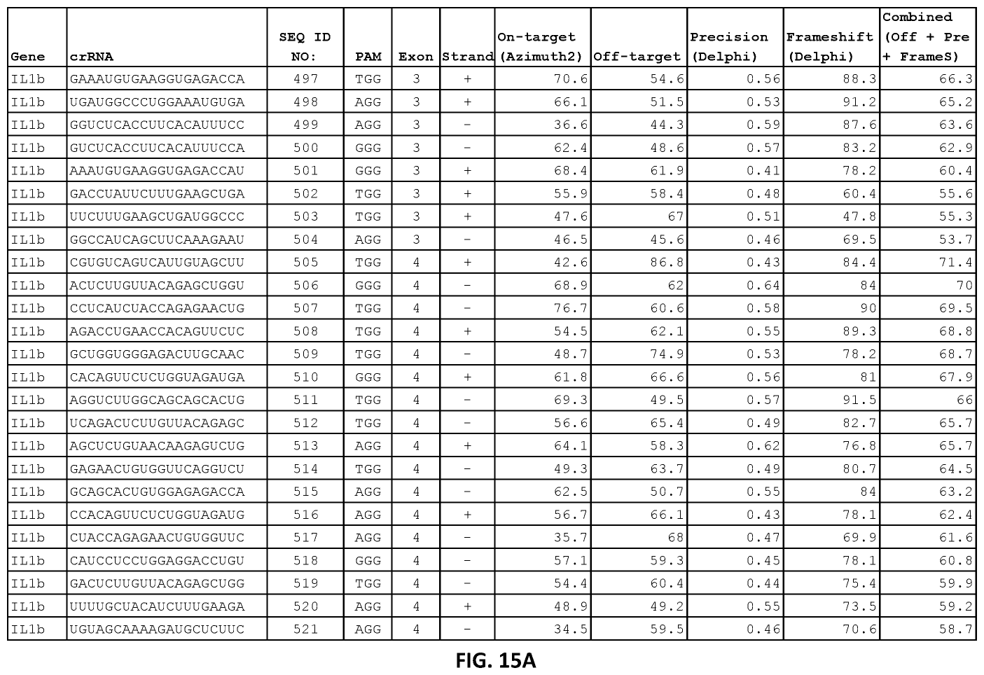

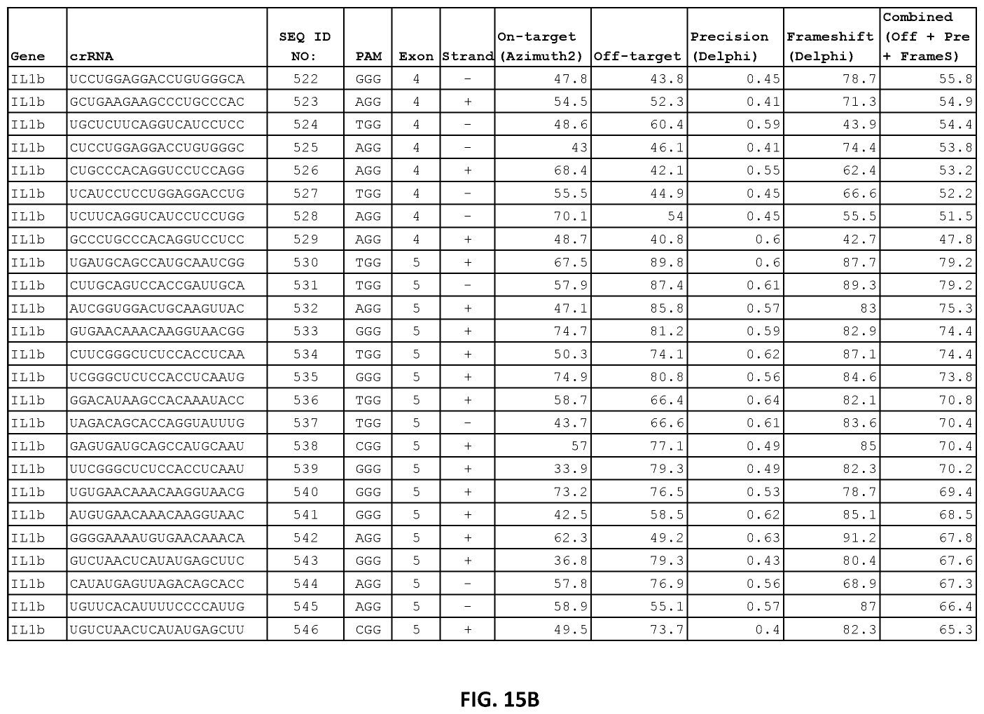

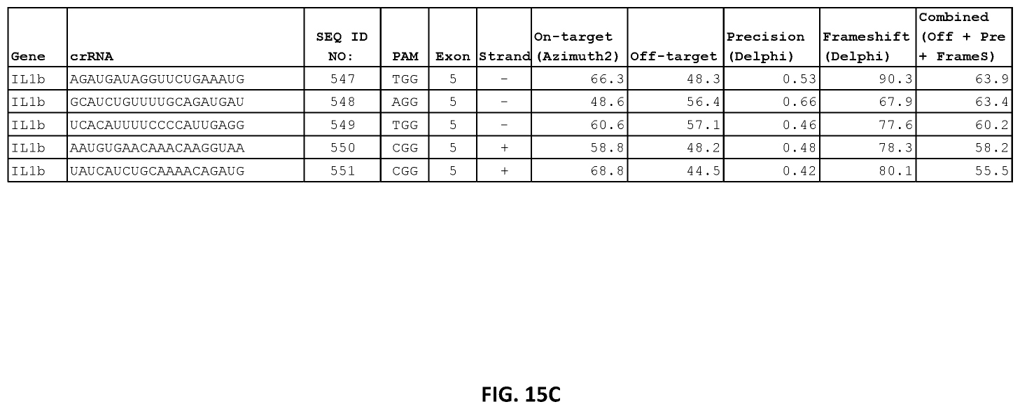

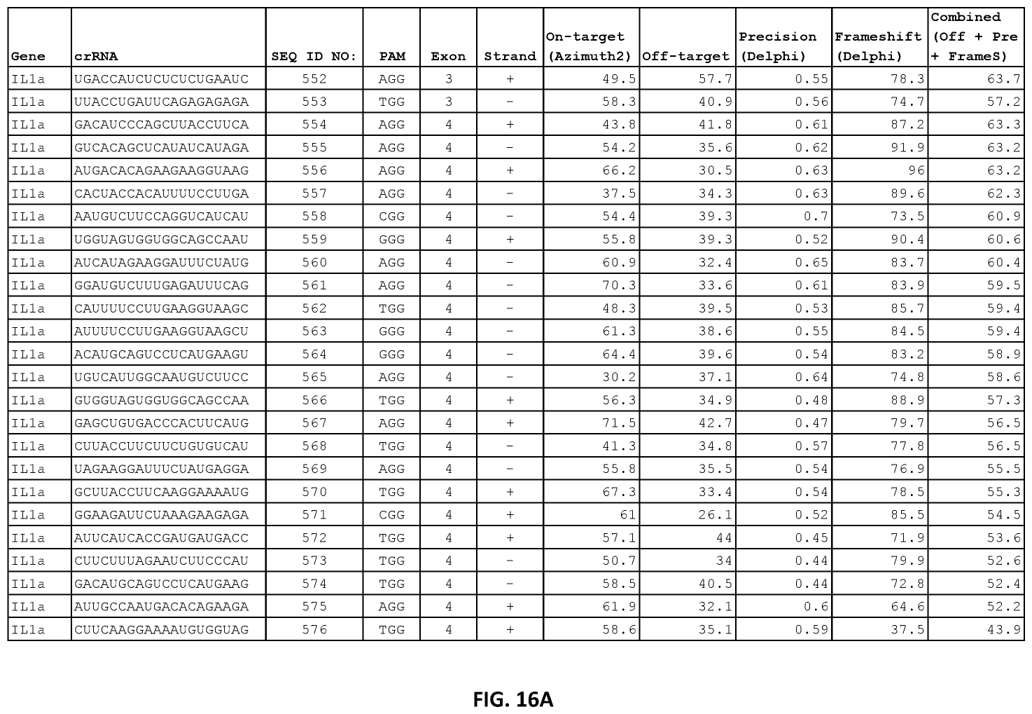

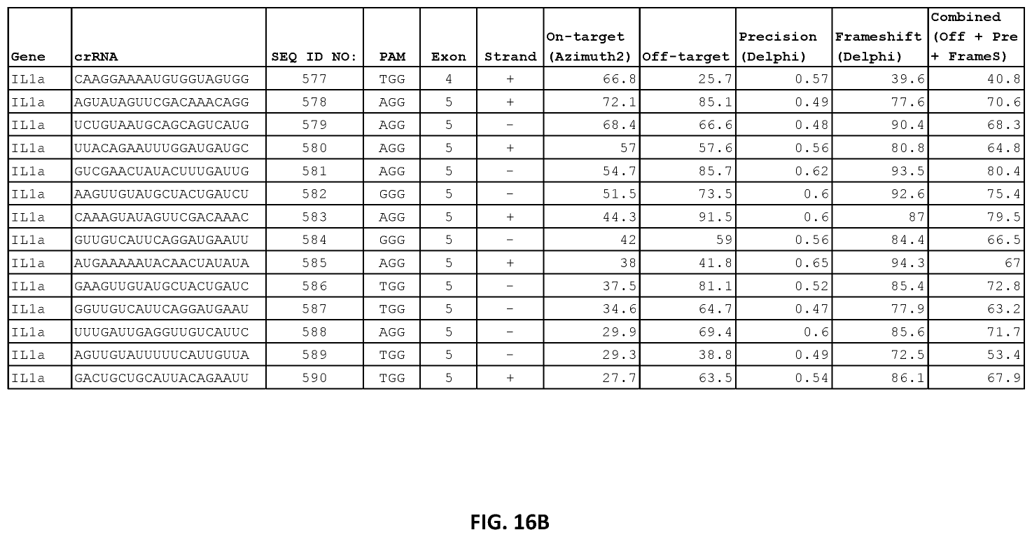

Potential crRNA sequences were identified for various exons of the human and canine interleukin-1 alpha (IL-1α) and interleukin-1 beta (IL-1β) genes. FIGS. 13A-13D show a ranked list of crRNA sequences identified from exons 2-7 of the human IL-1α gene. FIGS. 14A-14E show a ranked list of crRNA sequences identified from exons 2-7 of the human IL-1β gene. FIGS. 15A-15C show crRNA sequences identified from exons 3-5 of the canine IL-1β gene. FIGS. 16A-16B show a crRNA sequences identified from exons 3-5 of the canine IL-1α gene.

Publicly accessible genomes (human, hg38; dog, CanFam3.1), collapsed gene models (merged Ensembl/Havana), tissue-specific exon expression (gtexportal.org) and various gRNA models were then used to select two to five individual crRNA sequences per gene, targeting canine and human interleukin-1 alpha (IL-1α) and interleukin-1 beta (IL-1β). The following gRNA design rules were applied:
1. The gRNA target region was limited to the first 5-50% of the coding sequence (CDS).
2. Single gRNAs were ranked according to maximal on-target editing using Azimuth 2.0 model (10.1038/nbt.3437) and minimal off-target editing using Cutting Frequencing Determination (CFD) (10.1038/nbt.3437) and the specificity score from Hsu et al. (10.1038/nbt.2647).
3. Highly ranked sgRNA with high frameshift frequencies (>75%) and uniform DNA repair outcomes (>0.48) as predicted by inDelphi (10.1038/s41586-018-0686-x) were selected for in vitro synthesis.

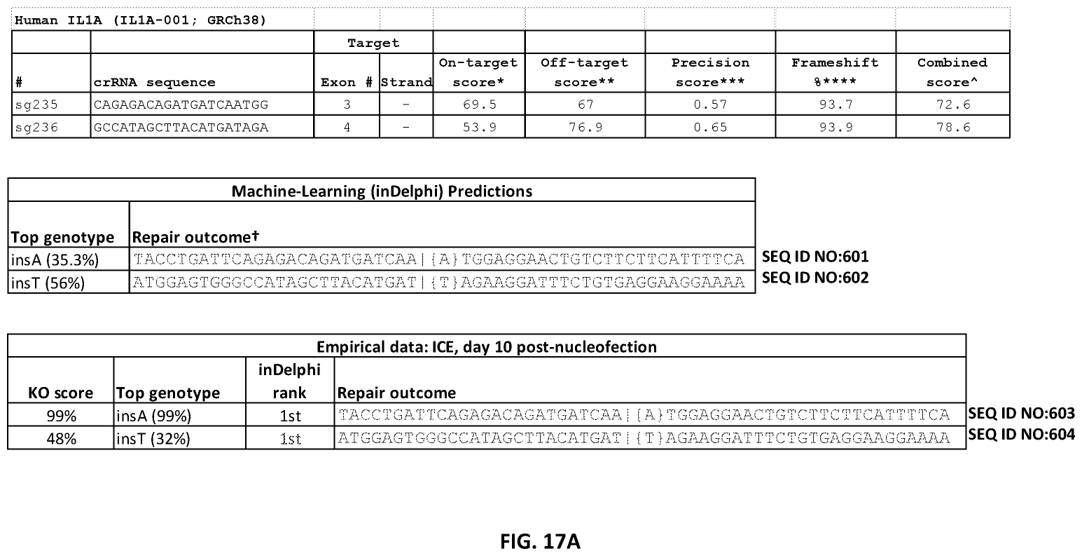

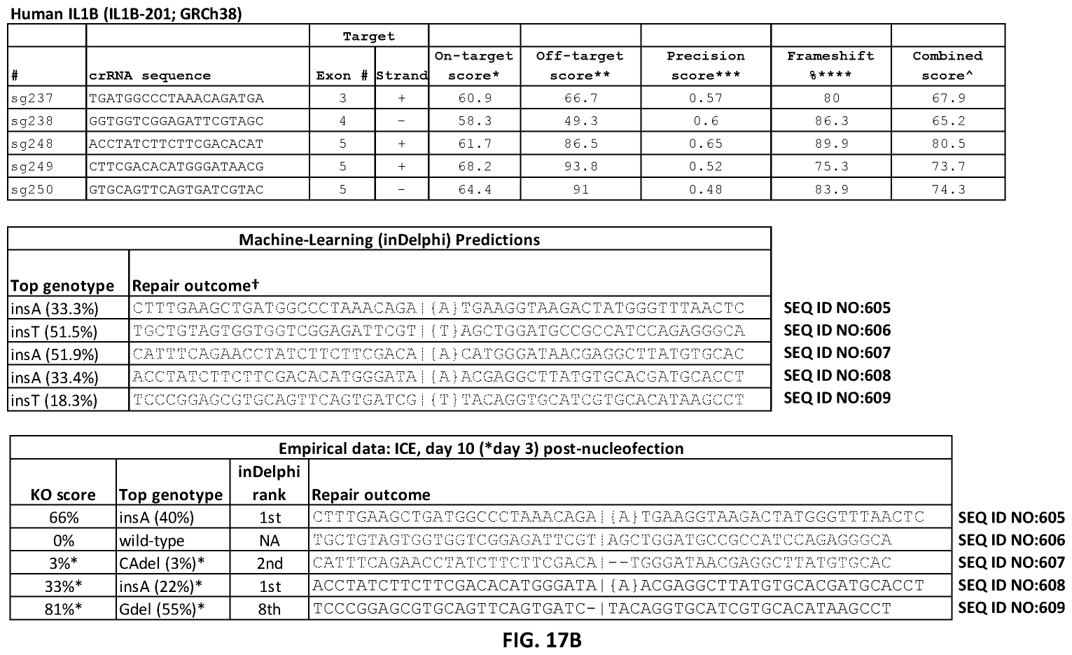

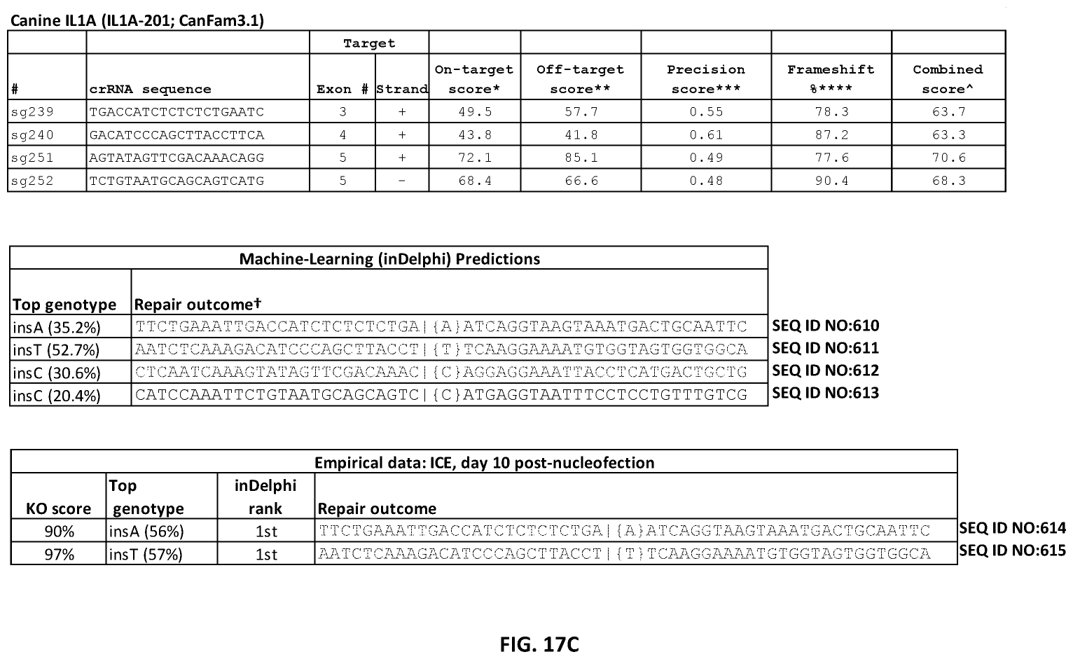

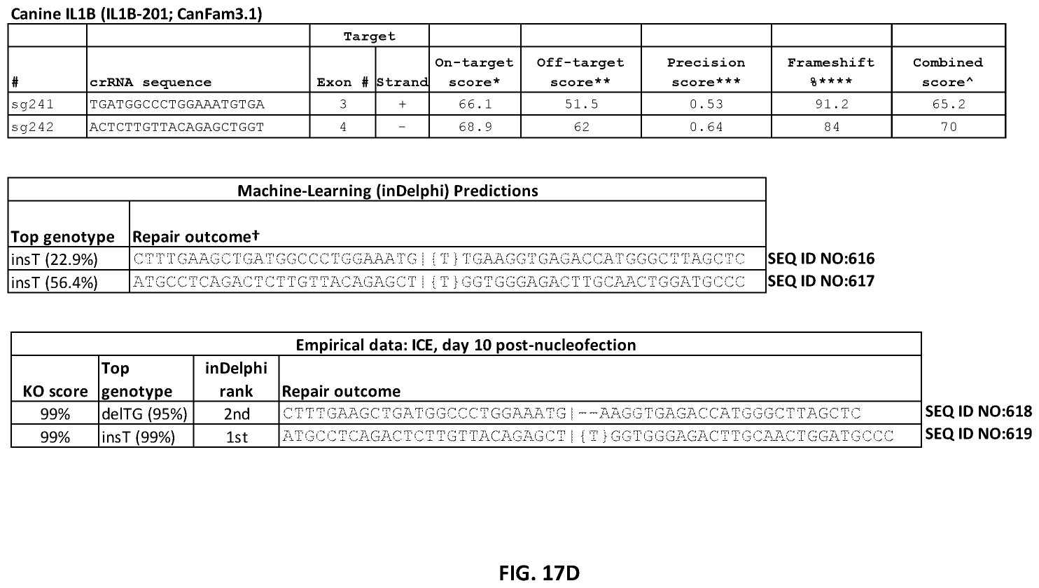

Using this selection criteria, crRNA guide sequences targeting different exons of the respective target genes were selected for further investigation. Specifically, as shown in FIG. 17A, sg235 (SEQ ID NO:301) and sg236 (SEQ ID NO:309) target exons 3 and 4 of the human IL-1α gene were selected. Likewise, as shown in FIG. 17B, sg237 (SEQ ID NO:462), sg238 (SEQ ID NO:391), sg248 (SEQ ID NO:393), sg249 (SEQ ID NO:388), and sg250 (SEQ ID NO:389) targeting exons 3, 4, and 5 of the human IL-1β gene were selected. As shown in FIG. 17C, sg239 (SEQ ID NO:552), sg240 (SEQ ID NO:554), sg251 (SEQ ID NO:578), and sg252 (SEQ ID NO:579) targeting exons 3, 4, and 5 of the canine IL-1α gene were selected. Likewise, as shown in FIG. 17D, sg241 (SEQ ID NO:498) and sg242 (SEQ ID NO:506) targeting exons 3 and 4 of the canine IL-1β gene were selected.

Single guide RNAs (sgRNAs), fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with 4 μg Cas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (ab1) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

Results of the empirical experiments and machine-learning prediction of gene editing using the selected guide sequences is shown in FIG. 17.

### Example 9.—Effect of Selected CRISPR/Cas9 RNA Guides on Canine and Human Interleukin-1 Alpha (IL-1α) and Interleukin-1 Beta (IL-1β) Release

The gRNA with the highest knockout (KO) scores from Example 8 (i.e., the highest frameshift frequency) were used to generate double IL-1α/IL-1β knock out (KO) cells. Specifically, human chondrocytes were edited to achieve >99% IL-1α KO using crRNA sequence CAGAGACAGAUGAUCAAUGG (SEQ ID NO:301) and 67% IL-1β KO using crRNA sequence GUGCAGUUCAGUGAUCGUAC (SEQ ID NO:389). Canine chondrocytes were edited to achieve 97% IL-1α KO using crRNA sequence GACAUCCCAGCUUACCUUCA (SEQ ID NO:554) and 99% IL-1β KO using crRNA sequence ACUCUUGUUACAGAGCUGGU (SEQ ID NO:506).

Canine chondrocytes (Catalog Cn402K-05), human chondrocytes (Catalog 402-05a) and human fibroblast-like synovial cells (Catalog 408-05a) were purchased as frozen stocks (5×10\hat{ }5 cells) from Cell Applications, Inc., San Diego, Calif. Chondrocytes were cultured in growth medium consisting of DMEM/Ham's F12 (Gibco, Catalog 21331-020) supplemented with 20% (v/v) untreated FBS (Gibco, Catalog 10270-106) and 1× GlutaMAX (Gibco, Catalog 35050-038). Synovial cells were cultured in growth medium consisting of DMEM (Gibco, Catalog 11960-044), 10% non-treated FBS (Gibco, Catalog 10270-106) and 1× GlutaMAX (Gibco, Catalog 35050-038). Cells were confirmed as being negative for Mycoplasma spp. and subjected to STR profiling prior to use. For electroporation and subculture, cells were dissociated using 0.25% trypsin (Gibco, Catalog 25200056). Trypsin was quenched with 9 volumes of growth medium and cells were spun at 1,000 g to remove the supernatant.

Induction of IL-1 by LPS. Interleukin-1 release was induced by challenging sub-confluent monolayers of cells (edited or wild-type non-edited) with lipopolysaccharide (LPS). In brief, non-edited (control) and double IL-1α/IL-1β KO (edited) human or canine chondrocytes were seeded at density of approximately 5×10⁴ cells per well in 24-well plates. After 24-48 hours, the medium was replaced with fresh, serum-free medium containing either LPS (50 μg/ml) or PBS vehicle and the plates returned to the incubator. Plates were harvested after 6 and 24 hours for the determination of IL-1 release. Media were snap-frozen in liquid nitrogen and stored at −20° C. until they were assayed.

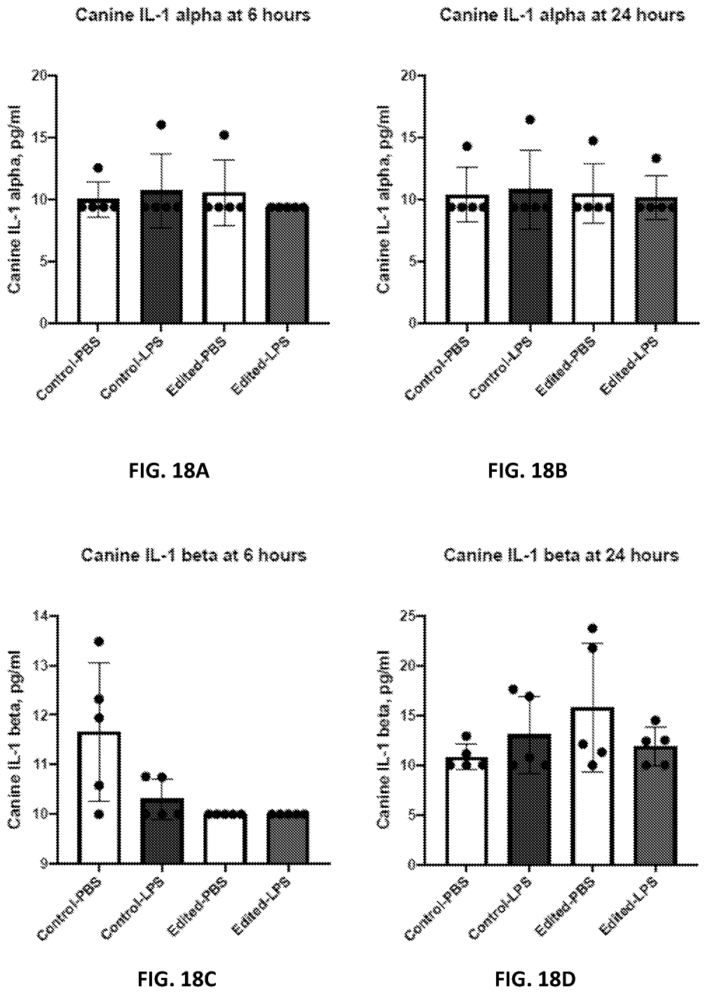

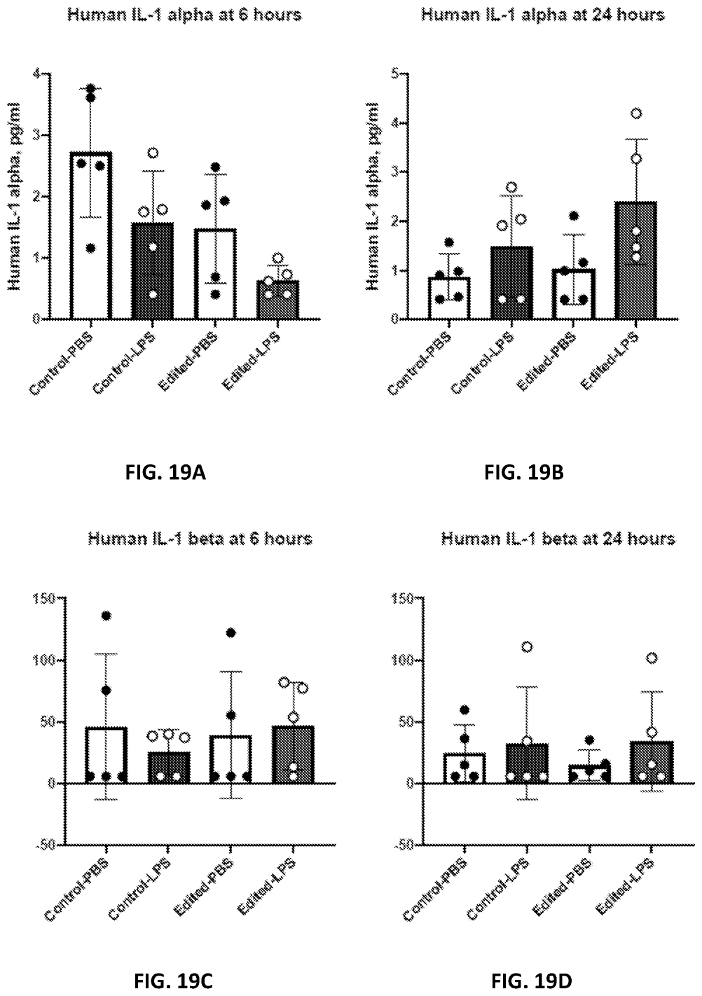

Measurement of IL-1 alpha and IL-1 beta release. The concentration of IL-1 alpha and IL-1 beta in culture medium was measured with species-specific commercial assays, following the manufacturer's instructions. Prior to measurement, frozen media were thawed and then centrifuged (1,500 g for 2 mins) in order to remove cellular debris. Aliquots of medium were measured in duplicate and the concentration of IL-1 determined from a standard curve of recombinant human or canine IL-1 alpha or beta, as appropriate. The results of IL-1 alpha release in canine cells are shown in FIGS. 18A (6 hours) and 18B (24 hours). The results of IL-1 beta release in canine cells are shown in FIGS. 18C (6 hours) and 18D (24 hours). The results of IL-1 alpha release in human cells are shown in FIGS. 19A (6 hours) and 19B (24 hours). The results of IL-1 beta release in human cells are shown in FIGS. 19C (6 hours) and 19D (24 hours).

P3 primary cell nucleofection reagents and nucleocuvette strips (Catalog V4XP-3032) were purchased from Lonza (Slough, UK). Cas9 nuclease (Catalog A36499) was purchased from Thermo Fisher Scientific. Lipopolysaccharide (LPS) from E. coli 055:B5 (Catalog L6529) was purchased from Merck. ELISA kits for human IL-1 alpha (Catalog ab214025) and human IL-1 beta (Catalog ab100560), canine IL-1 alpha (Catalog A4270) and canine IL-1 beta (Catalog ab273170) were purchased from Abcam (Cambridge, UK).

### Example 10.—Increased Specificity of CRISPR/Cas9 Mediated Gene Editing

The analysis of gene editing specificity reported in Example 8 was repeated using an enhanced Specificity CRISPR associated protein 9. The eSpCas9 includes three specificity enhancing mutations: K848A, K1003A, and R1060A, as described in Slaymaker et al., Science, 351:84-88 (2016). The eSpCas9 was expressed in E. coli and purified to homogeneity. The construct has a molecular weight of 161 kDa and contains N-terminal Flag-tags and a C-terminal hexa-His-tag. The sequence of the eSpCas9 is:

(SEQ ID NO: 680)
MDYKDHDGDYKDHDIDYKDDDDKMAPKKKRKVGIHGVPAADKKYSIGLDI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLADDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPALESEFVYG
DYKVYDVRKMIAKSEQEIGKATAKYFFYSNIMNFFKTEITLANGEIRKAP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDKRPAATKKAGQAKKKKAAALEHHHHHH.

Briefly, the same sgRNAs used in Example 8, and shown in FIG. 17, were complexed with the eSpCas9. As shown in FIG. 17A, sg235 (SEQ ID NO:301) and sg236 (SEQ ID NO:309) target exons 3 and 4 of the human IL-1α gene were used. Likewise, as shown in FIG. 17B, sg237 (SEQ ID NO:462), sg238 (SEQ ID NO:391), sg248 (SEQ ID NO:393), sg249 (SEQ ID NO:388), and sg250 (SEQ ID NO:389) targeting exons 3, 4, and 5 of the human IL-1β gene were used. As shown in FIG. 17C, sg239 (SEQ ID NO:552), sg240 (SEQ ID NO:554), sg251 (SEQ ID NO:578), and sg252 (SEQ ID NO:579) targeting exons 3, 4, and 5 of the canine IL-1α gene were used. Likewise, as shown in FIG. 17D, sg241 (SEQ ID NO:498) and sg242 (SEQ ID NO:506) targeting exons 3 and 4 of the canine IL-1β gene were used.

Single guide RNAs (sgRNAs), fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with eSpCas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (ab1) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

As compared to the Cas9 editing reported in Example 8, use of eSpCas9 reduces off-target editing without losing on-target activity. For example, the off-target editing by sgRNA #242 (targeting canine IL-1B) of three loci, having 2, 3, and 3 mismatches, respectively, were evaluated by amplifying and then sequencing the loci reported in Table 18. As shown in Table 18, the first off-target loci experienced no editing in the experiment described in Example 8, and was not tested here. The second off-target loci experienced almost complete off-target editing (98-99%) in the experiment described in Example 8, but experienced no editing when eSpCas9 was used. The third off-target loci experienced some editing (0-25%) in the experiment described in Example 8, but again experienced no editing when eSpCas9 was used. Further, as shown in Table 17, the “enhanced on-target score,” corresponding to editing using eSpCas9 as described in this example, for each sgRNA tested was as high, if not higher, than the “on-target score,” corresponding to the editing described in Example 8.

TABLE 17

| | | | On- | | | | Off- |
|---|---|---|---|---|---|---|---|
| | | | target | | | | target |
| | | | metrics | | | | metrics |
| | Target | | On- | Pre- | Frame- | Enhanced | Off- |
| | Exon | | target | cision | shift | on-target | target |
| # | # | Strand | score* | score** | %*** | score**** | score\hat{ } |
| sg235 | 3 | − | 69.5 | 0.57 | 93.7 | 72.4 | 67 |
| sg236 | 4 | − | 53.9 | 0.65 | 93.9 | 66.7 | 76.9 |

Human IL1B (IL1B-201; GRCh38)

| | | | | | | | |
|---|---|---|---|---|---|---|---|
| sg237 | 3 | + | 60.9 | 0.57 | 80 | 64.7 | 66.7 |
| sg238 | 4 | − | 58.3 | 0.6 | 86.3 | 65.7 | 49.3 |
| sg248 | 5 | + | 61.7 | 0.65 | 89.9 | 69.6 | 86.5 |
| sg249 | 5 | + | 68.2 | 0.52 | 75.3 | 65.9 | 93.8 |
| sg250 | 5 | − | 64.4 | 0.48 | 83.9 | 65.2 | 91 |

Canine IL1A (IL1A-201; CanFam3.1)

| | | | | | | | |
|---|---|---|---|---|---|---|---|
| sg239 | 3 | + | 49.5 | 0.55 | 78.3 | 58.1 | 57.7 |
| sg240 | 4 | + | 43.8 | 0.61 | 87.2 | 59.0 | 41.8 |
| sg251 | 5 | + | 72.1 | 0.49 | 77.6 | 67.7 | 85.1 |
| sg252 | 5 | − | 68.4 | 0.48 | 90.4 | 68.8 | 66.6 |

Canine IL1B (IL1B-201; CanFam3.1)

| | | | | | | | |
|---|---|---|---|---|---|---|---|
| sg241 | 3 | + | 66.1 | 0.53 | 91.2 | 69.1 | 51.5 |
| sg242 | 4 | − | 68.9 | 0.64 | 84 | 71.5 | 62 |

TABLE 18

| | | | | | | | | | CRISPR |
|---|---|---|---|---|---|---|---|---|---|
| | | | | | | | | CRISPR | Edits with |
| | | | | | | | | Edits | enhanced |
| | | | | Chromo- | | | Mis- | with | specificity |
| Sequence | PAM | Score | Gene | some | Strand | Position | matches | Cas9 | Cas9 |
| ACTCTTGTTACAGAGCTGGT | GGG | 100 | ENSCAFG | chr17 | 1 | 37022194 | 0 | | |
| ACTTTTGTTTCAGAGCTGGT | CAG | 6.16161972 | 00000007249 | chr33 | −1 | 20234937 | 2 | 0% | |
| CCTCATGCTACAGAGCTGGT | GGG | 2.76564774 | | chr1 | −1 | 47541563 | 3 | 98- | 0% |
| | | | | | | | | 99% | |
| GTGCTTGTTACAGAGCTGGT | GGG | 2.32143742 | | chr26 | −1 | 32323843 | 3 | 0-25% | 0% |

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls

What is claimed:

1. A pharmaceutical composition for the treatment or prevention of a joint disease or condition, comprising:
a therapeutically effective amount of one or more nucleic acids encoding a Clustered Regularly Interspaced Short Palindromic Repeats (CRISPR) gene-editing system, the system comprising:
(i) a CRISPR Associated Protein 9 (Cas9) protein; and
(ii) at least one guide RNA targeting an IL-1α or IL-1β gene, wherein the target sequence is adjacent to a protospacer adjacent motif (PAM) sequence for the Cas9 protein.

2. The pharmaceutical composition of claim 1, wherein:
the at least one guide RNA targets a human IL-1α gene, and
the at least one guide RNA comprises a crRNA sequence having at least 85% identity to a sequence selected from the group consisting of SEQ ID NOs: 298-387.

3. The pharmaceutical composition of claim 1, wherein:
the at least one guide RNA targets a human IL-1α gene, and
the at least one guide RNA comprises a crRNA sequence having at least 90% identity to a sequence selected from the group consisting of SEQ ID NOs: 298-387.

4. The pharmaceutical composition of claim 1, wherein:
the at least one guide RNA targets a human IL-1α gene, and
the at least one guide RNA comprises a crRNA sequence having at least 95% identity to a sequence selected from the group consisting of SEQ ID NOs: 298-387.

5. The pharmaceutical composition of claim 1, wherein:
the at least one guide RNA targets a human IL-1α gene, and
the at least one guide RNA comprises a crRNA sequence selected from the group consisting of SEQ ID NOs: 298-387.

6. The pharmaceutical composition of claim 1, wherein:
the at least one guide RNA targets a human IL-1β gene, and
the at least one guide RNA comprises a crRNA sequence having at least 85% identity to a sequence selected from the group consisting of SEQ ID NOs: 388-496.

7. The pharmaceutical composition of claim 1, wherein:
the at least one guide RNA targets a human IL-1β gene, and
the at least one guide RNA comprises a crRNA sequence having at least 90% identity to a sequence selected from the group consisting of SEQ ID NOs: 388-496.

8. The pharmaceutical composition of claim 1, wherein:
the at least one guide RNA targets a human IL-1β gene, and
the at least one guide RNA comprises a crRNA sequence having at least 95% identity to a sequence selected from the group consisting of SEQ ID NOs: 388-496.

9. The pharmaceutical composition of claim 1, wherein:
the at least one guide RNA targets a human IL-1β gene, and
the at least one guide RNA comprises a crRNA sequence selected from the group consisting of SEQ ID NOs: 388-496.

10. The pharmaceutical composition of claim 1, wherein:
the at least one guide RNA targets a canine IL-1α gene, and
the at least one guide RNA comprises a crRNA sequence having at least 85% identity to a sequence selected from the group consisting of SEQ ID NOs: 522-590.

11. The pharmaceutical composition of claim 1, wherein:
the at least one guide RNA targets a canine IL-1α gene, and
the at least one guide RNA comprises a crRNA sequence having at least 90% identity to a sequence selected from the group consisting of SEQ ID NOs: 522-590.

12. The pharmaceutical composition of claim 1, wherein:
the at least one guide RNA targets a canine IL-1α gene, and
the at least one guide RNA comprises a crRNA sequence having at least 95% identity to a sequence selected from the group consisting of SEQ ID NOs: 522-590.

13. The pharmaceutical composition of claim 1, wherein:
the at least one guide RNA targets a canine IL-1α gene, and
the at least one guide RNA comprises a crRNA sequence selected from the group consisting of SEQ ID NOs: 522-590.

14. The pharmaceutical composition of claim 1, wherein:
the at least one guide RNA targets a canine IL-1β gene, and
the at least one guide RNA comprises a crRNA sequence having at least 85% identity to a sequence selected from the group consisting of SEQ ID NOs: 497-551.

15. The pharmaceutical composition of claim 1, wherein:
the at least one guide RNA targets a canine IL-1β gene, and
the at least one guide RNA comprises a crRNA sequence having at least 90% identity to a sequence selected from the group consisting of SEQ ID NOs: 497-551.

16. The pharmaceutical composition of claim 1, wherein:
the at least one guide RNA targets a canine IL-1β gene, and
the at least one guide RNA comprises a crRNA sequence having at least 95% identity to a sequence selected from the group consisting of SEQ ID NOs: 497-551.

17. The pharmaceutical composition of claim 1, wherein:
the at least one guide RNA targets a canine IL-1β gene, and
the at least one guide RNA comprises a crRNA sequence selected from the group consisting of SEQ ID NOs: 497-551.

18. The pharmaceutical composition of any one of claims 1-17, wherein the composition comprises one or more viral vectors collectively comprising the one or more nucleic acids.

19. The pharmaceutical composition of claim 18, wherein the one of more viral vectors comprise a recombinant virus selected from a retrovirus, an adenovirus, an adeno-associated virus, a lentivirus, and a herpes simplex virus-1

20. The pharmaceutical composition of claim 18, wherein the one of more viral vectors comprise a recombinant adeno-associated virus (AAV).

21. The pharmaceutical composition of claim 20, wherein the recombinant AAV is of serotype 5 (AAV5).

22. The pharmaceutical composition of claim 20, wherein the recombinant AAV is of serotype 6 (AAV6).

23. The pharmaceutical composition of any one of claims 18-22, wherein the one of more viral vectors comprise:
a first viral vector comprising a first nucleic acid, in the one or more nucleic acids, encoding the Cas9 protein; and
a second viral vector comprising a second nucleic acid, in the one or more nucleic acids, encoding the at least one guide RNA.

24. The pharmaceutical composition of any one of claims 18-22, wherein the one of more viral vectors comprise a viral vector comprising a single nucleic acid, wherein the single nucleic acid encodes the Cas9 protein and the at least one guide RNA.

25. The pharmaceutical composition of any one of claims 1-17, wherein the composition comprises one or more liposomes collectively comprising the one or more nucleic acids.

26. The pharmaceutical composition of any one of claims 1-17, wherein the one or more nucleic acids are present in a naked state.

27. The pharmaceutical composition of any one of claims 1-26, wherein the Cas9 protein is an S. pyogenes Cas9 polypeptide.

28. The pharmaceutical composition of any one of claims 1-26, wherein the Cas9 protein is an S. aureus Cas9 polypeptide.

29. The pharmaceutical composition of any one of claims 1-28, wherein the composition is formulated for parenteral administration.

30. The pharmaceutical composition of any one of claims 1-28, wherein the composition is formulated for intra-articular injection within a joint of a subject.

31. A method for the treatment or prevention of a joint disease or condition in a subject in need thereof, the method comprising:
administering, to a joint of the subject, a pharmaceutical composition comprising a pharmaceutically effective amount of a composition comprising one or more nucleic acids encoding a Clustered Regularly Interspaced Short Palindromic Repeats (CRISPR) gene-editing system, the system comprising:
(i) a CRISPR Associated Protein 9 (Cas9) protein; and
(ii) at least one guide RNA targeting an IL-1α or IL-1β gene, wherein the target sequence is adjacent to a protospacer adjacent motif (PAM) sequence for the Cas9 protein.

32. The method of claim 31, wherein the joint disease or condition is arthritis.

33. The method of claim 32, wherein the arthritis is osteoarthritis.

34. The method of any one of claims 31-33, wherein the subject is a human.

35. The method of claim 34, wherein:
the at least one guide RNA targets a human IL-1α gene, and
the at least one guide RNA comprises a crRNA sequence having at least 85% identity to a sequence selected from the group consisting of SEQ ID NOs: 298-387.

36. The method of claim 34, wherein:
the at least one guide RNA targets a human IL-1α gene, and
the at least one guide RNA comprises a crRNA sequence having at least 90% identity to a sequence selected from the group consisting of SEQ ID NOs: 298-387.

37. The method of claim 34, wherein:
the at least one guide RNA targets a human IL-1α gene, and
the at least one guide RNA comprises a crRNA sequence having at least 95% identity to a sequence selected from the group consisting of SEQ ID NOs: 298-387.

38. The method of claim 34, wherein:
the at least one guide RNA targets a human IL-1α gene, and
the at least one guide RNA comprises a crRNA sequence selected from the group consisting of SEQ ID NOs: 298-387.

39. The method of claim 34, wherein:
the at least one guide RNA targets a human IL-1β gene, and
the at least one guide RNA comprises a crRNA sequence having at least 85% identity to a sequence selected from the group consisting of SEQ ID NOs: 388-496.

40. The method of claim 34, wherein:
the at least one guide RNA targets a human IL-1β gene, and
the at least one guide RNA comprises a crRNA sequence having at least 90% identity to a sequence selected from the group consisting of SEQ ID NOs: 388-496.

41. The method of claim 34, wherein:
the at least one guide RNA targets a human IL-1β gene, and
the at least one guide RNA comprises a crRNA sequence having at least 95% identity to a sequence selected from the group consisting of SEQ ID NOs: 388-496.

42. The method of claim 34, wherein:
the at least one guide RNA targets a human IL-1β gene, and
the at least one guide RNA comprises a crRNA sequence selected from the group consisting of SEQ ID NOs: 388-496.

43. The method of any one of claims 31-33, wherein the subject is a canine.

44. The method of claim 43, wherein:
the at least one guide RNA targets a canine IL-1α gene, and
the at least one guide RNA comprises a crRNA sequence having at least 85% identity to a sequence selected from the group consisting of SEQ ID NOs: 522-590.

45. The method of claim 43, wherein:
the at least one guide RNA targets a canine IL-1α gene, and
the at least one guide RNA comprises a crRNA sequence having at least 90% identity to a sequence selected from the group consisting of SEQ ID NOs: 522-590.

46. The method of claim 43, wherein:
the at least one guide RNA targets a canine IL-1α gene, and
the at least one guide RNA comprises a crRNA sequence having at least 95% identity to a sequence selected from the group consisting of SEQ ID NOs: 522-590.

47. The method of claim 43, wherein:
the at least one guide RNA targets a canine IL-1α gene, and
the at least one guide RNA comprises a crRNA sequence selected from the group consisting of SEQ ID NOs: 522-590.

48. The method of claim 43, wherein:
the at least one guide RNA targets a canine IL-1β gene, and
the at least one guide RNA comprises a crRNA sequence having at least 85% identity to a sequence selected from the group consisting of SEQ ID NOs: 497-551.

49. The method of claim 43, wherein:
the at least one guide RNA targets a canine IL-1β gene, and
the at least one guide RNA comprises a crRNA sequence having at least 90% identity to a sequence selected from the group consisting of SEQ ID NOs: 497-551.

50. The method of claim 43, wherein:
the at least one guide RNA targets a canine IL-1β gene, and
the at least one guide RNA comprises a crRNA sequence having at least 95% identity to a sequence selected from the group consisting of SEQ ID NOs: 497-551.

51. The method of claim 43, wherein:
the at least one guide RNA targets a canine IL-1β gene, and
the at least one guide RNA comprises a crRNA sequence selected from the group consisting of SEQ ID NOs: 497-551.

52. The method of any one of claims 31-51, wherein the administering comprises intra-articular injection of the pharmaceutical composition into the joint of the subject.

53. The method of any one of claims 31-52, wherein the pharmaceutical composition is administered during surgery.

54. The method of any one of claims 31-52, wherein the pharmaceutical composition is administered after surgery.

55. The method of any one of claims 31-54, wherein the pharmaceutical composition is a controlled release pharmaceutical composition.

56. The method of any one of claims 31-55, wherein the pharmaceutical composition comprises one or more viral vectors collectively comprising the one or more nucleic acids.

57. The method of claim 56, wherein the one of more viral vectors comprise a recombinant virus selected from a retrovirus, an adenovirus, an adeno-associated virus, a lentivirus, and a herpes simplex virus-1

58. The method of claim 57, wherein the one of more viral vectors comprise a recombinant adeno-associated virus (AAV).

59. The method of claim 58, wherein the recombinant AAV is of serotype 5 (AAV5).

60. The method of claim 58, wherein the recombinant AAV is of serotype 6 (AAV6).

61. The method of any one of claims 56-60, wherein the one of more viral vectors comprise:
a first viral vector comprising a first nucleic acid, in the one or more nucleic acids, encoding the Cas9 protein; and
a second viral vector comprising a second nucleic acid, in the one or more nucleic acids, encoding the at least one guide RNA.

62. The method of any one of claims 56-60, wherein the one of more viral vectors comprise a viral vector comprising a single nucleic acid, wherein the single nucleic acid encodes the Cas9 protein and the at least one guide RNA.

63. The method of any one of claims 31-55, wherein the pharmaceutical composition comprises one or more liposomes collectively comprising the one or more nucleic acids.

64. The method of any one of claims 31-55, wherein the one or more nucleic acids are present in a naked state.

65. The method of any one of claims 31-64, wherein the Cas9 protein is an S. pyogenes Cas9 polypeptide.

66. The method of any one of claims 31-64, wherein the Cas9 protein is an S. aureus Cas9 polypeptide.

67. A pharmaceutical composition for the treatment or prevention of a joint disease or condition comprising a gene-editing system, wherein said gene-editing system targets at least one locus related to joint function.

68. The pharmaceutical composition of claim 67, wherein the gene-editing system targets one or more of IL-1α, and/or IL-1β.

69. The pharmaceutical composition of claim 67 or 68, wherein the gene-editing system causes expression of the at least one locus related to joint function to be silenced or reduced in at least a portion of the cells comprising the joint.

70. The pharmaceutical composition of claim 67, wherein the at least one locus related to joint function is a cytokine and/or growth factor locus.

71. The pharmaceutical composition of claim 70, wherein the cytokine and/or growth factor locus is selected from the group consisting of IL-1α, IL-1β, TNF-α, IL-6, IL-8, IL-18, a matrix metalloproteinase (MMP), NLRP3, ASC (apoptosis-associated speck-like protein containing a CARD), caspase-1, and combinations thereof.

72. The pharmaceutical composition according to claim 67, wherein the gene-editing comprises the use of a programmable nuclease that mediates the generation of a double-strand or single-strand break at the at least one locus related to joint function.

73. The pharmaceutical composition according to any of claims 67 to 72, wherein the gene-editing causes expression of the at least one locus related to joint to be silenced or reduced in at least a portion of the cells comprising the joint.

74. The pharmaceutical composition according to claim 73, wherein said one or more cytokine and/or growth factor genes is/are selected from the group consisting of IL-1α, IL-1β, TNF-α, IL-6, IL-8, IL-18, a matrix metalloproteinase (MMP), NLRP3, ASC (apoptosis-associated speck-like protein containing a CARD), caspase-1, and combinations thereof.

75. The pharmaceutical composition according to any of claims 67 to 74, wherein the gene-editing causes expression of one or more cytokine and/or growth factor genes to be enhanced in at least a portion of the cells comprising the joint, the cytokine and/or growth factor gene(s) being selected from the group consisting of IL-1Ra, TIMP-1, TIMP-2, TIMP-3, TIMP-4, and combinations thereof.

76. The pharmaceutical composition according to any of claims 67 to 75, wherein the gene-editing comprises the use of a programmable nuclease that mediates the generation of a double-strand or single-strand break at said one or more cytokine and/or growth factor genes.

77. The pharmaceutical composition according to any of claims 67 to 76, wherein the gene-editing comprises one or more methods selected from a CRISPR method, a TALE method, a zinc finger method, and a combination thereof.

78. The pharmaceutical composition according to any of claims 67 to 76, wherein the gene-editing comprises a CRISPR method.

79. The pharmaceutical composition according to claim 78, wherein the CRISPR method is a CRISPR-Cas9 method.

80. The pharmaceutical composition according to any of claims 67 to 76, wherein the gene-editing comprises a TALE method.

81. The pharmaceutical composition according to any of claims 67 to 76, wherein the gene-editing comprises a zinc finger method.

82. A method for the treatment or prevention of a joint disease or condition the method comprising introducing a gene-editing system, wherein the gene-editing system targets at least one locus related to joint function.

83. The method of claim 82, wherein the gene-editing system targets one or more of IL-1α, and/or IL-1β.

84. The method of claim 82 or 83, wherein the gene-editing system causes expression of the at least one locus related to joint function to be silenced or reduced in at least a portion of the cells comprising the joint.

85. The method of claim 82, wherein the at least one locus related to joint function is a cytokine and/or growth factor locus.

86. The method of claim 85, wherein the cytokine and/or growth factor locus is selected from the group consisting of IL-1α, IL-1β, TNF-α, IL-6, IL-8, IL-18, a matrix metalloproteinase (MMP), NLRP3, ASC (apoptosis-associated speck-like protein containing a CARD), caspase-1, and combinations thereof.

87. The method according to any one of claims 82 to 86, wherein the gene-editing comprises the use of a programmable nuclease that mediates the generation of a double-strand or single-strand break at the at least one locus related to joint function.

88. The method according to any of claims 82 to 87, wherein the gene-editing causes expression of the at least one locus related to joint to be silenced or reduced in at least a portion of the cells comprising the joint.

89. The method according to claim 88, wherein said one or more cytokine and/or growth factor genes is/are selected from the group consisting of IL-1α, IL-1β, TNF-α, IL-6, IL-8, IL-18, a matrix metalloproteinase (MMP), NLRP3, ASC (apoptosis-associated speck-like protein containing a CARD), caspase-1, and combinations thereof.

90. The method according to any of claim 82 or 83, wherein the gene-editing causes expression of one or more cytokine and/or growth factor genes to be enhanced in at least a portion of the cells comprising the joint, the one or more cytokine and/or growth factor gene(s) being selected from the group consisting of IL-1Ra, TIMP-1, TIMP-2, TIMP-3, TIMP-4, and combinations thereof.

91. The method according to any of claims 82 to 86, wherein the gene-editing comprises the use of a programmable nuclease that mediates the generation of a double-strand or single-strand break at said one or more cytokine and/or growth factor genes.

92. The method according to any of claims 82 to 91, wherein the gene-editing comprises one or more methods selected from a CRISPR method, a TALE method, a zinc finger method, and a combination thereof.

93. The method according to any of claims 82 to 92, wherein the gene-editing comprises a CRISPR method.

94. The method according to claim 93, wherein the CRISPR method is a CRISPR-Cas9 method.

95. The method according to any of claims 82 to 91, wherein the gene-editing comprises a TALE method.

96. The method according to any of claims 82 to 91, wherein the gene-editing comprises a zinc finger method.

97. A method for treating canine lameness due to joint disease, the method comprising administering a composition of any one of claims 67 to 81 to a canine in need thereof.

98. The method of claim 97, wherein the composition is injected into a joint.

99. A method for treating equine lameness due to joint disease, the method comprising administering a composition of any one of claims 67 to 81 to an equine in need thereof.

100. The method of claim 99, wherein the composition is injected into a joint.

101. The method of any one of claims 97 to 100, wherein the joint disease is an inflammatory joint disease.

102. A method of treating a subject having arthritis, the method comprising administering to the subject a therapeutically effective amount of a Clustered Regularly Interspaced Short Palindromic Repeats (CRISPR) gene editing complex comprising a CRISPR Associated Protein 9 (Cas9) and at least one guide RNA targeting the gene,
wherein the gene is selected from the group consisting of IL-1α, IL-1β, and a combination thereof, and wherein the at least one guide RNA targeting the gene is an RNA sequence complementary to a DNA sequence selected from the group consisting of SEQ ID NO: 7-SEQ ID NO: 20.

103. The method of claim 102, wherein the arthritis is osteoarthritis.

104. The method of claim 102, wherein the CRISPR gene editing complex comprises a Cas9 protein and a single guide RNA.

105. The method of claim 104, wherein the CRISPR gene editing complex comprises Cas9 protein in a complex with the single guide RNA.

106. The method of claim 102, wherein the CRISPR gene editing complex comprises a Cas9 protein and a nucleic acid encoding at least one guide RNA targeting one or both of IL-1a and IL-1β.

107. The method of claim 102, wherein the Cas9 is administered as a nucleic acid comprising a sequence encoding a Cas9 protein.

108. The method of claim 107, wherein the nucleic acid comprising a sequence encoding the Cas9 protein is administered in a virus.

109. The method of claim 108, wherein the virus is selected from the group consisting of recombinant retroviruses, adenovirus, adeno-associated virus (AAV), and lentivirus.

110. The method of claim 109, wherein the virus is an adeno-associated virus (AAV).

111. The method of claim 108, comprising administering a nucleic acid comprising a sequence encoding at least one guide RNA targeting one or both of IL-1α and IL-1β.

112. The method of claim 111, wherein the nucleic acid comprising a sequence encoding the guide RNA is administered in a virus.

113. The method of claim 112, wherein the virus is selected from the group consisting of recombinant retroviruses, adenovirus, adeno-associated virus (AAV), and lentivirus.

114. The method of claim 113, wherein the virus is an adeno-associated virus (AAV).

115. The method of claim 102, wherein the complex is administered as a single nucleic acid, preferably a viral vector, comprising a sequence encoding the Cas9 protein and a sequence encoding the at least one guide RNA, and the Cas9 protein and the at least one guide RNA are expressed from the same nucleic acid.

116. The method of claim 102, wherein the complex is administered in more than one nucleic acid, preferably in more than one viral vector, wherein a first nucleic acid comprises a sequence encodes the at least one guide RNA and a second nucleic acid comprises a sequence encoding the Cas9 protein, and the at least one guide RNA and the Cas9 protein are expressed from separate nucleic acids.

117. The method of claim 115 or 116, wherein the nucleic acid is viral vector selected from the group consisting of a recombinant retrovirus vector, an adenovirus vector, an adeno-associated virus (AAV) vector, and a lentivirus vector.

118. The method of claim 117, wherein the viral vector is an adeno-associated virus (AAV) vector.

119. The method of claim 102, comprising administering a guide RNA targeting IL-1α.

120. The method of claim 102, comprising administering a guide RNA targeting IL-1β.

121. The method of claim 102, wherein the Cas9 is Streptococcus thermophilus (ST) Cas9 (StCas9); Treponema denticola (TD) (TdCas9); Streptococcus pyogenes (SP) (SpCas9); Staphylococcus aureus (SA) Cas9 (SaCas9); or Neisseria meningitidis (NM) Cas9 (NmCas9), or a variant thereof.

122. The method of claim 121, wherein the Cas9 is SpCas9, or a variant thereof.

123. The method of claim 122, wherein the SpCas9 is SpyCas9, or a variant thereof.

124. The method of claim 102, wherein the CRISPR gene editing complex is administered systemically or locally to a site of arthritis.

125. The method of claim 102, wherein the CRISPR gene editing complex is administered locally to the site of a treatment selected from the group consisting of a surgery, an application of topical ointment, and a combination thereof.

126. The method of claim 102, wherein the CRISPR gene editing complex is formulated to be administered in a composition comprising a biodegradable, and/or a biocompatible polymer.

127. The method of claim 126, wherein the biodegradable, and/or biocompatible polymer is selected from the group consisting of collagen, ethylene vinyl acetate, polyanhydrides, polyglycolic acid, collagen, polyorthoesters, polyethyleneglycol-coated liposomes, and polylactic acid.

128. The method of claim 102, wherein the subject is a human.

129. The method of claim 102, wherein the subject is a non-human subject selected from the group consisting of an ape, a baboon, a cow, a dog, a goat, a gorilla, a guinea pig, a hamster, a lemur, a mouse, an orangutan, a pig, a rat, a horse, and a sheep.

130. The method of 102, wherein the editing efficiency of the CRISPR gene editing complex is at least 50%, at least 60%, at least 70%, at least 80%, at least 90%, at least 95%, or at least 99%.

131. A pharmaceutical composition, comprising:
a therapeutically effective amount of a Clustered Regularly Interspaced Short Palindromic Repeats (CRISPR) gene editing complex comprising a CRISPR Associated Protein 9 (Cas9) and at least one guide RNA targeting the gene,
wherein the gene is selected from the group consisting of IL-1α, IL-1β, and a combination thereof, and wherein the at least one guide RNA targeting the gene is an RNA sequence complementary to a DNA sequence selected from the group consisting of SEQ ID NO: 7 to SEQ ID NO: 20.

132. A CRISPR/Cas9-mediated method for treating joint disease through genetic modification of a multicellular eukaryotic organism comprising local administration of a nucleic acid target sequence adjacent to a Protospacer Adjacent Motif (PAM) and a non naturally-occurring Cas9 protein comprising:
a) a first regulatory element operably linked to one or more nucleotide sequences encoding one or more CRISPR/Cas9 complex gRNAs that hybridize with the target sequence(s);
b) a second regulatory element operably linked to a nucleotide sequence encoding a Type II Cas protein, and
c) a viral vector capable of delivery of components (a) and (b) to a target joint cell, whereby co-expression of the nucleic acid components results in reduced inflammation in at least some cells of the joint.

133. A method of treating joint disease comprising local administration of:
a) a viral vector comprising a nucleotide sequence encoding a gRNA molecule,
wherein the gRNA molecule comprises a domain targeting an IL-1α gene or an IL-1β; and
b) a viral vector comprising a nucleotide sequence encoding a Cas9 molecule;
wherein said viral vector comprising a nucleotide sequence encoding a gRNA molecule and said viral vector comprising a Cas9 molecule are capable of delivery to a cell such that levels of IL-1α or IL-1β in at least some cells of the joint are reduced.

134. A CRISPR-Cas nuclease comprising a single guide RNA having a sequence selected from the group consisting of SEQ ID NO. 21 to 34 that binds to a target site of an IL-1α or IL-1β gene, wherein the nuclease cleaves and inactivates the gene.

135. A method of inactivating an endogenous IL-1α or IL-1β in the joint of a subject, the method comprising the steps of: administering to the joint a CRISPR/Cas nuclease according to claim 134, wherein the nuclease cleaves and inactivates the IL-1α gene or the IL-1β gene.

136. A clustered regularly interspaced palindromic repeats (CRISPR)/Cas guide RNA (gRNA) comprising a targeting domain that is complementary to genomic Interleukin-1 alpha (IL-1α), wherein the targeting domain is configured to destroy a wild type sequence.

137. A vector system comprising one or more packaged vector(s) comprising:
a) A first regulatory element operably linked to a sequence encoding a gRNA according to claim 136, and
b) a second regulatory element operably linked to a nucleic acid encoding a Cas protein

138. A method of altering a nucleic acid sequence encoding IL-1A in a cell comprising contacting said cell with
a) a viral vector comprising a nucleotide sequence encoding a gRNA molecule, wherein the gRNA molecule comprises a domain targeting an IL-1α gene or an IL-1β; and
b) a viral vector comprising a nucleotide sequence encoding a Cas9 molecule;
wherein said viral vector comprising a nucleotide sequence encoding a gRNA molecule and said viral vector comprising a Cas9 molecule are capable of delivery to a cell such that levels of IL-1α or IL-1β in at least some cells of the joint are reduced.

139. A method of treating osteoarthritis in a subject comprising locally administering to the subject a IL-1α gene or an IL-1β vector system of claim 137.

140. A method for reducing IL-1α gene or IL-1β expression in at least some cells of the joint comprising introducing or expressing in the cell the vector system of claim 137.

141. The composition, method, or system of any prior claim, wherein the the guide RNA is at least 50%, at least 55%, at least 60%, at least 65%, at least 70%, at least 75%, at least 80%, at least 85%, at least 90%, or at least 95% identical to a sequence as shown in SEQ ID NOs.: 21-34 and 168-297.

142. The composition, method, or system of any prior claim, wherein the AAV is selected from the group consisting of AAV-5 and AAV-6.
